(19) United States
(12) Patent Application Publication
(10) Pub. No.: US 2024/0390371 A1
(43) Pub. Date: Nov. 28, 2024

(54) HETEROARYL AND HETEROCYCLIC COMPOUNDS FOR TREATING ACUTE INFLAMMATION

(71) Applicant: TES Pharma S.r.l., Corciano (IT)

(72) Inventors: Roberto PELLICCIARI, Perugia (IT); Nicola GIACCHÈ, Perugia (IT); Francesca DE FRANCO, Magione (IT); Paride LISCIO, Perugia (IT)

(21) Appl. No.: 18/673,091

(22) Filed: May 23, 2024

Related U.S. Application Data
(60) Provisional application No. 63/468,751, filed on May 24, 2023.

(51) Int. Cl. A61K 31/506; A61K 31/4412; A61K 31/4418; A61K 31/4436; A61K 31/4439; A61K 31/513; A61K 31/5377; A61P 29/00

(52) U.S. Cl. CPC A61K 31/506; A61K 31/4412; A61K 31/4418; A61K 31/4436; A61K 31/4439; A61K 31/513; A61K 31/5377; A61P 29/00

(57) ABSTRACT

The present disclosure relates to a method of treating an acute inflammatory condition in a subject comprising administering to the subject a therapeutically effective amount of a heteroaryl or heterocyclic compound.

### CROSS REFERENCE TO RELATED APPLICATIONS

This application claims the benefit of U.S. Provisional Application No. 63/468,751, filed May 24, 2023, the contents of which is incorporated herein by reference in its entirety.

### FIELD OF THE DISCLOSURE

The present disclosure relates to a method of treating an acute inflammatory condition in a subject comprising administering to the subject a therapeutically effective amount of a heteroaryl or heterocyclic compound.

### BACKGROUND OF THE DISCLOSURE

Inflammation is a complex of sequential biological responses of body tissues to harmful stimuli, such as pathogens, damaged cells, or irritants. When tissue injury occurs, whether it is caused by bacteria, trauma, chemicals, heat, or any other phenomenon, histamine, along with other humoral substances, is liberated by the damaged tissue into the surrounding fluids and initiates the vascular phase of acute inflammation. This is a protective adaptation by the organism to remove the injurious stimuli as well as initiating the healing process.

There are two forms of inflammation, commonly referred to as acute inflammation and chronic inflammation. Acute inflammation is the initial response of the body to harmful stimuli and is achieved by the increased movement of plasma and leukocytes from the blood into the injured tissues. Acute inflammation can be divided into several phases. The earliest, event of an inflammatory response is temporary vasoconstriction, i.e., narrowing of blood vessels caused by contraction of smooth muscle in the vessel walls which can be seen as blanching (whitening) of the skin. This is followed by several phases that occur minutes, hours, and days later. The first is the acute vascular response which follows within seconds of the tissue injury and lasts for several minutes. This results from vasodilation and increased capillary permeability due to alterations in the vascular endothelium which leads to increased blood flow (hyperemia) that causes redness (erythema) and the entry of fluid into the tissues (edema).

The main features of the vascular phase of the inflammatory response are vasodilation, i.e. widening of the blood vessels to increase the blood flow to the infected area; increased vascular permeability which allows diffusible components to enter the site; cellular infiltration by chemotaxis; or the directed movement of inflammatory cells, including neutrophils, through the walls of blood vessels into the site of injury; changes in biosynthetic, metabolic, and catabolic profiles of many organs; and activation of cells of the immune system as well as of complex enzymatic systems of blood plasma. This leads to the cellular phase where neutrophils are attracted to the site of injury by the presence of chemotaxins neutrophils then recognise the foreign body and begin phagocytosis. Inflammation which runs unchecked can, however lead to a host of diseases including acute heptatitis, acute pancreatitis, acute kidney disease, inflammatory bowel disease, inflammatory liver diseases, rheumatoid arthritis, autoimmunity, sepsis, SIRS, and atherosclerosis.

The acute vascular response can be followed by an acute cellular response which takes place over the next few hours. The hallmark of this phase is the appearance of granulocytes, particularly neutrophils, in the tissues. These cells first attach themselves to the endothelial cells within the blood vessels (margination) and then cross into the surrounding tissue (diapedesis). During this phase erythrocytes may also leak into the tissues and a hemorrhage can occur. If the vessel is damaged, fibrinogen and fibronectin are deposited at the site of injury, platelets aggregate and become activated, and the red cells stack together in what are called “rouleau” to help stop bleeding and aid clot formation. The dead and dying cells contribute to pus formation. If the damage is sufficiently severe, a chronic cellular response may follow over the next few days. A characteristic of this phase of inflammation is the appearance of a mononuclear cell infiltrate composed of macrophages and lymphocytes. The macrophages are involved in microbial killing, in clearing up cellular and tissue debris, and in remodeling of tissues.

### SUMMARY OF THE DISCLOSURE

The present disclosure relates to a method of treating an acute inflammatory condition in a subject comprising administering to the subject a therapeutically effective amount of a compound represented by Formula (I):

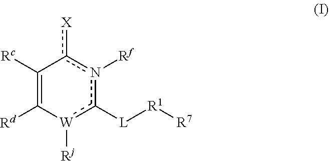

and pharmaceutically acceptable salts and tautomers thereof, wherein:
X is H, S, SR², NR², NR²R^{2′}, O, OH, OR^{h}, F, Br, or Cl;
W is N or C;
   (i) when W is N, then: L is —(C(R⁵)₂)ₘCH═CH(C(R⁵)₂)ₚ—,

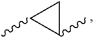

—(C(R⁵)₂)ₘY¹(C(R⁵)₂)ₚ—, —(C(R⁵)₂)ₘY¹(C(R⁵)₂)ₚ-cyclopropyl-, —(C(R⁵)₂)ₘY¹CH═CH—, —(C(R⁵)₂)ₘNR³C═(O)(C(R⁵)₂)ₚ—, —(C(R⁵)₂)ₘphenyl(C(R⁵)₂)ₚ—, —(C(R⁵)₂)ₘpyridinyl(C(R⁵)₂)ₚ—, or —(C(R⁵)₂)ₘthiophenyl(C(R⁵)₂)ₚ—;
(ii) when W is C, then: L is —(C(R⁵)₂)ₘCH═CH(C(R⁵)₂)ₚ—, —(C(R⁵)₂)ₒ—, —(C(R⁵)₂)ₘY¹(C(R⁵)₂)ₚ—,

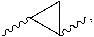

(C(R⁵)₂)ₘ Y¹CH═CH—, —(C(R⁵)₂)ₘC═(O)(CH₂)ₚ—, —(C(R⁵)₂)ₘC═(O)O(C(R⁵)₂)ₚ—, —(C(R⁵)₂)ₘC═(O)NR³(C(R⁵)₂)ₚ—, —(C(R⁵)₂)ₘNR³C═(O)(C(R⁵)₂)ₚ—, —(C(R⁵)₂)ₘphenyl(C(R⁵)₂)ₚ—, —(C(R⁵)₂)ₘpyridinyl(C(R⁵)₂)ₚ—, or —(C(R⁵)₂)ₘthiophenyl(C(R⁵)₂)ₚ—;
Y¹ is O, NR⁴, or S(O)_{q};
each Y² is independently O, NH or S;
R¹ is absent or C₆-C₁₀ arylene or heteroarylene, wherein the heteroarylene comprises one or two 5- to 7-membered rings and 1-4 heteroatoms selected from N, O and S, and wherein the C₆-C₁₀ arylene or heteroarylene are optionally substituted with one to two R^{e};
R² is H or C₁-C₄ alkyl;
R^{2′} is H, C₁-C₄ alkyl, or C₃-C₇ cycloalkyl; or
R² and R^{2′} together with the nitrogen atom to which they are attached form a 3- to 7-membered heterocycloalkyl ring comprising 1-3 additional heteroatoms selected from N, O and S;
R³ is H or C₁-C₄ alkyl;
R⁴ is H or C₁-C₄ alkyl;
each R⁵ is independently at each occurrence H or C₁-C₄ alkyl;
each R⁶ is independently at each occurrence H or C₁-C₄ alkyl;
R⁷ is H, A, B, or C;
A is —(C(R⁶)₂)ᵣCO₂R^{x}, —Y²(C(R⁶)₂)ᵣCO₂R^{x}, —(CH₂)ᵣtetrazole, —(CH₂)ᵣoxadiazolone, —(CH₂)ᵣtetrazolone, —(CH₂)ᵣthiadiazolol, —(CH₂)ᵣ isoxazol-3-ol, —(CH₂)ᵣP(O)(OH)OR^{x}, —(CH₂)ᵣS(O)₂OH, —(CH₂)ᵣC(O)NHCN, or —(CH₂)ᵣC(O)NHS(O)₂alkyl, wherein —(CH₂)ᵣtetrazole, —(CH₂)ᵣoxadiazolone, —(CH₂)ᵣtetrazolone, —(CH₂)ᵣthiadiazolol, —(CH₂)ᵣ isoxazol-3-ol are optionally substituted with C₁-C₆ alkyl,
B is —(C(R⁶)₂)ᵣS(O)₂OC₁-C₄ alkyl, —O(C(R⁶)₂)ᵣS(O)₂OC₁-C₄ alkyl, —Y²(C(R⁶)₂)ᵣC(O)NR^{g}R^{g′}, —Y²(C(R⁶)₂)ᵣS(O)₂NR^{g}R^{g′}, —(CH₂)ᵣC(O)NR^{g}R^{g′}, —(CH₂)ᵣS(O)₂NR^{g}R^{g′}, —(CH₂)ᵣC(O)NHS(O)₂NR^{g}R^{g′}, —(C(R⁶)₂)ᵣCO₂Rⁱ, —(C(R⁶)₂)ᵣNH₂CO₂R^{x}, —(C(R⁶)₂)ᵣP(O)(OR^{x})₂, —O(C(R⁶)₂)ᵣP(O)(OR^{x})₂, —(C(R⁶)₂)ᵣS(O)₂OH, —O(C(R⁶)₂)ᵣS(O)₂OH, —(C(R⁶)₂)ᵣP(O)₂OR^{x}, or —O(C(R⁶)₂)ᵣP(O)₂OR^{x},
C is —(CH₂)ᵣCN, —(CH₂)ₛOH, halogen, —(C(R⁶)₂)ᵣC₆-C₁₀ aryl, —(C(R⁶)₂)ᵣS—C₆-C₁₀ aryl, —(C(R⁶)₂)ᵣheteroaryl, —O(C(R⁶)₂)ᵣheteroaryl, —O(C(R⁶)₂)ᵣheterocycloalkyl, —O(C(R⁶)₂)ᵣOH, —OR^{y}, —(C(R⁶)₂)ᵣC(O)NHCN, —CH═CHCO₂R^{x}, or —(C(R⁶)₂)ᵣC(O)NHS(O)₂C₁-C₄ alkyl, wherein the aryl and heteroaryl are substituted with one to three substituents each independently selected from C₁-C₆ alkyl, C₁-C₆ haloalkyl, halogen, and OH, and wherein the heterocycloalkyl is substituted with one to two ═O or ═S;
R^{c} is H, C₁-C₆ alkyl, C₁-C₆ haloalkyl, halogen, —CN, —OR^{x}, or —CO₂R^{x};
R^{d} is methyl, CF₃, CR^{f}F₂, —(C(R⁶)₂)ₜC₆-C₁₀ aryl, —(C(R⁶)₂)ₜ-5- or 6-membered heteroaryl, —(C(R⁶)₂)ₜ-5- or 6-membered cycloalkyl, optionally substituted C₆-C₁₀ aryl, optionally substituted 5- or 6-membered heteroaryl, or optionally substituted 5- or 6-membered cycloalkyl;
each R^{e} is independently at each occurrence C₁-C₆ alkyl, C₂-C₆ alkenyl, C₂-C₆ alkynyl, halogen, C₁-C₆ haloalkyl, —NHR^{z}, —OH, or —CN;
R^{f} is absent, H, or methyl;
R^{g} is H, C₁-C₆ alkyl, OH, —S(O)₂(C₁-C₆ alkyl), or S(O)₂N(C₁-C₆ alkyl)₂;
R^{g′} is H, C₁-C₆ alkyl, C₃-C₇ cycloalkyl, 4- to 7-membered heterocycloalkyl ring comprising 1-3 heteroatoms selected from N, O and S, C₆-C₁₀ aryl, or 5- to 7-membered heteroaryl comprising 1-3 heteroatoms selected from N, O and S, wherein the alkyl is optionally substituted with one or more substituents independently selected from halogen and —OH, and wherein the cycloalkyl, heterocycloalkyl, aryl and heteroaryl are optionally substituted with one or more substituents independently selected from C₁-C₆ alkyl, halogen, and —OH;
R^{h} is H, C₁-C₄ alkyl, or 3- to 7-membered heterocycloalkyl ring comprising 1-3 heteroatoms selected from N, O and S, wherein the alkyl is optionally substituted with one or more substituents each independently selected from NH₂, C₁-C₄ alkylamino, C₁-C₄ dialkylamino, and C(O)NH₂; and wherein the heterocycloalkyl is optionally substituted with one or more substituents each independently selected from C₁-C₆ alkyl and C₁-C₆ haloalkyl;
Rⁱ is (i) —(CH₂)ₛOC(O)C₁-C₆ alkyl, wherein the alkyl is substituted with one or more NH₂; (ii) (CH₂CH₂O)ₙCH₂CH₂OH; or (iii) C₁-C₆ alkyl substituted with one or more substituents each independently selected from OH and 4- to 7-membered heterocycloalkyl comprising 1 to 3 heteroatoms selected from O, N, or S;
R^{j} is absent, H, C₁-C₆ alkyl, or —CN;
each R^{x} is independently at each occurrence H, C₁-C₆ alkyl, or C₆-C₁₀ aryl;
each R^{y} and R^{z} is independently H, C₁-C₆ alkyl, or C₁-C₆ haloalkyl;
each m, p, q, r, and t is independently 0, 1 or 2;
n is 0, 1, 2, or 3;
s is 1 or 2;
o is 0, 1, 2, 3, or 4; and
[symbol] represents a single bond or a double bond; and
provided that
when X is O; R^{f} is H; W is C; R^{j} is —CN; L is —SCH₂—; R¹ is phenylene or pyridine; then R⁷ is not —COOH;
when X is O; R^{f} is H; W is C; R^{j} is —CN; L is —SCH₂—; R¹ is phenylene or pyridine; and R⁷ is tetrazole; then R^{c} is not H;
when X is O; R^{f} is H; W is C; R^{j} is —CN; L is —S—C(R⁵)₂ or —SCH₂CH₂—; R¹ is absent; then R⁷ is not COOH or tetrazole;
when X is O, R^{f} is H; W is N; R^{j} is absent; R^{d} is methyl, optionally substituted 5- to 10-membered aryl, optionally substituted 5- or 6-membered heteroaryl, or optionally substituted 5- or 6-membered cycloalkyl; L is —SCH₂— or —OCH₂—; and R¹ is phenylene; then R⁷ is not —COOH, —CH₂COOH,

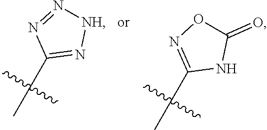 [SMILES: *c1nn[nH]n1 ; *c1noc(=O)[nH]1]

and
when X is O, R^{f} is H, W is N, R^{j} is absent, L is —NHCH₂—, —CH₂NH—, or —NH—C(O)—, and R¹ is phenylene, then R^{d} is not phenyl.

The present disclosure relates to a method of treating an acute inflammatory condition in a subject comprising administering to the subject a therapeutically effective amount of a compound represented by Formula (II):

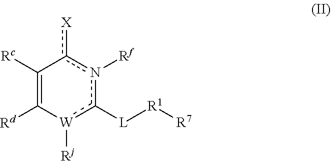

and pharmaceutically acceptable salts and tautomers thereof, wherein:
X is H, S, SR², NR², NR²R^{2′}, OH, OR^{h}, F, Br, or Cl;
W is N or C;
   (i) when W is N, then: L is —(C(R⁵)₂)ₘCH═CH(C(R⁵)₂)ₚ—,

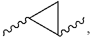

—(C(R⁵)₂)ₘY¹(C(R⁵)₂)ₚ—, —(C(R⁵)₂)ₘY¹(C(R⁵)₂)ₚ-cyclopropyl-, —(C(R⁵)₂)ₘY¹CH═CH—, —(C(R⁵)₂)ₘNR³C═(O)(C(R⁵)₂)ₚ—, —(C(R⁵)₂)ₘphenyl(C(R⁵)₂)ₚ—, —(C(R⁵)₂)ₘpyridinyl(C(R⁵)₂)ₚ—, or —(C(R⁵)₂)ₘthiophenyl(C(R⁵)₂)ₚ—;
(ii) when W is C, then: L is —(C(R⁵)₂)ₘCH═CH(C(R⁵)₂)ₚ—, —(C(R⁵)₂)ₒ—, —(C(R⁵)₂)ₘY¹(C(R⁵)₂)ₚ,

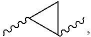

—(C(R⁵)₂)ₘ Y¹CH═CH—, —(C(R⁵)₂)ₘC═(O)(CH₂)ₚ—, —(C(R⁵)₂)ₘC═(O)O(C(R⁵)₂)ₚ—, —(C(R⁵)₂)ₘC═(O)NR³(C(R⁵)₂)ₚ—, —(C(R⁵)₂)ₘNR³C═(O)(C(R⁵)₂)ₚ—, —(C(R⁵)₂)ₘphenyl(C(R⁵)₂)ₚ—, —(C(R⁵)₂)ₘpyridinyl(C(R⁵)₂)ₚ—, or —(C(R⁵)₂)ₘthiophenyl(C(R⁵)₂)ₚ—;
Y¹ is O, NR⁴, or S(O)_{q};
each Y² is independently O, NH or S;
R¹ is absent, C₆-C₁₀ arylene, heteroarylene, or C₃-C₈cycloalkylene, wherein the heteroarylene comprises one or two 5- to 7-membered rings and 1-4 heteroatoms selected from N, O and S, and wherein the C₆-C₁₀ arylene, heteroarylene, and C₃-C₈cycloalkylene are optionally substituted with one to two R^{e};
R² is H or C₁-C₄ alkyl;
R^{2′} is H, C₁-C₄ alkyl, or C₃-C₇ cycloalkyl; or
R² and R^{2′} together with the nitrogen atom to which they are attached form a 3- to 7-membered heterocycloalkyl ring comprising 1-3 additional heteroatoms selected from N, O and S;
R³ is H or C₁-C₄ alkyl;
R⁴ is H or C₁-C₄ alkyl;
each R⁵ is independently at each occurrence H or C₁-C₄ alkyl;
each R⁶ is independently at each occurrence H or C₁-C₄ alkyl;
R⁷ is H, A, B, or C;
A is —(C(R⁶)₂)ᵣCO₂R^{x}, —Y²(C(R⁶)₂)ᵣCO₂R^{x}, —(C(R⁶)₂)ᵣtetrazole, —(C(R⁶)₂)ᵣoxadiazolone, —(C(R⁶)₂)ᵣtetrazolone, —(C(R⁶)₂)ᵣthiadiazolol, —(C(R⁶)₂)ᵣ isoxazol-3-ol, —(C(R⁶)₂)ᵣP(O)(OH)OR^{x}, —(C(R⁶)₂)ᵣS(O)₂OH, —(C(R⁶)₂)ᵣC(O)NHCN, or —(C(R⁶)₂)ᵣC(O)NHS(O)₂alkyl, wherein —(C(R⁶)₂)ᵣtetrazole, —(C(R⁶)₂)ᵣoxadiazolone, —(C(R⁶)₂)ᵣtetrazolone, —(C(R⁶)₂)ᵣthiadiazolol, —(C(R⁶)₂)ᵣ isoxazol-3-ol are optionally substituted with C₁-C₆ alkyl,
B is —(C(R⁶)₂)ᵣS(O)₂OC₁-C₄ alkyl, —O(C(R⁶)₂)ᵣS(O)₂OC₁-C₄ alkyl, —Y²(C(R⁶)₂)ᵣC(O)NR^{g}R^{g′}, —Y²(C(R⁶)₂)ᵣS(O)₂NR^{g}R^{g′}, —(C(R⁶)₂)ᵣC(O)NR^{g}R^{g′}, —(C(R⁶)₂)ᵣS(O)₂NR^{g}R^{g′}, —(C(R⁶)₂)ᵣC(O)NHS(O)₂NR^{g}R^{g′}, —(C(R⁶)₂)ᵣCO₂Rⁱ, —(C(R⁶)₂)ᵣNH₂CO₂R^{x}, —(C(R⁶)₂)ᵣP(O)(OR^{x})₂, —O(C(R⁶)₂)ᵣP(O)(OR^{x})₂, —(C(R⁶)₂)ᵣS(O)₂OH, —O(C(R⁶)₂)ᵣS(O)₂OH, —(C(R⁶)₂)ᵣP(O)₂OR^{x}, or —O(C(R⁶)₂)ᵣP(O)₂OR^{x},
C is —(CH₂)ᵣCN, —(CH₂)ₛOH, halogen, —(C(R⁶)₂)ᵣC₆-C₁₀ aryl, —(C(R⁶)₂)ᵣS—C₆-C₁₀ aryl, —(C(R⁶)₂)ᵣheteroaryl, —O(C(R⁶)₂)ᵣheteroaryl, —O(C(R⁶)₂)ᵣheterocycloalkyl, —O(C(R⁶)₂)ᵣOH, —OR^{y}, —(C(R⁶)₂)ᵣC(O)NHCN, —CH═CHCO₂R^{x}, or —(C(R⁶)₂)ᵣC(O)NHS(O)₂C₁-C₄ alkyl, wherein the aryl and heteroaryl are substituted with one to three substituents each independently selected from C₁-C₆ alkyl, C₁-C₆ haloalkyl, halogen, and OH, and wherein the heterocycloalkyl is substituted with one to two ═O or ═S;
R^{c} is H, C₁-C₆ alkyl, C₁-C₆ haloalkyl, halogen, —CN, —OR^{x}, or —CO₂R^{x};
R^{d} is methyl, CF₃, CR^{f}F₂, —(C(R⁶)₂)ₜC₆-C₁₀ aryl, —(C(R⁶)₂)ₜ-5- or 6-membered heteroaryl, —(C(R⁶)₂)ₜ-5- or 6-membered cycloalkyl, optionally substituted C₆-C₁₀ aryl, optionally substituted 5- or 6-membered heteroaryl, or optionally substituted 5- or 6-membered cycloalkyl;
each R^{e} is independently at each occurrence C₁-C₆ alkyl, C₂-C₆ alkenyl, C₂-C₆ alkynyl, halogen, C₁-C₆ haloalkyl, —NHR^{z}, —OH, or —CN;
R^{f} is absent, H, or methyl;
R^{g} is H, C₁-C₆ alkyl, OH, —S(O)₂(C₁-C₆ alkyl), or S(O)₂N(C₁-C₆ alkyl)₂;
R^{g′} is H, C₁-C₆ alkyl, C₃-C₇ cycloalkyl, 4- to 7-membered heterocycloalkyl ring comprising 1-3 heteroatoms selected from N, O and S, C₆-C₁₀ aryl, or 5- to 7-membered heteroaryl comprising 1-3 heteroatoms selected from N, O and S, wherein the alkyl is optionally substituted with one or more substituents independently selected from halogen and —OH, and wherein the cycloalkyl, heterocycloalkyl, aryl and heteroaryl are optionally substituted with one or more substituents independently selected from C₁-C₆ alkyl, halogen, and —OH;
R^{h} is H, C₁-C₄ alkyl, or 3- to 7-membered heterocycloalkyl ring comprising 1-3 heteroatoms selected from N, O and S, wherein the alkyl is optionally substituted with one or more substituents each independently selected from NH₂, C₁-C₄ alkylamino, C₁-C₄ dialkylamino, and C(O)NH₂; and wherein the heterocycloalkyl is optionally substituted with one or more substituents each independently selected from C₁-C₆ alkyl and C₁-C₆ haloalkyl;
Rⁱ is (i) —(CH₂)ₛOC(O)C₁-C₆ alkyl, wherein the alkyl is substituted with one or more NH₂; (ii) (CH₂CH₂O)ₙCH₂CH₂OH; or (iii) C₁-C₆ alkyl substituted with one or more substituents each independently selected from OH and 4- to 7-membered heterocycloalkyl comprising 1 to 3 heteroatoms selected from O, N, or S;
R^{j} is absent, H, C₁-C₆ alkyl, or —CN;
each R^{x} is independently at each occurrence H, C₁-C₆ alkyl, or C₆-C₁₀ aryl;
each R^{y} and R^{z} is independently H, C₁-C₆ alkyl, or C₁-C₆ haloalkyl;
each m, p, q, r, and t is independently 0, 1 or 2;
n is 0, 1, 2, or 3;
s is 1 or 2;
o is 0, 1, 2, 3, or 4; and
[symbol] represents a single bond or a double bond; and
provided that
when X is O; R^{f} is H; W is C; R^{j} is —CN; L is —SCH₂—; R¹ is phenylene or pyridine; then R⁷ is not —COOH;
when X is O; R^{f} is H; W is C; R^{j} is —CN; L is —SCH₂—; R¹ is phenylene or pyridine; and R⁷ is tetrazole; then R^{c} is not H;
when X is O; R^{f} is H; W is C; R^{j} is —CN; L is —S—C(R⁵)₂ or —SCH₂CH₂—; R¹ is absent; then R⁷ is not COOH or tetrazole;
when X is O, R^{f} is H; W is N; R^{j} is absent; R^{d} is methyl, optionally substituted 5- to 10-membered aryl, optionally substituted 5- or 6-membered heteroaryl, or optionally substituted 5- or 6-membered cycloalkyl; L is —SCH₂— or —OCH₂—; and R¹ is phenylene; then R⁷ is not —COOH, —CH₂COOH,

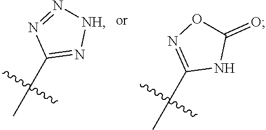 [SMILES: *c1nn[nH]n1 ; *c1noc(=O)[nH]1]

and
when X is O, R^{f} is H, W is N, R^{j} is absent, L is —NHCH₂—, —CH₂NH—, or —NH—C(O)—, and R¹ is phenylene, then R^{d} is not phenyl.

In further aspects, provided herein is a pharmaceutical composition, comprising a compound as described herein, or a pharmaceutically acceptable salt or tautomer thereof, and a pharmaceutically acceptable excipient. The present disclosure relates to treating an acute inflammatory condition comprising administering to a subject in need thereof, the pharmaceutical composition of the present disclosure.

The present disclosure provides a compound of the present disclosure, and pharmaceutically acceptable salts or tautomers thereof, or the pharmaceutical composition of the present disclosure for use in the treatment of an acute inflammatory condition in a subject in need thereof.

The present disclosure provides for use of a compound of the present disclosure, and pharmaceutically acceptable salts or tautomers thereof, for the treatment of an acute inflammatory condition in a subject in need thereof.

The present disclosure provides for use of a compound of the present disclosure, and pharmaceutically acceptable salts or tautomers thereof, in the manufacture of a medicament for the treatment of an acute inflammatory condition.

### BRIEF DESCRIPTION OF THE DRAWINGS

FIG. 1 shows the fold reduction in pro-inflammatory gene expression in Kupffer cells treated with LPS (50 ng/mL) followed by inhibition of ACMSD with compound I-34 (10 μM).

FIG. 2 shows the fold increase in anti-inflammatory gene expression in Kupffer cells treated with LPS (50 ng/mL) followed by inhibition of ACMSD with compound I-34 (10 μM).

FIG. 3 shows that ACMSD inhibition, with compound I-34, reversed LPS-induced reduction of QPRT expression in both HK-2 cells at 100 μM and Kupffer cells at 10 μM.

FIG. 4 shows fold change in expression of ACMSD in HK-2 cells treated with LPS (30 μg/ml) followed by treatment with an ACMSD inhibitor (compound I-34 100 μM).

FIG. 5 shows fold change in expression of IL6 and TNF-α measured in HK-2 cells treated with LPS (30 μg/ml) for 18 h, followed by treatment with an ACMSD inhibitor (compound I-34, 100 μM).

FIG. 6 shows the expression of genes involved in inflammation, performed in HK-2 cells incubated in the absence (DMSO) and in the presence of LPS or LPS with compound I-34.

FIG. 7 shows fold change in secretion of inflammatory cytokines, IL-1β, IL-6, and TNF-α measured in Kupffer cells treated with LPS (50 nmg/ml) for 24 h, followed by treatment with an ACMSD inhibitor (compound I-341 μM).

FIG. 8 shows the expression of genes involved in fibrosis performed in HK-2 cells incubated in the absence (DMSO) and in the presence of TGF-β or TGF-β together with compound I-34.

FIG. 9 shows fold change in expression of pro-fibrotic and pro-inflammatory genes measured in cocultured mouse primary hepatocytes and murine stellate cells treated with MIX or MIX together with an ACMSD inhibitor, compound I-34.

FIG. 10 shows fold change in secretion of pro-inflammatory genes measured in 3D human liver microtissues treated with MIX and LPS or MIX and LPS together with an ACMSD inhibitor, compound I-34.

FIG. 11 shows fold change in expression of pro-inflammatory genes measured in 3D human liver microtissues treated with MIX and LPS or MIX and LPS together with an ACMSD inhibitor, compound I-34.

FIG. 12 shows Caspases 3/7 activity in HK-2 cells induced by Cisplatin (50 μM): Three different doses 10, 50, and 100 μM of compound I-34 were used 1 h before Cisplatin injury.

### DETAILED DESCRIPTION OF THE DISCLOSURE

The following description sets forth numerous exemplary configurations, methods, parameters, and the like. It should be recognized, however, that such description is not intended as a limitation on the scope of the present disclosure, but is instead provided as a description of exemplary embodiments.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar to or equivalent to those described herein can be used in the practice and testing of the disclosure, suitable methods and materials are described below. All publications, patent applications, patents, and other references mentioned herein are incorporated by reference. The references cited herein are not admitted to be prior art to the claimed disclosure. In the case of conflict, the present specification, including definitions, will control. In addition, the materials, methods, and examples are illustrative only and not intended to be limiting.

Other features and advantages of the disclosure will be apparent from the following detailed description and claims.

The present disclosure relates to a method of treating an acute inflammatory condition in a subject comprising administering to the subject a therapeutically effective amount of a heteroaryl or heterocyclic compound.

As used herein, the terms “including,” “containing,” and “comprising” are used in their open, non-limiting sense. Throughout the description and claims of this specification, the words “comprise” and “contain” and variations of the words, for example “comprising” and “comprises”, mean “including but not limited to” and do not exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

The articles “a” and “an” as used in this disclosure may refer to one or more than one (i.e., to at least one) of the grammatical object of the article. By way of example, “an element” may mean one element or more than one element.

The term “and/or” as used in this disclosure may mean either “and” or “or” unless indicated otherwise.

To provide a more concise description, some of the quantitative expressions given herein are not qualified with the term “about.” It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. Whenever a yield is given as a percentage, such yield refers to a mass of the entity for which the yield is given with respect to the maximum amount of the same entity that could be obtained under the particular stoichiometric conditions. Concentrations that are given as percentages refer to mass ratios, unless indicated differently.

### Compounds

The present disclosure relates to compounds of Formula (I):

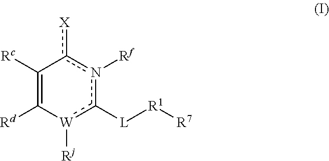

and pharmaceutically acceptable salts and tautomers thereof, wherein the substituents are as described herein.

The present disclosure relates to compounds of Formula (II):

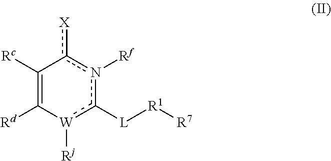

and pharmaceutically acceptable salts and tautomers thereof, wherein the substituents are as described herein.

In certain embodiments of Formula (I) or (II), wherein W is N, the present disclosure relates to compounds of Formula (I-I):

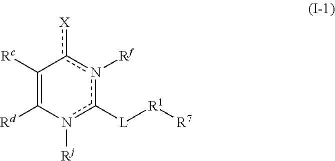

and pharmaceutically acceptable salts and tautomers thereof, wherein the substituents are as described herein for Formula (I) and (II).

In certain embodiments of Formula (I) or (II), wherein W is C, the present disclosure relates to compounds of Formula (I-2):

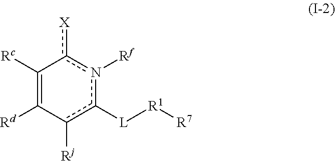

and pharmaceutically acceptable salts and tautomers thereof, wherein the substituents are as described herein for Formula (I) and (II).

In certain embodiments of Formula (I) or (II), wherein R¹ is phenyl, the present disclosure relates to compounds of Formula (I-3):

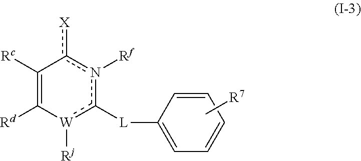

and pharmaceutically acceptable salts and tautomers thereof, wherein the substituents are as described herein for Formula (I) and (II).

In certain embodiments of Formula (I) or (II), wherein R¹ is absent, the present disclosure relates to compounds of Formula (I-4):

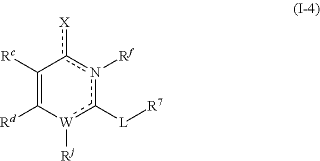

and pharmaceutically acceptable salts and tautomers thereof, wherein the substituents are as described herein for Formula (I) and (II).

As described above, X is H, S, SR², NR², NR²R^{2′}, O, OH, OR^{h}, F, Br, or Cl. In certain embodiments, X is O, OH, OR^{h}, F, Br, or Cl. In certain embodiments, X is H, S, SR², NR², or NR²R^{2′}. In certain embodiments, X is H. In certain embodiments, X is S. In certain embodiments, X is SR². In certain embodiments, X is NR². In certain embodiments, X is NR²R^{2′}. In certain embodiments, X is O. In certain embodiments, X is OH. In certain embodiments, X is OR^{h}. In certain embodiments, X is F. In certain embodiments, X is Br. In certain embodiments, X is Cl.

As described above, R² is H or C₁-C₄ alkyl. In certain embodiments, R² is H. In certain embodiments, R² is C₁-C₄ alkyl. In certain embodiments, R² is —CH₃.

As described above, R^{2′} is H, C₁-C₄ alkyl, or C₃-C₇ cycloalkyl. In certain embodiments, R^{2′} is H. In certain embodiments, R^{2′} is C₁-C₄ alkyl. In certain embodiments, R^{2′} is C₃-C₇ cycloalkyl.

As described above, R² and R^{2′} together with the nitrogen atom to which they are attached form a 3- to 7-membered heterocycloalkyl ring comprising 1-3 additional heteroatoms selected from N, O, and S. In certain embodiments, R² and R^{2′} together with the nitrogen atom to which they are attached form a 6-membered heterocycloalkyl ring.

As described above, R^{h} is H, C₁-C₄ alkyl, or 3- to 7-membered heterocycloalkyl ring comprising 1-3 heteroatoms selected from N, O, and S, wherein the alkyl is optionally substituted with one or more substituents each independently selected from NH₂, C₁-C₄ alkylamino, C₁-C₄ dialkylamino, and C(O)NH₂; and wherein the heterocycloalkyl is optionally substituted with one or more substituents each independently selected from C₁-C₆ alkyl and C₁-C₆ haloalkyl. In certain embodiments, R^{h} is H. In certain embodiments, R^{h} is C₁-C₄ alkyl, wherein the alkyl is optionally substituted with one or more substituents each independently selected from NH₂, C₁-C₄ alkylamino, C₁-C₄ dialkylamino, and C(O)NH₂. In certain embodiments, R^{h} is 3- to 7-membered heterocycloalkyl ring comprising 1-3 heteroatoms selected from N, O, and S, wherein the heterocycloalkyl is optionally substituted with one or more substituents each independently selected from C₁-C₆ alkyl and C₁-C₆ haloalkyl.

As described above, R^{f} is absent, H, or methyl. In certain embodiments, R^{f} is absent. In certain embodiments, R^{f} is H. In certain embodiments, R^{f} is methyl.

As described above, W is N or C. In certain embodiments, W is N. In certain embodiments, W is C.

As described above, R^{j} is absent, H, C₁-C₆ alkyl, or —CN. In certain embodiments, R^{j} is absent. In certain embodiments, R^{j} is H. In certain embodiments, R^{j} is C₁-C₆ alkyl. In certain embodiments, R^{j} is —CN.

In certain embodiments, W is N and R^{j} is absent. In certain embodiments, W is C and R^{j} is H, C₁-C₆ alkyl, or —CN. In certain embodiments, W is C and R^{j} is —CN.

As described above, R^{c} is H, C₁-C₆ alkyl, C₁-C₆ haloalkyl, halogen, —CN, —OR^{x}, or —CO₂R^{x}. In certain embodiments, R^{c} is H. In certain embodiments, R^{c} is C₁-C₆ alkyl. In certain embodiments, R^{c} is C₁-C₆ haloalkyl. In certain embodiments, R^{c} is halogen. In certain embodiments, R^{c} is —CN. In certain embodiments, R^{c} is OR^{h}. In certain embodiments, R^{c} is —CO₂R^{x}.

As described above, R^{x} is independently at each occurrence H, C₁-C₆ alkyl, or C₆-C₁₀ aryl. In certain embodiments, R^{x} is H. In certain embodiments, R^{x} is C₁-C₆ alkyl. In certain embodiments, R^{x} is C₆-C₁₀ aryl.

As described above, R^{d} is methyl, CF₃, CR^{f}F₂, —(C(R⁶)₂)ₜC₆-C₁₀ aryl, —(C(R⁶)₂)ₜ-5- or 6-membered heteroaryl, —(C(R⁶)₂)ₜ-5- or 6-membered cycloalkyl, optionally substituted C₆-C₁₀ aryl, optionally substituted 5- or 6-membered heteroaryl, or optionally substituted 5- or 6-membered cycloalkyl.

In certain embodiments, R^{d} is methyl. In certain embodiments, R^{d} is CF₃. In certain embodiments, R^{d} is CR^{f}F₂. In certain embodiments, R^{d} is —(C(R⁶)₂)ₜC₆-C₁₀ aryl. In certain embodiments, R^{d} is —CH₂C₆-C₁₀ aryl. In certain embodiments, R^{d} is —CH₂C₆ aryl. In certain embodiments, R^{d} is —(C(R⁶)₂)ₜ-5- or 6-membered heteroaryl. In certain embodiments, R^{d} is —(C(R⁶)₂)ₜ-5- or 6-membered cycloalkyl. In certain embodiments, R^{d} is optionally substituted C₆-C₁₀ aryl. In certain embodiments, R^{d} is optionally substituted 5- or 6-membered heteroaryl. In certain embodiments, R^{d} is optionally substituted 5- or 6-membered cycloalkyl.

As described above, R^{f} is absent, H, or methyl. In certain embodiments, R^{f} is absent. In certain embodiments, R^{f} is H. In certain embodiments, R^{f} is methyl.

As described above, t is 0, 1, or 2. In certain embodiments, t is 0. In certain embodiments, t is 1. In certain embodiments, t is 2.

As described above, when W is N, then L is —(C(R⁵)₂)ₘCH═CH(C(R⁵)₂)ₚ—,

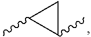

—(C(R⁵)₂)ₘY¹(C(R⁵)₂)ₚ—, —(C(R⁵)₂)ₘY¹(C(R⁵)₂)ₚ-cyclopropyl-, —(C(R⁵)₂)ₘY¹CH═CH—, —(C(R⁵)₂)ₘNR³C═(O)(C(R⁵)₂)ₚ—, —(C(R⁵)₂)ₘphenyl(C(R⁵)₂)ₚ—, —(C(R⁵)₂)ₘpyridinyl(C(R⁵)₂)ₚ—, or —(C(R⁵)₂)ₘthiophenyl(C(R⁵)₂)ₚ—.

In certain embodiments, W is N and L is —(C(R⁵)₂)ₘCH═CH(C(R⁵)₂)ₚ—. In certain embodiments, W is N and L is

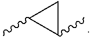

In certain embodiments, W is N and L is —(C(R⁵)₂)ₘY¹(C(R⁵)₂)ₚ—. In certain embodiments, W is N and L is —(C(R⁵)₂)ₘY¹(C(R⁵)₂)ₚ-cyclopropyl-. In certain embodiments, W is N and L is —(C(R⁵)₂)ₘY¹CH═CH—. In certain embodiments, W is N and L is —(C(R⁵)₂)ₘNR³C═(O)(C(R⁵)₂)ₚ—. In certain embodiments, W is N and L is —(C(R⁵)₂)ₘphenyl(C(R⁵)₂)ₚ—. In certain embodiments, W is N and L is —(C(R⁵)₂)ₘpyridinyl(C(R⁵)₂)ₚ—. In certain embodiments, W is N and L is —(C(R⁵)₂)ₘthiophenyl(C(R⁵)₂)ₚ—. In certain embodiments, W is N and L is —(C(R⁵)₂)ₘY¹(C(R⁵)₂)ₚ, such as —SCH₂— or —NHCH₂—. In certain embodiments, W is N and L is —SCH₂—. In certain embodiments, W is N and L is —NHCH₂—.

As described above, when W is C, L is —(C(R⁵)₂)ₘCH═CH(C(R⁵)₂)ₚ—, —(C(R⁵)₂)ₒ—, —(C(R⁵)₂)ₘY¹(C(R⁵)₂)ₚ—,

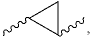

—(C(R⁵)₂)ₘ Y¹CH═CH—, —(C(R⁵)₂)ₘC═(O)(CH₂)ₚ—, —(C(R⁵)₂)ₘC═(O)O(C(R⁵)₂)ₚ—, —(C(R⁵)₂)ₘC═(O)NR³(C(R⁵)₂)ₚ—, —(C(R⁵)₂)ₘNR³C═(O)(C(R⁵)₂)ₚ—, —(C(R⁵)₂)ₘphenyl(C(R⁵)₂)ₚ—, —(C(R⁵)₂)ₘpyridinyl(C(R⁵)₂)ₚ—, or —(C(R⁵)₂)ₘthiophenyl(C(R⁵)₂)ₚ—.

In certain embodiments, W is C and L is —(C(R⁵)₂)ₘCH═CH(C(R⁵)₂)ₚ—. In certain embodiments, W is C and L is —(C(R⁵)₂)ₒ—. In certain embodiments, W is C and L is —(C(R⁵)₂)ₘY¹(C(R⁵)₂)ₚ—. In certain embodiments, W is C and L is

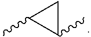

In certain embodiments, W is C and L is —(C(R⁵)₂)ₘ Y¹CH═CH—. In certain embodiments, W is C and L is —(C(R⁵)₂)ₘC═(O)(CH₂)ₚ—. In certain embodiments, W is C and L is —(C(R⁵)₂)ₘC═(O)O(C(R⁵)₂)ₚ—. In certain embodiments, W is C and L is —(C(R⁵)₂)ₘC═(O)NR³(C(R⁵)₂)ₚ—. In certain embodiments, W is C and L is —(C(R⁵)₂)ₘNR³C═(O)(C(R⁵)₂)ₚ—. In certain embodiments, W is C and L is —(C(R⁵)₂)ₘphenyl(C(R⁵)₂)ₚ—. In certain embodiments, W is C and L is —(C(R⁵)₂)ₘpyridinyl(C(R⁵)₂)ₚ—. In certain embodiments, W is C and L is —(C(R⁵)₂)ₘthiophenyl(C(R⁵)₂)ₚ—.

As described above, Y¹ is O, NR⁴, or S(O)_{q}. In certain embodiments, Y¹ is O. In certain embodiments, Y¹ is NR⁴. As described above, R⁴ is H or C₁-C₄ alkyl. In certain embodiments, R⁴ is H. In certain embodiments, R⁴ is C₁-C₄ alkyl.

In certain embodiments, Y¹ is S(O)_{q}. As described above, q is 0, 1, or 2. In certain embodiments, q is 0. In certain embodiments, Y¹ is S. In certain embodiments, q is 1. In certain embodiments, q is 2.

As described above, each R⁵ is independently at each occurrence H or C₁-C₄ alkyl. In certain embodiments, R⁵ is H. In certain embodiments, R⁵ is C₁-C₄ alkyl.

As described above, R³ is H or C₁-C₄ alkyl. In certain embodiments, R³ is H. In certain embodiments, R³ is C₁-C₄ alkyl.

As described above, each m and p is independently 0, 1 or 2. In certain embodiments, m is 0. In certain embodiments, m is 1. In certain embodiments, m is 2. In certain embodiments, p is 0. In certain embodiments, p is 1. In certain embodiments, p is 2.

As described above, o is 0, 1, 2, 3, or 4. In certain embodiments, o is 0. In certain embodiments, o is 1. In certain embodiments, o is 2. In certain embodiments, o is 3. In certain embodiments, o is 4.

As described above, R¹ is absent or C₆-C₁₀ arylene or heteroarylene, wherein the heteroarylene comprises one or two 5- to 7-membered rings and 1-4 heteroatoms selected from N, O and S, and wherein the C₆-C₁₀ arylene or heteroarylene are optionally substituted with one to two R^{e}. In certain embodiments, R¹ is absent. In certain embodiments, R¹ is C₆-C₁₀ arylene, which is optionally substituted with one to two R^{e}. In certain embodiments, R¹ is heteroarylene, wherein the heteroarylene comprises one or two 5- to 7-membered rings and 1 4 heteroatoms selected from N, O and S, and optionally substituted with one to two R^{e}. In certain embodiments of Formula (II), R¹ is C₃-C₈cycloalkylene, such as C₃cycloalkylene, C₄cycloalkylene, C₅cycloalkylene, C₆cycloalkylene, C₇cycloalkylene, or C₈cycloalkylene

As described above, each R^{e} is independently at each occurrence C₁-C₆ alkyl, C₂-C₆ alkenyl, C₂-C₆ alkynyl, halogen, C₁-C₆ haloalkyl, —NHR^{z}, —OH, or —CN.

As described above, R⁷ is H, A, B, or C. In certain embodiments, R⁷ is H. In certain embodiments, R⁷ is A. In certain embodiments, R⁷ is B. In certain embodiments, R⁷ is C.

As described above for Formula (I), A is —(C(R⁶)₂)ᵣCO₂R^{x}, —Y²(C(R⁶)₂)ᵣCO₂R^{x}, —(CH₂)ᵣtetrazole, —(CH₂)ᵣoxadiazolone, —(CH₂)ᵣtetrazolone, —(CH₂)ᵣthiadiazolol, —(CH₂)ᵣisoxazol-3-ol, —(CH₂)ᵣP(O)(OH)OR^{x}, —(CH₂)ᵣS(O)₂OH, —(CH₂)ᵣC(O)NHCN, or —(CH₂)ᵣC(O)NHS(O)₂alkyl, wherein —(CH₂)ᵣtetrazole, —(CH₂)ᵣoxadiazolone, —(CH₂)ᵣtetrazolone, —(CH₂)ᵣthiadiazolol, —(CH₂)ᵣ isoxazol-3-ol are optionally substituted with C₁-C₆ alkyl.

As described above for Formula (II), A is —(C(R⁶)₂)ᵣCO₂R^{x}, —Y²(C(R⁶)₂)ᵣCO₂R^{x}, —(C(R⁶)₂)ᵣtetrazole, —(C(R⁶)₂)ᵣoxadiazolone, —(C(R⁶)₂)ᵣtetrazolone, —(C(R⁶)₂)ᵣthiadiazolol, —(C(R⁶)₂)ᵣ isoxazol-3-ol, —(C(R⁶)₂)ᵣP(O)(OH)OR^{x}, —(C(R⁶)₂)ᵣS(O)₂OH, —(C(R⁶)₂)ᵣC(O)NHCN, or —(C(R⁶)₂)ᵣC(O)NHS(O)₂alkyl, wherein —(C(R⁶)₂)ᵣtetrazole, —(C(R⁶)₂)ᵣoxadiazolone, —(C(R⁶)₂)ᵣtetrazolone, —(C(R⁶)₂)ᵣthiadiazolol, —(C(R⁶)₂)ᵣ isoxazol-3-ol are optionally substituted with C₁-C₆ alkyl. In certain embodiments, A is —(C(R⁶)₂)ᵣtetrazole. In certain embodiments, A is —(C(R⁶)₂)ᵣoxadiazolone. In certain embodiments, A is —(C(R⁶)₂)ᵣtetrazolone. In certain embodiments, A is —(C(R⁶)₂)ᵣthiadiazolol. In certain embodiments, A is —(C(R⁶)₂)ᵣ isoxazol-3-ol. In certain embodiments, A is —(C(R⁶)₂)ᵣP(O)(OH)OR^{x}. In certain embodiments, A is —(C(R⁶)₂)ᵣS(O)₂OH. In certain embodiments, A is —(C(R⁶)₂)ᵣC(O)NHCN. In certain embodiments, A is —(C(R⁶)₂)ᵣC(O)NHS(O)₂alkyl.

In certain embodiments, A is —(C(R⁶)₂)ᵣCO₂R^{x}. In certain embodiments, A is —Y²(C(R⁶)₂)ᵣCO₂R^{x}. In certain embodiments, A is —(CH₂)ᵣtetrazole. In certain embodiments, A is —(CH₂)ᵣoxadiazolone. In certain embodiments, A is —(CH₂)ᵣtetrazolone. In certain embodiments, A is —(CH₂)ᵣthiadiazolol. In certain embodiments, A is —(CH₂)ᵣ isoxazol-3-ol. In certain embodiments, A is —(CH₂)ᵣP(O)(OH)OR^{x}. In certain embodiments, A is —(CH₂)ᵣS(O)₂OH. In certain embodiments, A is —(CH₂)ᵣC(O)NHCN. In certain embodiments, A is —(CH₂)ᵣC(O)NHS(O)₂alkyl. In certain embodiments, —(CH₂)ᵣtetrazole, —(CH₂)ᵣoxadiazolone, —(CH₂)ᵣtetrazolone, —(CH₂)ᵣthiadiazolol, —(CH₂)ᵣ isoxazol-3-ol are optionally substituted with C₁-C₆ alkyl. In certain embodiments, A is —(C(R⁶)₂)ᵣCO₂R^{x} or —(CH₂)ᵣtetrazole, wherein —(CH₂)ᵣtetrazole is optionally substituted with C₁-C₆ alkyl. In certain embodiments, A is —(C(R⁶)₂)ᵣCOOH or —(CH₂)ᵣtetrazole, wherein —(CH₂)ᵣtetrazole is optionally substituted with C₁-C₆ alkyl. In certain embodiments, A is —COOH, —CH₂COOH, -tetrazole, or —(CH₂)ᵣtetrazole, wherein tetrazole and —(CH₂)ᵣtetrazole are optionally substituted with C₁-C₆ alkyl.

As described above for Formula (I), B is —(C(R⁶)₂)ᵣS(O)₂OC₁-C₄ alkyl, —O(C(R⁶)₂)ᵣS(O)₂OC₁-C₄ alkyl, —Y²(C(R⁶)₂)ᵣC(O)NR^{g}R^{g′}, —Y²(C(R⁶)₂)ᵣS(O)₂NR^{g}R^{g′}, —(CH₂)ᵣC(O)NR^{g}R^{g′}, —(CH₂)ᵣS(O)₂NR^{g}R^{g′}, —(CH₂)ᵣC(O)NHS(O)₂NR^{g}R^{g′}, —(C(R⁶)₂)ᵣCO₂Rⁱ, —(C(R⁶)₂)ᵣNH₂CO₂R^{x}, —(C(R⁶)₂)ᵣP(O)(OR^{x})₂, —O(C(R⁶)₂)ᵣP(O)(OR^{x})₂, —(C(R⁶)₂)ᵣS(O)₂OH, —O(C(R⁶)₂)ᵣS(O)₂OH, —(C(R⁶)₂)ᵣP(O)₂OR^{x}, or —O(C(R⁶)₂)ᵣP(O)₂OR^{x}.

As described above for Formula (II), is —(C(R⁶)₂)ᵣS(O)₂OC₁-C₄ alkyl, —O(C(R⁶)₂)ᵣS(O)₂OC₁-C₄ alkyl, —Y²(C(R⁶)₂)ᵣC(O)NR^{g}R^{g′}, —Y²(C(R⁶)₂)ᵣS(O)₂NR^{g}R^{g′}, —(C(R⁶)₂)ᵣC(O)NR^{g}R^{g′}, —(C(R⁶)₂)ᵣS(O)₂NR^{g}R^{g′}, —(C(R⁶)₂)ᵣC(O)NHS(O)₂NR^{g}R^{g′}, —(C(R⁶)₂)ᵣCO₂R¹, —(C(R⁶)₂)ᵣNH₂CO₂R^{x}, —(C(R⁶)₂)ᵣP(O)(OR^{x})₂, —O(C(R⁶)₂)ᵣP(O)(OR^{x})₂, —(C(R⁶)₂)ᵣS(O)₂OH, —O(C(R⁶)₂)ᵣS(O)₂OH, —(C(R⁶)₂)ᵣP(O)₂OR^{x}, or —O(C(R⁶)₂)ᵣP(O)₂OR^{x}. In certain embodiments, B is —(C(R⁶)₂)ᵣC(O)NR^{g}R^{g′}. In certain embodiments, B is —(C(R⁶)₂)ᵣS(O)₂NR^{g}R^{g′}. In certain embodiments, B is —(C(R⁶)₂)ᵣC(O)NHS(O)₂NR^{g}R^{g′}.

In certain embodiments, B is —(C(R⁶)₂)ᵣS(O)₂OC₁-C₄ alkyl. In certain embodiments, B is —O(C(R⁶)₂)ᵣS(O)₂OC₁-C₄ alkyl. In certain embodiments, B is —Y²(C(R⁶)₂)ᵣC(O)NR^{g}R^{g′}. In certain embodiments, B is —Y²(C(R⁶)₂)ᵣS(O)₂NR^{g}R^{g′}. In certain embodiments, B is —(CH₂)ᵣC(O)NR^{g}R^{g′}. In certain embodiments, B is —(CH₂)ᵣS(O)₂NR^{g}R^{g′}. In certain embodiments, B is —(CH₂)ᵣC(O)NHS(O)₂NR^{g}R^{g′}. In certain embodiments, B is —(C(R⁶)₂)ᵣCO₂R¹. In certain embodiments, B is —(C(R⁶)₂)ᵣNH₂CO₂R^{x}. In certain embodiments, B is —(C(R⁶)₂)ᵣP(O)(OR^{x})₂. In certain embodiments, B is —O(C(R⁶)₂)ᵣP(O)(OR^{x})₂. In certain embodiments, B is —(C(R⁶)₂)ᵣS(O)₂OH. In certain embodiments, B is —O(C(R⁶)₂)ᵣS(O)₂OH. In certain embodiments, B is —(C(R⁶)₂)ᵣP(O)₂OR^{x}. In certain embodiments, B is —O(C(R⁶)₂)ᵣP(O)₂OR^{x}.

As described above, C is —(CH₂)ᵣCN, —(CH₂)ₛOH, halogen, —(C(R⁶)₂)ᵣC₆-C₁₀ aryl, —(C(R⁶)₂)ᵣS—C₆-C₁₀ aryl, —(C(R⁶)₂)ᵣheteroaryl, —O(C(R⁶)₂)ᵣheteroaryl, —O(C(R⁶)₂)ᵣheterocycloalkyl, —O(C(R⁶)₂)ᵣOH, OR, —(C(R⁶)₂)ᵣC(O)NHCN, —CH═CHCO₂R^{x}, or —(C(R⁶)₂)ᵣC(O)NHS(O)₂C₁-C₄ alkyl, wherein the aryl and heteroaryl are substituted with one to three substituents each independently selected from C₁-C₆ alkyl, C₁-C₆ haloalkyl, halogen, and OH, and wherein the heterocycloalkyl is substituted with one to two ═O or ═S;

In certain embodiments, C is —(CH₂)ᵣCN. In certain embodiments, C is —(CH₂)ₛOH. In certain embodiments, C is halogen. In certain embodiments, C is —(C(R⁶)₂)ᵣC₆-C₁₀ aryl. In certain embodiments, C is —(C(R⁶)₂)ᵣS—C₆-C₁₀ aryl. In certain embodiments, C is —(C(R⁶)₂)ᵣheteroaryl. In certain embodiments, C is —O(C(R⁶)₂)ᵣheteroaryl. In certain embodiments, C is-O(C(R⁶)₂)ᵣheterocycloalkyl. In certain embodiments, C is —O(C(R⁶)₂)ᵣOH. In certain embodiments, C is —OR^{y}. In certain embodiments, C is —(C(R⁶)₂)ᵣC(O)NHCN. In certain embodiments, C is —CH═CHCO₂R^{x}. In certain embodiments, C is —(C(R⁶)₂)ᵣC(O)NHS(O)₂C₁-C₄ alkyl. In the above, the aryl and heteroaryl are substituted with one to three substituents each independently selected from C₁-C₆ alkyl, C₁-C₆ haloalkyl, halogen, and OH, and wherein the heterocycloalkyl is substituted with one to two ═O or S.

As described above, each R⁶ is independently at each occurrence H or C₁-C₄ alkyl. In certain embodiments, R⁶ is H. In certain embodiments, R⁶ is C₁-C₄ alkyl.

As described above, each R^{x} is independently at each occurrence H, C₁-C₆ alkyl, or C₆-C₁₀ aryl. In certain embodiments, R^{x} is H. In certain embodiments, R^{x} is C₁-C₆ alkyl. In certain embodiments, R^{c} is C₆-C₁₀ aryl.

As described above, each Y² is independently O, NH or S. In certain embodiments, Y² is O. In certain embodiments, Y² is NH. In certain embodiments, Y² is S.

As described above, each r independently is 0, 1 or 2. In certain embodiments, r is 0. In certain embodiments, r is 1. In certain embodiments, r is 2.

As described above, s is 1 or 2. In certain embodiments, s is 1. In certain embodiments, s is 2.

As described above, R^{g} is H, C₁-C₆ alkyl, OH, —S(O)₂(C₁-C₆ alkyl), or —S(O)₂N(C₁-C₆ alkyl)₂.

As described above, R^{g′} is H, C₁-C₆ alkyl, C₃-C₇ cycloalkyl, 4- to 7-membered heterocycloalkyl ring comprising 1-3 heteroatoms selected from N, O and S, C₆-C₁₀ aryl, or 5- to 7-membered heteroaryl comprising 1-3 heteroatoms selected from N, O and S, wherein the alkyl is optionally substituted with one or more substituents independently selected from halogen and —OH, and wherein the cycloalkyl, heterocycloalkyl, aryl and heteroaryl are optionally substituted with one or more substituents independently selected from C₁-C₆ alkyl, halogen, and —OH.

In some embodiments, the present disclosure provides a compound of formula (I) having one, two, or three of the following features:
a) W is N;
b) R^{c} is CN;
C) R^{d} is 5- or 6-membered heteroaryl, such as thiophenyl;
d) L is —(C(R⁵)₂)ₘY¹(C(R⁵)₂)ₚ, such as —SCH₂—;
e) R¹ is phenylene;
f) R⁷ is A, such as COOH or tetrazole.

In some embodiments, the present disclosure provides a compound of formula (I) having one, two, or three of the following features:
a) W is N;
b) R^{d} is CF₃;
C) L is —(C(R⁵)₂)ₘY¹(C(R⁵)₂)ₚ, such as —SCH₂—;
d) R¹ is phenylene;
e) R⁷ is A, such as COOH or tetrazole.

In some embodiments, the present disclosure provides a compound of formula (I) having one, two, or three of the following features:
a) W is N;
b) R^{c} is CN;
C) R^{d} is 5- or 6-membered heteroaryl, such as thiophenyl;
d) L is —(C(R⁵)₂)ₘY¹(C(R⁵)₂)ₚ, such as —SCH₂—;
e) R¹ is absent;
f) R⁷ is A, such as COOH or tetrazole.

In some embodiments, the present disclosure provides a compound of formula (I) having one, two, or three of the following features:
a) W is N;
b) R^{d} is CF₃;
C) L is —(C(R⁵)₂)ₘY¹(C(R⁵)₂)ₚ, such as —SCH₂—;
d) R¹ is absent;
e) R⁷ is A, such as COOH or tetrazole.

In some embodiments, the present disclosure provides a compound of formula (I) having one, two, or three of the following features:
a) W is C;
b) R^{d} is —(C(R⁶)₂)ₜC₆-C₁₀ aryl or —(C(R⁶)₂)ₜ-5- or 6-membered heteroaryl); C) L is —(C(R⁵)₂)ₘY¹(C(R⁵)₂)ₚ, such as —SCH₂—;
d) R¹ is phenylene;
e) R⁷ is A, such as COOH or tetrazole.

In some embodiments, the present disclosure provides a compound of formula (I) having one, two, or three of the following features:
a) W is C;
b) R^{d} is —CF₃;
C) L is —(C(R⁵)₂)ₘY¹(C(R⁵)₂)ₚ, such as —SCH₂—;
d) R¹ is phenylene;
e) R⁷ is A, such as COOH or tetrazole.

In some embodiments, the present disclosure provides a compound of formula (I) having one, two, or three of the following features:
a) W is N;
b) R^{c} is CN;
C) R^{d} is 5- or 6-membered heteroaryl, such as thiophenyl;
d) L is —(C(R⁵)₂)ₘY¹(C(R⁵)₂)ₚ, such as —SCH₂—;
e) R¹ is phenylene;
f) R⁷ is A, such as —(C(R⁶)₂)ᵣCO₂R^{x} or —(CH₂)ᵣtetrazole.

In some embodiments, the present disclosure provides a compound of formula (I) having one, two, or three of the following features:
a) W is N;
b) R^{d} is CF₃;
C) L is —(C(R⁵)₂)ₘY¹(C(R⁵)₂)ₚ, such as —SCH₂—;
d) R¹ is phenylene;
e) R⁷ is A, such as —(C(R⁶)₂)ᵣCO₂R^{x} or —(CH₂)ᵣtetrazole.

In some embodiments, the present disclosure provides a compound of formula (I) having one, two, or three of the following features:
a) W is N;
b) R^{c} is CN;
C) R^{d} is 5- or 6-membered heteroaryl, such as thiophenyl;
d) L is —(C(R⁵)₂)ₘY¹(C(R⁵)₂)ₚ, such as —SCH₂—;
e) R¹ is absent;
f) R⁷ is A, such as —(C(R⁶)₂)ᵣCO₂R^{x} or —(CH₂)ᵣtetrazole.

In some embodiments, the present disclosure provides a compound of formula (I) having one, two, or three of the following features:
a) W is N;
b) R^{d} is CF₃;
C) L is —(C(R⁵)₂)ₘY¹(C(R⁵)₂)ₚ, such as —SCH₂—;
d) R¹ is absent;
e) R⁷ is A, such as —(C(R⁶)₂)ᵣCO₂R^{x} or —(CH₂)ᵣtetrazole.

In some embodiments, the present disclosure provides a compound of formula (I) having one, two, or three of the following features:
a) W is C;
b) R^{d} is —(C(R⁶)₂)ₜC₆-C₁₀ aryl or —(C(R⁶)₂)ₜ-5- or 6-membered heteroaryl); C) L is —(C(R⁵)₂)ₘY¹(C(R⁵)₂)ₚ, such as —SCH₂—;
d) R¹ is phenylene;
e) R⁷ is A, such as —(C(R⁶)₂)ᵣCO₂R^{x} or —(CH₂)ᵣtetrazole.

In some embodiments, the present disclosure provides a compound of formula (I) having one, two, or three of the following features:
a) W is C;
b) R^{d} is —CF₃;
C) L is —(C(R⁵)₂)ₘY¹(C(R⁵)₂)ₚ, such as —SCH₂—;
d) R¹ is phenylene;
e) R⁷ is A, such as —(C(R⁶)₂)ᵣCO₂R^{x} or —(CH₂)ᵣtetrazole.

In certain embodiments, with certain above features for Formula (I), the present disclosure provides a compound of formula (Ia) having at least one of the following features:

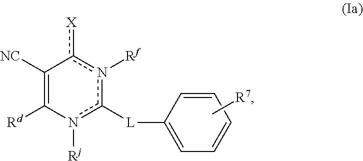

and pharmaceutically salts and tautomers thereof, wherein
R^{d} is 5- or 6-membered heteroaryl;
L is —(C(R⁵)₂)ₘY¹(C(R⁵)₂)ₚ;
C) R⁷ is A or C;
d) X, R^{d}, R^{f}, R^{j}, A, R⁵, Y¹, m, and p are defined for Formula (I).
   In certain embodiments, R^{d} is thiophenyl. In certain embodiments, L is —SCH₂— or —NHCH₂—. In certain embodiments, R^{c} is C. In certain embodiments, C is —(C(R⁶)₂)ᵣC₆-C₁₀ aryl, substituted with one to three substituents each independently selected from C₁-C₆ alkyl, C₁-C₆ haloalkyl, halogen and OH. In certain embodiments, R⁷ is A. In certain embodiments, A is
   —(C(R⁶)₂)ᵣCO₂R^{x} or —(CH₂)ᵣtetrazole, wherein —(CH₂)ᵣtetrazole is optionally substituted with C₁-C₆ alkyl. In certain embodiments, A is —(C(R⁶)₂)ᵣCOOH or —(CH₂)ᵣtetrazole, wherein —(CH₂)ᵣtetrazole is optionally substituted with C₁-C₆ alkyl. In certain embodiments, A is —COOH, —CH₂COOH, -tetrazole, or —(CH₂)ᵣtetrazole, wherein tetrazole and —(CH₂)ᵣtetrazole are optionally substituted with C₁-C₆ alkyl. In certain embodiments, Formula (Ia) has one, two, three or four of the features (a) to (d).

In certain embodiments, with certain above features for Formula (I), the present disclosure provides a compound of formula (Ib) having at least one of the following features:

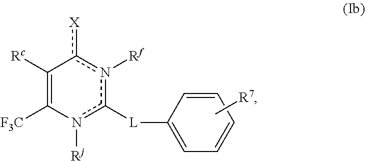

and pharmaceutically salts and tautomers thereof, wherein
a) L is —(C(R⁵)₂)ₘY¹(C(R⁵)₂)ₚ;
b) R⁷ is A;
c) X, R^{e}, R^{f}, R^{j}, A, R⁵, Y¹, m, and p are defined for Formula (I).
   In certain embodiments, L is —SCH₂— or —NHCH₂—. In certain embodiments, A is —(C(R⁶)₂)ᵣCO₂R^{x} or —(CH₂)ᵣtetrazole, wherein —(CH₂)ᵣtetrazole is optionally substituted with C₁-C₆ alkyl. In certain embodiments, A is —(C(R⁶)₂)ᵣCOOH or —(CH₂)ᵣtetrazole, wherein —(CH₂)ᵣtetrazole is optionally substituted with C₁-C₆ alkyl. In certain embodiments, A is —COOH, —CH₂COOH, -tetrazole, or —(CH₂)ᵣtetrazole, wherein tetrazole and —(CH₂)ᵣtetrazole are optionally substituted with C₁-C₆ alkyl. In certain embodiments, Formula (Ib) has one, two, or three of the features (a) to (c).

In certain embodiments, with certain above features for Formula (I), the present disclosure provides a compound of formula (Ic) having at least one of the following features:

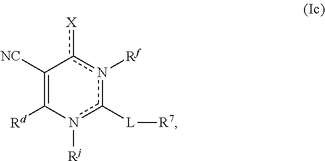

and pharmaceutically salts and tautomers thereof, wherein
a) R^{d} is 5- or 6-membered heteroaryl;
b) L is —(C(R⁵)₂)ₘY¹(C(R⁵)₂)ₚ;
c) R⁷ is A;
d) X, R^{d}, R^{f}, R^{j}, A, R⁵, Y¹, m, and p are defined for Formula (I).
   In certain embodiments, R^{d} is thiophenyl. In certain embodiments, L is —SCH₂— or —NHCH₂—. In certain embodiments, A is —(C(R⁶)₂)ᵣCO₂R^{x} or —(CH₂)ᵣtetrazole, wherein —(CH₂)ᵣtetrazole is optionally substituted with C₁-C₆ alkyl. In certain embodiments, A is —(C(R⁶)₂)ᵣCOOH or —(CH₂)ᵣtetrazole, wherein —(CH₂)ᵣtetrazole is optionally substituted with C₁-C₆ alkyl. In certain embodiments, A is —COOH, —CH₂COOH, -tetrazole, or —(CH₂)tetrazole, wherein tetrazole and —(CH₂)ᵣtetrazole are optionally substituted with C₁-C₆ alkyl. In certain embodiments, Formula (Ic) has one, two, three or four of the features (a) to (d).

In certain embodiments, with certain above features for Formula (I), the present disclosure provides a compound of formula (Id) having at least one of the following features:

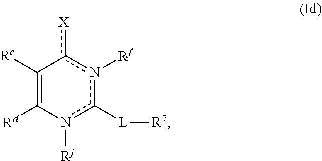

and pharmaceutically salts and tautomers thereof, wherein
a) L is —(C(R⁵)₂)ₘY¹(C(R⁵)₂)ₚ;
b) R⁷ is A;
c) X, R^{d}, R^{f}, R^{j}, A, R⁵, Y¹, m, and p are defined for Formula (I).
   In certain embodiments, L is —SCH₂— or —NHCH₂—. In certain embodiments, A is —(C(R⁶)₂)ᵣCO₂R^{x} or —(CH₂)ᵣtetrazole, wherein —(CH₂)ᵣtetrazole is optionally substituted with C₁-C₆ alkyl. In certain embodiments, A is —(C(R⁶)₂)ᵣCOOH or —(CH₂)ᵣtetrazole, wherein —(CH₂)ᵣtetrazole is optionally substituted with C₁-C₆ alkyl. In certain embodiments, A is —COOH, —CH₂COOH, -tetrazole, or —(CH₂)ᵣtetrazole, wherein tetrazole and —(CH₂)ᵣtetrazole are optionally substituted with C₁-C₆ alkyl. In certain embodiments, Formula (Id) has one, two, or three of the features (a) to (c).

In certain embodiments, with certain above features for Formula (I), the present disclosure provides a compound of formula (Ie) having at least one of the following features:

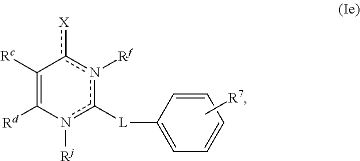

and pharmaceutically salts and tautomers thereof, wherein
a) R^{d} is —(C(R⁶)₂)ₜC₆-C₁₀ aryl or —(C(R⁶)₂)ₜ-5- or 6-membered heteroaryl);
b) L is —(C(R⁵)₂)ₘY¹(C(R⁵)₂)ₚ;
c) R⁷ is A.
d) X, R^{d}, R^{f}, R^{j}, A, R⁵, Y¹, m, and p are defined for Formula (I).
   In certain embodiments, L is —SCH₂— or —NHCH₂—. In certain embodiments, A is —(C(R⁶)₂)ᵣCO₂R^{x} or —(CH₂)ᵣtetrazole, wherein —(CH₂)ᵣtetrazole is optionally substituted with C₁-C₆ alkyl. In certain embodiments, A is —(C(R⁶)₂)ᵣCOOH or —(CH₂)ᵣtetrazole, wherein —(CH₂)ᵣtetrazole is optionally substituted with C₁-C₆ alkyl. In certain embodiments, A is —COOH, —CH₂COOH, -tetrazole, or —(CH₂)ᵣtetrazole, wherein tetrazole and —(CH₂)ᵣtetrazole are optionally substituted with C₁-C₆ alkyl. In certain embodiments, Formula (Ie) has one, two, three, or four of the features (a) to (d).

In certain embodiments, with certain above features for Formula (I), the present disclosure provides a compound of formula (If) having at least one of the following features:

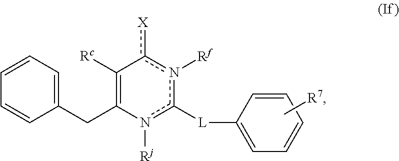

and pharmaceutically salts and tautomers thereof, wherein
a) L is —(C(R⁵)₂)ₘY¹(C(R⁵)₂)ₚ;
b) R⁷ is A;
c) X, R^{e}, R^{f}, R^{j}, A, R⁵, Y¹, m, and p are defined for Formula (I).
   In certain embodiments, L is —SCH₂— or —NHCH₂—. In certain embodiments, A is —(C(R⁶)₂)ᵣCO₂R^{x} or —(CH₂)ᵣtetrazole, wherein —(CH₂)ᵣtetrazole is optionally substituted with C₁-C₆ alkyl. In certain embodiments, A is —(C(R⁶)₂)ᵣCOOH or —(CH₂)ᵣtetrazole, wherein —(CH₂)ᵣtetrazole is optionally substituted with C₁-C₆ alkyl. In certain embodiments, A is —COOH, —CH₂COOH, -tetrazole, or —(CH₂)ᵣtetrazole, wherein tetrazole and —(CH₂)ᵣtetrazole are optionally substituted with C₁-C₆ alkyl. In certain embodiments, R^{c} is CN. In certain embodiments, Formula (If) has one, two, or three of the features (a) to (c).

In certain embodiments, with certain above features for Formula (I), the present disclosure provides a compound of formula (Ig) having at least one of the following features:

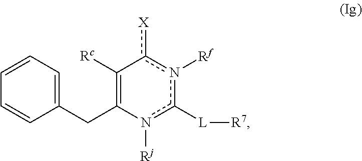

and pharmaceutically salts and tautomers thereof, wherein
a) L is —(C(R⁵)₂)ₘY¹(C(R⁵)₂)ₚ;
b) R⁷ is A;
c) X, R^{d}, R^{f}, R^{j}, A, R⁵, Y¹, m, and p are defined for Formula (I).
   In certain embodiments, L is —SCH₂— or —NHCH₂—. In certain embodiments, A is —(C(R⁶)₂)ᵣCO₂R^{x} or —(CH₂)ᵣtetrazole, wherein —(CH₂)ᵣtetrazole is optionally substituted with C₁-C₆ alkyl. In certain embodiments, A is —(C(R⁶)₂)ᵣCOOH or —(CH₂)ᵣtetrazole, wherein —(CH₂)ᵣtetrazole is optionally substituted with C₁-C₆ alkyl. In certain embodiments, A is —COOH, —CH₂COOH, -tetrazole, or —(CH₂)ᵣtetrazole, wherein tetrazole and —(CH₂)ᵣtetrazole are optionally substituted with C₁-C₆ alkyl. In certain embodiments, Formula (Ig) has one, two, or three of the features (a) to (c).

In some embodiments, the compound of Formula (I) is a compound selected from:

| Cpd | |
|---|---|
| No. | Structure |
| I-1 | 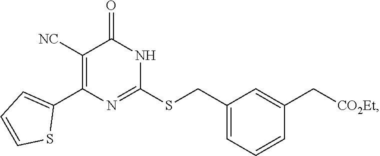 [SMILES: CCOC(=O)Cc1cccc(CSc2nc(-c3cccs3)c(C#N)c(=O)[nH]2)c1] |
| I-2 | 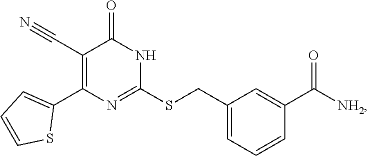 [SMILES: N#Cc1c(-c2cccs2)nc(SCc2cccc(C(N)=O)c2)[nH]c1=O] |
| I-3 | 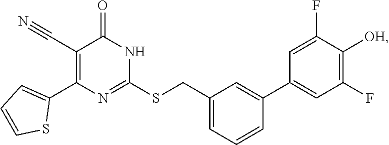 [SMILES: N#Cc1c(-c2cccs2)nc(SCc2cccc(-c3cc(F)c(O)c(F)c3)c2)[nH]c1=O] |
| I-4 | 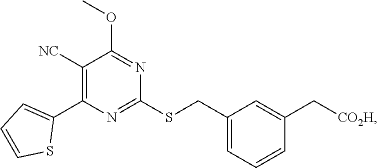 [SMILES: COc1nc(SCc2cccc(CC(=O)O)c2)nc(-c2cccs2)c1C#N] |
| I-5 | 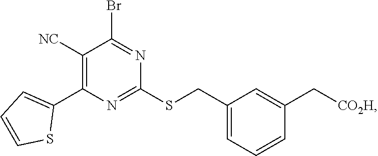 [SMILES: N#Cc1c(Br)nc(SCc2cccc(CC(=O)O)c2)nc1-c1cccs1] |
| I-6 | 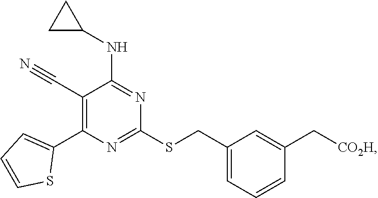 [SMILES: N#Cc1c(NC2CC2)nc(SCc2cccc(CC(=O)O)c2)nc1-c1cccs1] |
| I-7 | 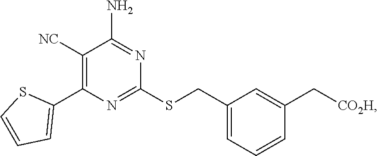 [SMILES: N#Cc1c(N)nc(SCc2cccc(CC(=O)O)c2)nc1-c1cccs1] |
| I-8 | 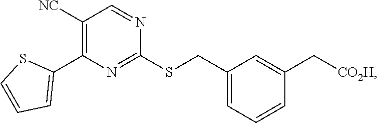 [SMILES: N#Cc1cnc(SCc2cccc(CC(=O)O)c2)nc1-c1cccs1] |
| I-9 | 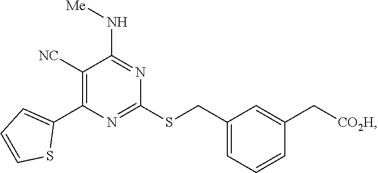 [SMILES: CNc1nc(SCc2cccc(CC(=O)O)c2)nc(-c2cccs2)c1C#N] |
| I-10 | 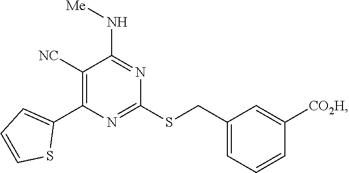 [SMILES: CNc1nc(SCc2cccc(C(=O)O)c2)nc(-c2cccs2)c1C#N] |
| I-11 | 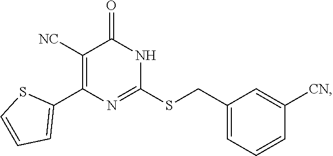 [SMILES: N#Cc1cccc(CSc2nc(-c3cccs3)c(C#N)c(=O)[nH]2)c1] |
| I-12 | 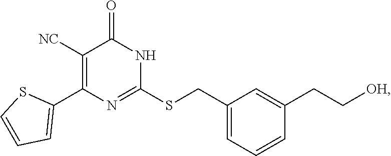 [SMILES: N#Cc1c(-c2cccs2)nc(SCc2cccc(CCO)c2)[nH]c1=O] |
| I-13 | 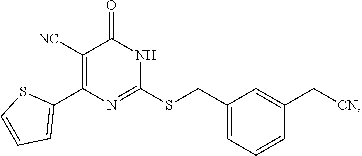 [SMILES: N#CCc1cccc(CSc2nc(-c3cccs3)c(C#N)c(=O)[nH]2)c1] |
| I-14 | 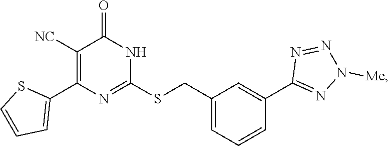 [SMILES: Cn1nnc(-c2cccc(CSc3nc(-c4cccs4)c(C#N)c(=O)[nH]3)c2)n1] |
| I-15 | 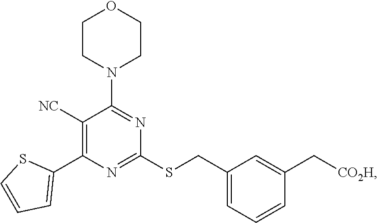 [SMILES: N#Cc1c(-c2cccs2)nc(SCc2cccc(CC(=O)O)c2)nc1N1CCOCC1] |
| I-16 | 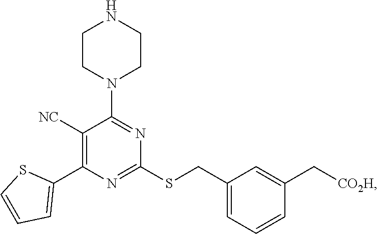 [SMILES: N#Cc1c(-c2cccs2)nc(SCc2cccc(CC(=O)O)c2)nc1N1CCNCC1] |
| I-17 | 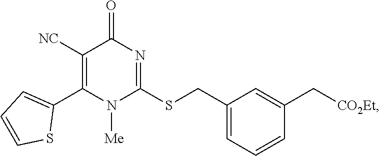 [SMILES: CCOC(=O)Cc1cccc(CSc2nc(=O)c(C#N)c(-c3cccs3)n2C)c1] |
| I-18 | 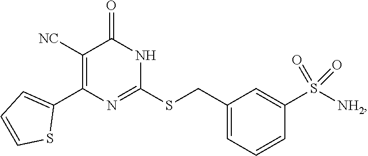 [SMILES: N#Cc1c(-c2cccs2)nc(SCc2cccc(S(N)(=O)=O)c2)[nH]c1=O] |
| I-19 | 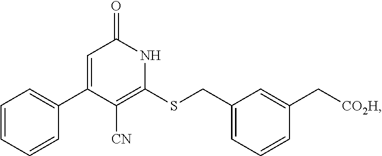 [SMILES: N#Cc1c(-c2ccccc2)cc(=O)[nH]c1SCc1cccc(CC(=O)O)c1] |
| I-20 | 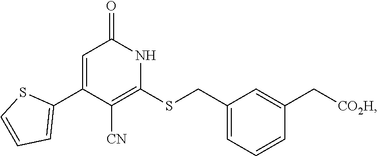 [SMILES: N#Cc1c(-c2cccs2)cc(=O)[nH]c1SCc1cccc(CC(=O)O)c1] |
| I-21 | 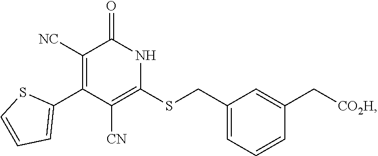 [SMILES: N#Cc1c(SCc2cccc(CC(=O)O)c2)[nH]c(=O)c(C#N)c1-c1cccs1] |
| I-22 | 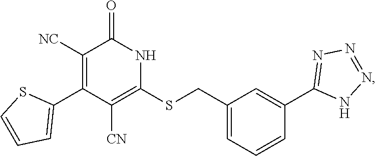 [SMILES: N#Cc1c(SCc2cccc(-c3nnn[nH]3)c2)[nH]c(=O)c(C#N)c1-c1cccs1] |
| I-23 | 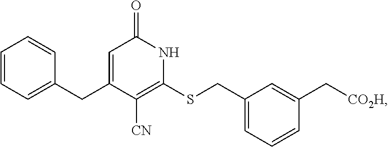 [SMILES: N#Cc1c(Cc2ccccc2)cc(=O)[nH]c1SCc1cccc(CC(=O)O)c1] |
| I-24 | 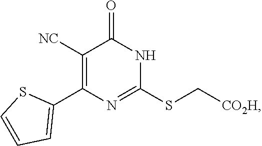 [SMILES: N#Cc1c(-c2cccs2)nc(SCC(=O)O)[nH]c1=O] |
| I-25 | 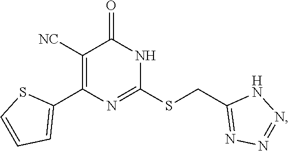 [SMILES: N#Cc1c(-c2cccs2)nc(SCc2nnn[nH]2)[nH]c1=O] |
| I-26 | 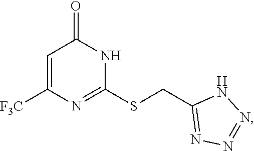 [SMILES: O=c1cc(C(F)(F)F)nc(SCc2nnn[nH]2)[nH]1] |
| I-27 | 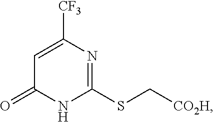 [SMILES: O=C(O)CSc1nc(C(F)(F)F)cc(=O)[nH]1] |
| I-28 | 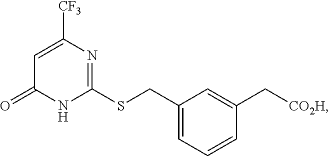 [SMILES: O=C(O)Cc1cccc(CSc2nc(C(F)(F)F)cc(=O)[nH]2)c1] |
| I-29 | 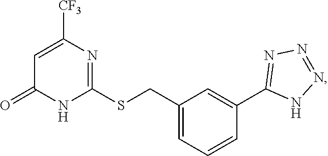 [SMILES: O=c1cc(C(F)(F)F)nc(SCc2cccc(-c3nnn[nH]3)c2)[nH]1] |
| I-30 | 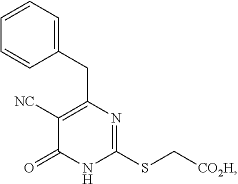 [SMILES: N#Cc1c(Cc2ccccc2)nc(SCC(=O)O)[nH]c1=O] |
| I-31 | 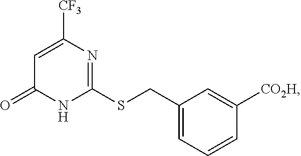 [SMILES: O=C(O)c1cccc(CSc2nc(C(F)(F)F)cc(=O)[nH]2)c1] |
| I-32 | 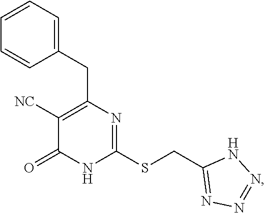 [SMILES: N#Cc1c(Cc2ccccc2)nc(SCc2nnn[nH]2)[nH]c1=O] |
| I-33 | 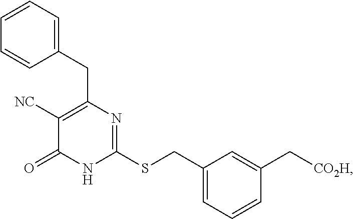 [SMILES: N#Cc1c(Cc2ccccc2)nc(SCc2cccc(CC(=O)O)c2)[nH]c1=O] |
| I-34 | 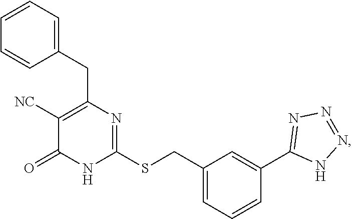 [SMILES: N#Cc1c(Cc2ccccc2)nc(SCc2cccc(-c3nnn[nH]3)c2)[nH]c1=O] |
| I-35 | 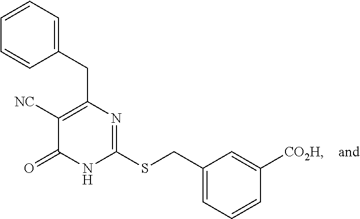 [SMILES: N#Cc1c(Cc2ccccc2)nc(SCc2cccc(C(=O)O)c2)[nH]c1=O ; "and"] |
| I-36 | 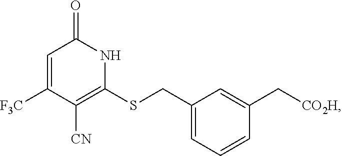 [SMILES: N#Cc1c(C(F)(F)F)cc(=O)[nH]c1SCc1cccc(CC(=O)O)c1] |

or a pharmaceutically acceptable salt or tautomer thereof.

In some embodiments, the compound of Formula (I) or (II) is a compound selected from:

| | |
|---|---|
| I-37 | 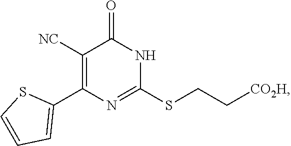 [SMILES: N#Cc1c(-c2cccs2)nc(SCCC(=O)O)[nH]c1=O] |
| I-38 | 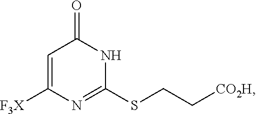 |
| I-39 | 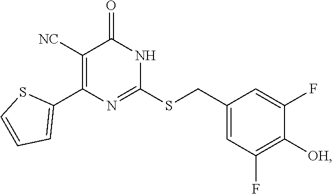 [SMILES: N#Cc1c(-c2cccs2)nc(SCc2cc(F)c(O)c(F)c2)[nH]c1=O] |
| I-40 | 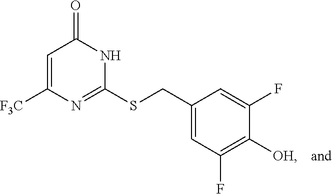 [SMILES: O=c1cc(C(F)(F)F)nc(SCc2cc(F)c(O)c(F)c2)[nH]1 ; "and"] |
| I-41 | 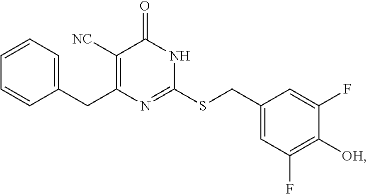 [SMILES: N#Cc1c(Cc2ccccc2)nc(SCc2cc(F)c(O)c(F)c2)[nH]c1=O] |
| I-42 | 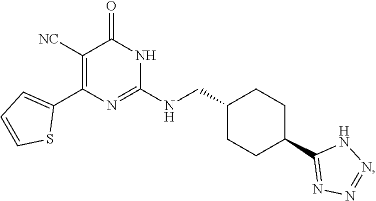 [SMILES: N#Cc1c(-c2cccs2)nc(NC[C@H]2CC[C@H](c3nnn[nH]3)CC2)[nH]c1=O] |
| I-43 | 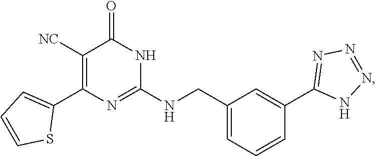 [SMILES: N#Cc1c(-c2cccs2)nc(NCc2cccc(-c3nnn[nH]3)c2)[nH]c1=O] |
| I-44 | 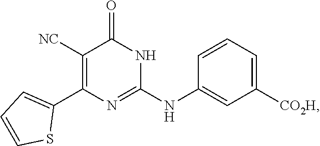 [SMILES: N#Cc1c(-c2cccs2)nc(Nc2cccc(C(=O)O)c2)[nH]c1=O] |
| I-45 | 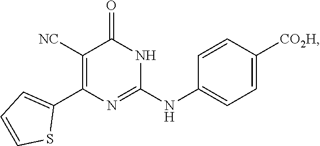 [SMILES: N#Cc1c(-c2cccs2)nc(Nc2ccc(C(=O)O)cc2)[nH]c1=O] |
| I-46 | 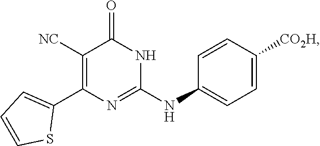 |
| I-47 | 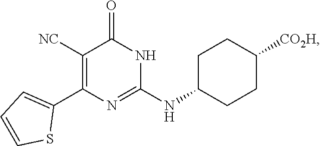 [SMILES: N#Cc1c(-c2cccs2)nc(N[C@H]2CC[C@@H](C(=O)O)CC2)[nH]c1=O] |
| I-48 | 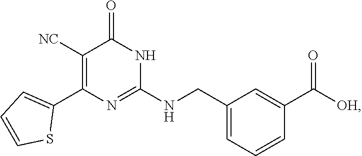 [SMILES: N#Cc1c(-c2cccs2)nc(NCc2cccc(C(=O)O)c2)[nH]c1=O] |
| I-49 | 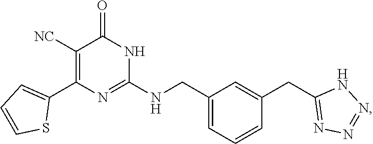 [SMILES: N#Cc1c(-c2cccs2)nc(NCc2cccc(Cc3nnn[nH]3)c2)[nH]c1=O] |
| I-50 | 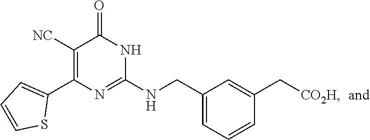 [SMILES: N#Cc1c(-c2cccs2)nc(NCc2cccc(CC(=O)O)c2)[nH]c1=O ; "and"] |
| I-51 | 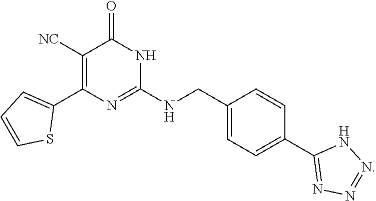 [SMILES: N#Cc1c(-c2cccs2)nc(NCc2ccc(-c3nnn[nH]3)cc2)[nH]c1=O] |

In some embodiments, the compound of Formula (I) is a compound, or a pharmaceutically acceptable salt or tautomer thereof, selected from:

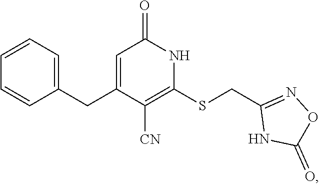 [SMILES: N#Cc1c(Cc2ccccc2)cc(=O)[nH]c1SCc1noc(=O)[nH]1]

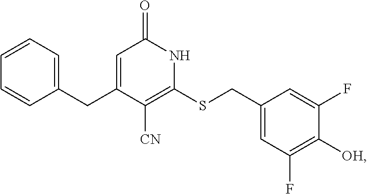 [SMILES: N#Cc1c(Cc2ccccc2)cc(=O)[nH]c1SCc1cc(F)c(O)c(F)c1]

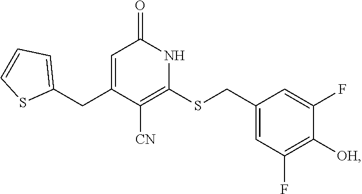 [SMILES: N#Cc1c(Cc2cccs2)cc(=O)[nH]c1SCc1cc(F)c(O)c(F)c1]

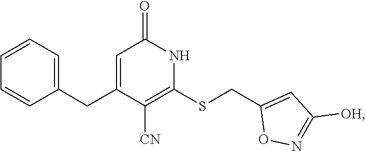 [SMILES: N#Cc1c(Cc2ccccc2)cc(=O)[nH]c1SCc1cc(O)no1]

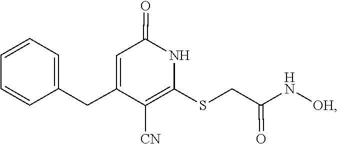 [SMILES: N#Cc1c(Cc2ccccc2)cc(=O)[nH]c1SCC(=O)NO]

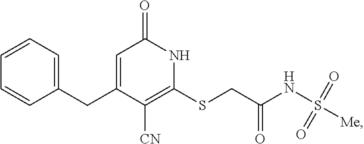 [SMILES: CS(=O)(=O)NC(=O)CSc1[nH]c(=O)cc(Cc2ccccc2)c1C#N]

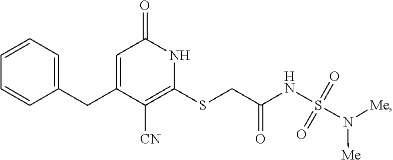 [SMILES: CN(C)S(=O)(=O)NC(=O)CSc1[nH]c(=O)cc(Cc2ccccc2)c1C#N]

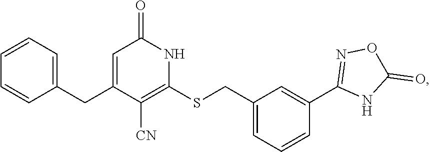 [SMILES: N#Cc1c(Cc2ccccc2)cc(=O)[nH]c1SCc1cccc(-c2noc(=O)[nH]2)c1]

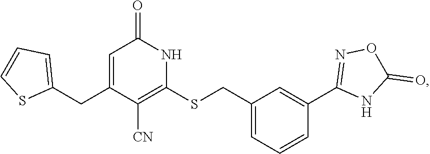 [SMILES: N#Cc1c(Cc2cccs2)cc(=O)[nH]c1SCc1cccc(-c2noc(=O)[nH]2)c1]

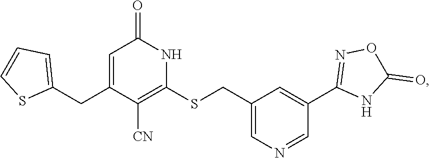 [SMILES: N#Cc1c(Cc2cccs2)cc(=O)[nH]c1SCc1cncc(-c2noc(=O)[nH]2)c1]

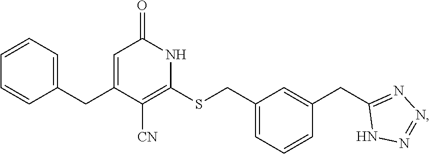 [SMILES: N#Cc1c(Cc2ccccc2)cc(=O)[nH]c1SCc1cccc(Cc2nnn[nH]2)c1]

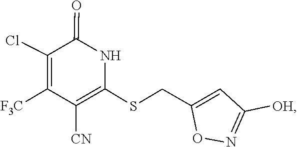 [SMILES: N#Cc1c(SCc2cc(O)no2)[nH]c(=O)c(Cl)c1C(F)(F)F]

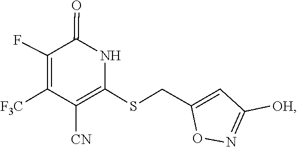 [SMILES: N#Cc1c(SCc2cc(O)no2)[nH]c(=O)c(F)c1C(F)(F)F]

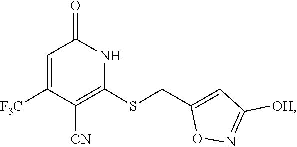 [SMILES: N#Cc1c(C(F)(F)F)cc(=O)[nH]c1SCc1cc(O)no1]

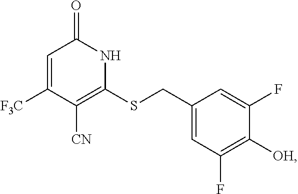 [SMILES: N#Cc1c(C(F)(F)F)cc(=O)[nH]c1SCc1cc(F)c(O)c(F)c1]

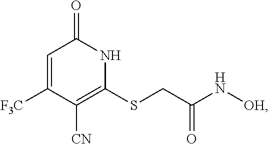 [SMILES: N#Cc1c(C(F)(F)F)cc(=O)[nH]c1SCC(=O)NO]

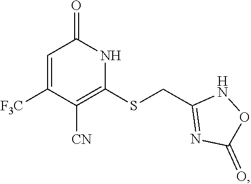 [SMILES: N#Cc1c(C(F)(F)F)cc(=O)[nH]c1SCc1nc(=O)o[nH]1]

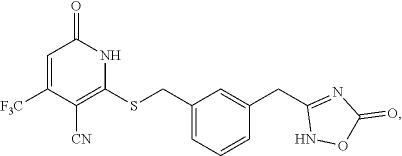 [SMILES: N#Cc1c(C(F)(F)F)cc(=O)[nH]c1SCc1cccc(Cc2nc(=O)o[nH]2)c1]

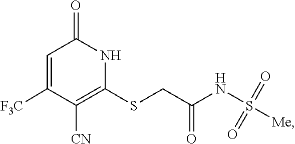 [SMILES: CS(=O)(=O)NC(=O)CSc1[nH]c(=O)cc(C(F)(F)F)c1C#N]

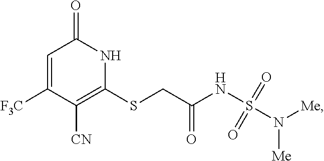

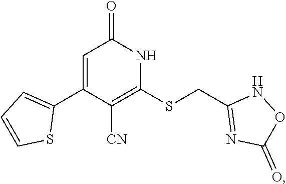 [SMILES: N#Cc1c(-c2cccs2)cc(=O)[nH]c1SCc1nc(=O)o[nH]1]

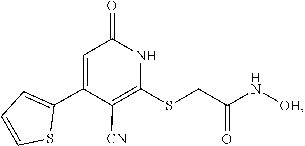 [SMILES: N#Cc1c(-c2cccs2)cc(=O)[nH]c1SCC(=O)NO]

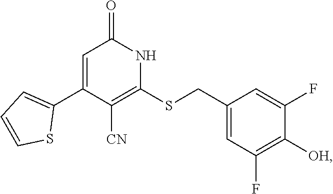 [SMILES: N#Cc1c(-c2cccs2)cc(=O)[nH]c1SCc1cc(F)c(O)c(F)c1]

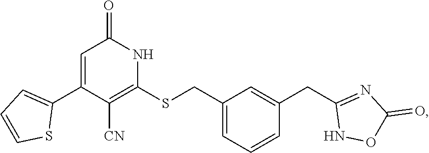 [SMILES: N#Cc1c(-c2cccs2)cc(=O)[nH]c1SCc1cccc(Cc2nc(=O)o[nH]2)c1]

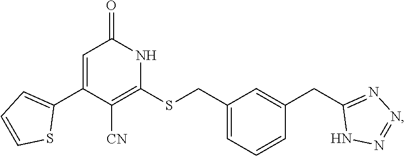 [SMILES: N#Cc1c(-c2cccs2)cc(=O)[nH]c1SCc1cccc(Cc2nnn[nH]2)c1]

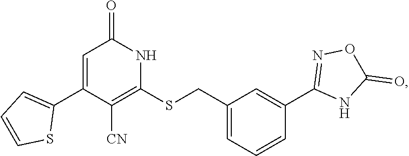 [SMILES: N#Cc1c(-c2cccs2)cc(=O)[nH]c1SCc1cccc(-c2noc(=O)[nH]2)c1]

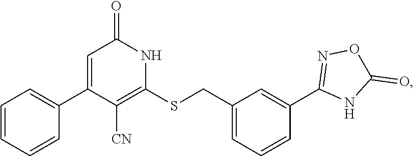 [SMILES: N#Cc1c(-c2ccccc2)cc(=O)[nH]c1SCc1cccc(-c2noc(=O)[nH]2)c1]

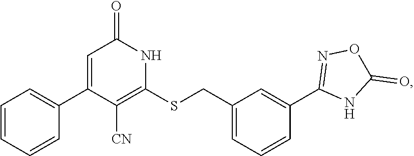 [SMILES: N#Cc1c(-c2ccccc2)cc(=O)[nH]c1SCc1cccc(-c2noc(=O)[nH]2)c1]

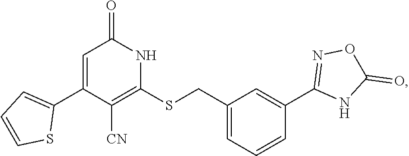 [SMILES: N#Cc1c(-c2cccs2)cc(=O)[nH]c1SCc1cccc(-c2noc(=O)[nH]2)c1]

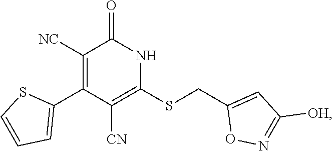 [SMILES: N#Cc1c(SCc2cc(O)no2)[nH]c(=O)c(C#N)c1-c1cccs1]

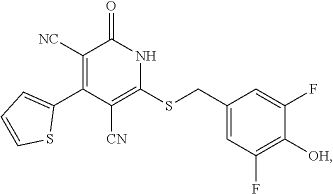 [SMILES: N#Cc1c(SCc2cc(F)c(O)c(F)c2)[nH]c(=O)c(C#N)c1-c1cccs1]

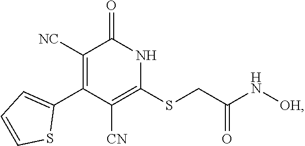 [SMILES: N#Cc1c(SCC(=O)NO)[nH]c(=O)c(C#N)c1-c1cccs1]

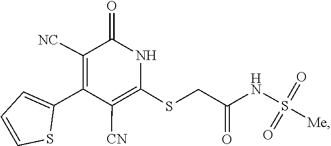 [SMILES: CS(=O)(=O)NC(=O)CSc1[nH]c(=O)c(C#N)c(-c2cccs2)c1C#N]

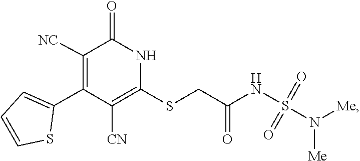

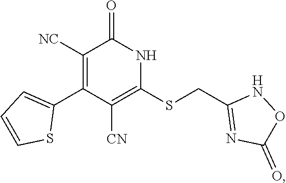 [SMILES: N#Cc1c(SCc2nc(=O)o[nH]2)[nH]c(=O)c(C#N)c1-c1cccs1]

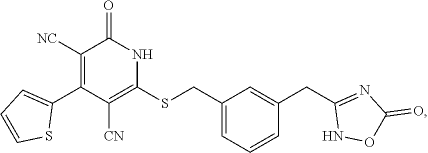 [SMILES: N#Cc1c(SCc2cccc(Cc3nc(=O)o[nH]3)c2)[nH]c(=O)c(C#N)c1-c1cccs1]

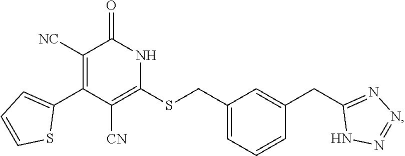 [SMILES: N#Cc1c(SCc2cccc(Cc3nnn[nH]3)c2)[nH]c(=O)c(C#N)c1-c1cccs1]

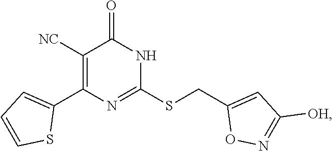 [SMILES: N#Cc1c(-c2cccs2)nc(SCc2cc(O)no2)[nH]c1=O]

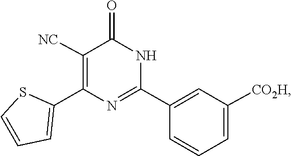 [SMILES: N#Cc1c(-c2cccs2)nc(-c2cccc(C(=O)O)c2)[nH]c1=O]

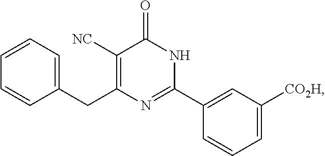 [SMILES: N#Cc1c(Cc2ccccc2)nc(-c2cccc(C(=O)O)c2)[nH]c1=O]

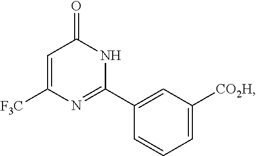 [SMILES: O=C(O)c1cccc(-c2nc(C(F)(F)F)cc(=O)[nH]2)c1]

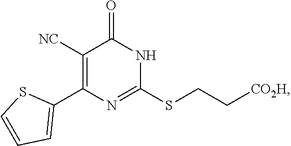 [SMILES: N#Cc1c(-c2cccs2)nc(SCCC(=O)O)[nH]c1=O]

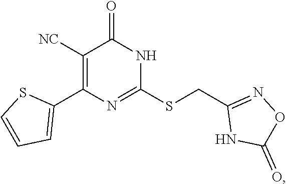 [SMILES: N#Cc1c(-c2cccs2)nc(SCc2noc(=O)[nH]2)[nH]c1=O]

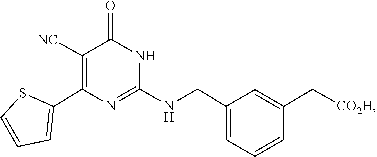 [SMILES: N#Cc1c(-c2cccs2)nc(NCc2cccc(CC(=O)O)c2)[nH]c1=O]

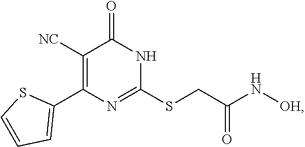 [SMILES: N#Cc1c(-c2cccs2)nc(SCC(=O)NO)[nH]c1=O]

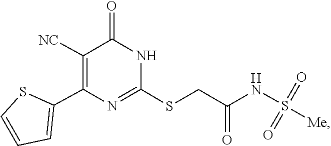 [SMILES: CS(=O)(=O)NC(=O)CSc1nc(-c2cccs2)c(C#N)c(=O)[nH]1]

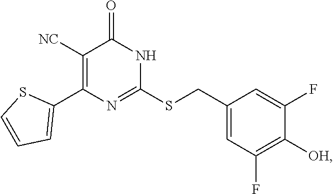 [SMILES: N#Cc1c(-c2cccs2)nc(SCc2cc(F)c(O)c(F)c2)[nH]c1=O]

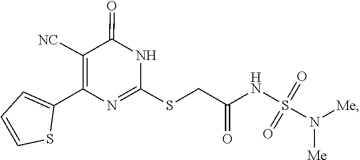 [SMILES: CN(C)S(=O)(=O)NC(=O)CSc1nc(-c2cccs2)c(C#N)c(=O)[nH]1]

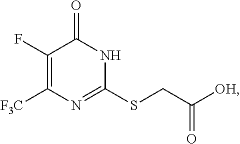 [SMILES: O=C(O)CSc1nc(C(F)(F)F)c(F)c(=O)[nH]1]

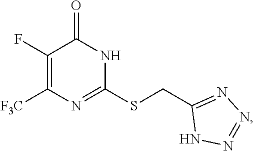 [SMILES: O=c1[nH]c(SCc2nnn[nH]2)nc(C(F)(F)F)c1F]

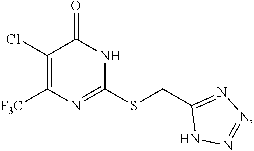 [SMILES: O=c1[nH]c(SCc2nnn[nH]2)nc(C(F)(F)F)c1Cl]

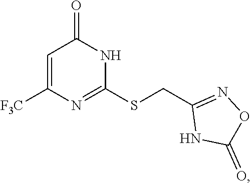 [SMILES: O=c1cc(C(F)(F)F)nc(SCc2noc(=O)[nH]2)[nH]1]

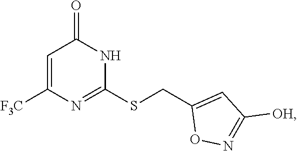 [SMILES: O=c1cc(C(F)(F)F)nc(SCc2cc(O)no2)[nH]1]

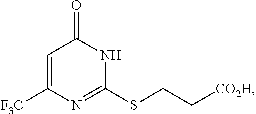 [SMILES: O=C(O)CCSc1nc(C(F)(F)F)cc(=O)[nH]1]

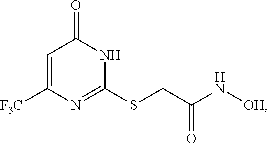 [SMILES: O=C(CSc1nc(C(F)(F)F)cc(=O)[nH]1)NO]

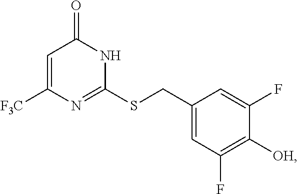 [SMILES: O=c1cc(C(F)(F)F)nc(SCc2cc(F)c(O)c(F)c2)[nH]1]

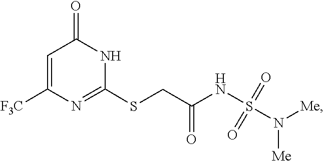 [SMILES: CN(C)S(=O)(=O)NC(=O)CSc1nc(C(F)(F)F)cc(=O)[nH]1]

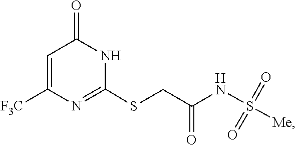 [SMILES: CS(=O)(=O)NC(=O)CSc1nc(C(F)(F)F)cc(=O)[nH]1]

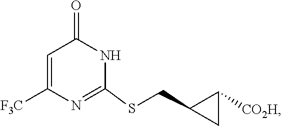 [SMILES: O=C(O)[C@@H]1C[C@H]1CSc1nc(C(F)(F)F)cc(=O)[nH]1]

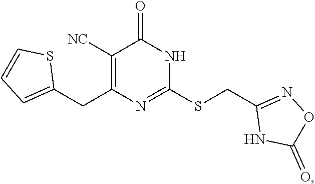 [SMILES: N#Cc1c(Cc2cccs2)nc(SCc2noc(=O)[nH]2)[nH]c1=O]

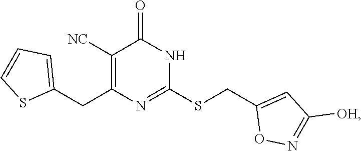 [SMILES: N#Cc1c(Cc2cccs2)nc(SCc2cc(O)no2)[nH]c1=O]

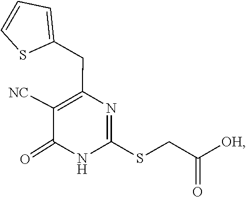 [SMILES: N#Cc1c(Cc2cccs2)nc(SCC(=O)O)[nH]c1=O]

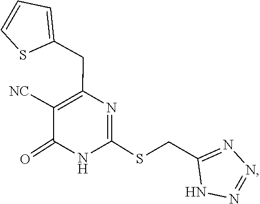 [SMILES: N#Cc1c(Cc2cccs2)nc(SCc2nnn[nH]2)[nH]c1=O]

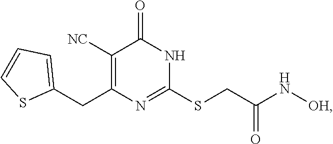 [SMILES: N#Cc1c(Cc2cccs2)nc(SCC(=O)NO)[nH]c1=O]

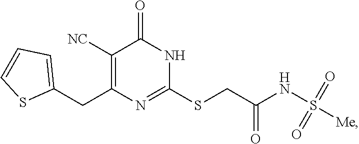 [SMILES: CS(=O)(=O)NC(=O)CSc1nc(Cc2cccs2)c(C#N)c(=O)[nH]1]

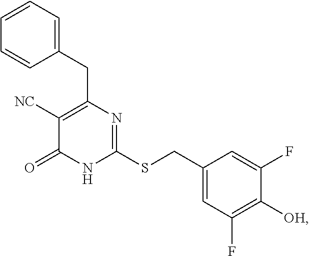 [SMILES: N#Cc1c(Cc2ccccc2)nc(SCc2cc(F)c(O)c(F)c2)[nH]c1=O]

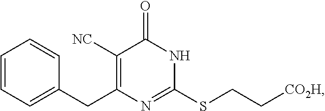 [SMILES: N#Cc1c(Cc2ccccc2)nc(SCCC(=O)O)[nH]c1=O]

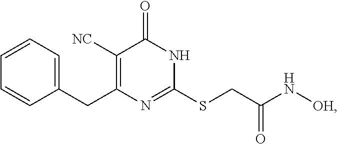 [SMILES: N#Cc1c(Cc2ccccc2)nc(SCC(=O)NO)[nH]c1=O]

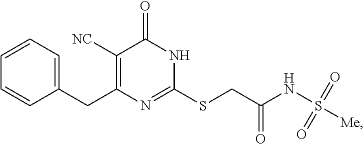 [SMILES: CS(=O)(=O)NC(=O)CSc1nc(Cc2ccccc2)c(C#N)c(=O)[nH]1]

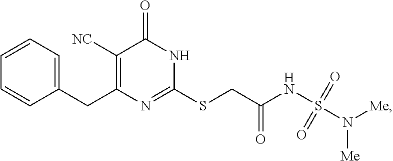 [SMILES: CN(C)S(=O)(=O)NC(=O)CSc1nc(Cc2ccccc2)c(C#N)c(=O)[nH]1]

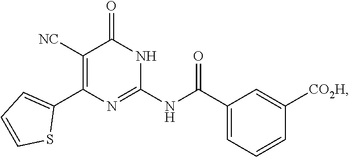 [SMILES: N#Cc1c(-c2cccs2)nc(NC(=O)c2cccc(C(=O)O)c2)[nH]c1=O]

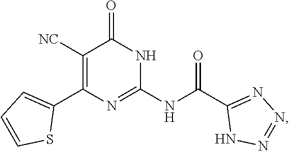 [SMILES: N#Cc1c(-c2cccs2)nc(NC(=O)c2nnn[nH]2)[nH]c1=O]

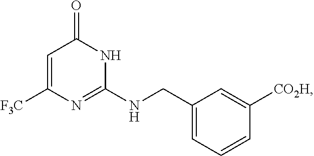 [SMILES: O=C(O)c1cccc(CNc2nc(C(F)(F)F)cc(=O)[nH]2)c1]

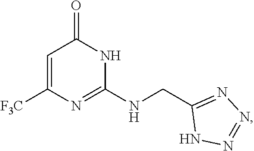 [SMILES: O=c1cc(C(F)(F)F)nc(NCc2nnn[nH]2)[nH]1]

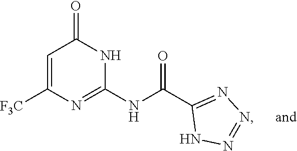 [SMILES: O=C(Nc1nc(C(F)(F)F)cc(=O)[nH]1)c1nnn[nH]1 ; "and"]

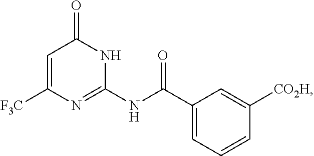 [SMILES: O=C(O)c1cccc(C(=O)Nc2nc(C(F)(F)F)cc(=O)[nH]2)c1]

or a pharmaceutically acceptable salt or tautomer thereof.

In some embodiments, the compound of Formula (I) or (II) is a compound, or a pharmaceutically acceptable salt or tautomer thereof, selected from:

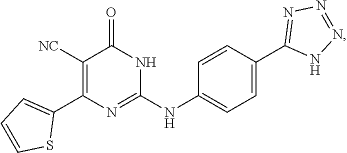 [SMILES: N#Cc1c(-c2cccs2)nc(Nc2ccc(-c3nnn[nH]3)cc2)[nH]c1=O]

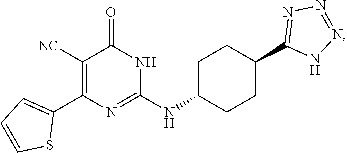 [SMILES: N#Cc1c(-c2cccs2)nc(N[C@H]2CC[C@H](c3nnn[nH]3)CC2)[nH]c1=O]

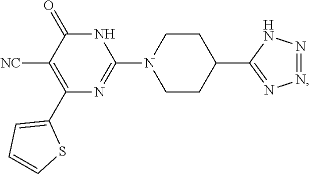 [SMILES: N#Cc1c(-c2cccs2)nc(N2CCC(c3nnn[nH]3)CC2)[nH]c1=O]

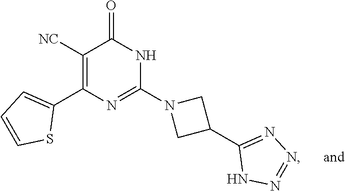 [SMILES: N#Cc1c(-c2cccs2)nc(N2CC(c3nnn[nH]3)C2)[nH]c1=O ; "and"]

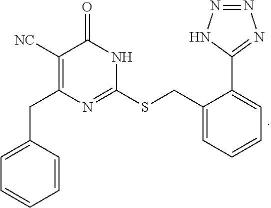 [SMILES: N#Cc1c(Cc2ccccc2)nc(SCc2ccccc2-c2nnn[nH]2)[nH]c1=O]

It should be understood, that such references are intended to encompass not only the above general formula, but also each and every of the embodiments, etc. discussed in the following. It should also be understood, that unless stated to the opposite, such references also encompass isomers, mixtures of isomers, pharmaceutically acceptable salts, solvates and prodrugs of the compounds of Formula (I) or (II).

The term “alkyl” as used herein refers to a saturated, straight, or branched hydrocarbon chain. The hydrocarbon chain preferably contains from one to eight carbon atoms (C₁₋₈-alkyl), more preferred from one to six carbon atoms (C₁₋₆-alkyl), in particular from one to four carbon atoms (C₁₋₄-alkyl), including methyl, ethyl, propyl, isopropyl, butyl, isobutyl, secondary butyl, tertiary butyl, pentyl, isopentyl, neopentyl, tertiary pentyl, hexyl, isohexyl, heptyl and octyl. In a preferred embodiment “alkyl” represents a C₁₋₄-alkyl group, which may in particular include methyl, ethyl, propyl, isopropyl, butyl, isobutyl, secondary butyl, and tertiary butyl. Correspondingly, the term “alkylene” means the corresponding biradical (-alkyl-).

The term “cycloalkyl” or “carbocycle” as used herein refers to a cyclic alkyl group, preferably containing from three to ten carbon atoms (C₃₋₁₀-cycloalkyl or C₃₋₁₀-carbocycle), such as from three to eight carbon atoms (C₃₋₈-cycloalkyl or C₃₋₁₀-carbocycle), preferably from three to six carbon atoms (C₃₋₆-cycloalkyl or C₃₋₁₀-carbocycle), including cyclopropyl, cyclobutyl, cyclopentyl, cyclohexyl, cycloheptyl, and cyclooctyl. Furthermore, the term “cycloalkyl” as used herein may also include polycyclic groups such as for example bicyclo[2.2.2]octyl, bicyclo[2.2.1]heptanyl, decalinyl and adamantyl. Correspondingly, the term “cycloalkylene” means the corresponding biradical (-cycloalkyl-). Alkyl and cycloalkyl groups may be optionally substituted with 1-4 substituents. Examples of substituents on alkyl groups include, but are not limited to, alkyl, alkenyl, alkynyl, halogen, haloalkyl, alkoxy, heteroaryl, aryl, carbocyclyl, hydroxyl, carbamoyl, oxo, and —CN.

The term “alkenyl” as used herein refers to a straight or branched hydrocarbon chain or cyclic hydrocarbons containing one or more double bonds, including di-enes, tri-enes, and poly-enes. Typically, the alkenyl group comprises from two to eight carbon atoms (C₂₋₈-alkenyl), such as from two to six carbon atoms (C₂₋₆-alkenyl), in particular from two to four carbon atoms (C₂₋₄-alkenyl), including at least one double bond. Examples of alkenyl groups include ethenyl; 1- or 2-propenyl; 1-, 2- or 3-butenyl, or 1,3-but-dienyl; 1-, 2-, 3-, 4- or 5-hexenyl, or 1,3-hex-dienyl, or 1,3,5-hex-trienyl; 1-, 2-, 3-, 4-, 5-, 6-, or 7-octenyl, or 1,3-octadienyl, or 1,3,5-octatrienyl, or 1,3,5,7-octatetraenyl, or cyclohexenyl. Correspondingly, the term “alkenylene” means the corresponding biradical (-alkenyl-). Alkenyl groups may be optionally substituted with 1-4 substituents. Examples of substituents on alkenyl groups include, but are not limited to, alkyl, alkenyl, alkynyl, halogen, haloalkyl, alkoxy, heteroaryl, aryl, carbocyclyl, hydroxyl, carbamoyl, oxo, and —CN.

The term “alkynyl” as used herein refers to a straight or branched hydrocarbon chain containing one or more triple bonds, including di-ynes, tri-ynes, and poly-ynes. Typically, the alkynyl group comprises of from two to eight carbon atoms (C₂₋₈-alkynyl), such as from two to six carbon atoms (C₂₋₆-alkynyl), in particular from two to four carbon atoms (C₂₋₄-alkynyl), including at least one triple bond. Examples of preferred alkynyl groups include ethynyl; 1- or 2-propynyl; 1-, 2- or 3-butynyl, or 1,3-but-diynyl; 1-, 2-, 3-, 4- or 5-hexynyl, or 1,3-hex-diynyl, or 1,3,5-hex-triynyl; 1-, 2-, 3-, 4-, 5-, 6-, or 7-octynyl, or 1,3-oct-diynyl, or 1,3,5-oct-triynyl, or 1,3,5,7-oct-tetraynyl. Correspondingly, the term “alkynylene” means the corresponding biradical (-alkynyl-). Alkynyl groups may be optionally substituted with 1-4 substituents. Examples of substituents on alkynyl groups include, but are not limited to, alkyl, alkenyl, alkynyl, halogen, haloalkyl, alkoxy, heteroaryl, aryl, carbocyclyl, hydroxyl, carbamoyl, oxo, and —CN.

The terms “halo” and “halogen” as used herein refer to fluoro, chloro, bromo or iodo. Thus a trihalomethyl group represents, e.g., a trifluoromethyl group, or a trichloromethyl group. Preferably, the terms “halo” and “halogen” designate fluoro or chloro.

The term “haloalkyl” as used herein refers to an alkyl group, as defined herein, which is substituted one or more times with one or more halogen. Examples of haloalkyl groups include, but are not limited to, trifluoromethyl, difluoromethyl, pentafluoroethyl, trichloromethyl, etc.

The term “alkoxy” as used herein refers to an “alkyl-O—” group, wherein alkyl is as defined above.

The term “hydroxyalkyl” as used herein refers to an alkyl group (as defined hereinabove), which alkyl group is substituted one or more times with hydroxy. Examples of hydroxyalkyl groups include HO—CH₂—, HO—CH₂—CH₂—, and CH₃—CH(OH)—.

The term “oxy” as used herein refers to an “—O—” group.

The term “oxo” as used herein refers to an “═O” group.

The term “amine” as used herein refers to primary (R—NH₂, R≠H), secondary ((R)₂—NH, (R)₂≠H) and tertiary ((R)₃—N, R≠H) amines. A substituted amine is intended to mean an amine where at least one of the hydrogen atoms has been replaced by the substituent.

The term “carbamoyl” as used herein refers to a “H₂N(C═O)—” group.

The term “aryl” as used herein, unless otherwise indicated, includes carbocyclic aromatic ring systems derived from an aromatic hydrocarbon by removal of a hydrogen atom. Aryl furthermore includes bi-, tri-, and polycyclic ring systems. Examples of preferred aryl moieties include phenyl, naphthyl, indenyl, indanyl, fluorenyl, biphenyl, indenyl, naphthyl, anthracenyl, phenanthrenyl, pentalenyl, azulenyl, and biphenylenyl. Preferred “aryl” is phenyl, naphthyl or indanyl, in particular phenyl, unless otherwise stated. Any aryl used may be optionally substituted. Correspondingly, the term “arylene” means the corresponding biradical (-aryl-). Aryl groups may be optionally substituted with 1-4 substituents. Examples of substituents on aryl groups include, but are not limited to, alkyl, alkenyl, alkynyl, halogen, haloalkyl, alkoxy, heteroaryl, aryl, carbocyclyl, hydroxyl, and —CN.

The term “heteroaryl” as used herein, refers to aromatic groups containing one or more heteroatoms selected from O, S, and N, preferably from one to four heteroatoms, and more preferably from one to three heteroatoms. Heteroaryl furthermore includes bi-, tri-, and polycyclic groups, wherein at least one ring of the group is aromatic, and at least one of the rings contains a heteroatom selected from O, S, and N. Heteroaryl also include ring systems substituted with one or more oxo moieties. Examples of preferred heteroaryl moieties include N-hydroxytetrazolyl, N-hydroxytriazolyl, N-hydroxyimidazolyl, furanyl, triazolyl, pyranyl, thiadiazinyl, benzothiophenyl, dihydro-benzo[b]thiophenyl, xanthenyl, isoindanyl, acridinyl, benzisoxazolyl, quinolinyl, isoquinolinyl, phteridinyl, azepinyl, diazepinyl, imidazolyl, thiazolyl, carbazolyl, pyridinyl, pyridazinyl, pyrimidinyl, pyrazolyl, pyrazinyl, tetrazolyl, fury, thienyl, isoxazolyl, oxazolyl, isothiazolyl, pyrrolyl, indolyl, benzimidazolyl, benzofuranyl, cinnolinyl, indazolyl, indolizinyl, phthalazinyl, triazinyl, isoindolyl, purinyl, oxadiazolyl, thiadiazolyl, furazanyl, benzofurazanyl, benzothiophenyl, benzotriazolyl, benzothiazolyl, benzoxazolyl, quinazolinyl, quinoxalinyl, naphthyridinyl, dihydroquinolyl, tetrahydroquinolyl, dihydroisoquinolyl, tetrahydroisoquinolyl, benzofuryl, furopyridinyl, pyrolopyrimidinyl, azaindolyl, pyrazolinyl, 1,2,4-oxadiazol-5(4H)-one, and pyrazolidinyl. Non-limiting examples of partially hydrogenated derivatives are 1,2,3,4-tetrahydronaphthyl, 1,4-dihydronaphthyl, and 1-octalin. Correspondingly, the term “heteroarylene” means the corresponding biradical (-heteroaryl-). Heteroaryl groups may be optionally substituted with 1-4 substituents. Examples of substituents on heteroaryl groups include, but are not limited to, alkyl, alkenyl, alkynyl, halogen, haloalkyl, alkoxy, heteroaryl, aryl, carbocyclyl, hydroxyl, and —CN.

The term “heterocyclyl” as used herein, refers to cyclic non-aromatic groups containing one or more heteroatoms selected from O, S, and N, preferably from one to four heteroatoms, and more preferably from one to three heteroatoms. Heterocyclyl furthermore includes bi-, tri- and polycyclic non-aromatic groups, and at least one of the rings contains a heteroatom selected from O, S, and N. Heterocyclyl also include ring systems substituted with one or more oxo moieties. Examples of heterocyclic groups are oxetane, pyrrolidinyl, pyrrolyl, 3H-pyrrolyl, oxolanyl, furanyl, thiolanyl, thiophenyl, pyrazolyl, pyrazolidinyl, imidazolyl, imidazolidinyl, 3H-pyrazolyl, 1,2-oxazolyl, 1,3-oxazolyl, 1,2-thiazolyl, 1,3-thiazolyl, 1,2,5-oxadiazolyl, piperidinyl, pyridinyl, oxanyl, 2-H-pyranyl, 4-H-pyranyl, thianyl, 2H-thiopyranyl, pyridazinyl, 1,2-diazinanyl, pyrimidinyl, 1,3-diazinanyl, pyrazinyl, piperazinyl, 1,4-dioxinyl, 1,4-dioxanyl, 1,3-diazinanyl, 1,4-oxazinyl, morpholino, thiomorpholino, 1,4-oxathianyl, benzofuranyl, isobenzofuranyl, indazolyl, benzimidazolyl, quinolinyl, isoquinolinyl, chromayl, isochromanyl, 4H-chromenyl, 1H-isochromenyl, cinnolinyl, quinazolinyl, quinoxalinyl, phthalazinyl, purinyl, naphthyridinyl, pteridinyl, indolizinyl, 1H-pyrrolizinyl, 4H-quinolizinyl and aza-8-bicyclo[3.2.1]octane. Correspondingly, the term “heterocyclylene” means the corresponding biradical (-heterocyclyl-). Heterocyclyl groups may be optionally substituted with 1-4 substituents. Examples of substituents on heterocyclyl groups include, but are not limited, to alkyl, alkenyl, alkynyl, halogen, haloalkyl, alkoxy, heteroaryl, aryl, carbocyclyl, hydroxyl, and —CN.

The term “N-heterocyclic ring” as used herein, refers to a heterocyclyl or a heteroaryl, as defined hereinabove, having at least one nitrogen atom, and being bound via a nitrogen atom. Examples of such N-heterocyclic rings are pyrrolidinyl, pyrrolyl, 3H-pyrrolyl, pyrazolyl, pyrazolidinyl, imidazolyl, imidazolidinyl, 3H-pyrazolyl, 1,2-oxazolyl, 1,2-thiazolyl, 1,3-thiazolyl, piperidinyl, pyridinyl, pyridazinyl, pyrazinyl, piperazinyl, morpholino, pyridinyl, pyridazinyl, pyrimidinyl, pyrazolyl, pyrazinyl, tetrazolyl, etc.

In the present specification, the structural formula of the compound represents a certain isomer for convenience in some cases, but the present disclosure includes all isomers, such as geometrical isomers, optical isomers based on an asymmetrical carbon, stereoisomers, tautomers, and the like. Accordingly, it should be understood that the definition of compounds of Formula (I) or (II) include each and every individual isomer corresponding to the Formula: Formula (I) or (II), including cis-trans isomers, stereoisomers and tautomers, as well as racemic mixtures of these and pharmaceutically acceptable salts thereof. Hence, the definition of compounds of Formula (I) or (II) are also intended to encompass all R- and S-isomers of a chemical structure in any ratio, e.g., with enrichment (i.e., enantiomeric excess or diastereomeric excess) of one of the possible isomers and corresponding smaller ratios of other isomers. In addition, a crystal polymorphism may be present for the compounds represented by Formula (I) or (II). It is noted that any crystal form, crystal form mixture, or anhydride or hydrate thereof is included in the scope of the present disclosure. Furthermore, so-called metabolite which is produced by degradation of the present compound in vivo is included in the scope of the present disclosure.

“Isomerism” means compounds that have identical molecular formulae but differ in the sequence of bonding of their atoms or in the arrangement of their atoms in space. Isomers that differ in the arrangement of their atoms in space are termed “stereoisomers”.

Stereoisomers that are not mirror images of one another are termed “diastereoisomers”, and stereoisomers that are non-superimposable mirror images of each other are termed “enantiomers” or sometimes optical isomers. A mixture containing equal amounts of individual enantiomeric forms of opposite chirality is termed a “racemic mixture”.

A carbon atom bonded to four non-identical substituents is termed a “chiral center”.

“Chiral isomer” means a compound with at least one chiral center. Compounds with more than one chiral center may exist either as an individual diastereomer or as a mixture of diastereomers, termed “diastereomeric mixture”. When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116).

Diastereoisomers, i.e., non-superimposable stereochemical isomers, can be separated by conventional means such as chromatography, distillation, crystallization or sublimation. The optical isomers can be obtained by resolution of the racemic mixtures according to conventional processes, for example by formation of diastereoisomeric salts by treatment with an optically active acid or base. Examples of appropriate acids include, without limitation, tartaric, diacetyltartaric, dibenzoyltartaric, ditoluoyltartaric and camphorsulfonic acid. The mixture of diastereomers can be separated by crystallization followed by liberation of the optically active bases from these salts. An alternative process for separation of optical isomers includes the use of a chiral chromatography column optimally chosen to maximize the separation of the enantiomers. Still another available method involves synthesis of covalent diastereoisomeric molecules by reacting compounds of Formula (I) or (II) with an optically pure acid in an activated form or an optically pure isocyanate. The synthesized diastereoisomers can be separated by conventional means such as chromatography, distillation, crystallization or sublimation, and then hydrolyzed to obtain the enantiomerically pure compound. The optically active compounds of Formulae (I) can likewise be obtained by utilizing optically active starting materials and/or by utilizing a chiral catalyst. These isomers may be in the form of a free acid, a free base, an ester, or a salt. Examples of chiral separation techniques are given in Chiral Separation Techniques, A Practical Approach, 2^{nd} ed. by G. Subramanian, Wiley-VCH, 2001.

“Geometric isomer” means the diastereomers that owe their existence to hindered rotation about double bonds. These configurations are differentiated in their names by the prefixes cis and trans, or Z and E, which indicate that the groups are on the same or opposite side of the double bond in the molecule according to the Cahn-Ingold-Prelog rules.

Furthermore, the structures and other compounds discussed in this disclosure include all atropic isomers thereof. “Atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques; it has been possible to separate mixtures of two atropic isomers in select cases.

“Tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solid form, usually one tautomer predominates. In solutions where tautomerization is possible, a chemical equilibrium of the tautomers will be reached. The exact ratio of the tautomers depends on several factors, including temperature, solvent and pH. The concept of tautomers that are interconvertible by tautomerizations is called tautomerism.

Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

Common tautomeric pairs are: ketone-enol, amide-nitrile, lactam-lactim, amide-imidic acid tautomerism in heterocyclic rings (e.g., in nucleobases such as guanine, thymine and cytosine), amine-enamine, and enamine-enamine. It is to be understood that the compounds of the present disclosure may be depicted as different tautomers. It should also be understood that when compounds have tautomeric forms, all tautomeric forms are intended to be included in the scope of the present disclosure, and the naming of the compounds does not exclude any tautomer form.

The term “crystal polymorphs”, “polymorphs” or “crystal forms” means crystal structures in which a compound (or a salt or solvate thereof) can crystallize in different crystal packing arrangements, all of which have the same elemental composition. Different crystal forms usually have different X-ray diffraction patterns, infrared spectral, melting points, density hardness, crystal shape, optical, and electrical properties, stability and solubility. Recrystallization solvent, rate of crystallization, storage temperature, and other factors may cause one crystal form to dominate. Crystal polymorphs of the compounds can be prepared by crystallization under different conditions.

Additionally, the compounds of the present disclosure, for example, the salts of the compounds, can exist in either hydrated or unhydrated (the anhydrous) form or as solvates with other solvent molecules. Nonlimiting examples of hydrates include monohydrates, dehydrates, etc. Nonlimiting examples of solvates include ethanol solvates, acetone solvates, etc.

“Solvate” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

As used herein, a “subject” or “subject in need thereof” is a subject having a disease or disorder that is an acute inflammatory condition. In other embodiments, a subject has a disease or disorder associated with α-amino-β-carboxymuconate-ε-semialdehyde decarboxylase (ACMSD) dysfunction or inhibited by α-amino-β-carboxymuconate-ε-semialdehyde decarboxylase (ACMSD). A “subject” includes a mammal. The mammal can be e.g., any mammal, e.g., a human, primate, bird, mouse, rat, fowl, dog, cat, cow, horse, goat, camel, sheep or a pig. Preferably, the mammal is a human.

The present disclosure is intended to include all isotopes of atoms occurring in the present compounds. Isotopes include those atoms having the same atomic number but different mass numbers. By way of general example and without limitation, isotopes of hydrogen include tritium and deuterium, and isotopes of carbon include C-13 and C-14.

### Methods for the Preparation of Compounds

The compounds of the present disclosure (e.g., compounds of Formula (I)) can be prepared in a number of ways well known to those skilled in the art of organic synthesis. By way of example, compounds of the present disclosure can be synthesized using the methods described below, together with synthetic methods known in the art of synthetic organic chemistry, or variations thereon as appreciated by those skilled in the art. Preferred methods include but are not limited to those methods described below. The final products of the reactions described herein may be isolated by conventional techniques, e.g., by extraction, crystallisation, distillation, chromatography, etc.

Compounds of the present disclosure can be synthesized by following the steps outlined in General Scheme A to F which comprise different sequences of assembling intermediates Ia-Ih and Ij-Io. Starting materials are either commercially available or made by known procedures in the reported literature or as illustrated. Useful steps that may be used in the preparation steps of the compounds will be known to the skilled person. The method below is given as a non-limiting example on how the compounds may be prepared.

### General Scheme A

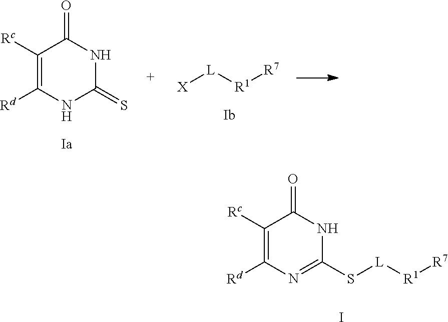

wherein R¹, R^{c}, R^{d}, and L are defined as in Formula (I).

The general way of preparing compounds of Formula (I) by using intermediates Ia, and Ib is outlined in General Scheme A. Coupling of Ia with Ib using a base, i.e., potassium carbonate (K₂CO₃), in a solvent, i.e., acetonitrile (CH₃CN), optionally at elevated temperature provides the desired produce of Formula (I). Bases that can be used include, but are not limited to, sodium carbonate (Na₂CO₃), potassium carbonate (K₂CO₃), N,N-diisopropylethylamine (DIPEA) and triethylamine. Solvents used in the coupling reaction can be polar or non-polar solvents. For example, the solvent can be acetonitrile (CH₃CN), acetone, or dimethylsulfoxide (DMSO).

### General Scheme B

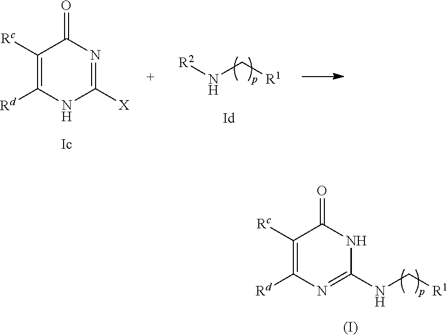

wherein X is a good leaving group, i.e., Cl, Br, —SCH₃, or S(O)₂CH₃, and R¹, R², R^{c}, R^{d}, and p are defined as in Formula (I).

Alternatively, compounds of Formula (I) can be prepared using intermediates Ic and Id as outlined in General Scheme B. Amination of Intermediate Ic with Ie using a base, i.e., sodium hydroxide (NaOH), potassium hydroxide (KOH), etc., in a solvent, i.e., methanol (MeOH), ethanol (EtOH), water (H₂O), etc., provides compounds of Formula (I).

### General Scheme C

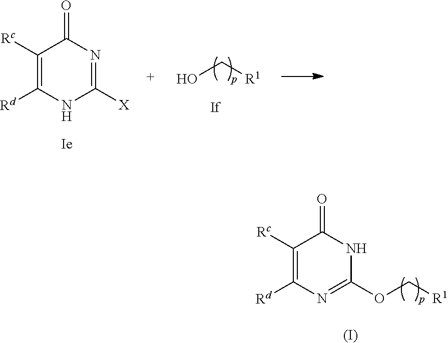

wherein X is a good leaving group, i.e., Cl, Br, —SCH₃, or S(O)₂CH₃, and R¹, R², R^{c}, R^{d}, and p are defined as in Formula (I).

Compounds of Formula (I) can also be prepared using intermediates Ie and If as outlined in General Scheme C. Amination of Intermediate Ie with If using a base, i.e., sodium hydroxide (NaOH), potassium hydroxide (KOH), etc., in a solvent, i.e., methanol (MeOH), ethanol (EtOH), water (H₂O), etc., provides compounds of Formula (I).

### General Scheme D

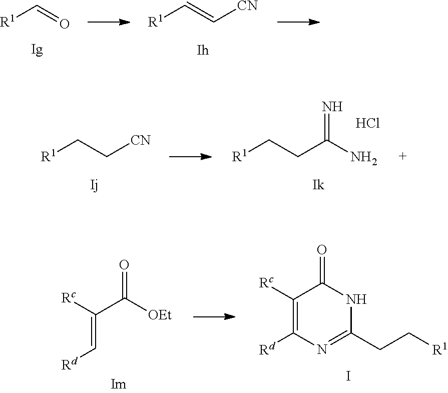

wherein and R¹, R^{c}, and R^{d} are defined as in Formula (I).

Alternatively, compounds of Formula (I) can also be prepared using intermediates Ig, Ih, Ij, Ik, and Im as outlined in General Scheme D. Olefination of intermediate Ig using a base i.e., potassium carbonate (K₂CO₃) and diethyl (cyanomethyl)phosphonate in a solvent, i.e., tetrahydrofuran (THF), water (H₂O), optionally at an elevated temperature provides Intermediate Ih. Hydrogenation of Ih using a metal catalyst, i.e., palladium on carbon (Pd/C), platinum dioxide (PtO₂), etc., and hydrogen (H₂) gas in a solvent, i.e., ethanol (EtOH) and/or tetrahydrofuran (THF), provides Intermediate Ij. Intermediate Ik is obtained by treating Intermediate Ij with an acid, i.e., hydrochloric acid (HCl) in a solvent, i.e., ethanol (EtOH), dichloromethane (CH₂Cl₂), etc., and then subsequent treatment with a base, i.e., ammonia (NH₃). Cyclization of Intermediate Ik and Im using a base, i.e., sodium hydroxide (NaOH), potassium hydroxide (KOH), etc., in a solvent, i.e., dimethylacetamide (DMA), optionally at elevated temperature provides compounds of Formula (I).

### General Scheme E

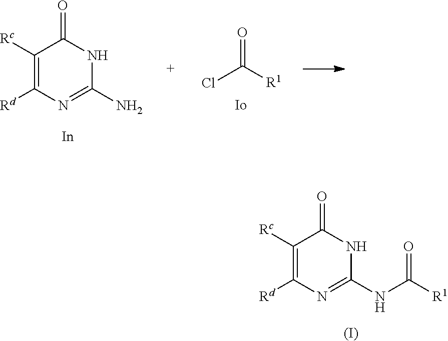

wherein and R¹, R^{c}, and R^{d} are defined as in Formula (I).

Alternatively, compounds of Formula (I) can be prepared using intermediates In and Io as outlined in General Scheme D. Acylation of Intermediate In with Io using a base, i.e., sodium hydroxide (NaOH), potassium hydroxide (KOH), etc., in a solvent, i.e., methanol (MeOH), ethanol (EtOH), water (H₂O), etc., provides compounds of Formula (I).

### General Procedure F

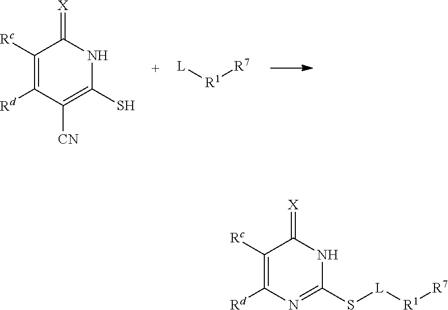

wherein and L, R^{c}, R^{d}, R¹, and R⁷ are defined as in Formula (I).

The general procedure for the synthesis of compounds (e.g., I-17 to I-30) with general Formula I include the final coupling between one equivalent of the corresponding substituted 6-mercapto-2-oxo-4,5-disubstituted-1,2-dihydro-pyridine derivative and a stoichiometric amount of the L-R¹-R⁷ intermediates using two equivalent of DIPEA as base and acetone as solvent to provide the final compound.

Alternatively, certain compounds of Formula (I) or (II) can be prepared using the schemes shown below and compounds of Formula (I) or (II) in general can be prepared based on the schemes shown below.

### General Scheme G, 6-Oxo-2-[4-(1H-tetrazol-5-yl)-phenylamino]-4-thiophen-2-yl-1,6-dihydro-pyrimidine-5-carbonitrile

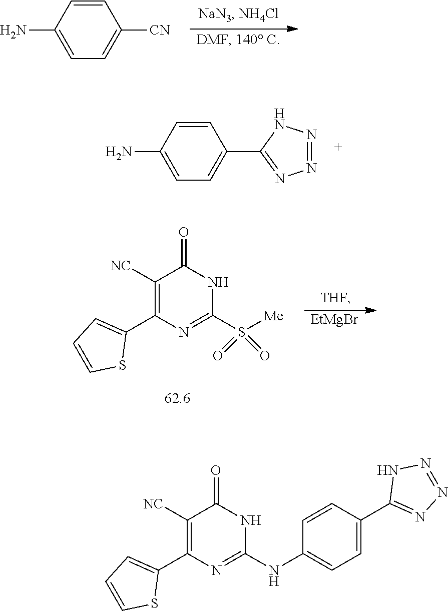 [SMILES: "EtMgBr" ; N#Cc1ccc(N)cc1 ; Nc1ccc(-c2nnn[nH]2)cc1 ; CS(=O)(=O)c1nc(-c2cccs2)c(C#N)c(=O)[nH]1 ; N#Cc1c(-c2cccs2)nc(Nc2ccc(-c3nnn[nH]3)cc2)[nH]c1=O]

### General Scheme H, 6-Oxo-2-[4-(1H-tetrazol-5-yl)-cyclohexylamino]-4-thiophen-2-yl-1,6-dihydro-pyrimidine-5-carbonitrile

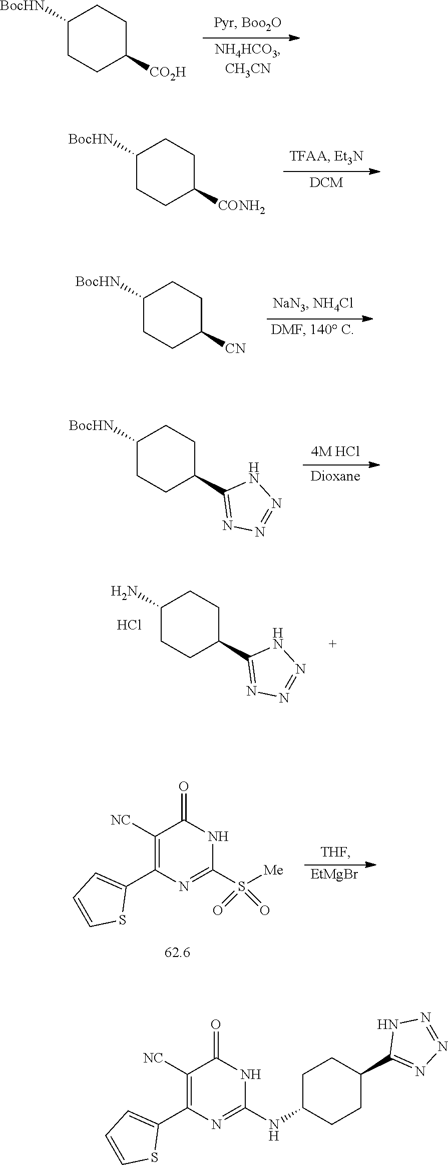

### General Scheme I, 6-Oxo-2-[4-(1H-tetrazol-5-yl)-piperidin-1-yl]-4-thiophen-2-yl-1,6-dihydro-pyrimidine-5-carbonitrile

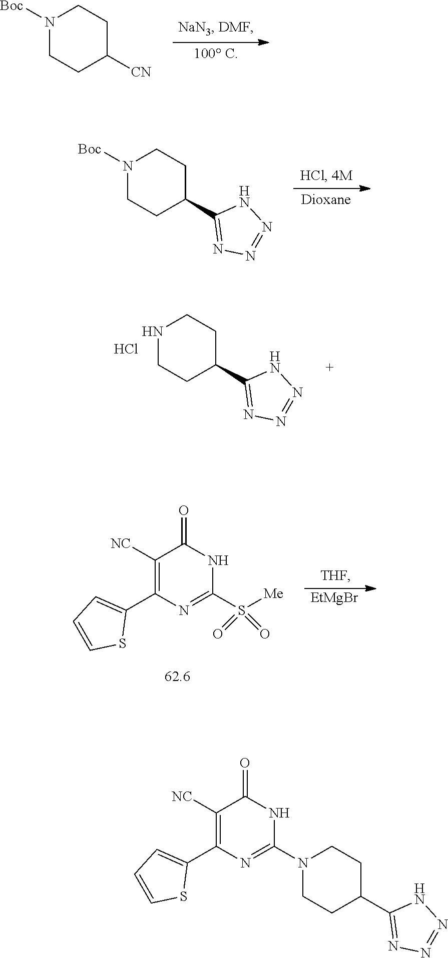

### General Scheme J, 6-Oxo-2-[3-(1H-tetrazol-5-yl)-azetidin-1-yl]-4-thiophen-2-yl-1,6-dihydro-pyrimidine-5-carbonitrile

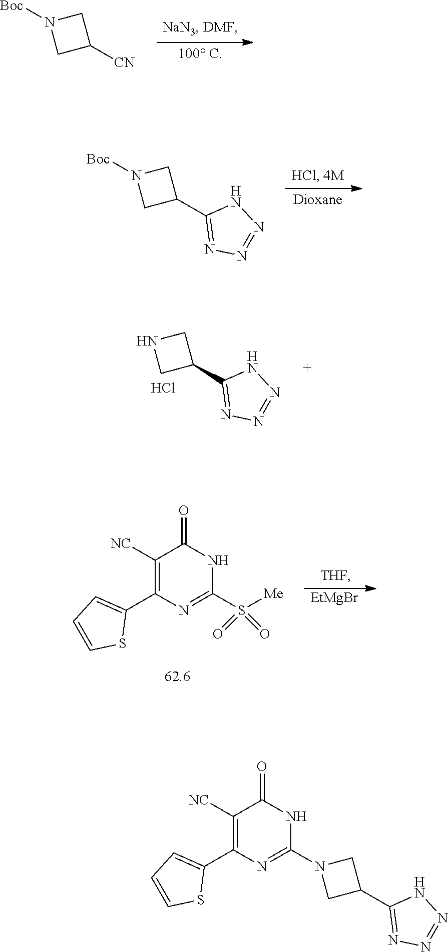

### General Scheme K, 4-Benzyl-6-oxo-2-[2-(1H-tetrazol-5-yl)-benzylsulfanyl]-1,6-dihydro-pyrimidine-5-carbonitrile

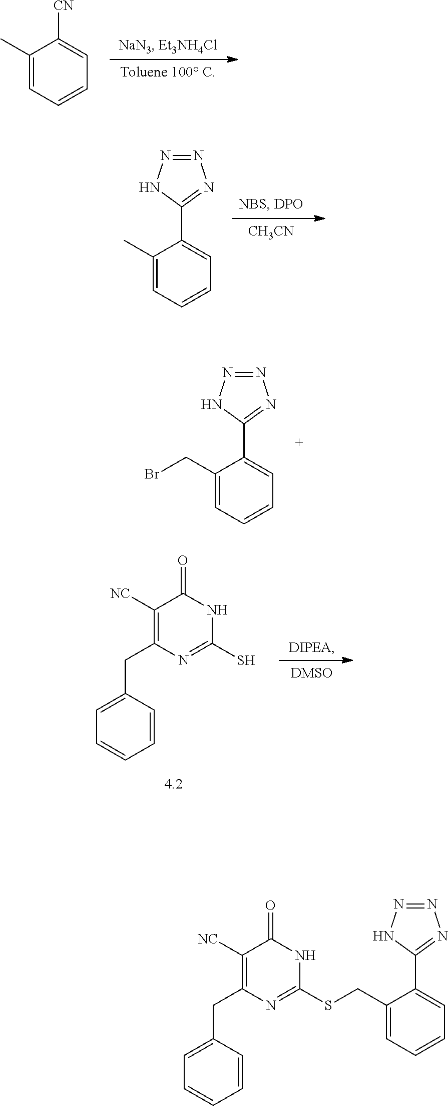

A mixture of enantiomers, diastereomers, cis/trans isomers resulting from the process described above can be separated into their single components by chiral salt technique, chromatography using normal phase, reverse phase or chiral column, depending on the nature of the separation.

It should be understood that in the description and formula shown above, the various groups R¹, R², X, L, Y, R^{a}, R^{b}, R^{c}, R^{d}, R^{e}, R^{f}, R^{x}, R^{y}, R^{z}, m, n, p, q, r and other variables are as defined herein above, except where otherwise indicated. Furthermore, for synthetic purposes, the compounds of General Schemes A-E are merely representative with elected radicals to illustrate the general synthetic methodology of the compounds of Formula (I) as defined herein.

### Method of Treatment

The present disclosure provides a method of treating an acute inflammatory condition in a subject comprising administering a compound of Formula (I) or (II).

Inflammation is a complex of sequential changes expressing the response to damage of cells and vascularized tissues. When tissue injury occurs, whether it is caused by bacteria, trauma, chemicals, heat, or any other phenomenon, the substance histamine, along with other humoral substances, is liberated by the damaged tissue into the surrounding fluids. It is a protective attempt by the organism to remove the injurious stimuli as well as initiating the healing process.

The main features of the inflammatory response are vasodilation, i.e. widening of the blood vessels to increase the blood flow to the infected area; increased vascular permeability which allows diffusible components to enter the site; cellular infiltration by chemotaxis; or the directed movement of inflammatory cells through the walls of blood vessels into the site of injury; changes in biosynthetic, metabolic, and catabolic profiles of many organs; and activation of cells of the immune system as well as of complex enzymatic systems of blood plasma. Inflammation which runs unchecked can, however, lead to a host of diseases, including acute heptatitis, acute pancreatitis, acute kidney disease, inflammatory bowel disease, inflammatory liver diseases, rheumatoid arthritis, autoimmunity, sepsis, SIRS, and atherosclerosis.

Acute inflammation is the initial response of the body to harmful stimuli and is achieved by the increased movement of plasma and leukocytes from the blood into the injured tissues. Acute inflammation can be divided into several phases. The earliest, event of an inflammatory response is temporary vasoconstriction, i.e., narrowing of blood vessels caused by contraction of smooth muscle in the vessel walls which can be seen as blanching (whitening) of the skin. This is followed by several phases that occur minutes, hours, and days later. The first is the acute vascular response which follows within seconds of the tissue injury and lasts for several minutes. This results from vasodilation and increased capillary permeability due to alterations in the vascular endothelium which leads to increased blood flow (hyperemia) that causes redness (erythema) and the entry of fluid into the tissues (edema).

The acute vascular response can be followed by an acute cellular response which takes place over the next few hours. The hallmark of this phase is the appearance of granulocytes, particularly neutrophils, in the tissues. These cells first attach themselves to the endothelial cells within the blood vessels (margination) and then cross into the surrounding tissue (diapedesis). During this phase erythrocytes may also leak into the tissues and a hemorrhage can occur. If the vessel is damaged, fibrinogen and fibronectin are deposited at the site of injury, platelets aggregate and become activated, and the red cells stack together in what are called “rouleau” to help stop bleeding and aid clot formation. The dead and dying cells contribute to pus formation. If the damage is sufficiently severe, a chronic cellular response may follow over the next few days. A characteristic of this phase of inflammation is the appearance of a mononuclear cell infiltrate composed of macrophages and lymphocytes. The macrophages are involved in microbial killing, in clearing up cellular and tissue debris, and in remodeling of tissues.

Acute inflammation occurs immediately upon injury and is a relatively short-term process, lasting up to a few days. Wherein, cytokines and chemokines promote the migration of neutrophils and macrophages to the site of inflammation.

Resident liver macrophages (Kupffer cells) are the first innate immune cells and protect the liver from bacterial infections. Under pathological conditions, they are activated by different components and can differentiate into M1-like (pro-inflammatory) or M2-like (anti-inflammatory) macrophages.

Kupffer cells can be activated to produce a variety of cytokines, eicosanoids, nitric oxide, and oxygen radicals and play divergent roles in tissue injury and tissue repair. Once the acute injury has been controlled, Kupffer cells and other macrophages play a role in suppressing inflammation and initiating wound repair by clearing debris and producing growth factors and mediators that provide trophic support to the tissue in which they reside.

Kupffer cell activation is involved in the response of the liver to infection or injury; the ensuing inflammatory response protects from infection, as well as limits cellular and organ damage to the host organism.

However, in other types of insults to the liver, the Kupffer cell is unable to appropriately control or resolve its state of activation. The controlled and appropriate resolution of inflammation is an essential feature of the innate immune response. This failure to resolve Kupffer cell activation contributes to a number of chronic inflammatory diseases in the liver.

M1 and M2 macrophage populations differ from their capacity to respond to different stimuli and the repertoire of chemokines/cytokines and receptors they express after their activation. However, both of them become active macrophages with high synthesis and secretion of inflammatory mediators including cytokines, superoxide, nitric oxide, eicosanoids, chemokines, and lysosomal and proteolytic enzymes. Moreover, they exhibit high phagocytic and secretory activities.

Under physiological conditions, Kupffer cells are the first innate immune cells and protect the liver from bacterial infections. Under pathological conditions, they are activated by different components and can differentiate into M1-like (classical) or M2-like (alternative) macrophages. The metabolism of classical or alternative activated Kupffer cells will determine their functions in liver damage.

Kupffer cells are derived from monocytes and differentiate into liver resident macrophages.

Liver-resident Kupffer cells initiate inflammation and help recruit blood-derived monocytes; both differentiate into pro-inflammatory macrophages and further promote NAFLD progression.

While Kupffer Cells display M1-like features in acute liver injury, with protracted chronic inflammation, due to exhaustion of M1-like macrophages and immune cells, M2-like macrophages emerge and secrete protective cytokines upon chronic cytotoxic stimulation such as IL-4, IL-10, and TGF-β.

The immediate resulting effects of liver injuries are increased hepatocellular necrosis, which is one of the principal sources of Kupffer cell activator.

Chronic inflammation is inflammation that lasts for months or years. Macrophages, lymphocytes, and plasma cells predominate in chronic inflammation, in contrast to the neutrophils that predominate in acute inflammation. Examples of diseases mediated by chronic inflammation include diabetes, cardiovascular disease, allergies, and chronic obstructive pulmonary disease (COPD).

Inflammatory cytokines can be divided into two groups: those involved in acute inflammation and those responsible for chronic inflammation. Those involved in acute inflammation include, for example, IL-1, TNF-α, IL-6, IL-11, IL-8, and other chemokines, G-CSF, and GM-CSF. Cytokines in chronic inflammation can be subdivided into cytokines that mediate humoral responses, such as IL-4, IL-5, IL-6, IL-7, and IL-13, and those mediating cellular responses, such as IL-1, IL-2, IL-3, IL-4, IL-7, IL-9, IL-10, IL-12, interferons, transforming growth factor-β, and tumor necrosis factor α and β. Some cytokines contribute to both acute and chronic inflammation.

As used herein, a “cytokine” is a molecule which is released by cells in response to infection or injury that stimulates an inflammatory or healing response. Cytokines are produced by various cells of the body. The cytokine superfamily includes interleukins, chemokines, colony-stimulating factors (CSF), interferons, and the transforming growth factors (TNF) and tumor necrosis factor (TGF) families.

Cytokines are small secreted proteins released by cells have a specific effect on the interactions and communications between cells. Subclasses of cytokines include lymphokine (cytokines made by lymphocytes), monokine (cytokines made by monocytes), chemokine (cytokines with chemotactic activities), and interleukin (cytokines made by one leukocyte and acting on other leukocytes). Cytokines may act on the cells that secrete them (autocrine action), on nearby cells (paracrine action), or in some instances on distant cells (endocrine action). There are both pro-inflammatory cytokines and anti-inflammatory cytokines.

A variety of cytokines are known to induce chemotaxis. One subgroup of cytokines is known as chemokines. These factors represent a family of low molecular weight secreted proteins that primarily function in the activation and migration of leukocytes although some of them also possess a variety of other functions. Chemokines have conserved cysteine residues that allow them to be assigned to four groups: C-C chemokines (RANTES, monocyte chemoattractant protein or MCP-1, monocyte inflammatory protein or MIP-1α, and MIP-1β), C-X-C chemokines (IL-8 also called growth related oncogene or GRO/KC), C chemokines (lymphotactin), and CXXXC chemokines (fractalkine).

The net effect of an inflammatory response can be determined by a balance between proinflammatory and anti-inflammatory cytokines. A proinflammatory cytokine is a cytokine which promotes inflammation. Proinflammatory cytokines are produced predominantly by activated macrophages and are involved in the up-regulation of inflammatory reactions. Anti-inflammatory cytokines are a series of immunoregulatory molecules that control the proinflammatory cytokine response. Cytokines act in concert with certain cytokine inhibitors and soluble cytokine receptors to regulate the human immune response.

A proinflammatory cytokine is a cytokine which promotes inflammation. Major proinflammatory cytokines that play roles for early responses are IL-1α, IL-1β, IL-6, and TNF-α. Other proinflammatory mediators include members of the IL-20 family, IL-33 LIF, IFN-γ, OSM, CNTF, TGF-β, GM-CSF, IL-11, IL-12, IL-17, IL-18, IL-8, and a variety of other chemokines that chemoattract inflammatory cells. These cytokines either act as endogenous pyrogens (IL-1, IL-6, TNF-α), upregulate the synthesis of secondary mediators and proinflammatory cytokines by both macrophages and mesenchymal cells (including fibroblasts, epithelial and endothelial cells), stimulate the production of acute phase proteins, or attract inflammatory cells.

Anti-inflammatory cytokines are a series of immunoregulatory molecules that control the proinflammatory cytokine response. Major anti-inflammatory cytokines include interleukin (IL)-1 receptor antagonist, IL-4, IL-10, IL-11, and IL-13. Leukemia inhibitory factor, interferon-α, IL-6, and TGF-β are categorized as either anti-inflammatory or pro-inflammatory cytokines, under various circumstances.

Amongst anti-inflammatory cytokines, IL-10 is a cytokine with anti-inflammatory properties, repressing the expression of inflammatory cytokines, such as TNF-α, IL-6, and IL-1 by activated macrophages. In addition, IL-10 can up-regulate endogenous anti-cytokines and down-regulate pro-inflammatory cytokine receptors.

IL-6 is produced at the site of inflammation and plays a role in the acute phase response as defined by a variety of clinical and biological features such as the production of acute phase proteins. Also, IL-6 in combination with its soluble receptor sIL-6Rα, can dictate the transition from acute to chronic inflammation by changing the nature of leucocyte infiltrate (from polymorphonuclear neutrophils to monocyte/macrophages).

In certain embodiments, the method reduces a pro-inflammatory cytokine or increases an anti-inflammatory cytokine.

In certain embodiments, the pro-inflammatory cytokine is IL-1β, IL-6, IL-18, TNF-α, or TGF-β. In certain embodiments, the pro-inflammatory cytokine is IL-18 or TNF-α. In certain embodiments, the pro-inflammatory cytokine is IL-6. In certain embodiments, the pro-inflammatory cytokine is TGF-β or TNF-α. In certain embodiments, the pro-inflammatory cytokine is IL-1β, IL-6, or TNF-α.

M1 macrophages, also called classically activated, can respond to stimuli, such as LPS or IFN-γ, and are producers of pro-inflammatory cytokines. M2 macrophages, also called alternatively activated, can respond to stimuli such as IL-4 or IL-13, are producer of anti-inflammatory cytokines.

In certain embodiments, the pro-inflammatory cytokine is MCP-1, TNF-α, or IL-1β and the pro-inflammatory cytokine is reduced.

In certain embodiments, the pro-inflammatory cytokine is IL-6 and the pro-inflammatory cytokine is reduced.

In certain embodiments, the anti-inflammatory cytokine is IL-10 (interleukin 10) and the anti-inflammatory cytokine is increased.

SIRT1, a known key metabolic regulator, can reprogram inflammation by altering histones and transcription factors such as NFκB and AP1. Mounting evidence supports that inflammation sequentially links immune, metabolic, and mitochondrial bioenergy networks; sirtuins are essential regulators of these networks.

In certain embodiments, expression of sirtuin-1 modulated genes is increased. In certain embodiments, expression of sirtuin-1 modulated genes is increased in the liver. In certain embodiments, expression of sirtuin-1 modulated genes sod2, tfam, or dda1 are increased. In certain embodiments, expression of sirtuin-1 modulated genes sod2, tfam, or dda1 are increased in the liver.

Inflammation is a cascading event that involves many cellular and humoral mediators. On one hand, suppression of inflammatory responses can leave a subject immunocompromised. However, if left unchecked, inflammation can lead to serious complications including chronic inflammatory diseases (e.g. asthma, psoriasis, arthritis, rheumatoid arthritis, and multiple sclerosis, and the like), septic shock and multiple organ failure. These diverse conditions share common inflammatory mediators, such as cytokines, chemokines, inflammatory cells and other mediators secreted by these cells.

Inflammation may be systemic or may affect a tissue.

In certain embodiments, the acute inflammatory condition is a systemic inflammatory condition. A systemic inflammatory condition refers to a disease or condition with involvement of at least two organ systems.

In one embodiment, systemic inflammatory condition includes SIRS and sepsis. In certain embodiments, the systemic inflammatory condition is one or more of SIRS and sepsis. In certain embodiments, the systemic inflammatory condition is one or more of SIRS, abdominal sepsis, and pulmonary sepsis. In certain embodiments, the systemic inflammatory condition may be associated various infections, including bacterial, viral, or fungal infections. In certain embodiments, the systemic inflammatory condition may be associated with a viral infection, such as COVID.

Systemic inflammatory response syndrome (SIRS) refers to a systemic inflammatory response syndrome with no signs of infection. This condition may also be referred to as “non-infective SIRS” or “infection-free SIRS.” SIRS may be characterized by the presence of at least two of the four following clinical symptoms: fever or hypothermia (temperature of 38.0° C. (100.4° F.) or more, or temperature of 36.0° C. (96.8° F.) or less); tachycardia (at least 90 beats per minute); tachypnea (at least 20 breaths per minute or PaCC>2 less than 4.3 kPa (32.0 mm Hg) or the need for mechanical ventilation); and an altered white blood cell (WBC) count of 12×10⁶ cells/mL or more, or an altered WBC count of 4×10⁶ cells/mL or less, or the presence of more than 10% band forms (immature neutrophils). Sepsis refers to the systemic inflammatory condition that occurs as a result of infection. Defined focus of infection is indicated by either (i) an organism grown in blood or sterile site; or (ii) an abscess or infected tissue (e.g., pneumonia, peritonitis, urinary tract, vascular line infection, soft tissue). In one embodiment, the infection may be a bacterial infection. The presence of sepsis is also characterised by the presence of at least two (of the four) systemic inflammatory response syndrome (SIRS) criteria defined above.

Cytokine storm or hypercytokinemia is a potentially fatal immune reaction and involves a positive feedback loop between cytokines and immune cells, which causes in the body highly elevated levels of various cytokines. Cytokine storm typically involves increased concentration of cytokines, such as interferons, interleukins, chemokines, colony-stimulating factors, and tumor necrosis factors. Such immune dysregulation can be an underlying factor in mortality resulting from many infections.

Catastrophic antiphospholipid syndrome is a potentially life-threatening condition characterized by diffuse vascular thrombosis, leading to multiple organ failure developing over a short period of time in the presence of positive antiphospholipid antibodies (aPL). It is acute in onset, with majority of cases developing thrombocytopenia, less frequently hemolytic anemia, and disseminated intravascular coagulation. The syndrome is caused by antiphospholipid antibodies that target a group of proteins in the body that are associated with phospholipids. These antibodies activate endothelial cells, platelets, and immune cells, ultimately causing a large inflammatory immune response and widespread clotting.

Graft versus host disease (GVHD) is a syndrome, characterized by inflammation in different organs, with the specificity of epithelial cell apoptosis and crypt drop out. GVHD is commonly associated with bone marrow transplants, stem cell transplants, and other forms of transplanted tissues such as solid organ transplants. White blood cells of a donor's immune system which remain within the donated tissue (the graft) recognize the recipient (the host) as foreign (non-self). The white blood cells present within the transplanted tissue then attack the recipient's body's cells, which leads to GVHD.

In certain embodiments, the acute inflammatory condition is systemic inflammatory response syndrome (SIRS), shock, sepsis, a cytokine storm, or hypercytokinemia, catastrophic anti-phospholipid syndrome, or graft versus host disease (GVHD).

An inflammatory condition includes an inflammatory pulmonary condition. Certain inflammatory pulmonary conditions include infection-induced pulmonary conditions including those associated with viral, bacterial, fungal, parasite, or prion infections. The inflammatory conditions also include community-acquired pneumonia, nosocomial pneumonia, ventilator-associated pneumonia, sepsis, viral pneumonia, influenza infection, parainfluenza infection, rotavirus infection, human metapneumovirus infection, respiratory syncitial virus infection, and aspergillus or other fungal infections. Certain infection-associated inflammatory diseases may include viral or bacterial pneumonia, including severe pneumonia, and acute respiratory distress syndrome (ARDS). Such infection-associated conditions may involve multiple infections such as a primary viral infection and a secondary bacterial infection.

In certain embodiments, the acute inflammatory condition is acute respiratory distress syndrome (ARDS), severe acute respiratory distress syndrome (SARS), viral infections, bacterial infections, fungal infections, influenza, or pneumonia.

In certain embodiments, the acute inflammatory condition is a cytokine storm or hypercytokinemia, systemic inflammatory response syndrome (SIRS), graft versus host disease (GVHD), acute respiratory distress syndrome (ARDS), severe acute respiratory distress syndrome (SARS), catastrophic anti-phospholipid syndrome, viral infections, bacterial infections, fungal infections, influenza, pneumonia, shock, or sepsis.

In certain embodiments, the acute inflammatory condition is an organ-specific or tissue-specific condition. Certain affected tissues are pancreas, hepatic tissue, the respiratory tract, lung, the gastrointestinal tract, small intestine, large intestine, colon, rectum, the cardiovascular system, cardiac tissue, blood vessels, joint, bone and synovial tissue, cartilage, epithelium, endothelium, or adipose tissue.

In certain embodiments, the acute inflammatory condition is acute pancreatitis, hepatitis, respiratory condition, or enterocolitis.

### Pharmaceutical Compositions

The compound of Formula (I) or (II) may be provided in any form suitable for the intended administration, in particular including pharmaceutically acceptable salts, solvates and prodrugs of the compound of Formula (I) or (II).

Pharmaceutically acceptable salts refer to salts of the compounds of Formula (I) or (II) which are considered to be acceptable for clinical and/or veterinary use. Typical pharmaceutically acceptable salts include those salts prepared by reaction of the compounds of Formula (I) or (II) and a mineral or organic acid or an organic or inorganic base. Such salts are known as acid addition salts and base addition salts, respectively. It will be recognized that the particular counter-ion forming a part of any salt is not of a critical nature, so long as the salt as a whole is pharmaceutically acceptable and as long as the counter-ion does not contribute undesired qualities to the salt as a whole. These salts may be prepared by methods known to the skilled person. Pharmaceutically acceptable salts are, e.g., those described and discussed in Remington's Pharmaceutical Sciences, 17. Ed. Alfonso R. Gennaro (Ed.), Mack Publishing Company, Easton, PA, U.S.A., 1985 and more recent editions and in Encyclopedia of Pharmaceutical Technology.

Examples of pharmaceutically acceptable addition salts include acid addition salts formed with inorganic acids, e.g., hydrochloric, hydrobromic, sulfuric, nitric, hydroiodic, metaphosphoric, or phosphoric acid; and organic acids e.g., succinic, maleic, acetic, fumaric, citric, tartaric, benzoic, trifluoroacetic, malic, lactic, formic, propionic, glycolic, gluconic, camphorsulfuric, isothionic, mucic, gentisic, isonicotinic, saccharic, glucuronic, furoic, glutamic, ascorbic, anthranilic, salicylic, phenylacetic, mandelic, embonic (pamoic), ethanesulfonic, pantothenic, stearic, sulfinilic, alginic and galacturonic acid; and arylsulfonic, for example benzenesulfonic, p-toluenesulfonic, methanesulfonic, or naphthalenesulfonic acid; and base addition salts formed with alkali metals and alkaline earth metals and organic bases such as N,N-dibenzylethylenediamine, chloroprocaine, choline, diethanolamine, ethylenediamine, meglumine (N-methylglucamine), lysine and procaine; and internally formed salts. It should be understood that all references to pharmaceutically acceptable salts include solvent addition forms (solvates) or crystal forms (polymorphs) as defined herein, of the same salt.

The compound of Formula (I) or (II), or a pharmaceutically acceptable salt thereof, may be provided in dissoluble or indissoluble forms together with a pharmaceutically acceptable solvent such as water, ethanol, and the like. Dissoluble forms may also include hydrated forms such as the mono-hydrate, the dehydrate, the hemihydrate, the trihydrate, the tetrahydrate, and the like.

The compound of Formula (I) or (II), or a pharmaceutically acceptable salt thereof, may be provided as a prodrug. The term “prodrug” used herein is intended to mean a compound which—upon exposure to certain physiological conditions—will liberate the compound of Formula (I) or (II), or a pharmaceutically acceptable salt thereof, which then will be able to exhibit the desired biological action. A typical example is a labile carbamate of an amine.

Since prodrugs are known to enhance numerous desirable qualities of pharmaceuticals (e.g., solubility, bioavailability, manufacturing, etc.), the compounds of the present disclosure can be delivered in prodrug form. Thus, the present disclosure is intended to cover prodrugs of the presently claimed compounds, methods of delivering the same and compositions containing the same. “Prodrugs” are intended to include any covalently bonded carriers that release an active parent drug of the present disclosure in vivo when such prodrug is administered to a subject. Prodrugs in the present disclosure are prepared by modifying functional groups present in the compound in such a way that the modifications are cleaved, either in routine manipulation or in vivo, to the parent compound. Prodrugs include compounds of the present disclosure wherein a hydroxy, amino, sulfhydryl, carboxy or carbonyl group is bonded to any group that may be cleaved in vivo to form a free hydroxyl, free amino, free sulfhydryl, free carboxy, or free carbonyl group, respectively.

Examples of prodrugs include, but are not limited to, esters (e.g., acetate, dialkylaminoacetates, formates, phosphates, sulfates and benzoate derivatives) and carbamates (e.g., N,N-dimethylaminocarbonyl) of hydroxy functional groups, esters (e.g., C₁₋₆ alkyl esters, e.g., methyl esters, ethyl esters, 2-propyl esters, phenyl esters, 2-aminoethyl esters, morpholinoethanol esters, etc.) of carboxyl functional groups, N-acyl derivatives (e.g., N-acetyl) N-Mannich bases, Schiff bases and enaminones of amino functional groups, oximes, acetals, ketals and enol esters of ketone and aldehyde functional groups in compounds of the disclosure, and the like. See Bundegaard, H., Design of Prodrugs, p1-92, Elesevier, New York-Oxford (1985).

The compounds, or pharmaceutically acceptable salts, esters or prodrugs thereof, are administered orally, nasally, transdermally, pulmonary, inhalationally, buccally, sublingually, intraperitoneally, subcutaneously, intramuscularly, intravenously, rectally, intrapleurally, intrathecally, and parenterally. In one embodiment, the compound is administered orally. One skilled in the art will recognize the advantages of certain routes of administration.

The dosage regimen utilizing the compounds is selected in accordance with a variety of factors including type, species, age, weight, sex, and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter or arrest the progress of the condition.

Techniques for formulation and administration of the disclosed compounds of the disclosure can be found in Remington: the Science and Practice of Pharmacy, 19^{th} edition, Mack Publishing Co., Easton, PA (1995). In an embodiment, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

In one aspect of this disclosure, there is provided a pharmaceutical composition comprising at, as an active ingredient, at least one compound of Formula (I) or (II), or a pharmaceutically acceptable salt thereof, as defined herein, and optionally one or more pharmaceutically acceptable excipients, diluents and/or carriers. The compounds of Formula (I) or (II), or a pharmaceutically acceptable salt thereof, may be administered alone or in combination with pharmaceutically acceptable carriers, diluents or excipients, in either single or multiple doses. Suitable pharmaceutically acceptable carriers, diluents, and excipients include inert solid diluents or fillers, sterile aqueous solutions and various organic solvents.

A “pharmaceutical composition” is a formulation containing the compounds of the present disclosure in a form suitable for administration to a subject. The pharmaceutical compositions may be formulated with pharmaceutically acceptable carriers or diluents as well as any other known adjuvants and excipients in accordance with conventional techniques such as those disclosed in Remington: The Science and Practice of Pharmacy, 21st Edition, 2000, Lippincott Williams & Wilkins.

As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

The pharmaceutical compositions formed by combining a compound of Formula (I) or (II), or a pharmaceutically acceptable salt thereof, as defined herein, with pharmaceutically acceptable carriers, diluents or excipients can be readily administered in a variety of dosage forms such as tablets, powders, lozenges, syrups, suppositories, injectable solutions and the like. In powders, the carrier is a finely divided solid such as talc or starch which is in a mixture with the finely divided active component. In tablets, the active component is mixed with the carrier having the necessary binding properties in suitable proportions and compacted in the shape and size desired.

The pharmaceutical compositions may be specifically prepared for administration by any suitable route such as the oral and parenteral (including subcutaneous, intramuscular, intrathecal, intravenous and intradermal) route. It will be appreciated that the preferred route will depend on the general condition and age of the subject to be treated, the nature of the condition to be treated and the active ingredient chosen.

Pharmaceutical compositions for oral administration include solid dosage forms such as capsules, tablets, dragees, pills, lozenges, powders, and granules. Where appropriate, they can be prepared with coatings such as enteric coatings or they can be prepared so as to provide controlled release of the active ingredient such as sustained or prolonged release according to methods well known in the art.

For oral administration in the form of a tablet or capsule, a compound of Formula (I) or (II), or a pharmaceutically acceptable salt thereof, as defined herein, may suitably be combined with an oral, non-toxic, pharmaceutically acceptable carrier such as ethanol, glycerol, water, or the like. Furthermore, suitable binders, lubricants, disintegrating agents, flavouring agents, and colourants may be added to the mixture, as appropriate. Suitable binders include, e.g., lactose, glucose, starch, gelatin, acacia gum, tragacanth gum, sodium alginate, carboxymethylcellulose, polyethylene glycol, waxes, or the like. Lubricants include, e.g., sodium oleate, sodium stearate, magnesium stearate, sodium benzoate, sodium acetate, sodium chloride, or the like. Disintegrating agents include, e.g., starch, methyl cellulose, agar, bentonite, xanthan gum, sodium starch glycolate, crospovidone, croscarmellose sodium, or the like. Additional excipients for capsules include macrogels or lipids.

For the preparation of solid compositions such as tablets, the active compound of Formula (I) or (II), or a pharmaceutically acceptable salt thereof, is mixed with one or more excipients, such as the ones described above, and other pharmaceutical diluents such as water to make a solid pre-formulation composition containing a homogenous mixture of a compound of Formula (I) or (II), or a pharmaceutically acceptable salt thereof. The term “homogenous” is understood to mean that the compound of Formula (I) or (II), or a pharmaceutically acceptable salt thereof, is dispersed evenly throughout the composition so that the composition may readily be subdivided into equally effective unit dosage forms such as tablets or capsules.

Liquid compositions for either oral or parenteral administration of the compound of Formula (I) or (II), or a pharmaceutically acceptable salt thereof, include, e.g., aqueous solutions, syrups, elixirs, aqueous or oil suspensions and emulsion with edible oils such as cottonseed oil, sesame oil, coconut oil, or peanut oil. Suitable dispersing or suspending agents for aqueous suspensions include synthetic or natural gums such as tragacanth, alginate, acacia, dextran, sodium carboxymethylcellulose, gelatin, methylcellulose, or polyvinylpyrrolidone.

Pharmaceutical compositions for parenteral administration include sterile aqueous and non-aqueous injectable solutions, dispersions, suspensions or emulsions as well as sterile powders to be reconstituted in sterile injectable solutions or dispersions prior to use.

For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, and sodium chloride in the composition. Prolonged absorption of the injectable compositions can be brought about by including in the composition an agent which delays absorption, for example, aluminum monostearate and gelatin.

The preparation of all these solutions under sterile conditions is readily accomplished by standard pharmaceutical techniques well known to those skilled in the art.

For example, sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof. Depot injectable compositions are also contemplated as being within the scope of the present disclosure.

For parenteral administration, solutions containing a compound of Formula (I) or (II), or a pharmaceutically acceptable salt thereof, in sesame or peanut oil, aqueous propylene glycol, or in sterile aqueous solution may be employed. Such aqueous solutions should be suitably buffered if necessary and the liquid diluent first rendered isotonic with sufficient saline or glucose. These particular aqueous solutions are especially suitable for intravenous, intramuscular, subcutaneous, and intraperitoneal administration. The oily solutions are suitable for intra-articular, intra-muscular and subcutaneous injection purposes.

In addition to the aforementioned ingredients, the compositions of a compound of Formula (I) or (II), or a pharmaceutically acceptable salt thereof, may include one or more additional ingredients such as diluents, buffers, flavouring agents, colourant, surface active agents, thickeners, preservatives, e.g., methyl hydroxybenzoate (including anti-oxidants), emulsifying agents and the like.

The term “therapeutically effective amount”, as used herein, refers to an amount of a pharmaceutical agent to treat, ameliorate, or prevent an identified disease, disorder, or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician. In a preferred aspect, the disease or disorder to be treated is a disease or disorder associated with α-amino-β-carboxymuconate-ε-semialdehyde decarboxylase (ACMSD) dysfunction. In a preferred aspect, the disease or disorder is treated by inhibition of α-amino-β-carboxymuconate-ε-semialdehyde decarboxylase (ACMSD). In certain embodiments, the disease or disorder is an acute inflammatory condition.

For any compound, the therapeutically effective amount can be estimated initially either in cell culture assays, e.g., in cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

A suitable dosage of the compound of Formula (I) or (II), or a pharmaceutically acceptable salt thereof, will depend on the age and condition of the patient, the severity of the disease to be treated and other factors well known to the practicing physician. The compound may be administered for example either orally, parenterally or topically according to different dosing schedules, e.g., daily or with intervals, such as weekly intervals. In general a single dose will be in the range from 0.01 to 500 mg/kg body weight, preferably from about 0.05 to 100 mg/kg body weight, more preferably between 0.1 to 50 mg/kg body weight, and most preferably between 0.1 to 25 mg/kg body weight. The compound may be administered as a bolus (i.e., the entire daily dose is administered at once) or in divided doses two or more times a day. Variations based on the aforementioned dosage ranges may be made by a physician of ordinary skill taking into account known considerations such as weight, age, and condition of the person being treated, the severity of the affliction, and the particular route of administration.

As used herein, a “subject” or “subject in need thereof” is a subject having a disease or disorder that is an acute inflammatory condition. In other embodiments, a subject has a disease or disorder associated with α-amino-β-carboxymuconate-ε-semialdehyde decarboxylase (ACMSD) modulation. A “subject” includes a mammal. The mammal can be e.g., any mammal, e.g., a human, primate, bird, mouse, rat, fowl, dog, cat, cow, horse, goat, camel, sheep, or a pig. Preferably, the mammal is a human.

The compounds of Formula (I) or (II), or a pharmaceutically acceptable salt thereof, may also be prepared in a pharmaceutical composition comprising one or more further active substances alone, or in combination with pharmaceutically acceptable carriers, diluents, or excipients in either single or multiple doses. The suitable pharmaceutically acceptable carriers, diluents and excipients are as described herein above, and the one or more further active substances may be any active substances, or preferably an active substance as described in the section “combination treatment” herein below.

### EXEMPLARY EMBODIMENTS

Embodiment I-1. A method of treating an acute inflammatory condition in a subject comprising administering to the subject a therapeutically effective amount of a compound represented by Formula (II):

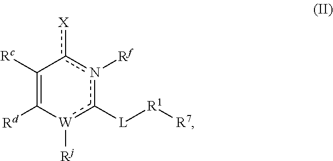

or a pharmaceutically acceptable salt or tautomer thereof, wherein:
X is H, S, SR², NR², NR²R^{2′}, O, OH, OR^{h}, F, Br, or Cl;
W is N or C;
(i) when W is N, then: L is —(C(R⁵)₂)ₘCH═CH(C(R⁵)₂)ₚ—,

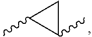

—(C(R⁵)₂)ₘY¹(C(R⁵)₂)ₚ—, —(C(R⁵)₂)ₘY¹(C(R⁵)₂)ₚ-cyclopropyl-, —(C(R⁵)₂)ₘY¹CH═CH—, —(C(R⁵)₂)ₘNR³C═(O)(C(R⁵)₂)ₚ—, —(C(R⁵)₂)ₘphenyl(C(R⁵)₂)ₚ—, —(C(R⁵)₂)ₘpyridinyl(C(R⁵)₂)ₚ—, or —(C(R⁵)₂)ₘthiophenyl(C(R⁵)₂)ₚ—;
(ii) when W is C, then: L is —(C(R⁵)₂)ₘCH═CH(C(R⁵)₂)ₚ—, —(C(R⁵)₂)ₒ—, —(C(R⁵)₂)ₘY¹(C(R⁵)₂)ₚ—,

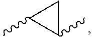

—(C(R⁵)₂)ₘ Y¹CH═CH—, —(C(R⁵)₂)ₘC═(O)(CH₂)ₚ—, —(C(R⁵)₂)ₘC═(O)O(C(R⁵)₂)ₚ—, —(C(R⁵)₂)ₘC═(O)NR³(C(R⁵)₂)ₚ—, —(C(R⁵)₂)ₘNR³C═(O)(C(R⁵)₂)ₚ—, —(C(R⁵)₂)ₘphenyl(C(R⁵)₂)ₚ—, —(C(R⁵)₂)ₘpyridinyl(C(R⁵)₂)ₚ—, or —(C(R⁵)₂)ₘthiophenyl(C(R⁵)₂)ₚ—;
Y¹ is O, NR, or S(O)_{q};
each Y² is independently O, NH or S;
R¹ is absent, C₆-C₁₀ arylene, heteroarylene, or C₃-C₈cycloalkylene, wherein the heteroarylene comprises one or two 5- to 7-membered rings and 1-4 heteroatoms selected from N, O and S, and wherein the C₆-C₁₀ arylene, heteroarylene, and C₃-C₈cycloalkylene are optionally substituted with one to two R^{e};
R² is H or C₁-C₄ alkyl;
R^{2′} is H, C₁-C₄ alkyl, or C₃-C₇ cycloalkyl; or
R² and R^{2′} together with the nitrogen atom to which they are attached form a 3- to 7-membered heterocycloalkyl ring comprising 1-3 additional heteroatoms selected from N, O and S;
R³ is H or C₁-C₄ alkyl;
R⁴ is H or C₁-C₄ alkyl;
each R⁵ is independently at each occurrence H or C₁-C₄ alkyl;
each R⁶ is independently at each occurrence H or C₁-C₄ alkyl;
R⁷ is H, A, B, or C;
A is —(C(R⁶)₂)ᵣCO₂R^{x}, —Y²(C(R⁶)₂)ᵣCO₂R^{x}, —(C(R⁶)₂)ᵣtetrazole, —(C(R⁶)₂)ᵣoxadiazolone, —(C(R⁶)₂)ᵣtetrazolone, —(C(R⁶)₂)ᵣthiadiazolol, —(C(R⁶)₂)ᵣ isoxazol-3-ol, —(C(R⁶)₂)ᵣP(O)(OH)OR^{x}, —(C(R⁶)₂)ᵣS(O)₂OH, —(C(R⁶)₂)ᵣC(O)NHCN, or —(C(R⁶)₂)ᵣC(O)NHS(O)₂alkyl, wherein —(C(R⁶)₂)ᵣtetrazole, —(C(R⁶)₂)ᵣoxadiazolone, —(C(R⁶)₂)ᵣtetrazolone, —(C(R⁶)₂)ᵣthiadiazolol, —(C(R⁶)₂)ᵣ isoxazol-3-ol are optionally substituted with C₁-C₆ alkyl,
B is —(C(R⁶)₂)ᵣS(O)₂OC₁-C₄ alkyl, —O(C(R⁶)₂)ᵣS(O)₂OC₁-C₄ alkyl, —Y²(C(R⁶)₂)ᵣC(O)NR^{g}R^{g′}, —Y²(C(R⁶)₂)ᵣS(O)₂NR^{g}R^{g′}, —(C(R⁶)₂)ᵣC(O)NR^{g}R^{g′}, —(C(R⁶)₂)ᵣS(O)₂NR^{g}R^{g′}, —(C(R⁶)₂)ᵣC(O)NHS(O)₂NR^{g}R^{g′}, —(C(R⁶)₂)ᵣCO₂Rⁱ, —(C(R⁶)₂)ᵣNH₂CO₂R^{x}, —(C(R⁶)₂)ᵣP(O)(OR^{x})₂, —O(C(R⁶)₂)ᵣP(O)(OR^{x})₂, —(C(R⁶)₂)ᵣS(O)₂OH, —O(C(R⁶)₂)ᵣS(O)₂OH, —(C(R⁶)₂)ᵣP(O)₂OR^{x}, or —O(C(R⁶)₂)ᵣP(O)₂OR^{x},
C is —(CH₂)ᵣCN, —(CH₂)ₛOH, halogen, —(C(R⁶)₂)ᵣC₆-C₁₀ aryl, —(C(R⁶)₂)ᵣS—C₆-C₁₀ aryl, —(C(R⁶)₂)ᵣheteroaryl, —O(C(R⁶)₂)ᵣheteroaryl, —O(C(R⁶)₂)ᵣheterocycloalkyl, —O(C(R⁶)₂)ᵣOH, —OR^{y}, —(C(R⁶)₂)ᵣC(O)NHCN, —CH═CHCO₂R^{x}, or —(C(R⁶)₂)ᵣC(O)NHS(O)₂C₁-C₄ alkyl, wherein the aryl and heteroaryl are substituted with one to three substituents each independently selected from C₁-C₆ alkyl, C₁-C₆ haloalkyl, halogen, and OH, and wherein the heterocycloalkyl is substituted with one to two ═O or ═S;
R^{c} is H, C₁-C₆ alkyl, C₁-C₆ haloalkyl, halogen, —CN, —OR^{x}, or —CO₂R^{x};
R^{d} is methyl, CF₃, CR^{f}F₂, —(C(R⁶)₂)ₜC₆-C₁₀ aryl, —(C(R⁶)₂)ₜ-5- or 6-membered heteroaryl, —(C(R⁶)₂)ₜ-5- or 6-membered cycloalkyl, optionally substituted C₆-C₁₀ aryl, optionally substituted 5- or 6-membered heteroaryl, or optionally substituted 5- or 6-membered cycloalkyl;
each R^{e} is independently at each occurrence C₁-C₆ alkyl, C₂-C₆ alkenyl, C₂-C₆ alkynyl, halogen, C₁-C₆ haloalkyl, —NHR^{z}, —OH, or —CN;
R^{f} is absent, H, or methyl;
R^{g} is H, C₁-C₆ alkyl, OH, —S(O)₂(C₁-C₆ alkyl), or S(O)₂N(C₁-C₆ alkyl)₂;
R^{g′} is H, C₁-C₆ alkyl, C₃-C₇ cycloalkyl, 4- to 7-membered heterocycloalkyl ring comprising 1-3 heteroatoms selected from N, O and S, C₆-C₁₀ aryl, or 5- to 7-membered heteroaryl comprising 1-3 heteroatoms selected from N, O and S, wherein the alkyl is optionally substituted with one or more substituents independently selected from halogen and —OH, and wherein the cycloalkyl, heterocycloalkyl, aryl and heteroaryl are optionally substituted with one or more substituents independently selected from C₁-C₆ alkyl, halogen, and —OH;
R^{h} is H, C₁-C₄ alkyl, or 3- to 7-membered heterocycloalkyl ring comprising 1-3 heteroatoms selected from N, O and S, wherein the alkyl is optionally substituted with one or more substituents each independently selected from NH₂, C₁-C₄ alkylamino, C₁-C₄ dialkylamino, and C(O)NH₂; and wherein the heterocycloalkyl is optionally substituted with one or more substituents each independently selected from C₁-C₆ alkyl and C₁-C₆ haloalkyl;
Rⁱ is (i) —(CH₂)ₛOC(O)C₁-C₆ alkyl, wherein the alkyl is substituted with one or more NH₂; (ii) (CH₂CH₂O)ₙCH₂CH₂OH; or (iii) C₁-C₆ alkyl substituted with one or more substituents each independently selected from OH and 4- to 7-membered heterocycloalkyl comprising 1 to 3 heteroatoms selected from O, N, or S;
R^{j} is absent, H, C₁-C₆ alkyl, or —CN;
each R^{x} is independently at each occurrence H, C₁-C₆ alkyl, or C₆-C₁₀ aryl;
each R^{y} and R^{z} is independently H, C₁-C₆ alkyl, or C₁-C₆ haloalkyl;
each m, p, q, r, and t is independently 0, 1 or 2;
n is 0, 1, 2, or 3;
s is 1 or 2;
o is 0, 1, 2, 3, or 4; and
[symbol] represents a single bond or a double bond; and
provided that
when X is O; R^{f} is H; W is C; R^{j} is —CN; L is —SCH₂—; R¹ is phenylene or pyridine; then R⁷ is not —COOH;
when X is O; R^{f} is H; W is C; R^{j} is —CN; L is —SCH₂—; R¹ is phenylene or pyridine; and R⁷ is tetrazole; then R^{c} is not H;
when X is O; R^{f} is H; W is C; R^{j} is —CN; L is —S—C(R⁵)₂ or —SCH₂CH₂—; R¹ is absent; then R⁷ is not COOH or tetrazole;
when X is O, R^{f} is H; W is N; R^{j} is absent; R^{d} is methyl, optionally substituted 5- to 10-membered aryl, optionally substituted 5- or 6-membered heteroaryl, or optionally substituted 5- or 6-membered cycloalkyl; L is —SCH₂— or —OCH₂—; and R¹ is phenylene; then R⁷ is not —COOH, —CH₂COOH,

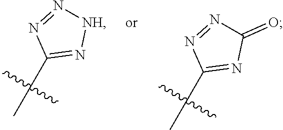 [SMILES: *c1nn[nH]n1 ; "or" ; *C1=NC(=O)N=N1]

and
when X is O, R^{f} is H, W is N, R^{j} is absent, L is —NHCH₂—, —CH₂NH—, or —NH—C(O)—, and R¹ is phenylene, then R^{d} is not phenyl.

Embodiment I-2. A method of treating an acute inflammatory condition in a subject comprising administering to the subject a therapeutically effective amount of a compound represented by Formula (I):

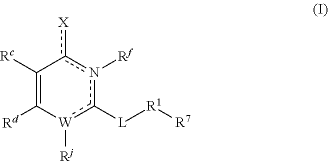

or a pharmaceutically acceptable salt or tautomer thereof,
wherein:
X is H, S, SR², NR², NR²R^{2′}, OH, OR^{h}, F, Br, or Cl;
W is N or C;
   (i) when W is N, then:
   L is —(C(R⁵)₂)ₘCH═CH(C(R⁵)₂)ₚ—,

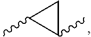

—(C(R⁵)₂)ₘY¹(C(R⁵)₂)ₚ—, —(C(R⁵)₂)ₘY¹(C(R⁵)₂)ₚ-cyclopropyl-, —(C(R⁵)₂)ₘY¹CH═CH—, —(C(R⁵)₂)ₘNR³C═(O)(C(R⁵)₂)ₚ—, —(C(R⁵)₂)ₘphenyl(C(R⁵)₂)ₚ—, —(C(R⁵)₂)ₘpyridinyl(C(R⁵)₂)ₚ—, or —(C(R⁵)₂)ₘthiophenyl(C(R⁵)₂)ₚ—;
(ii) when W is C, then:
L is —(C(R⁵)₂)ₘCH═CH(C(R⁵)₂)ₚ—,

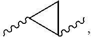

—(C(R⁵)₂)ₘY¹(C(R⁵)₂)ₚ—, (C(R⁵)₂)ₘ Y¹CH═CH—, —(C(R⁵)₂)ₘC═(O)(CH₂)ₚ—, —(C(R⁵)₂)ₘC═(O)O(C(R⁵)₂)ₚ—, —(C(R⁵)₂)ₘC═(O)NR³(C(R⁵)₂)ₚ—, —(C(R⁵)₂)ₘNR³C═(O)(C(R⁵)₂)ₚ—, —(C(R⁵)₂)ₘphenyl(C(R⁵)₂)ₚ—, —(C(R⁵)₂)ₘpyridinyl(C(R⁵)₂)ₚ—, or —(C(R⁵)₂)ₘthiophenyl(C(R⁵)₂)ₚ—;
Y¹ is O, NR⁴, or S(O)_{q};
each Y² is independently O, NH or S;
R¹ is absent or C₆-C₁₀ arylene or heteroarylene, wherein the heteroarylene comprises one or two 5- to 7-membered rings and 1-4 heteroatoms selected from N, O and S, and wherein the C₆-C₁₀ arylene or heteroarylene are optionally substituted with one to two R^{e};
R² is H or C₁-C₄ alkyl;
R^{2′} is H, C₁-C₄ alkyl, or C₃-C₇ cycloalkyl; or
R² and R^{2′} together with the nitrogen atom to which they are attached form a 3- to 7-membered heterocycloalkyl ring comprising 1-3 additional heteroatoms selected from N, O and S;
R³ is H or C₁-C₄ alkyl;
R⁴ is H or C₁-C₄ alkyl;
each R⁵ is independently at each occurrence H or C₁-C₄ alkyl;
each R⁶ is independently at each occurrence H or C₁-C₄ alkyl;
R⁷ is H, A, B, or C;
A is —(C(R⁶)₂)ᵣCO₂R^{x}, —Y²(C(R⁶)₂)ᵣCO₂R^{x}, —(CH₂)ᵣtetrazole, —(CH₂)ᵣoxadiazolone, —(CH₂)ᵣtetrazolone, —(CH₂)ᵣthiadiazolol, —(CH₂)ᵣ isoxazol-3-ol, —(CH₂)ᵣP(O)(OH)OR^{x}, —(CH₂)ᵣS(O)₂OH, —(CH₂)ᵣC(O)NHCN, or —(CH₂)ᵣC(O)NHS(O)₂alkyl, wherein —(CH₂)ᵣtetrazole, —(CH₂)ᵣoxadiazolone, —(CH₂)ᵣtetrazolone, —(CH₂)ᵣthiadiazolol, —(CH₂)ᵣ isoxazol-3-ol are optionally substituted with C₁-C₆ alkyl,
B is —(C(R⁶)₂)ᵣS(O)₂OC₁-C₄ alkyl, —O(C(R⁶)₂)ᵣS(O)₂OC₁-C₄ alkyl, —Y²(C(R⁶)₂)ᵣC(O)NR^{g}R^{g′}, —Y²(C(R⁶)₂)ᵣS(O)₂NR^{g}R^{g′}, —(CH₂)ᵣC(O)NR^{g}R^{g′}, —(CH₂)ᵣS(O)₂NR^{g}R^{g′}, —(CH₂)ᵣC(O)NHS(O)₂NR^{g}R^{g′}, —(C(R⁶)₂)ᵣCO₂R¹, —(C(R⁶)₂)ᵣNH₂CO₂R^{x}, —(C(R⁶)₂)ᵣP(O)(OR^{x})₂, —O(C(R⁶)₂)ᵣP(O)(OR^{x})₂, —(C(R⁶)₂)ᵣS(O)₂OH, —O(C(R⁶)₂)ᵣS(O)₂OH, —(C(R⁶)₂)ᵣP(O)₂OR^{x}, or —O(C(R⁶)₂)ᵣP(O)₂OR^{x},
C is —(CH₂)ᵣCN, —(CH₂)ₛOH, halogen, —(C(R⁶)₂)ᵣC₆-C₁₀ aryl, —(C(R⁶)₂)ᵣS—C₆-C₁₀ aryl, —(C(R⁶)₂)ᵣheteroaryl, —O(C(R⁶)₂)ᵣheteroaryl, —O(C(R⁶)₂)ᵣheterocycloalkyl, —O(C(R⁶)₂)ᵣOH, —OR^{y}, —(C(R⁶)₂)ᵣC(O)NHCN, —CH═CHCO₂R^{x}, or —(C(R⁶)₂)ᵣC(O)NHS(O)₂C₁-C₄ alkyl,
wherein the aryl and heteroaryl are substituted with one to three substituents each independently selected from C₁-C₆ alkyl, C₁-C₆ haloalkyl, halogen, and OH, and wherein the heterocycloalkyl is substituted with one to two ═O or ═S;
R^{c} is H, C₁-C₆ alkyl, C₁-C₆ haloalkyl, halogen, —CN, —OR^{x}, or —CO₂R^{x};
R^{d} is methyl, CF₃, CR^{f}F₂, —(C(R⁶)₂)ₜC₆-C₁₀ aryl, —(C(R⁶)₂)ₜ-5- or 6-membered heteroaryl, —(C(R⁶)₂)ₜ-5- or 6-membered cycloalkyl, optionally substituted C₆-C₁₀ aryl, optionally substituted 5- or 6-membered heteroaryl, or optionally substituted 5- or 6-membered cycloalkyl;
each R^{e} is independently at each occurrence C₁-C₆ alkyl, C₂-C₆ alkenyl, C₂-C₆ alkynyl, halogen, C₁-C₆ haloalkyl, —NHR^{z}, —OH, or —CN;
R^{f} is absent, H, or methyl;
R^{g} is H, C₁-C₆ alkyl, OH, —S(O)₂(C₁-C₆ alkyl), or S(O)₂N(C₁-C₆ alkyl)₂;
R^{g′} is H, C₁-C₆ alkyl, C₃-C₇ cycloalkyl, 4- to 7-membered heterocycloalkyl ring comprising 1-3 heteroatoms selected from N, O and S, C₆-C₁₀ aryl, or 5- to 7-membered heteroaryl comprising 1-3 heteroatoms selected from N, O and S, wherein the alkyl is optionally substituted with one or more substituents independently selected from halogen and —OH, and wherein the cycloalkyl, heterocycloalkyl, aryl and heteroaryl are optionally substituted with one or more substituents independently selected from C₁-C₆ alkyl, halogen, and —OH;
R^{h} is H, C₁-C₄ alkyl, or 3- to 7-membered heterocycloalkyl ring comprising 1-3 heteroatoms selected from N, O and S, wherein the alkyl is optionally substituted with one or more substituents each independently selected from NH₂, C₁-C₄ alkylamino, C₁-C₄ dialkylamino, and C(O)NH₂; and wherein the heterocycloalkyl is optionally substituted with one or more substituents each independently selected from C₁-C₆ alkyl and C₁-C₆ haloalkyl;
Rⁱ is (i) —(CH₂)ₛOC(O)C₁-C₆ alkyl, wherein the alkyl is substituted with one or more NH₂; (ii) (CH₂CH₂O)ₙCH₂CH₂OH; or (iii) C₁-C₆ alkyl substituted with one or more substituents each independently selected from OH and 4- to 7-membered heterocycloalkyl comprising 1 to 3 heteroatoms selected from O, N, or S;
R^{j} is absent, H, C₁-C₆ alkyl, or —CN;
each R^{x} is independently at each occurrence H, C₁-C₆ alkyl, or C₆-C₁₀ aryl;
each R^{y} and R^{z} is independently H, C₁-C₆ alkyl, or C₁-C₆ haloalkyl;
each m, p, q, r, and t is independently 0, 1 or 2;
n is 0, 1, 2, or 3;
s is 1 or 2;
o is 0, 1, 2, 3, or 4; and
[symbol] represents a single bond or a double bond; and
provided that
when X is O; R^{f} is H; W is C; R^{j} is —CN; L is —SCH₂—; R¹ is phenylene or pyridine; then R⁷ is not —COOH;
when X is O; R^{f} is H; W is C; R^{j} is —CN; L is —SCH₂—; R¹ is phenylene or pyridine; and R⁷ is tetrazole; then R^{c} is not H;
when X is O; R^{f} is H; W is C; R^{j} is —CN; L is —S—C(R⁵)₂ or —SCH₂CH₂—; R¹ is absent; then R⁷ is not COOH or tetrazole;
when X is O, R^{f} is H; W is N; R^{j} is absent; R^{d} is methyl, optionally substituted 5- to 10-membered aryl, optionally substituted 5- or 6-membered heteroaryl, or optionally substituted 5- or 6-membered cycloalkyl; L is —SCH₂— or —OCH₂—; and R¹ is phenylene; then R⁷ is not —COOH, —CH₂COOH,

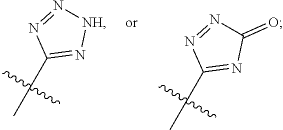 [SMILES: *c1nn[nH]n1 ; "or" ; *C1=NC(=O)N=N1]

and
when X is O, R^{f} is H, W is N, R^{j} is absent, L is —NHCH₂—, —CH₂NH—, or —NH—C(O)—, and R¹ is phenylene, then R^{d} is not phenyl.

Embodiment I-3. The method of Embodiment I-1 or I-2, wherein X is O, OH, OR^{h}, F, Br, or Cl.

Embodiment I-4. The method of Embodiment I-1 or I-2, wherein X is H, S, SR², NR², or NR²R^{2′}.

Embodiment I-5 The method of Embodiment I-1 or I-4, wherein R^{f} is absent.

Embodiment I-6. The method of Embodiment I-1 or I-4, wherein R^{f} is H or methyl.

Embodiment I-7. The method of Embodiment I-1 or I-6, wherein W is N.

Embodiment I-8. The method of Embodiment I-7, wherein R^{j} is absent.

Embodiment I-9. The method of any one of Embodiments I-1 to I-6, wherein W is C.

Embodiment I-10. The method of Embodiment I-9, wherein R^{j} is H, C₁-C₆ alkyl, or —CN.

Embodiment I-11. The method of Embodiment I-9 or I-10, wherein R^{j} is —CN.

Embodiment I-12. The method of any one of Embodiments I-1 to I-11, wherein R^{c} is C₁-C₆ alkyl, —CN, or halogen.

Embodiment I-13. The method of any one of Embodiments I-1 to I-12, wherein R^{c} is —CN or halogen.

Embodiment I-14. The method of any one of Embodiments I-1 to I-12, wherein R^{c} is —CN.

Embodiment I-15. The method of any one of Embodiments I-1 to I-14, wherein R^{d} is methyl.

Embodiment I-16. The method of any one of Embodiments I-1 to I-14, wherein R^{d} is optionally substituted 5- to 10-membered aryl.

Embodiment I-17. The method of any one of Embodiments I-1 to I-14, wherein R^{d} is optionally substituted 5- or 6-membered heteroaryl.

Embodiment I-18. The method of any one of Embodiments I-1 to I-14, wherein R^{d} is optionally substituted 5- or 6-membered cycloalkyl.

Embodiment I-19. The method of any one of Embodiments I-1 to I-14, wherein R^{d} is methyl, cyclohexyl, pyridinyl, thiazolyl, phenyl, or thienyl.

Embodiment I-20. The method of any one of Embodiments I-1 to I-14, wherein R^{d} is methyl, cyclohexyl, pyridinyl, thiazolyl, thienyl, or optionally substituted phenyl.

Embodiment I-21. The method of any one of Embodiments I-1 to I-14, wherein R^{d} is methyl.

Embodiment I-22. The method of any one of Embodiments I-1 to I-14, wherein R^{d} is —CF₃.

Embodiment I-23. The method of any one of Embodiments I-1 to I-14, wherein R^{d} is CR^{f}F₂.

Embodiment I-24. The method of any one of Embodiments I-1 to I-14, wherein R^{d} is —(C(R⁶)₂)ₜC₆-C₁₀ aryl, —(C(R⁶)₂)ₜ-5- or 6-membered heteroaryl, —(C(R⁶)₂)ₜ-5- or 6-membered cycloalkyl.

Embodiment I-25. The method of any one of Embodiments I-1 to I-14, wherein R^{d} is —(C(R⁶)₂)ₜC₆-C₁₀ aryl.

Embodiment I-26. The method of any one of Embodiments I-1 to I-25, wherein L is —(C(R⁵)₂)ₘY¹(C(R⁵)₂)ₚ—.

Embodiment I-27. The method of Embodiment I-26, wherein Y¹ is S.

Embodiment I-28. The method of any one of Embodiment I-1 to I-25, wherein L is —(C(R⁵)₂)ₘNR³C═(O)(C(R⁵)₂)ₚ— or —(C(R⁵)₂)ₘY¹(C(R⁵)₂)ₚ-cyclopropyl-.

Embodiment I-29. The method of any one of Embodiments I-1 to I-28, wherein R¹ is C₆-C₁₀ arylene.

Embodiment I-30. The method of any one of Embodiments I-1 to I-28, wherein R¹ is heteroarylene.

Embodiment I-31. The method of any one of Embodiments I-1 to I-28, wherein R¹ is absent.

Embodiment I-32. The method of any one of Embodiments I-1 to I-31, wherein R⁷ is A.

Embodiment I-33. The method of Embodiment I-32, wherein A is —(C(R⁶)₂)ᵣCO₂R^{x} or —(CH₂)ᵣtetrazole, wherein the —(CH₂)ᵣtetrazole is optionally substituted with C₁-C₆ alkyl.

Embodiment I-34. The method of any one of Embodiments I-1 to I-31, wherein R⁷ is B.

Embodiment I-35. The method of Embodiment I-32, wherein B is —(CH₂)ᵣC(O)NR^{g}R^{g′}, or —(CH₂)ᵣS(O)₂NR^{g}R^{g′},

Embodiment I-36. The method of any one of Embodiments I-1 to I-31, wherein R⁷ is C.

Embodiment I-37. The method of Embodiment I-32, wherein C is —(CH₂)ᵣCN, —(CH₂)ₛOH, or —(C(R⁶)₂)ᵣC₆-C₁₀ aryl, wherein the aryl is substituted with one to three substituents each independently selected from C₁-C₆ alkyl, C₁-C₆ haloalkyl, halogen, and OH.

Embodiment I-38. The method of any one of Embodiment I-1 to I-37, wherein the compound is

| | |
|---|---|
| I-34 | 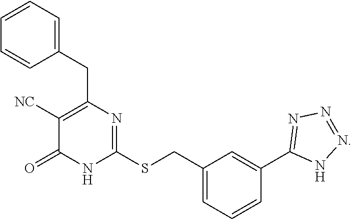 [SMILES: N#Cc1c(Cc2ccccc2)nc(SCc2cccc(-c3nnn[nH]3)c2)[nH]c1=O] |

Embodiment I-39. The method of any one of Embodiments I-1 to I-38, wherein the acute inflammatory condition is a systemic inflammatory condition.

Embodiment I-40. The method of any one of Embodiments I-1 to I-38, wherein the acute inflammatory condition is an organ-specific condition.

Embodiment I-41. The method of any one of Embodiments I-1 to I-38, wherein the acute inflammatory condition is cytokine storm or hypercytokinemia, systemic inflammatory response syndrome (SIRS), graft versus host disease (GVHD), acute respiratory distress syndrome (ARDS), severe acute respiratory distress syndrome (SARS), catastrophic anti-phospholipid syndrome, viral infections, bacterial infections, fungal infections, influenza, pneumonia, shock, or sepsis.

Embodiment I-42. The method of any one of Embodiments I-1 to I-38, wherein the acute inflammatory condition is acute pancreatitis, hepatitis, respiratory condition, or enterocolitis.

Embodiment I-43. The method of any one of Embodiments I-1 to I-42, wherein the method reduces a pro-inflammatory cytokine or increases an anti-inflammatory cytokine.

Embodiment I-44. The method of Embodiment I-43, wherein the pro-inflammatory cytokine is IL-1β, IL-6, IL-18, TNF-α, or TGF-β.

Embodiment I-45. The method of Embodiment I-43, wherein pro-inflammatory cytokine is MCP-1, TNF-α, or IL-1β and the pro-inflammatory cytokine is increased.

Embodiment I-46. The method of Embodiment I-43, wherein pro-inflammatory cytokine is IL-6 and the pro-inflammatory cytokine is increased.

Embodiment I-47. The method of Embodiment I-43, wherein the anti-inflammatory cytokine is IL-10.

Embodiment I-48. The method of any one of Embodiments I-1 to I-42, wherein expression of sirtuin-1 modulated genes sod2, tfam, dda1 genes are increased in the liver.

Embodiment I-49. The method of any one of Embodiments I-1 to I-48, wherein the administration to the subject occurs at least 12 hours after an injury.

Embodiment I-50. The method of any one of Embodiments I-1 to I-49, wherein the administration to the subject occurs for at least 6 days after an injury.

Embodiment I-1A. A compound represented by Formula (II):

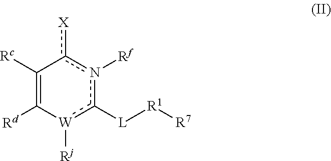

or a pharmaceutically acceptable salt or tautomer thereof, wherein:
X is H, S, SR², NR², NR²R^{2′}, O, OH, OR^{h}, F, Br, or Cl;
W is N or C;
(i) when W is N, then: L is —(C(R⁵)₂)ₘCH═CH(C(R⁵)₂)ₚ—,

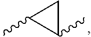

—(C(R⁵)₂)ₘY¹(C(R⁵)₂)ₚ—, —(C(R⁵)₂)ₘY¹(C(R⁵)₂)ₚ-cyclopropyl-, —(C(R⁵)₂)ₘY¹CH═CH—, —(C(R⁵)₂)ₘNR³C═(O)(C(R⁵)₂)ₚ—, —(C(R⁵)₂)ₘphenyl(C(R⁵)₂)ₚ—, —(C(R⁵)₂)ₘpyridinyl(C(R⁵)₂)ₚ—, or —(C(R⁵)₂)ₘthiophenyl(C(R⁵)₂)ₚ—;
(ii) when W is C, then: L is —(C(R⁵)₂)ₘCH═CH(C(R⁵)₂)ₚ—, —(C(R⁵)₂)ₒ—, —(C(R⁵)₂)ₘY¹(C(R⁵)₂)ₚ—,

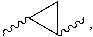

—(C(R⁵)₂)ₘ Y¹CH═CH—, —(C(R⁵)₂)ₘC═(O)(CH₂)ₚ—, —(C(R⁵)₂)ₘC═(O)O(C(R⁵)₂)ₚ—, —(C(R⁵)₂)ₘC═(O)NR³(C(R⁵)₂)ₚ—, —(C(R⁵)₂)ₘNR³C═(O)(C(R⁵)₂)ₚ—, —(C(R⁵)₂)ₘphenyl(C(R⁵)₂)ₚ—, —(C(R⁵)₂)ₘpyridinyl(C(R⁵)₂)ₚ—, or —(C(R⁵)₂)ₘthiophenyl(C(R⁵)₂)ₚ—;
Y¹ is O, NR⁴, or S(O)_{q};
each Y² is independently O, NH or S;
R¹ is absent, C₆-C₁₀ arylene, heteroarylene, or C₃-C₈cycloalkylene, wherein the heteroarylene comprises one or two 5- to 7-membered rings and 1-4 heteroatoms selected from N, O and S, and wherein the C₆-C₁₀ arylene, heteroarylene, and C₃-C₈cycloalkylene are optionally substituted with one to two R^{e};
R² is H or C₁-C₄ alkyl;
R^{2′} is H, C₁-C₄ alkyl, or C₃-C₇ cycloalkyl; or
R² and R^{2′} together with the nitrogen atom to which they are attached form a 3- to 7-membered heterocycloalkyl ring comprising 1-3 additional heteroatoms selected from N, O and S;
R³ is H or C₁-C₄ alkyl;
R⁴ is H or C₁-C₄ alkyl;
each R⁵ is independently at each occurrence H or C₁-C₄ alkyl;
each R⁶ is independently at each occurrence H or C₁-C₄ alkyl;
R⁷ is H, A, B, or C;
A is —(C(R⁶)₂)ᵣCO₂R^{x}, —Y²(C(R⁶)₂)ᵣCO₂R^{x}, —(C(R⁶)₂)ᵣtetrazole, —(C(R⁶)₂)ᵣoxadiazolone, —(C(R⁶)₂)ᵣtetrazolone, —(C(R⁶)₂)ᵣthiadiazolol, —(C(R⁶)₂)ᵣ isoxazol-3-ol, —(C(R⁶)₂)ᵣP(O)(OH)OR^{x}, —(C(R⁶)₂)ᵣS(O)₂OH, —(C(R⁶)₂)ᵣC(O)NHCN, or —(C(R⁶)₂)ᵣC(O)NHS(O)₂alkyl, wherein —(C(R⁶)₂)ᵣtetrazole, —(C(R⁶)₂)ᵣoxadiazolone, —(C(R⁶)₂)ᵣtetrazolone, —(C(R⁶)₂)ᵣthiadiazolol, —(C(R⁶)₂)ᵣ isoxazol-3-ol are optionally substituted with C₁-C₆ alkyl,
B is —(C(R⁶)₂)ᵣS(O)₂OC₁-C₄ alkyl, —O(C(R⁶)₂)ᵣS(O)₂OC₁-C₄ alkyl, —Y²(C(R⁶)₂)ᵣC(O)NR^{g}R^{g′}, —Y²(C(R⁶)₂)ᵣS(O)₂NR^{g}R^{g′}, —(C(R⁶)₂)ᵣC(O)NR^{g}R^{g′}, —(C(R⁶)₂)ᵣS(O)₂NR^{g}R^{g′}, —(C(R⁶)₂)ᵣC(O)NHS(O)₂NR^{g}R^{g′}, —(C(R⁶)₂)ᵣCO₂Rⁱ, —(C(R⁶)₂)ᵣNH₂CO₂R^{x}, —(C(R⁶)₂)ᵣP(O)(OR^{x})₂, —O(C(R⁶)₂)ᵣP(O)(OR^{x})₂, —(C(R⁶)₂)ᵣS(O)₂OH, —O(C(R⁶)₂)ᵣS(O)₂OH, —(C(R⁶)₂)ᵣP(O)₂OR^{x}, or —O(C(R⁶)₂)ᵣP(O)₂OR^{x},
C is —(CH₂)ᵣCN, —(CH₂)ₛOH, halogen, —(C(R⁶)₂)ᵣC₆-C₁₀ aryl, —(C(R⁶)₂)ᵣS—C₆-C₁₀ aryl, —(C(R⁶)₂)ᵣheteroaryl, —O(C(R⁶)₂)ᵣheteroaryl, —O(C(R⁶)₂)ᵣheterocycloalkyl, —O(C(R⁶)₂)ᵣOH, —OR^{y}, —(C(R⁶)₂)ᵣC(O)NHCN, —CH═CHCO₂R^{x}, or —(C(R⁶)₂)ᵣC(O)NHS(O)₂C₁-C₄ alkyl, wherein the aryl and heteroaryl are substituted with one to three substituents each independently selected from C₁-C₆ alkyl, C₁-C₆ haloalkyl, halogen, and OH, and wherein the heterocycloalkyl is substituted with one to two ═O or ═S;
R^{c} is H, C₁-C₆ alkyl, C₁-C₆ haloalkyl, halogen, —CN, —OR^{x}, or —CO₂R^{x};
R^{d} is methyl, CF₃, CR^{f}F₂, —(C(R⁶)₂)ₜC₆-C₁₀ aryl, —(C(R⁶)₂)ₜ-5- or 6-membered heteroaryl, —(C(R⁶)₂)ₜ-5- or 6-membered cycloalkyl, optionally substituted C₆-C₁₀ aryl, optionally substituted 5- or 6-membered heteroaryl, or optionally substituted 5- or 6-membered cycloalkyl;
each R^{e} is independently at each occurrence C₁-C₆ alkyl, C₂-C₆ alkenyl, C₂-C₆ alkynyl, halogen, C₁-C₆ haloalkyl, —NHR^{z}, —OH, or —CN;
R^{f} is absent, H, or methyl;
R^{g} is H, C₁-C₆ alkyl, OH, —S(O)₂(C₁-C₆ alkyl), or S(O)₂N(C₁-C₆ alkyl)₂;
R^{g′} is H, C₁-C₆ alkyl, C₃-C₇ cycloalkyl, 4- to 7-membered heterocycloalkyl ring comprising 1-3 heteroatoms selected from N, O and S, C₆-C₁₀ aryl, or 5- to 7-membered heteroaryl comprising 1-3 heteroatoms selected from N, O and S, wherein the alkyl is optionally substituted with one or more substituents independently selected from halogen and —OH, and wherein the cycloalkyl, heterocycloalkyl, aryl and heteroaryl are optionally substituted with one or more substituents independently selected from C₁-C₆ alkyl, halogen, and —OH;
R^{h} is H, C₁-C₄ alkyl, or 3- to 7-membered heterocycloalkyl ring comprising 1-3 heteroatoms selected from N, O and S, wherein the alkyl is optionally substituted with one or more substituents each independently selected from NH₂, C₁-C₄ alkylamino, C₁-C₄ dialkylamino, and C(O)NH₂; and wherein the heterocycloalkyl is optionally substituted with one or more substituents each independently selected from C₁-C₆ alkyl and C₁-C₆ haloalkyl;
Rⁱ is (i) —(CH₂)ₛOC(O)C₁-C₆ alkyl, wherein the alkyl is substituted with one or more NH₂; (ii) (CH₂CH₂O)ₙCH₂CH₂OH; or (iii) C₁-C₆ alkyl substituted with one or more substituents each independently selected from OH and 4- to 7-membered heterocycloalkyl comprising 1 to 3 heteroatoms selected from O, N, or S;
R^{j} is absent, H, C₁-C₆ alkyl, or —CN;
each R^{x} is independently at each occurrence H, C₁-C₆ alkyl, or C₆-C₁₀ aryl;
each R^{y} and R^{z} is independently H, C₁-C₆ alkyl, or C₁-C₆ haloalkyl;
each m, p, q, r, and t is independently 0, 1 or 2;
n is 0, 1, 2, or 3;
s is 1 or 2;
o is 0, 1, 2, 3, or 4; and
[symbol] represents a single bond or a double bond; and
provided that
when X is O; R^{f} is H; W is C; R^{j} is —CN; L is —SCH₂—; R¹ is phenylene or pyridine; then R⁷ is not —COOH;
when X is O; R^{f} is H; W is C; R^{j} is —CN; L is —SCH₂—; R¹ is phenylene or pyridine; and R⁷ is tetrazole; then R^{c} is not H;
when X is O; R^{f} is H; W is C; R^{j} is —CN; L is —S—C(R⁵)₂ or —SCH₂CH₂—; R¹ is absent; then R⁷ is not COOH or tetrazole;
when X is O, R^{f} is H; W is N; R^{j} is absent; R^{d} is methyl, optionally substituted 5- to 10-membered aryl, optionally substituted 5- or 6-membered heteroaryl, or optionally substituted 5- or 6-membered cycloalkyl; L is —SCH₂— or —OCH₂—; and R¹ is phenylene; then R⁷ is not —COOH, —CH₂COOH,

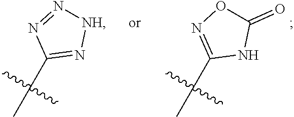 [SMILES: *c1nn[nH]n1 ; *c1noc(=O)[nH]1]

and
when X is O, R^{f} is H, W is N, R is absent, L is —NHCH₂—, —CH₂NH—, or —NH—C(O)—, and R¹ is phenylene, then R^{d} is not phenyl;
for use in treating an acute inflammatory condition.

Embodiment I-2A. A compound represented by Formula (I):

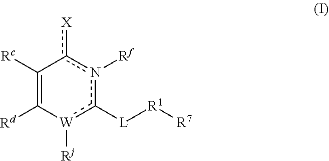

or a pharmaceutically acceptable salt or tautomer thereof,
wherein:
X is H, S, SR², NR², NR²R^{2′}, OH, OR^{h}, F, Br, or Cl;
W is N or C;
   (i) when W is N, then:
   L is —(C(R⁵)₂)ₘCH═CH(C(R⁵)₂)ₚ—,

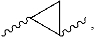

—(C(R⁵)₂)ₘY¹(C(R⁵)₂)ₚ—, —(C(R⁵)₂)ₘY¹ (C(R⁵)₂)ₚ-cyclopropyl-, —(C(R⁵)₂)ₘY¹CH═CH—, —(C(R⁵)₂)ₘNR³C═(O)(C(R⁵)₂)ₚ—, —(C(R⁵)₂)ₘphenyl(C(R⁵)₂)ₚ—, —(C(R⁵)₂)ₘpyridinyl(C(R⁵)₂)ₚ—, or —(C(R⁵)₂)ₘthiophenyl(C(R⁵)₂)ₚ—;
(ii) when W is C, then:
L is —(C(R⁵)₂)ₘCH═CH(C(R⁵)₂)ₚ—, —(C(R⁵)₂)ₒ, —(C(R⁵)₂)ₘY¹(C(R⁵)₂)ₚ—,

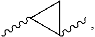

(C(R⁵)₂)ₘ Y¹CH═CH—, —(C(R⁵)₂)ₘC═(O)(CH₂)ₚ—, —(C(R⁵)₂)ₘC═(O)O(C(R⁵)₂)ₚ—, —(C(R⁵)₂)ₘC═(O)NR³(C(R⁵)₂)ₚ—, —(C(R⁵)₂)ₘNR³C═(O)(C(R⁵)₂)ₚ—, —(C(R⁵)₂)ₘphenyl(C(R⁵)₂)ₚ—, —(C(R⁵)₂)ₘpyridinyl(C(R⁵)₂)ₚ—, or —(C(R⁵)₂)ₘthiophenyl(C(R⁵)₂)ₚ—;
Y¹ is O, NR⁴, or S(O)_{q};
each Y² is independently O, NH or S;
R¹ is absent or C₆-C₁₀ arylene or heteroarylene, wherein the heteroarylene comprises one or two 5- to 7-membered rings and 1-4 heteroatoms selected from N, O and S, and wherein the C₆-C₁₀ arylene or heteroarylene are optionally substituted with one to two R^{e};
R² is H or C₁-C₄ alkyl;
R^{2′} is H, C₁-C₄ alkyl, or C₃-C₇ cycloalkyl; or
R² and R^{2′} together with the nitrogen atom to which they are attached form a 3- to 7-membered heterocycloalkyl ring comprising 1-3 additional heteroatoms selected from N, O and S;
R³ is H or C₁-C₄ alkyl;
R⁴ is H or C₁-C₄ alkyl;
each R⁵ is independently at each occurrence H or C₁-C₄ alkyl;
each R⁶ is independently at each occurrence H or C₁-C₄ alkyl;
R⁷ is H, A, B, or C;
A is —(C(R⁶)₂)ᵣCO₂R^{x}, —Y²(C(R⁶)₂)ᵣCO₂R^{x}, —(CH₂)ᵣtetrazole, —(CH₂)ᵣoxadiazolone, —(CH₂)ᵣtetrazolone, —(CH₂)ᵣthiadiazolol, —(CH₂)ᵣ isoxazol-3-ol, —(CH₂)ᵣP(O)(OH)OR^{x}, —(CH₂)ᵣS(O)₂OH, —(CH₂)ᵣC(O)NHCN, or —(CH₂)ᵣC(O)NHS(O)₂alkyl, wherein —(CH₂)ᵣtetrazole, —(CH₂)ᵣoxadiazolone, —(CH₂)ᵣtetrazolone, —(CH₂)ᵣthiadiazolol, —(CH₂)ᵣ isoxazol-3-ol are optionally substituted with C₁-C₆ alkyl,
B is —(C(R⁶)₂)ᵣS(O)₂OC₁-C₄ alkyl, —O(C(R⁶)₂)ᵣS(O)₂OC₁-C₄ alkyl, —Y²(C(R⁶)₂)ᵣC(O)NR^{g}R^{g′}, —Y²(C(R⁶)₂)ᵣS(O)₂NR^{g}R^{g′}, —(CH₂)ᵣC(O)NR^{g}R^{g′}, —(CH₂)ᵣS(O)₂NR^{g}R^{g′}, —(CH₂)ᵣC(O)NHS(O)₂NR^{g}R^{g′}, —(C(R⁶)₂)ᵣCO₂R¹, —(C(R⁶)₂)ᵣNH₂CO₂R^{x}, —(C(R⁶)₂)ᵣP(O)(OR^{x})₂, —O(C(R⁶)₂)ᵣP(O)(OR^{x})₂, —(C(R⁶)₂)ᵣS(O)₂OH, —O(C(R⁶)₂)ᵣS(O)₂OH, —(C(R⁶)₂)ᵣP(O)₂OR^{x}, or —O(C(R⁶)₂)ᵣP(O)₂OR^{x},
C is —(CH₂)ᵣCN, —(CH₂)ₛOH, halogen, —(C(R⁶)₂)ᵣC₆-C₁₀ aryl, —(C(R⁶)₂)ᵣS—C₆-C₁₀ aryl, —(C(R⁶)₂)ᵣheteroaryl, —O(C(R⁶)₂)ᵣheteroaryl, —O(C(R⁶)₂)ᵣheterocycloalkyl, —O(C(R⁶)₂)ᵣOH, —OR^{y}, —(C(R⁶)₂)ᵣC(O)NHCN, —CH═CHCO₂R^{x}, or —(C(R⁶)₂)ᵣC(O)NHS(O)₂C₁-C₄ alkyl, wherein the aryl and heteroaryl are substituted with one to three substituents each independently selected from C₁-C₆ alkyl, C₁-C₆ haloalkyl, halogen, and OH, and wherein the heterocycloalkyl is substituted with one to two ═O or ═S;
R^{c} is H, C₁-C₆ alkyl, C₁-C₆ haloalkyl, halogen, —CN, —OR^{x}, or —CO₂R^{x};
R^{d} is methyl, CF₃, CR^{f}F₂, —(C(R⁶)₂)ₜC₆-C₁₀ aryl, —(C(R⁶)₂)ₜ-5- or 6-membered heteroaryl, —(C(R⁶)₂)ₜ-5- or 6-membered cycloalkyl, optionally substituted C₆-C₁₀ aryl, optionally substituted 5- or 6-membered heteroaryl, or optionally substituted 5- or 6-membered cycloalkyl;
each R^{e} is independently at each occurrence C₁-C₆ alkyl, C₂-C₆ alkenyl, C₂-C₆ alkynyl, halogen, C₁-C₆ haloalkyl, —NHR^{z}, —OH, or —CN;
R^{f} is absent, H, or methyl;
R^{g} is H, C₁-C₆ alkyl, OH, —S(O)₂(C₁-C₆ alkyl), or S(O)₂N(C₁-C₆ alkyl)₂;
R^{g′} is H, C₁-C₆ alkyl, C₃-C₇ cycloalkyl, 4- to 7-membered heterocycloalkyl ring comprising 1-3 heteroatoms selected from N, O and S, C₆-C₁₀ aryl, or 5- to 7-membered heteroaryl comprising 1-3 heteroatoms selected from N, O and S, wherein the alkyl is optionally substituted with one or more substituents independently selected from halogen and —OH, and wherein the cycloalkyl, heterocycloalkyl, aryl and heteroaryl are optionally substituted with one or more substituents independently selected from C₁-C₆ alkyl, halogen, and —OH;
R^{h} is H, C₁-C₄ alkyl, or 3- to 7-membered heterocycloalkyl ring comprising 1-3 heteroatoms selected from N, O and S, wherein the alkyl is optionally substituted with one or more substituents each independently selected from NH₂, C₁-C₄ alkylamino, C₁-C₄ dialkylamino, and C(O)NH₂; and wherein the heterocycloalkyl is optionally substituted with one or more substituents each independently selected from C₁-C₆ alkyl and C₁-C₆ haloalkyl;
Rⁱ is (i) —(CH₂)ₛOC(O)C₁-C₆ alkyl, wherein the alkyl is substituted with one or more NH₂; (ii) (CH₂CH₂O)ₙCH₂CH₂OH; or (iii) C₁-C₆ alkyl substituted with one or more substituents each independently selected from OH and 4- to 7-membered heterocycloalkyl comprising 1 to 3 heteroatoms selected from O, N, or S;
R^{j} is absent, H, C₁-C₆ alkyl, or —CN;
each R^{x} is independently at each occurrence H, C₁-C₆ alkyl, or C₆-C₁₀ aryl;
each R^{y} and R^{z} is independently H, C₁-C₆ alkyl, or C₁-C₆ haloalkyl;
each m, p, q, r, and t is independently 0, 1 or 2;
n is 0, 1, 2, or 3;
s is 1 or 2;
o is 0, 1, 2, 3, or 4; and
[symbol] represents a single bond or a double bond; and
provided that
when X is O; R^{f} is H; W is C; R^{j} is —CN; L is —SCH₂—; R¹ is phenylene or pyridine; then R⁷ is not —COOH;
when X is O; R^{f} is H; W is C; R^{j} is —CN; L is —SCH₂—; R¹ is phenylene or pyridine; and R⁷ is tetrazole; then R^{c} is not H;
when X is O; R^{f} is H; W is C; R^{j} is —CN; L is —S—C(R⁵)₂ or —SCH₂CH₂—; R¹ is absent; then R⁷ is not COOH or tetrazole;
when X is O, R^{f} is H; W is N; R^{j} is absent; R^{d} is methyl, optionally substituted 5- to 10-membered aryl, optionally substituted 5- or 6-membered heteroaryl, or optionally substituted 5- or 6-membered cycloalkyl; L is —SCH₂— or —OCH₂—; and R¹ is phenylene; then R⁷ is not —COOH, —CH₂COOH,

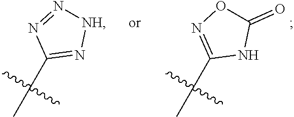 [SMILES: *c1nn[nH]n1 ; *c1noc(=O)[nH]1]

and
when X is O, R^{f} is H, W is N, R^{j} is absent, L is —NHCH₂—, —CH₂NH—, or —NH—C(O)—, and R¹ is phenylene, then R^{d} is not phenyl;
for use in treating an acute inflammatory condition.

Embodiment I-3A. The compound for use of Embodiment I-1A or I-2A, wherein X is O, OH, OR^{h}, F, Br, or Cl.

Embodiment I-4A. The compound for use of Embodiment I-1A or I-2A, wherein X is H, S, SR², NR², or NR²R^{2′}.

Embodiment I-5A. The compound for use of any one of Embodiments I-1A to I-4A, wherein R^{f} is absent.

Embodiment I-6A. The compound for use of any one of Embodiments I-1A to I-4A, wherein R^{f} is H or methyl.

Embodiment I-7A. The compound for use of any one of Embodiments I-1A to I-6A, wherein W is N.

Embodiment I-8A. The compound for use of Embodiment I-7A, wherein R^{j} is absent.

Embodiment I-9A. The compound for use of any one of Embodiments I-1A to I-6A, wherein W is C.

Embodiment I-10A. The compound for use of Embodiment I-9A, wherein R^{j} is H, C₁-C₆ alkyl, or —CN.

Embodiment I-11A. The compound for use of Embodiment I-9A or I-10A, wherein R^{j} is —CN.

Embodiment I-12A. The compound for use of any one of Embodiments I-1A to I-11A, wherein R^{c} is C₁-C₆ alkyl, —CN, or halogen.

Embodiment I-13A. The compound for use of any one of Embodiments I-1A to I-12A, wherein R^{c} is —CN or halogen.

Embodiment I-14A. The compound for use of any one of Embodiments I-1A to I-12A, wherein R^{c} is —CN.

Embodiment I-15A. The compound for use of any one of Embodiments I-1A to I-14A, wherein R^{d} is methyl.

Embodiment I-16A. The compound for use of any one of Embodiments I-1A to I-14A, wherein R^{d} is optionally substituted 5- to 10-membered aryl.

Embodiment I-17A. The compound for use of any one of Embodiments I-1A to I-14A, wherein R^{d} is optionally substituted 5- or 6-membered heteroaryl.

Embodiment I-18A. The compound for use of any one of Embodiments I-1A to I-14A, wherein R^{d} is optionally substituted 5- or 6-membered cycloalkyl.

Embodiment I-19A. The compound for use of any one of Embodiments I-1A to I-14A, wherein R^{d} is methyl, cyclohexyl, pyridinyl, thiazolyl, phenyl, or thienyl.

Embodiment I-20A. The compound for use of any one of Embodiments I-1A to I-14A, wherein R^{d} is methyl, cyclohexyl, pyridinyl, thiazolyl, thienyl, or optionally substituted phenyl.

Embodiment I-21A. The compound for use of any one of Embodiments I-1A to I-14A, wherein R^{d} is methyl.

Embodiment I-22A. The compound for use of any one of Embodiments I-1A to I-14A, wherein R^{d} is —CF₃.

Embodiment I-23A. The compound for use of any one of Embodiments I-1A to I-14A, wherein R^{d} is CR^{f}F₂.

Embodiment I-24A. The compound for use of any one of Embodiments I-1A to I-14A, wherein R^{d} is —(C(R⁶)₂)ₜC₆-C₁₀ aryl, —(C(R⁶)₂)ₜ-5- or 6-membered heteroaryl, —(C(R⁶)₂)ₜ-5- or 6-membered cycloalkyl.

Embodiment I-25A. The compound for use of any one of Embodiments I-1A to I-14A, wherein R^{d} is —(C(R⁶)₂)ₜC₆-C₁₀ aryl.

Embodiment I-26A. The compound for use of any one of Embodiments I-1A to I-25A, wherein L is —(C(R⁵)₂)ₘY¹(C(R⁵)₂)ₚ—.

Embodiment I-27A. The compound for use of Embodiment I-26A, wherein Y¹ is S.

Embodiment I-28A. The compound for use of any one of Embodiment I-1A to I-25A, wherein L is —(C(R⁵)₂)ₘNR³C═(O)(C(R⁵)₂)ₚ— or —(C(R⁵)₂)ₘY¹(C(R⁵)₂)ₚ-cyclopropyl-.

Embodiment I-29A. The compound for use of any one of Embodiments I-1A to I-28A, wherein R¹ is C₆-C₁₀ arylene.

Embodiment I-30A. The compound for use of any one of Embodiments I-1A to I-28A, wherein R¹ is heteroarylene.

Embodiment I-31A. The compound for use of any one of Embodiments I-1A to I-28A, wherein R¹ is absent.

Embodiment I-32A. The compound for use of any one of Embodiments I-1A to I-31A, wherein R⁷ is A.

Embodiment I-33A. The compound for use of Embodiment I-32A, wherein A is —(C(R⁶)₂)ᵣCO₂R^{x} or —(CH₂)ᵣtetrazole, wherein the —(CH₂)ᵣtetrazole is optionally substituted with C₁-C₆ alkyl.

Embodiment I-34A. The compound for use of any one of Embodiments I-1A to I-31A, wherein R⁷ is B.

Embodiment I-35A. The compound for use of Embodiment I-32A, wherein B is —(CH₂)ᵣC(O)NR^{g}R^{g′}, or —(CH₂)ᵣS(O)₂NR^{g}R^{g′},

Embodiment I-36A. The compound for use of any one of Embodiments I-1A to I-31A, wherein R⁷ is C.

Embodiment I-37A. The compound for use of Embodiment I-32A, wherein C is —(CH₂)ᵣCN, —(CH₂)ₛOH, or —(C(R⁶)₂)ᵣC₆-C₁₀ aryl, wherein the aryl is substituted with one to three substituents each independently selected from C₁-C₆ alkyl, C₁-C₆ haloalkyl, halogen, and OH.

Embodiment I-38A. The compound for use of any one of Embodiment I-1A to I-37A, wherein the compound is

| | |
|---|---|
| I-34 | 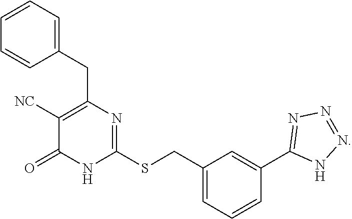 [SMILES: N#Cc1c(Cc2ccccc2)nc(SCc2cccc(-c3nnn[nH]3)c2)[nH]c1=O] |

Embodiment I-39A. The compound for use of any one of Embodiments I-1A to I-38A, wherein the acute inflammatory condition is a systemic inflammatory condition.

Embodiment I-40A. The compound for use of any one of Embodiments I-1A to I-38A, wherein the acute inflammatory condition is an organ-specific condition.

Embodiment I-41A. The compound for use of any one of Embodiments I-1A to I-38A, wherein the acute inflammatory condition is cytokine storm or hypercytokinemia, systemic inflammatory response syndrome (SIRS), graft versus host disease (GVHD), acute respiratory distress syndrome (ARDS), severe acute respiratory distress syndrome (SARS), catastrophic anti-phospholipid syndrome, viral infections, bacterial infections, fungal infections, influenza, pneumonia, shock, or sepsis.

Embodiment I-42A. The compound for use of any one of Embodiments I-1A to I-38A, wherein the acute inflammatory condition is acute pancreatitis, hepatitis, respiratory condition, or enterocolitis.

Embodiment I-43A. The compound for use of any one of Embodiments I-1A to I-42A, wherein the method reduces a pro-inflammatory cytokine or increases an anti-inflammatory cytokine.

Embodiment I-44A. The compound for use of Embodiment I-43A, wherein the pro-inflammatory cytokine is IL-1β, IL-6, IL-18, TNF-α, or TGF-β.

Embodiment I-45A. The compound for use of Embodiment I-43A, wherein pro-inflammatory cytokine is MCP-1, TNF-α, or IL-1β and the pro-inflammatory cytokine is increased.

Embodiment I-46A. The compound for use of Embodiment I-43A, wherein pro-inflammatory cytokine is IL-6 and the pro-inflammatory cytokine is increased.

Embodiment I-47A. The compound for use of Embodiment I-43A, wherein the anti-inflammatory cytokine is IL-10.

Embodiment I-48A. The compound for use of any one of Embodiments I-1A to I-42A, wherein expression of sirtuin-1 modulated genes sod2, tfam, dda1 genes are increased in the liver.

Embodiment I-49A. The compound for use of any one of Embodiments I-1A to I-48A, wherein the administration to the subject occurs at least 12 hours after an injury.

Embodiment I-50A. The compound for use of any one of Embodiments I-1A to I-49A, wherein the administration to the subject occurs for at least 6 days after an injury.

Embodiment I-1B. A use of a compound represented by Formula (II):

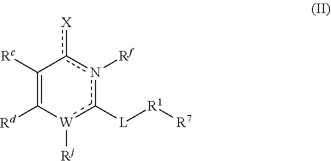

or a pharmaceutically acceptable salt or tautomer thereof, wherein:
X is H, S, SR², NR², NR²R^{2′}, OH, OR^{h}, F, Br, or Cl;
W is N or C;
(i) when W is N, then: L is —(C(R⁵)₂)ₘCH═CH(C(R⁵)₂)ₚ—,

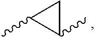

—(C(R⁵)₂)ₘY¹(C(R⁵)₂)ₚ—, —(C(R⁵)₂)ₘY¹(C(R⁵)₂)ₚ-cyclopropyl-, —(C(R⁵)₂)ₘY¹CH═CH—, —(C(R⁵)₂)ₘNR³C═(O)(C(R⁵)₂)ₚ—, —(C(R⁵)₂)ₘphenyl(C(R⁵)₂)ₚ—, —(C(R⁵)₂)ₘpyridinyl(C(R⁵)₂)ₚ—, or —(C(R⁵)₂)ₘthiophenyl(C(R⁵)₂)ₚ—;
(ii) when W is C, then: L is —(C(R⁵)₂)ₘCH═CH(C(R⁵)₂)ₚ—, —(C(R⁵)₂)ₒ—, —(C(R⁵)₂)ₘY¹(C(R⁵)₂)ₚ—,

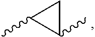

—(C(R⁵)₂)ₘ Y¹CH═CH—, —(C(R⁵)₂)ₘC═(O)(CH₂)ₚ—, —(C(R⁵)₂)ₘC═(O)O(C(R⁵)₂)ₚ—, —(C(R⁵)₂)ₘC═(O)NR³(C(R⁵)₂)ₚ—, —(C(R⁵)₂)ₘNR³C═(O)(C(R⁵)₂)ₚ—, —(C(R⁵)₂)ₘphenyl(C(R⁵)₂)ₚ—, —(C(R⁵)₂)ₘpyridinyl(C(R⁵)₂)ₚ—, or —(C(R⁵)₂)ₘthiophenyl(C(R⁵)₂)ₚ—;
Y¹ is O, NR, or S(O)_{q};
each Y² is independently O, NH or S;
R¹ is absent, C₆-C₁₀ arylene, heteroarylene, or C₃-C₈cycloalkylene, wherein the heteroarylene comprises one or two 5- to 7-membered rings and 1-4 heteroatoms selected from N, O and S, and wherein the C₆-C₁₀ arylene, heteroarylene, and C₃-C₈cycloalkylene are optionally substituted with one to two R^{e};
R² is H or C₁-C₄ alkyl;
R^{2′} is H, C₁-C₄ alkyl, or C₃-C₇ cycloalkyl; or
R² and R^{2′} together with the nitrogen atom to which they are attached form a 3- to 7-membered heterocycloalkyl ring comprising 1-3 additional heteroatoms selected from N, O and S;
R³ is H or C₁-C₄ alkyl;
R⁴ is H or C₁-C₄ alkyl;
each R⁵ is independently at each occurrence H or C₁-C₄ alkyl;
each R⁶ is independently at each occurrence H or C₁-C₄ alkyl;
R⁷ is H, A, B, or C;
A is —(C(R⁶)₂)ᵣCO₂R^{x}, —Y²(C(R⁶)₂)ᵣCO₂R^{x}, —(C(R⁶)₂)ᵣtetrazole, —(C(R⁶)₂)ᵣoxadiazolone, —(C(R⁶)₂)ᵣtetrazolone, —(C(R⁶)₂)ᵣthiadiazolol, —(C(R⁶)₂)ᵣ isoxazol-3-ol, —(C(R⁶)₂)ᵣP(O)(OH)OR^{x}, —(C(R⁶)₂)ᵣS(O)₂OH, —(C(R⁶)₂)ᵣC(O)NHCN, or —(C(R⁶)₂)ᵣC(O)NHS(O)₂alkyl, wherein —(C(R⁶)₂)ᵣtetrazole, —(C(R⁶)₂)ᵣoxadiazolone, —(C(R⁶)₂)ᵣtetrazolone, —(C(R⁶)₂)ᵣthiadiazolol, —(C(R⁶)₂)ᵣ isoxazol-3-ol are optionally substituted with C₁-C₆ alkyl,
B is —(C(R⁶)₂)ᵣS(O)₂OC₁-C₄ alkyl, —O(C(R⁶)₂)ᵣS(O)₂OC₁-C₄ alkyl, —Y²(C(R⁶)₂)ᵣC(O)NR^{g}R^{g′}, —Y²(C(R⁶)₂)ᵣS(O)₂NR^{g}R^{g′}, —(C(R⁶)₂)ᵣC(O)NR^{g}R^{g′}, —(C(R⁶)₂)ᵣS(O)₂NR^{g}R^{g′}, —(C(R⁶)₂)ᵣC(O)NHS(O)₂NR^{g}R^{g′}, —(C(R⁶)₂)ᵣCO₂Rⁱ, —(C(R⁶)₂)ᵣNH₂CO₂R^{x}, —(C(R⁶)₂)ᵣP(O)(OR^{x})₂, —O(C(R⁶)₂)ᵣP(O)(OR^{x})₂, —(C(R⁶)₂)ᵣS(O)₂OH, —O(C(R⁶)₂)ᵣS(O)₂OH, —(C(R⁶)₂)ᵣP(O)₂OR^{x}, or —O(C(R⁶)₂)ᵣP(O)₂OR^{x},
C is —(CH₂)ᵣCN, —(CH₂)ₛOH, halogen, —(C(R⁶)₂)ᵣC₆-C₁₀ aryl, —(C(R⁶)₂)ᵣS—C₆-C₁₀ aryl, —(C(R⁶)₂)ᵣheteroaryl, —O(C(R⁶)₂)ᵣheteroaryl, —O(C(R⁶)₂)ᵣheterocycloalkyl, —O(C(R⁶)₂)ᵣOH, —OR^{y}, —(C(R⁶)₂)ᵣC(O)NHCN, —CH═CHCO₂R^{x}, or —(C(R⁶)₂)ᵣC(O)NHS(O)₂C₁-C₄ alkyl, wherein the aryl and heteroaryl are substituted with one to three substituents each independently selected from C₁-C₆ alkyl, C₁-C₆ haloalkyl, halogen, and OH, and wherein the heterocycloalkyl is substituted with one to two ═O or ═S;
R^{c} is H, C₁-C₆ alkyl, C₁-C₆ haloalkyl, halogen, —CN, —OR^{x}, or —CO₂R^{x};
R^{d} is methyl, CF₃, CR^{f}F₂, —(C(R⁶)₂)ₜC₆-C₁₀ aryl, —(C(R⁶)₂)ₜ-5- or 6-membered heteroaryl, —(C(R⁶)₂)ₜ-5- or 6-membered cycloalkyl, optionally substituted C₆-C₁₀ aryl, optionally substituted 5- or 6-membered heteroaryl, or optionally substituted 5- or 6-membered cycloalkyl;
each R^{e} is independently at each occurrence C₁-C₆ alkyl, C₂-C₆ alkenyl, C₂-C₆ alkynyl, halogen, C₁-C₆ haloalkyl, —NHR^{z}, —OH, or —CN;
R^{f} is absent, H, or methyl;
R^{g} is H, C₁-C₆ alkyl, OH, —S(O)₂(C₁-C₆ alkyl), or S(O)₂N(C₁-C₆ alkyl)₂;
R^{g′} is H, C₁-C₆ alkyl, C₃-C₇ cycloalkyl, 4- to 7-membered heterocycloalkyl ring comprising 1-3 heteroatoms selected from N, O and S, C₆-C₁₀ aryl, or 5- to 7-membered heteroaryl comprising 1-3 heteroatoms selected from N, O and S, wherein the alkyl is optionally substituted with one or more substituents independently selected from halogen and —OH, and wherein the cycloalkyl, heterocycloalkyl, aryl and heteroaryl are optionally substituted with one or more substituents independently selected from C₁-C₆ alkyl, halogen, and —OH;
R^{h} is H, C₁-C₄ alkyl, or 3- to 7-membered heterocycloalkyl ring comprising 1-3 heteroatoms selected from N, O and S, wherein the alkyl is optionally substituted with one or more substituents each independently selected from NH₂, C₁-C₄ alkylamino, C₁-C₄ dialkylamino, and C(O)NH₂; and wherein the heterocycloalkyl is optionally substituted with one or more substituents each independently selected from C₁-C₆ alkyl and C₁-C₆ haloalkyl;
Rⁱ is (i) —(CH₂)ₛOC(O)C₁-C₆ alkyl, wherein the alkyl is substituted with one or more NH₂; (ii) (CH₂CH₂O)ₙCH₂CH₂OH; or (iii) C₁-C₆ alkyl substituted with one or more substituents each independently selected from OH and 4- to 7-membered heterocycloalkyl comprising 1 to 3 heteroatoms selected from O, N, or S;
R^{j} is absent, H, C₁-C₆ alkyl, or —CN;
each R^{x} is independently at each occurrence H, C₁-C₆ alkyl, or C₆-C₁₀ aryl;
each R^{y} and R^{z} is independently H, C₁-C₆ alkyl, or C₁-C₆ haloalkyl;
each m, p, q, r, and t is independently 0, 1 or 2;
n is 0, 1, 2, or 3;
s is 1 or 2;
o is 0, 1, 2, 3, or 4; and
[symbol] represents a single bond or a double bond; and
provided that
when X is O; R^{f} is H; W is C; R^{j} is —CN; L is —SCH₂—; R¹ is phenylene or pyridine; then R⁷ is not —COOH;
when X is O; R^{f} is H; W is C; R^{j} is —CN; L is —SCH₂—; R¹ is phenylene or pyridine; and R⁷ is tetrazole; then R^{c} is not H;
when X is O; R^{f} is H; W is C; R^{j} is —CN; L is —S—C(R⁵)₂ or —SCH₂CH₂—; R¹ is absent; then R⁷ is not COOH or tetrazole;
when X is O, R^{f} is H; W is N; R^{j} is absent; R^{d} is methyl, optionally substituted 5- to 10-membered aryl, optionally substituted 5- or 6-membered heteroaryl, or optionally substituted 5- or 6-membered cycloalkyl; L is —SCH₂— or —OCH₂—; and R¹ is phenylene; then R⁷ is not —COOH, —CH₂COOH,

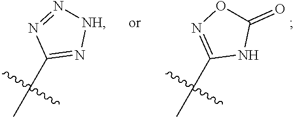 [SMILES: *c1nn[nH]n1 ; *c1noc(=O)[nH]1]

and
when X is O, R^{f} is H, W is N, R^{j} is absent, L is —NHCH₂—, —CH₂NH—, or —NH—C(O)—, and R¹ is phenylene, then R^{d} is not phenyl;
for treating an acute inflammatory condition.

Embodiment I-2B. A use of compound represented by Formula (I):

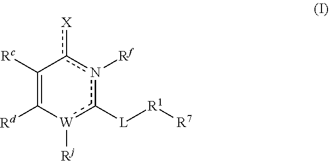

or a pharmaceutically acceptable salt or tautomer thereof,
wherein:
X is H, S, SR², NR², NR²R^{2′}, OH, OR^{h}, F, Br, or Cl;
W is N or C;
   (i) when W is N, then:
   L is —(C(R⁵)₂)ₘCH═CH(C(R⁵)₂)ₚ—,

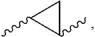

—(C(R⁵)₂)ₘY¹(C(R⁵)₂)ₚ—, —(C(R⁵)₂)ₘY¹(C(R⁵)₂)ₚ-cyclopropyl-, —(C(R⁵)₂)ₘY¹CH═CH—, —(C(R⁵)₂)ₘNR³C═(O)(C(R⁵)₂)ₚ—, —(C(R⁵)₂)ₘphenyl(C(R⁵)₂)ₚ—, —(C(R⁵)₂)ₘpyridinyl(C(R⁵)₂)ₚ—, or —(C(R⁵)₂)ₘthiophenyl(C(R⁵)₂)ₚ—;
(ii) when W is C, then:
L is —(C(R⁵)₂)ₘCH═CH(C(R⁵)₂)ₚ—, —(C(R⁵)₂)ₒ—, —(C(R⁵)₂)ₘY¹(C(R⁵)₂)ₚ—,

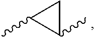

(C(R⁵)₂)ₘ Y¹CH═CH—, —(C(R⁵)₂)ₘC═(O)(CH₂)ₚ—, —(C(R⁵)₂)ₘC═(O)O(C(R⁵)₂)ₚ—, —(C(R⁵)₂)ₘC═(O)NR³(C(R⁵)₂)ₚ—, —(C(R⁵)₂)ₘNR³C═(O)(C(R⁵)₂)ₚ—, —(C(R⁵)₂)ₘphenyl(C(R⁵)₂)ₚ—, —(C(R⁵)₂)ₘpyridinyl(C(R⁵)₂)ₚ—, or —(C(R⁵)₂)ₘthiophenyl(C(R⁵)₂)ₚ—;
Y¹ is O, NR⁴, or S(O)_{q};
each Y² is independently O, NH or S;
R¹ is absent or C₆-C₁₀ arylene or heteroarylene, wherein the heteroarylene comprises one or two 5- to 7-membered rings and 1-4 heteroatoms selected from N, O and S, and wherein the C₆-C₁₀ arylene or heteroarylene are optionally substituted with one to two R^{e};
R² is H or C₁-C₄ alkyl;
R^{2′} is H, C₁-C₄ alkyl, or C₃-C₇ cycloalkyl; or
R² and R^{2′} together with the nitrogen atom to which they are attached form a 3- to 7-membered heterocycloalkyl ring comprising 1-3 additional heteroatoms selected from N, O and S;
R³ is H or C₁-C₄ alkyl;
R⁴ is H or C₁-C₄ alkyl;
each R⁵ is independently at each occurrence H or C₁-C₄ alkyl;
each R⁶ is independently at each occurrence H or C₁-C₄ alkyl;
R⁷ is H, A, B, or C;
A is —(C(R⁶)₂)ᵣCO₂R^{x}, —Y²(C(R⁶)₂)ᵣCO₂R^{x}, —(CH₂)ᵣtetrazole, —(CH₂)ᵣoxadiazolone, —(CH₂)ᵣtetrazolone, —(CH₂)ᵣthiadiazolol, —(CH₂)ᵣ isoxazol-3-ol, —(CH₂)ᵣP(O)(OH)OR^{x}, —(CH₂)ᵣS(O)₂OH, —(CH₂)ᵣC(O)NHCN, or —(CH₂)ᵣC(O)NHS(O)₂alkyl, wherein —(CH₂)ᵣtetrazole, —(CH₂)ᵣoxadiazolone, —(CH₂)ᵣtetrazolone, —(CH₂)ᵣthiadiazolol, —(CH₂)ᵣ isoxazol-3-ol are optionally substituted with C₁-C₆ alkyl,
B is —(C(R⁶)₂)ᵣS(O)₂OC₁-C₄ alkyl, —O(C(R⁶)₂)ᵣS(O)₂OC₁-C₄ alkyl, —Y²(C(R⁶)₂)ᵣC(O)NR^{g}R^{g′}, —Y²(C(R⁶)₂)ᵣS(O)₂NR^{g}R^{g′}, —(CH₂)ᵣC(O)NR^{g}R^{g′}, —(CH₂)ᵣS(O)₂NR^{g}R^{g′}, —(CH₂)ᵣC(O)NHS(O)₂NR^{g}R^{g′}, —(C(R⁶)₂)ᵣCO₂R¹, —(C(R⁶)₂)ᵣNH₂CO₂R^{x}, —(C(R⁶)₂)ᵣP(O)(OR^{x})₂, —O(C(R⁶)₂)ᵣP(O)(OR^{x})₂, —(C(R⁶)₂)ᵣS(O)₂OH, —O(C(R⁶)₂)ᵣS(O)₂OH, —(C(R⁶)₂)ᵣP(O)₂OR^{x}, or —O(C(R⁶)₂)ᵣP(O)₂OR^{x},
C is —(CH₂)ᵣCN, —(CH₂)ₛOH, halogen, —(C(R⁶)₂)ᵣC₆-C₁₀ aryl, —(C(R⁶)₂)ᵣS—C₆-C₁₀ aryl, —(C(R⁶)₂)ᵣheteroaryl, —O(C(R⁶)₂)ᵣheteroaryl, —O(C(R⁶)₂)ᵣheterocycloalkyl, —O(C(R⁶)₂)ᵣOH, —OR^{y}, —(C(R⁶)₂)ᵣC(O)NHCN, —CH═CHCO₂R^{x}, or —(C(R⁶)₂)ᵣC(O)NHS(O)₂C₁-C₄ alkyl, wherein the aryl and heteroaryl are substituted with one to three substituents each independently selected from C₁-C₆ alkyl, C₁-C₆ haloalkyl, halogen, and OH, and wherein the heterocycloalkyl is substituted with one to two ═O or ═S;
R^{c} is H, C₁-C₆ alkyl, C₁-C₆ haloalkyl, halogen, —CN, —OR^{x}, or —CO₂R^{x};
R^{d} is methyl, CF₃, CR^{f}F₂, —(C(R⁶)₂)ₜC₆-C₁₀ aryl, —(C(R⁶)₂)ₜ-5- or 6-membered heteroaryl, —(C(R⁶)₂)ₜ-5- or 6-membered cycloalkyl, optionally substituted C₆-C₁₀ aryl, optionally substituted 5- or 6-membered heteroaryl, or optionally substituted 5- or 6-membered cycloalkyl;
each R^{e} is independently at each occurrence C₁-C₆ alkyl, C₂-C₆ alkenyl, C₂-C₆ alkynyl, halogen, C₁-C₆ haloalkyl, —NHR^{z}, —OH, or —CN;
R^{f} is absent, H, or methyl;
R^{g} is H, C₁-C₆ alkyl, OH, —S(O)₂(C₁-C₆ alkyl), or S(O)₂N(C₁-C₆ alkyl)₂;
R^{g′} is H, C₁-C₆ alkyl, C₃-C₇ cycloalkyl, 4- to 7-membered heterocycloalkyl ring comprising 1-3 heteroatoms selected from N, O and S, C₆-C₁₀ aryl, or 5- to 7-membered heteroaryl comprising 1-3 heteroatoms selected from N, O and S, wherein the alkyl is optionally substituted with one or more substituents independently selected from halogen and —OH, and wherein the cycloalkyl, heterocycloalkyl, aryl and heteroaryl are optionally substituted with one or more substituents independently selected from C₁-C₆ alkyl, halogen, and —OH;
R^{h} is H, C₁-C₄ alkyl, or 3- to 7-membered heterocycloalkyl ring comprising 1-3 heteroatoms selected from N, O and S, wherein the alkyl is optionally substituted with one or more substituents each independently selected from NH₂, C₁-C₄ alkylamino, C₁-C₄ dialkylamino, and C(O)NH₂; and wherein the heterocycloalkyl is optionally substituted with one or more substituents each independently selected from C₁-C₆ alkyl and C₁-C₆ haloalkyl;
Rⁱ is (i) —(CH₂)ₛOC(O)C₁-C₆ alkyl, wherein the alkyl is substituted with one or more NH₂; (ii) (CH₂CH₂O)ₙCH₂CH₂OH; or (iii) C₁-C₆ alkyl substituted with one or more substituents each independently selected from OH and 4- to 7-membered heterocycloalkyl comprising 1 to 3 heteroatoms selected from O, N, or S;
R^{j} is absent, H, C₁-C₆ alkyl, or —CN;
each R^{x} is independently at each occurrence H, C₁-C₆ alkyl, or C₆-C₁₀ aryl;
each R^{y} and R^{z} is independently H, C₁-C₆ alkyl, or C₁-C₆ haloalkyl;
each m, p, q, r, and t is independently 0, 1 or 2;
n is 0, 1, 2, or 3;
s is 1 or 2;
o is 0, 1, 2, 3, or 4; and
[symbol] represents a single bond or a double bond; and
provided that
when X is O; R^{f} is H; W is C; R^{j} is —CN; L is —SCH₂—; R¹ is phenylene or pyridine; then R⁷ is not —COOH;
when X is O; R^{f} is H; W is C; R^{j} is —CN; L is —SCH₂—; R¹ is phenylene or pyridine; and R⁷ is tetrazole; then R^{c} is not H;
when X is O; R^{f} is H; W is C; R^{j} is —CN; L is —S—C(R⁵)₂ or —SCH₂CH₂—; R¹ is absent; then R⁷ is not COOH or tetrazole;
when X is O, R^{f} is H; W is N; R^{j} is absent; R^{d} is methyl, optionally substituted 5- to 10-membered aryl, optionally substituted 5- or 6-membered heteroaryl, or optionally substituted 5- or 6-membered cycloalkyl; L is —SCH₂— or —OCH₂—; and R¹ is phenylene; then R⁷ is not —COOH, —CH₂COOH,

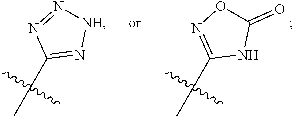 [SMILES: *c1nn[nH]n1 ; *c1noc(=O)[nH]1]

and
when X is O, R^{f} is H, W is N, R^{j} is absent, L is —NHCH₂—, —CH₂NH—, or —NH—C(O)—, and R¹ is phenylene, then R^{d} is not phenyl;
for treating an acute inflammatory condition.

Embodiment I-3B. The use of Embodiment I-1B or I-2B, wherein X is O, OH, OR^{h}, F, Br, or Cl.

Embodiment I-4B. The use of Embodiment I-1B or I-2B, wherein X is H, S, SR², NR², or NR²R².

Embodiment I-5B. The use of any one of Embodiments I-1B to I-4B, wherein R^{f} is absent.

Embodiment I-6B. The use of any one of Embodiments I-1B to I-4B, wherein R^{f} is H or methyl.

Embodiment I-7B. The use of any one of Embodiments I-1B to I-6B, wherein W is N.

Embodiment I-8B. The use of Embodiment I-7B, wherein R^{j} is absent.

Embodiment I-9B. The use of any one of Embodiments I-1B to I-6B, wherein W is C.

Embodiment I-10B. The use of Embodiment I-9B, wherein R^{j} is H, C₁-C₆ alkyl, or —CN.

Embodiment I-11B. The use of Embodiment I-9B or I-10B, wherein R^{j} is —CN.

Embodiment I-12B. The use of any one of Embodiments I-1B to I-11B, wherein R^{c} is C₁-C₆ alkyl, —CN, or halogen.

Embodiment I-13B. The use of any one of Embodiments I-1B to I-12B, wherein R^{c} is —CN or halogen.

Embodiment I-14B. The use of any one of Embodiments I-1B to I-12B, wherein R^{c} is —CN.

Embodiment I-15B. The use of any one of Embodiments I-1B to I-14B, wherein R^{d} is methyl.

Embodiment I-16B. The use of any one of Embodiments I-1B to I-14B, wherein R^{d} is optionally substituted 5- to 10-membered aryl.

Embodiment I-17B. The use of any one of Embodiments I-1B to I-14B, wherein R^{d} is optionally substituted 5- or 6-membered heteroaryl.

Embodiment I-18B. The use of any one of Embodiments I-1B to I-14B, wherein R^{d} is optionally substituted 5- or 6-membered cycloalkyl.

Embodiment I-19B. The use of any one of Embodiments I-1B to I-14B, wherein R^{d} is methyl, cyclohexyl, pyridinyl, thiazolyl, phenyl, or thienyl.

Embodiment I-20B. The use of any one of Embodiments I-1B to I-14B, wherein R^{d} is methyl, cyclohexyl, pyridinyl, thiazolyl, thienyl, or optionally substituted phenyl.

Embodiment I-21B. The use of any one of Embodiments I-1B to I-14B, wherein R^{d} is methyl.

Embodiment I-22B. The use of any one of Embodiments I-1B to I-14B, wherein R^{d} is —CF₃.

Embodiment I-23B. The use of any one of Embodiments I-1B to I-14B, wherein R^{d} is CR^{f}F₂.

Embodiment I-24B. The use of any one of Embodiments I-1B to I-14B, wherein R^{d} is —(C(R⁶)₂)ₜC₆-C₁₀ aryl, —(C(R⁶)₂)ₜ-5- or 6-membered heteroaryl, —(C(R⁶)₂)ₜ-5- or 6-membered cycloalkyl.

Embodiment I-25B. The use of any one of Embodiments I-1B to I-14B, wherein R^{d} is —(C(R⁶)₂)ₜC₆-C₁₀ aryl.

Embodiment I-26B. The use of any one of Embodiments I-1B to I-25B, wherein L is —(C(R⁵)₂)ₘY¹(C(R⁵)₂)ₚ—.

Embodiment I-27B. The use of Embodiment I-26B, wherein Y¹ is S.

Embodiment I-28B. The use of any one of Embodiment I-1B to I-25B, wherein L is —(C(R⁵)₂)ₘNR³C═(O)(C(R⁵)₂)ₚ— or —(C(R⁵)₂)ₘY¹(C(R⁵)₂)ₚ-cyclopropyl-.

Embodiment I-29B. The use of any one of Embodiments I-1B to I-28B, wherein R¹ is C₆-C₁₀ arylene.

Embodiment I-30B. The use of any one of Embodiments I-1B to I-28B, wherein R¹ is heteroarylene.

Embodiment I-31B. The use of any one of Embodiments I-1B to I-28B, wherein R¹ is absent.

Embodiment I-32B. The use of any one of Embodiments I-1B to I-31B, wherein R⁷ is A.

Embodiment I-33B. The use of Embodiment I-32B, wherein A is —(C(R⁶)₂)ᵣCO₂R^{x} or —(CH₂)ᵣtetrazole, wherein the —(CH₂)ᵣtetrazole is optionally substituted with C₁-C₆ alkyl.

Embodiment I-34B. The use of any one of Embodiments I-1B to I-31B, wherein R⁷ is B.

Embodiment I-35B. The use of Embodiment I-32B, wherein B is —(CH₂)ᵣC(O)NR^{g}R^{g′}, or —(CH₂)ᵣS(O)₂NR^{g}R^{g′},

Embodiment I-36B. The use of any one of Embodiments I-1B to I-31B, wherein R⁷ is C.

Embodiment I-37B. The use of Embodiment I-32B, wherein C is —(CH₂)ᵣCN, —(CH₂)ₛOH, or —(C(R⁶)₂)ᵣC₆-C₁₀ aryl, wherein the aryl is substituted with one to three substituents each independently selected from C₁-C₆ alkyl, C₁-C₆ haloalkyl, halogen, and OH.

Embodiment I-38B. The use of any one of Embodiment I-1B to I-37B, wherein the compound is

| | |
|---|---|
| I-34 | 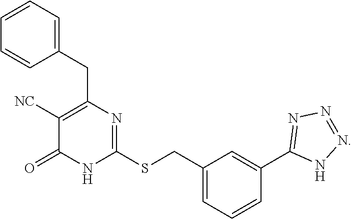 [SMILES: N#Cc1c(Cc2ccccc2)nc(SCc2cccc(-c3nnn[nH]3)c2)[nH]c1=O] |

Embodiment I-39B. The use of any one of Embodiments I-1B to I-38B, wherein the acute inflammatory condition is a systemic inflammatory condition.

Embodiment I-40B. The use of any one of Embodiments I-1B to I-38B, wherein the acute inflammatory condition is an organ-specific condition.

Embodiment I-41B. The use of any one of Embodiments I-1B to I-38B, wherein the acute inflammatory condition is cytokine storm or hypercytokinemia, systemic inflammatory response syndrome (SIRS), graft versus host disease (GVHD), acute respiratory distress syndrome (ARDS), severe acute respiratory distress syndrome (SARS), catastrophic anti-phospholipid syndrome, viral infections, bacterial infections, fungal infections, influenza, pneumonia, shock, or sepsis.

Embodiment I-42B. The use of any one of Embodiments I-1B to I-38B, wherein the acute inflammatory condition is acute pancreatitis, hepatitis, respiratory condition, or enterocolitis.

Embodiment I-43B. The use of any one of Embodiments I-1B to I-42B, wherein the method reduces a pro-inflammatory cytokine or increases an anti-inflammatory cytokine.

Embodiment I-44B. The use of Embodiment I-43B, wherein the pro-inflammatory cytokine is IL-1β, IL-6, IL-18, TNF-α, or TGF-β.

Embodiment I-45B. The use of Embodiment I-43B, wherein pro-inflammatory cytokine is MCP-1, TNF-α, or IL-1β and the pro-inflammatory cytokine is increased.

Embodiment I-46B. The use of Embodiment I-43B, wherein pro-inflammatory cytokine is IL-6 and the pro-inflammatory cytokine is increased.

Embodiment I-47B. The use of Embodiment I-43B, wherein the anti-inflammatory cytokine is IL-10.

Embodiment I-48B. The use of any one of Embodiments I-1B to I-42B, wherein expression of sirtuin-1 modulated genes sod2, tfam, dda1 genes are increased in the liver.

Embodiment I-49B. The use of any one of Embodiments I-1B to I-48B, wherein the administration to the subject occurs at least 12 hours after an injury.

Embodiment I-50B. The use of any one of Embodiments I-1B to I-49B, wherein the administration to the subject occurs for at least 6 days after an injury.

Embodiment I-1C. Use of a compound represented by Formula (II):

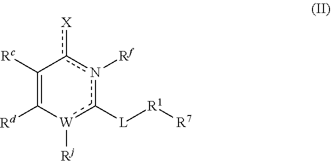

or a pharmaceutically acceptable salt or tautomer thereof, wherein:
X is H, S, SR², NR², NR²R^{2′}, OH, OR^{h}, F, Br, or Cl;
W is N or C;
(i) when W is N, then: L is —(C(R⁵)₂)ₘCH═CH(C(R⁵)₂)ₚ—,

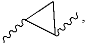

—(C(R⁵)₂)ₘY¹(C(R⁵)₂)ₚ—, —(C(R⁵)₂)ₘY¹(C(R⁵)₂)ₚ-cyclopropyl-, —(C(R⁵)₂)ₘY¹CH═CH—, —(C(R⁵)₂)ₘNR³C═(O)(C(R⁵)₂)ₚ—, —(C(R⁵)₂)ₘphenyl(C(R⁵)₂)ₚ—, —(C(R⁵)₂)ₘpyridinyl(C(R⁵)₂)ₚ—, or —(C(R⁵)₂)ₘthiophenyl(C(R⁵)₂)ₚ—;
(ii) when W is C, then: L is —(C(R⁵)₂)ₘCH═CH(C(R⁵)₂)ₚ—, —(C(R⁵)₂)ₒ—, —(C(R⁵)₂)ₘY¹(C(R⁵)₂)ₚ—,

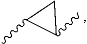

(C(R⁵)₂)ₘ Y¹CH═CH—, —(C(R⁵)₂)ₘC═(O)(CH₂)ₚ—, —(C(R⁵)₂)ₘC═(O)O(C(R⁵)₂)ₚ—, —(C(R⁵)₂)ₘC═(O)NR³(C(R⁵)₂)ₚ—, —(C(R⁵)₂)ₘNR³C═(O)(C(R⁵)₂)ₚ—, —(C(R⁵)₂)ₘphenyl(C(R⁵)₂)ₚ—, —(C(R⁵)₂)ₘpyridinyl(C(R⁵)₂)ₚ—, or —(C(R⁵)₂)ₘthiophenyl(C(R⁵)₂)ₚ—;
Y¹ is O, NR⁴, or S(O)_{q};
each Y² is independently O, NH or S;
R¹ is absent, C₆-C₁₀ arylene, heteroarylene, or C₃-C₈cycloalkylene, wherein the heteroarylene comprises one or two 5- to 7-membered rings and 1-4 heteroatoms selected from N, O and S, and wherein the C₆-C₁₀ arylene, heteroarylene, and C₃-C₈cycloalkylene are optionally substituted with one to two R^{e};
R² is H or C₁-C₄ alkyl;
R^{2′} is H, C₁-C₄ alkyl, or C₃-C₇ cycloalkyl; or
R² and R^{2′} together with the nitrogen atom to which they are attached form a 3- to 7-membered heterocycloalkyl ring comprising 1-3 additional heteroatoms selected from N, O and S;
R³ is H or C₁-C₄ alkyl;
R⁴ is H or C₁-C₄ alkyl;
each R⁵ is independently at each occurrence H or C₁-C₄ alkyl;
each R⁶ is independently at each occurrence H or C₁-C₄ alkyl;
R⁷ is H, A, B, or C;
A is —(C(R⁶)₂)ᵣCO₂R^{x}, —Y²(C(R⁶)₂)ᵣCO₂R^{x}, —(C(R⁶)₂)ᵣtetrazole, —(C(R⁶)₂)ᵣoxadiazolone, —(C(R⁶)₂)ᵣtetrazolone, —(C(R⁶)₂)ᵣthiadiazolol, —(C(R⁶)₂)ᵣ isoxazol-3-ol, —(C(R⁶)₂)ᵣP(O)(OH)OR^{x}, —(C(R⁶)₂)ᵣS(O)₂OH, —(C(R⁶)₂)ᵣC(O)NHCN, or —(C(R⁶)₂)ᵣC(O)NHS(O)₂alkyl, wherein —(C(R⁶)₂)ᵣtetrazole, —(C(R⁶)₂)ᵣoxadiazolone, —(C(R⁶)₂)ᵣtetrazolone, —(C(R⁶)₂)ᵣthiadiazolol, —(C(R⁶)₂)ᵣ isoxazol-3-ol are optionally substituted with C₁-C₆ alkyl,
B is —(C(R⁶)₂)ᵣS(O)₂OC₁-C₄ alkyl, —O(C(R⁶)₂)ᵣS(O)₂OC₁-C₄ alkyl, —Y²(C(R⁶)₂)ᵣC(O)NR^{g}R^{g′}, —Y²(C(R⁶)₂)ᵣS(O)₂NR^{g}R^{g′}, —(C(R⁶)₂)ᵣC(O)NR^{g}R^{g′}, —(C(R⁶)₂)ᵣS(O)₂NR^{g}R^{g′}, —(C(R⁶)₂)ᵣC(O)NHS(O)₂NR^{g}R^{g′}, —(C(R⁶)₂)ᵣCO₂Rⁱ, —(C(R⁶)₂)ᵣNH₂CO₂R^{x}, —(C(R⁶)₂)ᵣP(O)(OR^{x})₂, —O(C(R⁶)₂)ᵣP(O)(OR^{x})₂, —(C(R⁶)₂)ᵣS(O)₂OH, —O(C(R⁶)₂)ᵣS(O)₂OH, —(C(R⁶)₂)ᵣP(O)₂OR^{x}, or —O(C(R⁶)₂)ᵣP(O)₂OR^{x},
C is —(CH₂)ᵣCN, —(CH₂)ₛOH, halogen, —(C(R⁶)₂)ᵣC₆-C₁₀ aryl, —(C(R⁶)₂)ᵣS—C₆-C₁₀ aryl, —(C(R⁶)₂)ᵣheteroaryl, —O(C(R⁶)₂)ᵣheteroaryl, —O(C(R⁶)₂)ᵣheterocycloalkyl, —O(C(R⁶)₂)ᵣOH, —OR^{y}, —(C(R⁶)₂)ᵣC(O)NHCN, —CH═CHCO₂R^{x}, or —(C(R⁶)₂)ᵣC(O)NHS(O)₂C₁-C₄ alkyl, wherein the aryl and heteroaryl are substituted with one to three substituents each independently selected from C₁-C₆ alkyl, C₁-C₆ haloalkyl, halogen, and OH, and wherein the heterocycloalkyl is substituted with one to two ═O or ═S;
R^{c} is H, C₁-C₆ alkyl, C₁-C₆ haloalkyl, halogen, —CN, —OR^{x}, or —CO₂R^{x};
R^{d} is methyl, CF₃, CR^{f}F₂, —(C(R⁶)₂)ₜC₆-C₁₀ aryl, —(C(R⁶)₂)ₜ-5- or 6-membered heteroaryl, —(C(R⁶)₂)ₜ-5- or 6-membered cycloalkyl, optionally substituted C₆-C₁₀ aryl, optionally substituted 5- or 6-membered heteroaryl, or optionally substituted 5- or 6-membered cycloalkyl;
each R^{e} is independently at each occurrence C₁-C₆ alkyl, C₂-C₆ alkenyl, C₂-C₆ alkynyl, halogen, C₁-C₆ haloalkyl, —NHR^{z}, —OH, or —CN;
R^{f} is absent, H, or methyl;
R^{g} is H, C₁-C₆ alkyl, OH, —S(O)₂(C₁-C₆ alkyl), or S(O)₂N(C₁-C₆ alkyl)₂;
R^{g′} is H, C₁-C₆ alkyl, C₃-C₇ cycloalkyl, 4- to 7-membered heterocycloalkyl ring comprising 1-3 heteroatoms selected from N, O and S, C₆-C₁₀ aryl, or 5- to 7-membered heteroaryl comprising 1-3 heteroatoms selected from N, O and S, wherein the alkyl is optionally substituted with one or more substituents independently selected from halogen and —OH, and wherein the cycloalkyl, heterocycloalkyl, aryl and heteroaryl are optionally substituted with one or more substituents independently selected from C₁-C₆ alkyl, halogen, and —OH;
R^{h} is H, C₁-C₄ alkyl, or 3- to 7-membered heterocycloalkyl ring comprising 1-3 heteroatoms selected from N, O and S, wherein the alkyl is optionally substituted with one or more substituents each independently selected from NH₂, C₁-C₄ alkylamino, C₁-C₄ dialkylamino, and C(O)NH₂; and wherein the heterocycloalkyl is optionally substituted with one or more substituents each independently selected from C₁-C₆ alkyl and C₁-C₆ haloalkyl;
Rⁱ is (i) —(CH₂)ₛOC(O)C₁-C₆ alkyl, wherein the alkyl is substituted with one or more NH₂; (ii) (CH₂CH₂O)ₙCH₂CH₂OH; or (iii) C₁-C₆ alkyl substituted with one or more substituents each independently selected from OH and 4- to 7-membered heterocycloalkyl comprising 1 to 3 heteroatoms selected from O, N, or S;
R^{j} is absent, H, C₁-C₆ alkyl, or —CN;
each R^{x} is independently at each occurrence H, C₁-C₆ alkyl, or C₆-C₁₀ aryl;
each R^{y} and R^{z} is independently H, C₁-C₆ alkyl, or C₁-C₆ haloalkyl;
each m, p, q, r, and t is independently 0, 1 or 2;
n is 0, 1, 2, or 3;
s is 1 or 2;
o is 0, 1, 2, 3, or 4; and
[symbol] represents a single bond or a double bond; and
provided that
when X is O; R^{f} is H; W is C; R^{j} is —CN; L is —SCH₂—; R¹ is phenylene or pyridine; then R⁷ is not —COOH;
when X is O; R^{f} is H; W is C; R^{j} is —CN; L is —SCH₂—; R¹ is phenylene or pyridine; and R⁷ is tetrazole; then R^{c} is not H;
when X is O; R^{f} is H; W is C; R^{j} is —CN; L is —S—C(R⁵)₂ or —SCH₂CH₂—; R¹ is absent; then R⁷ is not COOH or tetrazole;
when X is O, R^{f} is H; W is N; R^{j} is absent; R^{d} is methyl, optionally substituted 5- to 10-membered aryl, optionally substituted 5- or 6-membered heteroaryl, or optionally substituted 5- or 6-membered cycloalkyl; L is —SCH₂— or —OCH₂—; and R¹ is phenylene; then R⁷ is not —COOH, —CH₂COOH,

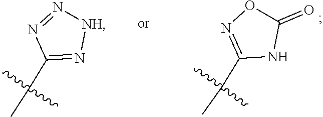 [SMILES: *c1nn[nH]n1 ; *c1noc(=O)[nH]1]

and
when X is O, R^{f} is H, W is N, R^{j} is absent, L is —NHCH₂—, —CH₂NH—, or —NH—C(O)—, and R¹ is phenylene, then R^{d} is not phenyl;
in the manufacture of a medicament for treating an acute inflammatory condition.

Embodiment I-2C. Use of compound represented by Formula (I):

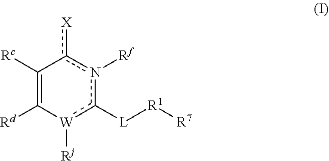

or a pharmaceutically acceptable salt or tautomer thereof,
wherein:
X is H, S, SR², NR², NR²R^{2′}, O, OH, OR^{h}, F, Br, or Cl;
W is N or C;
   (i) when W is N, then:
   L is —(C(R⁵)₂)ₘCH═CH(C(R⁵)₂)ₚ—,

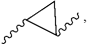

—(C(R⁵)₂)ₘY¹(C(R⁵)₂)ₚ—, —(C(R⁵)₂)ₘY¹(C(R⁵)₂)ₚ-cyclopropyl-, —(C(R⁵)₂)ₘY¹CH═CH—, —(C(R⁵)₂)ₘNR³C═(O)(C(R⁵)₂)ₚ—, —(C(R⁵)₂)ₘphenyl(C(R⁵)₂)ₚ—, —(C(R⁵)₂)ₘpyridinyl(C(R⁵)₂)ₚ—, or —(C(R⁵)₂)ₘthiophenyl(C(R⁵)₂)ₚ—;
(ii) when W is C, then:
L is —(C(R⁵)₂)ₘCH═CH(C(R⁵)₂)ₚ—, —(C(R⁵)₂)ₒ, —(C(R⁵)₂)ₘY¹(C(R⁵)₂)ₚ—,

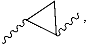

(C(R⁵)₂)ₘ Y¹CH═CH—, —(C(R⁵)₂)ₘC═(O)(CH₂)ₚ—, —(C(R⁵)₂)ₘC═(O)O(C(R⁵)₂)ₚ—, —(C(R⁵)₂)ₘC═(O)NR³(C(R⁵)₂)ₚ—, —(C(R⁵)₂)ₘNR³C═(O)(C(R⁵)₂)ₚ—, —(C(R⁵)₂)ₘphenyl(C(R⁵)₂)ₚ—, —(C(R⁵)₂)ₘpyridinyl(C(R⁵)₂)ₚ—, or —(C(R⁵)₂)ₘthiophenyl(C(R⁵)₂)ₚ—;
Y¹ is O, NR⁴, or S(O)_{q};
each Y² is independently O, NH or S;
R¹ is absent or C₆-C₁₀ arylene or heteroarylene, wherein the heteroarylene comprises one or two 5- to 7-membered rings and 1-4 heteroatoms selected from N, O and S, and wherein the C₆-C₁₀ arylene or heteroarylene are optionally substituted with one to two R^{e};
R² is H or C₁-C₄ alkyl;
R^{2′} is H, C₁-C₄ alkyl, or C₃-C₇ cycloalkyl; or
R² and R^{2′} together with the nitrogen atom to which they are attached form a 3- to 7-membered heterocycloalkyl ring comprising 1-3 additional heteroatoms selected from N, O and S;
R³ is H or C₁-C₄ alkyl;
R⁴ is H or C₁-C₄ alkyl;
each R⁵ is independently at each occurrence H or C₁-C₄ alkyl;
each R⁶ is independently at each occurrence H or C₁-C₄ alkyl;
R⁷ is H, A, B, or C;
A is —(C(R⁶)₂)ᵣCO₂R^{x}, —Y²(C(R⁶)₂)ᵣCO₂R^{x}, —(CH₂)ᵣtetrazole, —(CH₂)ᵣoxadiazolone, —(CH₂)ᵣtetrazolone, —(CH₂)ᵣthiadiazolol, —(CH₂)ᵣ isoxazol-3-ol, —(CH₂)ᵣP(O)(OH)OR^{x}, —(CH₂)ᵣS(O)₂OH, —(CH₂)ᵣC(O)NHCN, or —(CH₂)ᵣC(O)NHS(O)₂alkyl, wherein —(CH₂)ᵣtetrazole, —(CH₂)ᵣoxadiazolone, —(CH₂)ᵣtetrazolone, —(CH₂)ᵣthiadiazolol, —(CH₂)ᵣ isoxazol-3-ol are optionally substituted with C₁-C₆ alkyl,
B is —(C(R⁶)₂)ᵣS(O)₂OC₁-C₄ alkyl, —O(C(R⁶)₂)ᵣS(O)₂OC₁-C₄ alkyl, —Y²(C(R⁶)₂)ᵣC(O)NR^{g}R^{g′}, —Y²(C(R⁶)₂)ᵣS(O)₂NR^{g}R^{g′}, —(CH₂)ᵣC(O)NR^{g}R^{g′}, —(CH₂)ᵣS(O)₂NR^{g}R^{g′}, —(CH₂)ᵣC(O)NHS(O)₂NR^{g}R^{g′}, —(C(R⁶)₂)ᵣCO₂R¹, —(C(R⁶)₂)ᵣNH₂CO₂R^{x}, —(C(R⁶)₂)ᵣP(O)(OR^{x})₂, —O(C(R⁶)₂)ᵣP(O)(OR^{x})₂, —(C(R⁶)₂)ᵣS(O)₂OH, —O(C(R⁶)₂)ᵣS(O)₂OH, —(C(R⁶)₂)ᵣP(O)₂OR^{x}, or —O(C(R⁶)₂)ᵣP(O)₂OR^{x},
C is —(CH₂)ᵣCN, —(CH₂)ₛOH, halogen, —(C(R⁶)₂)ᵣC₆-C₁₀ aryl, —(C(R⁶)₂)ᵣS—C₆-C₁₀ aryl, —(C(R⁶)₂)ᵣheteroaryl, —O(C(R⁶)₂)ᵣheteroaryl, —O(C(R⁶)₂)ᵣheterocycloalkyl, —O(C(R⁶)₂)ᵣOH, —OR^{y}, —(C(R⁶)₂)ᵣC(O)NHCN, —CH═CHCO₂R^{x}, or —(C(R⁶)₂)ᵣC(O)NHS(O)₂C₁-C₄ alkyl, wherein the aryl and heteroaryl are substituted with one to three substituents each independently selected from C₁-C₆ alkyl, C₁-C₆ haloalkyl, halogen, and OH, and wherein the heterocycloalkyl is substituted with one to two ═O or ═S;
R^{c} is H, C₁-C₆ alkyl, C₁-C₆ haloalkyl, halogen, —CN, —OR^{x}, or —CO₂R^{x};
R^{d} is methyl, CF₃, CR^{f}F₂, —(C(R⁶)₂)ₜC₆-C₁₀ aryl, —(C(R⁶)₂)ₜ-5- or 6-membered heteroaryl, —(C(R⁶)₂)ₜ-5- or 6-membered cycloalkyl, optionally substituted C₆-C₁₀ aryl, optionally substituted 5- or 6-membered heteroaryl, or optionally substituted 5- or 6-membered cycloalkyl;
each R^{e} is independently at each occurrence C₁-C₆ alkyl, C₂-C₆ alkenyl, C₂-C₆ alkynyl, halogen, C₁-C₆ haloalkyl, —NHR^{z}, —OH, or —CN;
R^{f} is absent, H, or methyl;
R^{g} is H, C₁-C₆ alkyl, OH, —S(O)₂(C₁-C₆ alkyl), or S(O)₂N(C₁-C₆ alkyl)₂;
R^{g′} is H, C₁-C₆ alkyl, C₃-C₇ cycloalkyl, 4- to 7-membered heterocycloalkyl ring comprising 1-3 heteroatoms selected from N, O and S, C₆-C₁₀ aryl, or 5- to 7-membered heteroaryl comprising 1-3 heteroatoms selected from N, O and S, wherein the alkyl is optionally substituted with one or more substituents independently selected from halogen and —OH, and wherein the cycloalkyl, heterocycloalkyl, aryl and heteroaryl are optionally substituted with one or more substituents independently selected from C₁-C₆ alkyl, halogen, and —OH;
R^{h} is H, C₁-C₄ alkyl, or 3- to 7-membered heterocycloalkyl ring comprising 1-3 heteroatoms selected from N, O and S, wherein the alkyl is optionally substituted with one or more substituents each independently selected from NH₂, C₁-C₄ alkylamino, C₁-C₄ dialkylamino, and C(O)NH₂; and wherein the heterocycloalkyl is optionally substituted with one or more substituents each independently selected from C₁-C₆ alkyl and C₁-C₆ haloalkyl;
Rⁱ is (i) —(CH₂)ₛOC(O)C₁-C₆ alkyl, wherein the alkyl is substituted with one or more NH₂; (ii) (CH₂CH₂O)ₙCH₂CH₂OH; or (iii) C₁-C₆ alkyl substituted with one or more substituents each independently selected from OH and 4- to 7-membered heterocycloalkyl comprising 1 to 3 heteroatoms selected from O, N, or S;
R^{j} is absent, H, C₁-C₆ alkyl, or —CN;
each R^{x} is independently at each occurrence H, C₁-C₆ alkyl, or C₆-C₁₀ aryl;
each R^{y} and R^{z} is independently H, C₁-C₆ alkyl, or C₁-C₆ haloalkyl;
each m, p, q, r, and t is independently 0, 1 or 2;
n is 0, 1, 2, or 3;
s is 1 or 2;
o is 0, 1, 2, 3, or 4; and
[symbol] represents a single bond or a double bond; and
provided that
when X is O; R^{f} is H; W is C; R^{j} is —CN; L is —SCH₂—; R¹ is phenylene or pyridine; then R⁷ is not —COOH;
when X is O; R^{f} is H; W is C; R^{j} is —CN; L is —SCH₂—; R¹ is phenylene or pyridine; and R⁷ is tetrazole; then R^{c} is not H;
when X is O; R^{f} is H; W is C; R^{j} is —CN; L is —S—C(R⁵)₂ or —SCH₂CH₂—; R¹ is absent; then R⁷ is not COOH or tetrazole;
when X is O, R^{f} is H; W is N; R^{j} is absent; R^{d} is methyl, optionally substituted 5- to 10-membered aryl, optionally substituted 5- or 6-membered heteroaryl, or optionally substituted 5- or 6-membered cycloalkyl; L is —SCH₂— or —OCH₂—; and R¹ is phenylene; then R⁷ is not —COOH, —CH₂COOH,

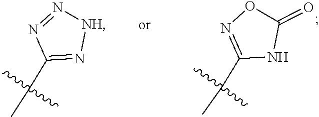 [SMILES: *c1nn[nH]n1 ; *c1noc(=O)[nH]1]

and
when X is O, R^{f} is H, W is N, R is absent, L is —NHCH₂—, —CH₂NH—, or —NH—C(O)—, and R¹ is phenylene, then R^{d} is not phenyl;
in the manufacture of a medicament for treating an acute inflammatory condition.

Embodiment I-3C. The use of Embodiment I-1C or I-2C, wherein X is O, OH, OR^{h}, F, Br, or Cl.

Embodiment I-4C. The use of Embodiment I-1C or I-2C, wherein X is H, S, SR², NR², or NR²R².

Embodiment I-5C. The use of any one of Embodiments I-1C to I-4C, wherein R^{f} is absent.

Embodiment I-6C. The use of any one of Embodiments I-1C to I-4C, wherein R^{f} is H or methyl.

Embodiment I-7C. The use of any one of Embodiments I-1C to I-6C, wherein W is N.

Embodiment I-8C. The use of Embodiment I-7C, wherein R^{j} is absent.

Embodiment I-9C. The use of any one of Embodiments I-1C to I-6C, wherein W is C.

Embodiment I-10C. The use of Embodiment I-9C, wherein R^{j} is H, C₁-C₆ alkyl, or —CN.

Embodiment I-11C. The use of Embodiment I-9C or I-10C, wherein R^{j} is —CN.

Embodiment I-12C. The use of any one of Embodiments I-1C to I-11C, wherein R^{c} is C₁-C₆ alkyl, —CN, or halogen.

Embodiment I-13C. The use of any one of Embodiments I-1C to I-12C, wherein R^{c} is —CN or halogen.

Embodiment I-14C. The use of any one of Embodiments I-1C to I-12C, wherein R^{c} is —CN.

Embodiment I-15C. The use of any one of Embodiments I-1C to I-14C, wherein R^{d} is methyl.

Embodiment I-16C. The use of any one of Embodiments I-1C to I-14C, wherein R^{d} is optionally substituted 5- to 10-membered aryl.

Embodiment I-17C. The use of any one of Embodiments I-1C to I-14C, wherein R^{d} is optionally substituted 5- or 6-membered heteroaryl.

Embodiment I-18C. The use of any one of Embodiments I-1C to I-14C, wherein R^{d} is optionally substituted 5- or 6-membered cycloalkyl.

Embodiment I-19C. The use of any one of Embodiments I-1C to I-14C, wherein R^{d} is methyl, cyclohexyl, pyridinyl, thiazolyl, phenyl, or thienyl.

Embodiment I-20C. The use of any one of Embodiments I-1C to I-14C, wherein R^{d} is methyl, cyclohexyl, pyridinyl, thiazolyl, thienyl, or optionally substituted phenyl.

Embodiment I-21C. The use of any one of Embodiments I-1C to I-14C, wherein R^{d} is methyl.

Embodiment I-22C. The use of any one of Embodiments I-1C to I-14C, wherein R^{d} is —CF₃.

Embodiment I-23C. The use of any one of Embodiments I-1C to I-14C, wherein R^{d} is CR^{f}F₂.

Embodiment I-24C. The use of any one of Embodiments I-1C to I-14C, wherein R^{d} is —(C(R⁶)₂)ₜC₆-C₁₀ aryl, —(C(R⁶)₂)ₜ-5- or 6-membered heteroaryl, —(C(R⁶)₂)ₜ-5- or 6-membered cycloalkyl.

Embodiment I-25C. The use of any one of Embodiments I-1C to I-14C, wherein R^{d} is —(C(R⁶)₂)ₜC₆-C₁₀ aryl.

Embodiment I-26C. The use of any one of Embodiments I-1C to I-25C, wherein L is —(C(R⁵)₂)ₘY¹(C(R⁵)₂)ₚ—.

Embodiment I-27C. The use of Embodiment I-26C, wherein Y¹ is S.

Embodiment I-28C. The use of any one of Embodiment I-1C to I-25C, wherein L is —(C(R⁵)₂)ₘNR³C═(O)(C(R⁵)₂)ₚ— or —(C(R⁵)₂)ₘY¹(C(R⁵)₂)ₚ-cyclopropyl-.

Embodiment I-29C. The use of any one of Embodiments I-1C to I-28C, wherein R¹ is C₆-C₁₀ arylene.

Embodiment I-30C. The use of any one of Embodiments I-1C to I-28C, wherein R¹ is heteroarylene.

Embodiment I-31C. The use of any one of Embodiments I-1C to I-28C, wherein R¹ is absent.

Embodiment I-32C. The use of any one of Embodiments I-1C to I-31C, wherein R⁷ is A.

Embodiment I-33C. The use of Embodiment I-32C, wherein A is —(C(R⁶)₂)ᵣCO₂R^{x} or —(CH₂)ᵣtetrazole, wherein the —(CH₂)ᵣtetrazole is optionally substituted with C₁-C₆ alkyl.

Embodiment I-34C. The use of any one of Embodiments I-1B to I-31B, wherein R⁷ is B.

Embodiment I-35C. The use of Embodiment I-32C, wherein B is —(CH₂)ᵣC(O)NR^{g}R^{g′}, or —(CH₂)ᵣS(O)₂NR^{g}R^{g′},

Embodiment I-36C. The use of any one of Embodiments I-1C to I-31C, wherein R⁷ is C.

Embodiment I-37C. The use of Embodiment I-32C, wherein C is —(CH₂)ᵣCN, —(CH₂)ₛOH, or —(C(R⁶)₂)ᵣC₆-C₁₀ aryl, wherein the aryl is substituted with one to three substituents each independently selected from C₁-C₆ alkyl, C₁-C₆ haloalkyl, halogen, and OH.

Embodiment I-38C. The use of any one of Embodiment I-1C to I-37C, wherein the compound is

| | |
|---|---|
| I-34 | 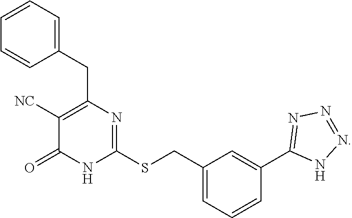 [SMILES: N#Cc1c(Cc2ccccc2)nc(SCc2cccc(-c3nnn[nH]3)c2)[nH]c1=O] |

Embodiment I-39C. The use of any one of Embodiments I-1C to I-38C, wherein the acute inflammatory condition is a systemic inflammatory condition.

Embodiment I-40C. The use of any one of Embodiments I-1C to I-38C, wherein the acute inflammatory condition is an organ-specific condition.

Embodiment I-41C. The use of any one of Embodiments I-1C to I-38C, wherein the acute inflammatory condition is cytokine storm or hypercytokinemia, systemic inflammatory response syndrome (SIRS), graft versus host disease (GVHD), acute respiratory distress syndrome (ARDS), severe acute respiratory distress syndrome (SARS), catastrophic anti-phospholipid syndrome, viral infections, bacterial infections, fungal infections, influenza, pneumonia, shock, or sepsis.

Embodiment I-42C. The use of any one of Embodiments I-1C to I-38C, wherein the acute inflammatory condition is acute pancreatitis, hepatitis, respiratory condition, or enterocolitis.

Embodiment I-43C. The use of any one of Embodiments I-1C to I-42C, wherein the method reduces a pro-inflammatory cytokine or increases an anti-inflammatory cytokine.

Embodiment I-44C. The use of Embodiment I-43C, wherein the pro-inflammatory cytokine is IL-1β, IL-6, IL-18, TNF-α, or TGF-β.

Embodiment I-45C. The use of Embodiment I-43C, wherein pro-inflammatory cytokine is MCP-1, TNF-α, or IL-1β and the pro-inflammatory cytokine is increased.

Embodiment I-46C. The use of Embodiment I-43C, wherein pro-inflammatory cytokine is IL-6 and the pro-inflammatory cytokine is increased.

Embodiment I-47C. The use of Embodiment I-43C, wherein the anti-inflammatory cytokine is IL-10.

Embodiment I-48C. The use of any one of Embodiments I-1C to I-42C, wherein expression of sirtuin-1 modulated genes sod2, tfam, dda1 genes are increased in the liver.

Embodiment I-49C. The use of any one of Embodiments I-1C to I-48C, wherein the administration to the subject occurs at least 12 hours after an injury.

Embodiment I-50C. The use of any one of Embodiments I-1C to I-49C, wherein the administration to the subject occurs for at least 6 days after an injury.

Embodiment II-1. A method of treating an acute inflammatory condition in a subject comprising administering to the subject a therapeutically effective amount of a compound represented by Formula (II):

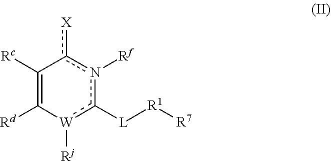

or a pharmaceutically acceptable salt or tautomer thereof, wherein:
X is O, OR^{h};
W is N;
L is —(C(R⁵)₂)ₘY¹(C(R⁵)₂)ₚ;
Y¹ is S;
R¹ is absent, C₆-C₁₀ arylene or heteroarylene, wherein the heteroarylene comprises one or two 5- to 7-membered rings and 1-4 heteroatoms selected from N, O, and S, and wherein the C₆-C₁₀ arylene and heteroarylene are unsubstituted or substituted with one to two R^{e};
each R⁵ is independently at each occurrence H or C₁-C₄ alkyl;
each R⁶ is independently at each occurrence H or C₁-C₄ alkyl;
R⁷ is A or C;
A is —(C(R⁶)₂)ᵣtetrazole, —(C(R⁶)₂)ᵣoxadiazolone, —(C(R⁶)₂)ᵣtetrazolone, —(C(R⁶)₂)ᵣthiadiazolol, —(C(R⁶)₂)ᵣisoxazol-3-ol;
C is —(C(R⁶)₂)ᵣC₆-C₁₀ aryl, wherein the aryl is substituted with one to three substituents each independently selected from C₁-C₆ alkyl, C₁-C₆ haloalkyl, chloro, bromo, and OH;
R^{c} is H, halogen, or —CN;
R^{d} is —CF₃, —CR^{f}F₂, —(C(R⁶)₂)ₜ—C₆-C₁₀ aryl, —(C(R⁶)₂)ₜ-5- or 6-membered heteroaryl;
each R^{e} is independently at each occurrence C₁-C₆ alkyl, chloro, bromo, C₁-C₆ haloalkyl, —NHR^{z}, —OH, or —CN;
R^{h} is H;
R^{j} is absent;
R^{z} is H, C₁-C₆ alkyl, or C₁-C₆ haloalkyl;
each m, p, and r are independently 0, 1, or 2;
t is 1 or 2; and
[symbol] represents a single bond or a double bond.

Embodiment II-2. The method of Embodiment II-1, wherein L is —SCH₂—.

Embodiment II-3. The method of any one of Embodiments II-1 to II-2, wherein R^{c} is H.

Embodiment II-4. The method of any one of Embodiments II-1 to II-2, wherein R^{c} is —CN.

Embodiment II-5. The method of any one of Embodiments II-1 to II-4, wherein R^{d} is —CF₃.

Embodiment II-6. The method of any one of Embodiments II-1 to II-4, wherein R^{d} is —CR^{f}F₂.

Embodiment II-7. The method of any one of Embodiments II-1 to II-4, wherein R^{d} is —(C(R⁶)₂)ₜ—C₆-C₁₀ aryl or —(C(R⁶)₂)ₜ-5- or 6-membered heteroaryl.

Embodiment II-8. The method of Embodiment II-7, wherein R^{d} is —CH₂—C₆-C₁₀ aryl.

Embodiment II-9. The method of any one of Embodiments II-1 to II-8, wherein R¹ is C₆-C₁₀ arylene.

Embodiment II-10. The method of any one of Embodiments II-1 to II-8, wherein R¹ is heteroarylene.

Embodiment II-11. The method of any one of Embodiments II-1 to II-8, wherein R¹ is absent.

Embodiment II-12. The method of any one of Embodiments II-1 to II-11, wherein R⁷ is A.

Embodiment II-13. The method of Embodiment II-12, wherein A is —(CH₂)ᵣtetrazole.

Embodiment II-14. The method of any one of Embodiments II-1 to II-11, wherein R⁷ is C.

Embodiment II-15. The method of Embodiment II-14, wherein C is —(C(R⁶)₂)ᵣC₆-C₁₀ aryl, wherein the aryl is substituted with one to three substituents each independently selected from C₁-C₆ alkyl, C₁-C₆ haloalkyl, halogen, and OH.

Embodiment I-16. The method of Embodiment II-1, wherein R^{c} is C₆-C₁₀ arylene; R⁷ is A; and A is —(CH₂)ᵣtetrazole.

Embodiment II-17. The method of Embodiment II-1, wherein R¹ is heteroarylene; R⁷ is A; and A is —(CH₂)ᵣtetrazole.

Embodiment II-18. The method of Embodiment II-1, wherein R¹ is absent; R⁷ is A; and A is —(CH₂)ᵣtetrazole.

Embodiment II-19. The method of Embodiment II-1, wherein R¹ is absent; R⁷ is C; and C is —(C(R⁶)₂)ᵣC₆-C₁₀ aryl, wherein the aryl is substituted with one to three substituents each independently selected from C₁-C₆ alkyl, C₁-C₆ haloalkyl, halogen, and OH.

Embodiment II-1A. A compound represented by Formula (II):

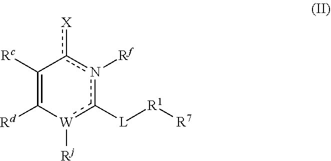

or a pharmaceutically acceptable salt or tautomer thereof, wherein:
X is O, OR^{h};
W is N;
L is —(C(R⁵)₂)ₘY¹(C(R⁵)₂)ₚ;
Y¹ is S;
R¹ is absent, C₆-C₁₀ arylene or heteroarylene, wherein the heteroarylene comprises one or two 5- to 7-membered rings and 1-4 heteroatoms selected from N, O, and S, and wherein the C₆-C₁₀ arylene and heteroarylene are unsubstituted or substituted with one to two R^{e};
each R⁵ is independently at each occurrence H or C₁-C₄ alkyl;
each R⁶ is independently at each occurrence H or C₁-C₄ alkyl;
R⁷ is A or C;
A is —(C(R⁶)₂)ᵣtetrazole, —(C(R⁶)₂)ᵣoxadiazolone, —(C(R⁶)₂)ᵣtetrazolone, —(C(R⁶)₂)ᵣthiadiazolol, —(C(R⁶)₂)ᵣisoxazol-3-ol;
C is —(C(R⁶)₂)ᵣC₆-C₁₀ aryl, wherein the aryl is substituted with one to three substituents each independently selected from C₁-C₆ alkyl, C₁-C₆ haloalkyl, chloro, bromo, and OH;
R^{c} is H, halogen, or —CN;
R^{d} is —CF₃, —CR^{f}F₂, —(C(R⁶)₂)ₜ—C₆-C₁₀ aryl, —(C(R⁶)₂)ₜ-5- or 6-membered heteroaryl;
each R^{e} is independently at each occurrence C₁-C₆ alkyl, chloro, bromo, C₁-C₆ haloalkyl, —NHR^{z}, —OH, or —CN;
R^{f} is H;
R^{h} is H;
R^{j} is absent;
R^{z} is H, C₁-C₆ alkyl, or C₁-C₆ haloalkyl;
each m, p, and r are independently 0, 1, or 2;
t is 1 or 2; and
[symbol] represents a single bond or a double bond;
for use in treating an acute inflammatory condition.

Embodiment II-2A. The compound of Embodiment II-1A, wherein L is —SCH₂—.

Embodiment II-3A. The compound of any one of Embodiments II-1A to II-2A, wherein R^{c} is H.

Embodiment II-4A. The compound of any one of Embodiments II-1A to II-2A, wherein R^{c} is —CN.

Embodiment II-5A. The compound of any one of Embodiments II-1A to II-4A, wherein R^{d} is —CF₃.

Embodiment II-6A. The compound of any one of Embodiments II-1A to II-4A, wherein R^{d} is —CR^{f}F₂.

Embodiment II-7A. The compound of any one of Embodiments II-1A to II-4A, wherein R^{d} is —(C(R⁶)₂)ₜ—C₆-C₁₀ aryl or —(C(R⁶)₂)ₜ-5- or 6-membered heteroaryl.

Embodiment II-8A. The compound of Embodiment II-7A, wherein R^{d} is —CH₂—C₆-C₁₀ aryl.

Embodiment II-9A. The compound of any one of Embodiments II-1A to II-8A, wherein R¹ is C₆-C₁₀ arylene.

Embodiment II-10A. The compound of any one of Embodiments II-1A to II-8A, wherein R¹ is heteroarylene.

Embodiment II-11A. The compound of any one of Embodiments II-1A to II-8A, wherein R¹ is absent.

Embodiment II-12A. The compound of any one of Embodiments II-1A to II-11A, wherein R⁷ is A.

Embodiment II-13A. The compound of Embodiment II-12A, wherein A is —(CH₂)ᵣtetrazole.

Embodiment II-14A. The compound of any one of Embodiments II-1A to II-11A, wherein R⁷ is C.

Embodiment II-15A. The compound of Embodiment II-14A, wherein C is —(C(R⁶)₂)ᵣC₆-C₁₀ aryl, wherein the aryl is substituted with one to three substituents each independently selected from C₁-C₆ alkyl, C₁-C₆ haloalkyl, halogen, and OH.

Embodiment II-16A. The compound of Embodiment II-1A, wherein R¹ is C₆-C₁₀ arylene; R⁷ is A; and A is —(CH₂)ᵣtetrazole.

Embodiment II-17A. The compound of Embodiment II-1A, wherein R¹ is heteroarylene; R⁷ is A; and A is —(CH₂)ᵣtetrazole.

Embodiment II-18A. The compound of Embodiment II-1A, wherein R¹ is absent; R⁷ is A; and A is —(CH₂)ᵣtetrazole.

Embodiment II-19A. The compound of Embodiment II-1A, wherein R¹ is absent; R⁷ is C; and C is —(C(R⁶)₂)ᵣC₆-C₁₀ aryl, wherein the aryl is substituted with one to three substituents each independently selected from C₁-C₆ alkyl, C₁-C₆ haloalkyl, halogen, and OH.

Embodiment II-1B. A use of a compound represented by Formula (II):

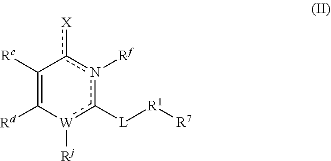

or a pharmaceutically acceptable salt or tautomer thereof, wherein:
X is O, OR^{h};
W is N;
L is —(C(R⁵)₂)ₘY¹(C(R⁵)₂)ₚ;
Y¹ is S;
R¹ is absent, C₆-C₁₀ arylene or heteroarylene, wherein the heteroarylene comprises one or two 5- to 7-membered rings and 1-4 heteroatoms selected from N, O, and S, and wherein the C₆-C₁₀ arylene and heteroarylene are unsubstituted or substituted with one to two R^{e};
each R⁵ is independently at each occurrence H or C₁-C₄ alkyl;
each R⁶ is independently at each occurrence H or C₁-C₄ alkyl;
R⁷ is A or C;
A is —(C(R⁶)₂)ᵣtetrazole, —(C(R⁶)₂)ᵣoxadiazolone, —(C(R⁶)₂)ᵣtetrazolone, —(C(R⁶)₂)ᵣthiadiazolol, —(C(R⁶)₂)ᵣisoxazol-3-ol;
C is —(C(R⁶)₂)ᵣC₆-C₁₀ aryl, wherein the aryl is substituted with one to three substituents each independently selected from C₁-C₆ alkyl, C₁-C₆ haloalkyl, chloro, bromo, and OH;
R^{c} is H, halogen, or —CN;
R^{d} is —CF₃, —CR^{f}F₂, —(C(R⁶)₂)ₜ—C₆-C₁₀ aryl, —(C(R⁶)₂)ₜ-5- or 6-membered heteroaryl;
each R^{e} is independently at each occurrence C₁-C₆ alkyl, chloro, bromo, C₁-C₆ haloalkyl, —NHR^{z}, —OH, or —CN;
R^{f} is H;
R^{h} is H;
R^{j} is absent;
R^{z} is H, C₁-C₆ alkyl, or C₁-C₆ haloalkyl;
each m, p, and r are independently 0, 1, or 2;
t is 1 or 2; and
[symbol] represents a single bond or a double bond;
for treating an acute inflammatory condition.

Embodiment II-2B. The use of Embodiment II-1B, wherein L is —SCH₂—.

Embodiment II-3B. The use of any one of Embodiments II-1B to II-2B, wherein R^{c} is H.

Embodiment II-4B. The use of any one of Embodiments II-1B to II-2B, wherein R^{c} is —CN.

Embodiment II-5B. The use of any one of Embodiments II-1B to II-4B, wherein R^{d} is —CF₃.

Embodiment II-6B. The use of any one of Embodiments II-1B to II-4B, wherein R^{d} is —CR^{f}F₂.

Embodiment II-7B. The use of any one of Embodiments II-1B to II-4B, wherein R^{d} is —(C(R⁶)₂)ₜ—C₆-C₁₀ aryl or —(C(R⁶)₂)ₜ-5- or 6-membered heteroaryl.

Embodiment II-8B. The use of Embodiment II-7B, wherein R^{d} is —CH₂—C₆-C₁₀ aryl.

Embodiment II-9B. The use of any one of Embodiments II-1B to II-8B, wherein R¹ is C₆-C₁₀ arylene.

Embodiment II-10B. The use of any one of Embodiments II-1B to II-8B, wherein R¹ is heteroarylene.

Embodiment II-11B. The use of any one of Embodiments II-1B to II-8B, wherein R¹ is absent.

Embodiment II-12B. The use of any one of Embodiments II-1B to II-11B, wherein R⁷ is A.

Embodiment II-13B. The use of Embodiment II-12B, wherein A is —(CH₂)ᵣtetrazole.

Embodiment II-14B. The use of any one of Embodiments II-1B to II-11B, wherein R⁷ is C.

Embodiment II-15B. The use of Embodiment II-14B, wherein C is —(C(R⁶)₂)ᵣC₆-C₁₀ aryl, wherein the aryl is substituted with one to three substituents each independently selected from C₁-C₆ alkyl, C₁-C₆ haloalkyl, halogen, and OH.

Embodiment II-16B. The use of Embodiment II-1B, wherein R¹ is C₆-C₁₀ arylene; R⁷ is A; and A is —(CH₂)ᵣtetrazole.

Embodiment II-17B. The use of Embodiment II-1B, wherein R¹ is heteroarylene; R⁷ is A; and A is —(CH₂)ᵣtetrazole.

Embodiment II-18B. The use of Embodiment II-1B, wherein R¹ is absent; R⁷ is A; and A is —(CH₂)ᵣtetrazole.

Embodiment II-19B. The use of Embodiment II-1B, wherein R¹ is absent; R⁷ is C; and C is —(C(R⁶)₂)ᵣC₆-C₁₀ aryl, wherein the aryl is substituted with one to three substituents each independently selected from C₁-C₆ alkyl, C₁-C₆ haloalkyl, halogen, and OH.

Embodiment II-1C. Use of a compound represented by Formula (II):

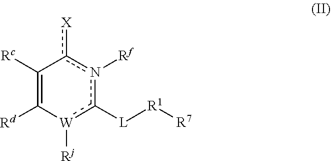

or a pharmaceutically acceptable salt or tautomer thereof, wherein:
X is O, OR^{h};
W is N;
L is —(C(R⁵)₂)ₘY¹(C(R⁵)₂)ₚ;
Y¹ is S;
R¹ is absent, C₆-C₁₀ arylene or heteroarylene, wherein the heteroarylene comprises one or two 5- to 7-membered rings and 1-4 heteroatoms selected from N, O, and S, and wherein the C₆-C₁₀ arylene and heteroarylene are unsubstituted or substituted with one to two R^{e};
each R⁵ is independently at each occurrence H or C₁-C₄ alkyl;
each R⁶ is independently at each occurrence H or C₁-C₄ alkyl;
R⁷ is A or C;
A is —(C(R⁶)₂)ᵣtetrazole, —(C(R⁶)₂)ᵣoxadiazolone, —(C(R⁶)₂)ᵣtetrazolone, —(C(R⁶)₂)ᵣthiadiazolol, —(C(R⁶)₂)ᵣisoxazol-3-ol;
C is —(C(R⁶)₂)ᵣC₆-C₁₀ aryl, wherein the aryl is substituted with one to three substituents each independently selected from C₁-C₆ alkyl, C₁-C₆ haloalkyl, chloro, bromo, and OH;
R^{c} is H, halogen, or —CN;
R^{d} is —CF₃, —CR^{f}F₂, —(C(R⁶)₂)ₜ—C₆-C₁₀ aryl, —(C(R⁶)₂)ₜ-5- or 6-membered heteroaryl;
each R^{e} is independently at each occurrence C₁-C₆ alkyl, chloro, bromo, C₁-C₆ haloalkyl, —NHR^{z}, —OH, or —CN;
R^{f} is H;
R^{h} is H;
R^{j} is absent;
R^{z} is H, C₁-C₆ alkyl, or C₁-C₆ haloalkyl;
each m, p, and r are independently 0, 1, or 2;
t is 1 or 2; and
[symbol] represents a single bond or a double bond;
in the manufacture of a medicament for treating an acute inflammatory condition.

Embodiment II-2C. The use of Embodiment II-1C, wherein L is —SCH₂—.

Embodiment II-3C. The use of any one of Embodiments II-1C to II-2C, wherein R^{c} is H.

Embodiment II-4C. The use of any one of Embodiments II-1C to II-2C, wherein R^{c} is —CN.

Embodiment II-5C. The use of any one of Embodiments II-1C to II-4C, wherein R^{d} is —CF₃.

Embodiment II-6C. The use of any one of Embodiments II-1C to II-4C, wherein R^{d} is —CR^{f}F₂.

Embodiment II-7C. The use of any one of Embodiments II-1C to II-4C, wherein R^{d} is —(C(R⁶)₂)ₜ—C₆-C₁₀ aryl or —(C(R⁶)₂)ₜ-5- or 6-membered heteroaryl.

Embodiment II-8C. The use of Embodiment II-7C, wherein R^{d} is —CH₂—C₆-C₁₀ aryl.

Embodiment II-9C. The use of any one of Embodiments II-1C to II-8C, wherein R¹ is C₆-C₁₀ arylene.

Embodiment II-10C. The use of any one of Embodiments II-1C to II-8C, wherein R¹ is heteroarylene.

Embodiment II-11C. The use of any one of Embodiments II-1C to II-8C, wherein R¹ is absent.

Embodiment II-12C. The use of any one of Embodiments II-1C to II-11C, wherein R⁷ is A.

Embodiment II-13C. The use of Embodiment II-12C, wherein A is —(CH₂)ᵣtetrazole.

Embodiment II-14C. The use of any one of Embodiments II-1C to II-11C, wherein R⁷ is C.

Embodiment II-15C. The use of Embodiment II-14C, wherein C is —(C(R⁶)₂)ᵣC₆-C₁₀ aryl, wherein the aryl is substituted with one to three substituents each independently selected from C₁-C₆ alkyl, C₁-C₆ haloalkyl, halogen, and OH.

Embodiment II-16C. The use of Embodiment II-1C, wherein R¹ is C₆-C₁₀ arylene; R¹ is A; and A is —(CH₂)ᵣtetrazole.

Embodiment II-17C. The use of Embodiment II-1C, wherein R¹ is heteroarylene; R¹ is A; and A is —(CH₂)ᵣtetrazole.

Embodiment II-18C. The use of Embodiment II-1C, wherein R¹ is absent; R⁷ is A; and A is —(CH₂)ᵣtetrazole.

Embodiment II-19C. The use of Embodiment II-1C, wherein R¹ is absent; R⁷ is C; and C is —(C(R⁶)₂)ᵣC₆-C₁₀ aryl, wherein the aryl is substituted with one to three substituents each independently selected from C₁-C₆ alkyl, C₁-C₆ haloalkyl, halogen, and OH.

### EXAMPLES

All percentages and ratios used herein, unless otherwise indicated, are by weight. Other features and advantages of the present disclosure will become apparent from the different examples. The provided examples illustrate different components and methodology useful in practicing the present disclosure. Generally speaking, the disclosure extends to any novel one, or any novel combination, of the features disclosed in this specification (including the accompanying claims and drawings). The examples do not limit the claimed disclosure. Thus, features, integers, characteristics, compounds or chemical moieties described in conjunction with a particular aspect, embodiment or example of the disclosure are to be understood to be applicable to any other aspect, embodiment or example described herein, unless incompatible therewith. Based on the present disclosure the skilled artisan can identify and employ other components and methodology useful for practicing the present disclosure. Moreover, unless stated otherwise, any feature disclosed herein may be replaced by an alternative feature serving the same or a similar purpose.

The Disclosure will now be described by way of example only with reference to the Examples below:

### Compound Preparation

### General Methods and Materials

All chemicals were purchased from Sigma-Aldrich, Alfa Aesar. ¹H NMR spectra were recorded at 200 and 400 MHz and ¹³C NMR spectra were recorded at 100.6 and 50.3 MHz by using deuterated solvents indicated below. TLC were performed on aluminium backed silica plates (silica gel 60 F254). All the reactions were performed under nitrogen atmosphere using distilled solvents. All tested compounds were found to have >95% purity determined by HPLC analysis. HPLC-grade water was obtained from a tandem Milli-Ro/Milli-Q apparatus. The analytical HPLC measurements were made on a Shimadzu LC-20AProminence equipped with a CBM-20A communication bus module, two LC-20AD dual piston pumps, a SPD-M20A photodiode array detector and a Rheodyne 7725i injector with a 20 μL stainless steel loop.

Abbreviations used in the following examples and elsewhere herein are:

| | |
|---|---|
| Ac₂O | acetic anhydride |
| AcOH | acetic acid |
| AIBN | Azobisisobutyronitrile |
| atm | atmosphere |
| br | broad |
| DIPEA | N,N-diisopropylethylamine |
| DCM | dichloromethane |
| DME | dimethoxyethane |
| DMF | N,N-dimethylformamide |
| DMSO | dimethyl sulfoxide |
| BPO | Dibenzoylperoxide |
| EDC | N-(3-Dimethylaminopropyl)-N′-ethylcarbodiimide |
| | hydrochloride |
| ESI | electrospray ionization |
| EtOAc | ethyl acetate |
| EtO₂ | diethyl ether |
| EtOH | ethanol |
| EtO⁻Na⁺ | sodium ethoxide |
| Et₃NH⁺Cl⁻ | triethylamine hydrochloride |
| h | hour(s) |
| HPLC | high-performance liquid chromatography |
| LCMS | liquid chromatography-mass spectrometry |
| m | multiplet |
| MeI | methyl iodide |
| MeOH | methanol |
| MHz | megahertz |
| min | minutes |
| MS | molecular sieves |
| MTBE | 2-methoxy-2-methylpropane |
| MW | microwave |
| NBS | N-bromosuccinamide |
| NMR | nuclear magnetic resonance |
| PET | petroleum ether |
| ppm | parts per million |
| p-TSA | para-toluenesulfonic acid |
| r.t. | room temperature |
| TLC | thin layer chromatography |

### Example 1: Intermediate 1.4. 4-Oxo-6-thiophen-2-yl-2-thioxo-1,2,3,4-tetrahydro-pyrimidine-5-carbonitrile

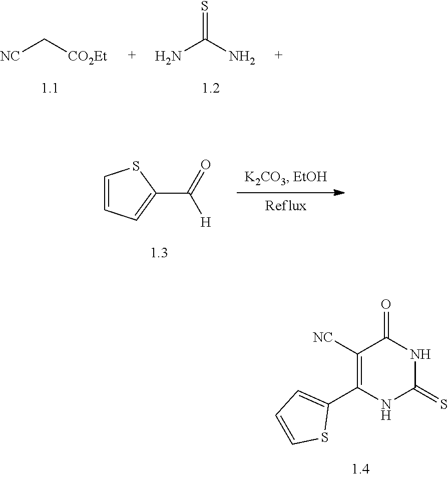

To a stirred solution of compound 1.1 (0.96 g, 8.8 mmol), 1.2 (672 mg, 8.8 mmol) and 1.3 (1 g, 0.83 mL) in ethanol (55 mL) was added K₂CO₃ (1.57 g, 11.44 mmol). Stirring was continued at reflux overnight. The yellowish solid formed was collected after cooling, taken up with hot water and filtered again. The aqueous phase was acidified to pH1, the precipitate was filtered and dried under reduced pressure. The title compound 1.4 was obtained as a yellowish solid (1 g, 4.25 mmol). Yield 49%. ¹H NMR (200 MHz, DMSO-d₆) δ 7.22 (m, 1H), 7.68 (m, 1H), 7.85 (d, J=4.8 Hz, 1H), 8.05 (s, 1H).

### Example 2: Intermediate 2.2. Sodium; 6-oxo-4-trifluoromethyl-1,6-dihydro-pyrimidine-2-thiolate

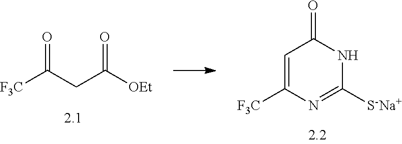

Sodium (0.35 g, 16.29 mmol) was dissolved in abs. EtOH (25 mL) under N₂ atmosphere. To the resulting solution ethyl trifluoroacetoacetate 2.1 (1.59 mL, 10.86 mmol) and thiourea 1.2 (0.91 g, 11.94 g) were added. The mixture was stirred and refluxed for 4 h. Once cooled at room temperature the obtained precipitate was collected by filtration under vacuum and washed with cold EtOH (2×5 mL), to afford (1.34 g, 6.14 mmol) of intermediate 2.2. Yield 38%. MS-ESI(−) m/z: 194.8 [M−H].

### Example 3: Intermediate 3.3. 2-Mercapto-6-oxo-4-phenyl-1,6-dihydro-pyridine-3-carbonitrile

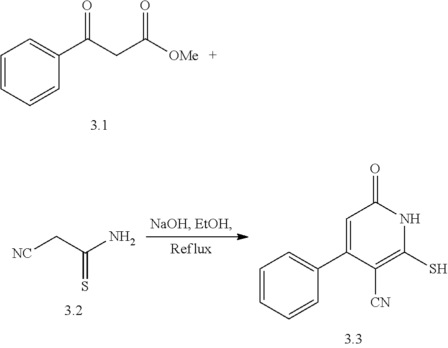

To a stirred solution of KOH (0.58 g, 10.41 mmol) in abs. EtOH (20 mL), ethyl 3-oxo-3-phenyl-propionate 3.1 (1.80 mL, 10.41 mmol) and 2-cyanothioacetamide 3.2 (1.04 g, 10.41 mmol) were added, and the resulting mixture was stirred and refluxed for 3 hours. Then it was cooled at room temperature and concentrated under reduced pressure. The crude was poured in H₂O (20 mL) and washed with AcOEt (2×15 mL). The organic phase was acidified un to pH=2 by adding aq. HCl 37%, and the resulting precipitate was collected by filtration under vacuum and washed with H₂O (2×5 mL). The solid was then tritured with AcMe, to give intermediate 3.3 (0.49 g, 2.14 mmol) as a yellowish solid. Yield 21%. MS-ESI(−) m/z: 227.3 [M−H]⁻

### Example 4: Intermediate 4.2. 4-Benzyl-2-mercapto-6-oxo-1,6-dihydro-pyrimidine-5-carbonitrile

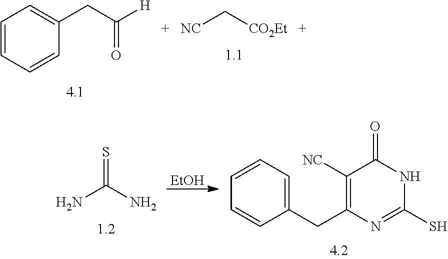

To a solution of phenyl acetaldehyde 4.1 (1.5 g, 16.65 mmol), ethylcyanoacetate 1.1 (1.41 g, 16.65 mmol) and thiourea 1.2 (950 mg, 16.65 mmol) in EtOH (35 mL) was added K₂CO₃ (2.2 g, 21.6 mmol). Stirring was continued at reflux 16 h. The mixture was cooled to r.t. The white solid was collected, dissolved in water. The pH was adjusted to 3 by the addition of 3N HCl. The aqueous phase was extracted with EtOAc (3×50 mL). The combined organic phase was washed with brine and dried over anhydrous Na₂SO₄. The title intermediate 4.2 (800 mg, 3.28 mmol) was obtained as a light yellow solid. Yield: 20%. ¹H NMR (200 MHz, DMSO-d₆) δ 3.93 (s, 2H), 7.26-7.41 (m, 5H), 13.15 (brs, 1H).

### Example 5: Intermediate 5.2. 2-Mercapto-6-oxo-4-thiophen-2-yl-1,6-dihydro-pyridine-3-carbonitrile

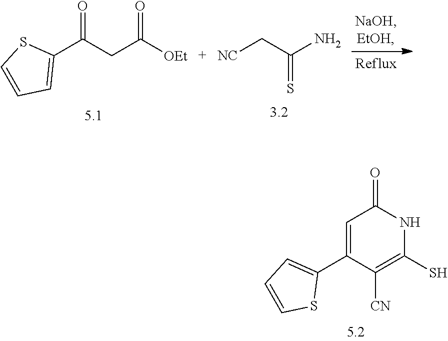

To a stirred solution of KOH (0.28 g, 5.04 mmol) in abs. EtOH (10 mL), ethyl 3-oxo-3-thiophen-2-yl-propionate 5.1 (0.77 mL, 5.04 mmol) and 2-cyanothioacetamide 3.2 (0.50 g, 5.04 mmol) were added, and the resulting mixture was stirred at reflux for 8 hours. Then it was cooled at room temperature and the precipitate formed was collected by filtration under vacuum and washed with EtOH (2×5 mL), to give intermediate 5.2 (0.17 g, 0.72) as a yellowish solid. Yield 12%. MS-ESI(−) m/z: 233.3 [M−H]⁻.

### Example 6: Intermediate 6.4. 6-Mercapto-2-oxo-4-thiophen-2-yl-1,2-dihydro-pyridine-3,5-dicarbonitrile

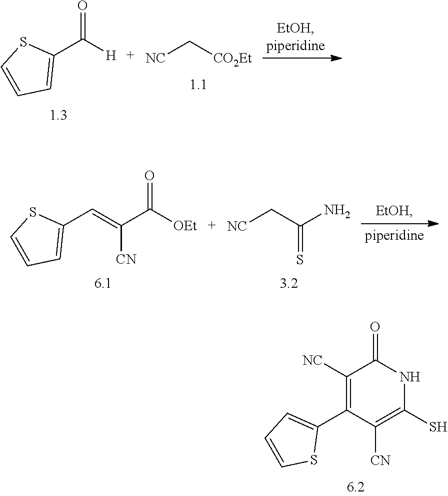

### Step 1: 2-Cyano-3-thiophen-2-yl-acrylic acid ethyl ester (6.1)

To a solution of thiophene-2-carboxaldehyde 1.3 (1 g, 8.9 mmol), ethylcyanoacetate 1.1 (0.94 mL, 8.9 mmol) in EtOH (20 mL) was added piperidine (3 drops). Stirring was continued ar r.t. 16 h. The solvent was removed under vacuo. The crude was taken up with water, extracted with EtOAc (3×50 mL). The organic phase was collected, washed with brine and dried over anhydrous Na₂SO₄. The title intermediate 6.1 (1.3 g, 6.27 mmol) was obtained as a white solid. Yield 70%.

### Step 2: 6-Mercapto-2-oxo-4-thiophen-2-yl-1,2-dihydro-pyridine-3,5-dicarbonitrile (6.2)

To a solution of intermediate 6.1 (1.2 g, 5.79 mmol) in EtOH (15 mL) was added piperidine (4 drops). Stirring was continued at reflux 16 h. Upon cooling a red precipitate was formed. The precipitate was collected, washed with cold EtOH, and dried under vacuo. The title intermediate 6.2 (640 mg, 2.46 mmol) as a red powder. Yield 42%. ¹H NMR (200 MHz, DMSO-d₆) δ 7.24-7.27 (m, 1H), 7.53-7.55 (m, 1H), 7.94-7.95 (m, 1H), 13.0 (brs, 1H).

### Example 7: Intermediate 7.1. Potassium; 3-cyano-6-oxo-4-trifluoromethyl-1,6-dihydro-pyridine-2-thiolate

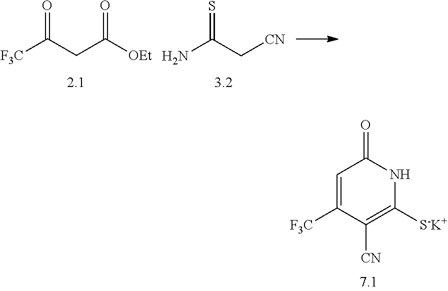

To a stirred solution of KOH (0.91 g, 16.29 mmol) in abs. EtOH (32 mL), ethyl trifluoroacetoacetate 2.1 (2.38 mL, 16.29 mmol) and 2-cyanothioacetamide 3.2 (1.63 g, 16.29 mmol) were added, and the resulting mixture was stirred and refluxed for 7 hours. Then it was cooled at room temperature and left to stand overnight. The copious precipitate thus formed was collected by filtration under vacuum and washed with EtOH (2×5 mL), to give intermediate 7.1 (2.01 g, 7.78 mmol) as a white solid. Yield 48%. MS-ESI(−) m/z: 218.9 [M H]⁻

### Example 8: Intermediate 8.3. (3-Bromomethyl-phenyl)-acetic acid ethyl ester

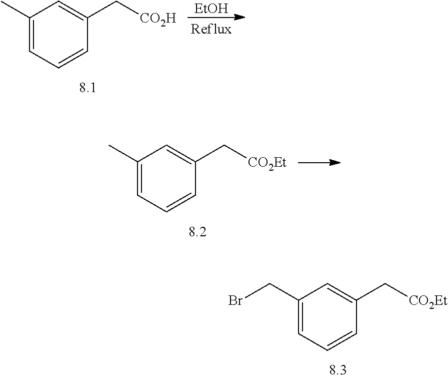

### Step 1: m-Tolyl-acetic acid ethyl ester (8.2)

To a solution of 8.1 (15 g, 99.88 mmol) in EtOH (absolute) (400 mL) was added HCl (conc.) (0.3 mL, 9.9 mmol) and stirring was continued at reflux for 4 h. The volatiles were removed under reduced pressure. The crude was taken up with DCM (200 mL) dried over Na₂SO₄ and evaporated under reduced pressure. The title compound 8.2 was obtained as a colorless oil (17 g, 95.39 mmol). Yield 96%. ¹H NMR (200 MHz, CDCl₃) δ 1.28 (t, J=7.1 Hz, 3H), 2.37 (s, 2H), 3.6 (s, 2H), 4.18 (q, J=7.11 Hz, 2H), 7.20-7.35 (m, 4H). GC/MS m/z 178.1 (M+).

### Step 2: (3-Bromomethyl-phenyl)-acetic acid ethyl ester (8.3)

NBS (10.1 g, 58.9 mmol) and BPO (70%) (68 mg, 0.28 mmol) were added to a solution of intermediate 8.2 (10 g, 56.11 mmol) in CH₃CN (300 mL). Stirring was continued at reflux for 4 h. The volatile were removed under reduced pressure. The crude residue was partitioned between EtOAc (300 mL) and a saturated NaHCO₃ aqueous solution (300 mL). The organic phase was collected and dried over Na₂SO₄. The crude product was purified by flash chromatography (dry load) eluting with PET/Et₂O from 2% to 4% for product. The title compound 8.3 (10 g, 38.89 mmol) was obtained as a yellowish oil. Yield 66%. ¹H NMR (200 MHz, CDCl₃) δ 1.27 (t, J=7.1 Hz, 3H), 3.62 (s, 2H), 4.17 (q, J=7.13 Hz, 2H), 4.50 (s, 2H), 7.09-7.13 (m, 3H), 7.21-7.28 (m, 1H).

### Example 9: Intermediate 9.3. 3-Bromomethyl-benzamide

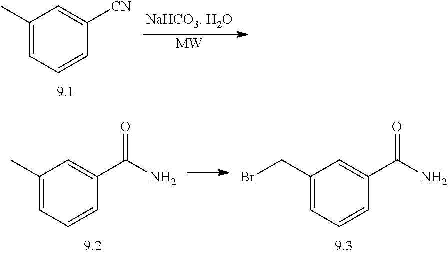

### Step 1: 3-Methyl-benzamide (9.2)

A solution of compound 9.1 (1.54 mL, 12.8 mmol) and K₂CO₃ (707 mg, 5.12 mmol) in H₂O (5 mL) was heated under microwave irradiation at 130° C., 200 psi, 200 W for 20 minutes. Upon cooling, the resulting white precipitate was collected and dried under reduced pressure to afford the title compound 9.2 as white crystals (870 mg, 6.4 mmol). Yield 50%. GC-MS (m/z) 135.1 (M+).

### Step 2: 3-Bromomethyl-benzamide (9.3)

NBS (434.6 mg, 2.4 mmol) and BPO (70%) (8 mg, 0.022 mmol) were added to a solution of intermediate 9.2 (300 mg, 2.22 mmol) in CH₃CN (20 mL). Stirring was continued at reflux for 4 h. The volatile were removed under reduced pressure. The crude product was partitioned between EtOAc (300 mL) and a saturated NaHCO₃ aqueous solution (300 mL). The organic phase was collected and dried over Na₂SO₄. The title compound 9.3 (250 mg, 1.16 mmol) was obtained as a yellowish solid. Yield 53%.

### Example 10: Intermediate 10.4. 3′-Bromomethyl-3,5-difluoro-4-methoxy-biphenyl

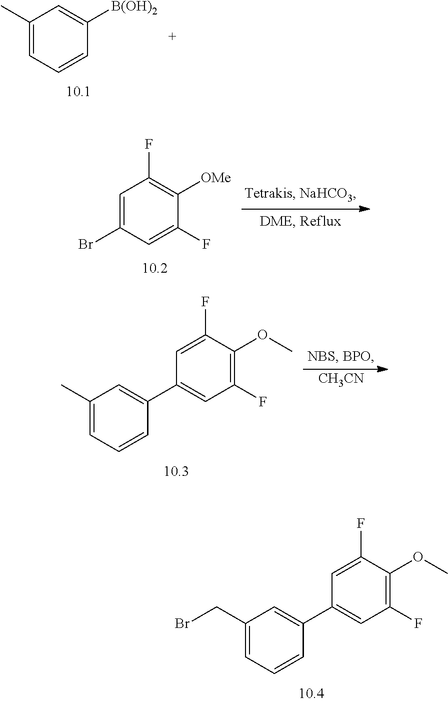

### Step 1: 3,5-Difluoro-4-methoxy-3′-methyl-biphenyl (10.3)

To a solution of compound 10.1 (0.18 mL, 1.33 mmol) in DME (15 mL) was added palladium tetrakis (50 mg, 0.039 mmol). Stirring was continued at r.t. for 5 min. m-Tolyl boronic acid 10.2 (202 mg, 1.35 mmol) and K₂CO₃ (745 mg, 3.56 mmol) were added in turn. Stirring was continued at reflux for 4 h. The solvent was removed under reduced pressure. The crude residue was taken up in water and extracted with DCM (3×20 ml). The organic phase was washed with brine, dried over Na₂SO₄, filtered and concentrated under reduced pressure. Pure title compound 10.3 (282 mg, 1.22 mmol) was obtained as a colorless oil and it was used for the next step without further purification. Yield 91%. ¹H NMR (400 MHz, CDCl₃) δ 2.43 (s, 3H), 4.04 (s, 3H), 7.14 (d, J=9.3, 2H), 7.32-7.33 (m, 4H).

### Step 2: 3′-Bromomethyl-3,5-difluoro-4-methoxy-biphenyl (10.4)

To a solution of the intermediate 10.3 (260 mg, 1.1 mmol) in CH₃CN (15 mL) was added BPO (4 mg, 0.0055 mmol) and NBS (210 mg, 1.22 mmol). Stirring was continued at reflux overnight. The solvent was removed under reduced pressure. The reaction partitioned between NaHCO₃₍ₛₛ₎ and DCM. The organic phase was washed with brine and dried over Na₂SO₄. The crude was purified by flash chromatography, eluting with PET/Et₂O affording the title compound 10.4 (250 mg, 0.77 mmol) as a yellow oil. Yield 72%. ¹H NMR (400 MHz, CDCl₃) δ 4.06 (d, J=3.7 Hz, 3H), 4.55 (s, 2H), 7.14 (d, J=6.2 Hz, 1H), 7.16 (d, J=6.1 Hz, 1H), 7.41-7.47 (m, 3H), 7.54 (s, 1H).

### Example 11: Intermediate 11.2. (3-Bromomethyl-phenyl)-acetic acid

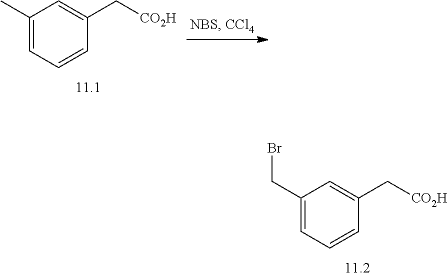

To a suspension of compound 11.1 (750 mg, 5 mmol) in CCl₄ (15 mL) was added AIBN (41 mg, 0.25 mmol) and NBS (933.7 mg, 5.24 mmol). Stirring was continued at reflux overnight. The solvent was removed under reduced pressure. The reaction was taken up with water, extracted with EtOAc (3×20 mL) washed with brine, and dried over Na₂SO₄. The crude was purified by flash chromatogaphy, eluting with CH₂Cl₂/MeOH (3% for product) affording the title intermediate 11.2 (800 mg, 3.49 mmol) as a white solid. Yield 70%. GC/MS (m/z) 227.9 (M+).

### Example 12: Intermediate 12.2. [3-(4-Chloro-5-cyano-6-thiophen-2-yl-pyrimidin-2-ylsulfanylmethyl)-phenyl]-acetic acid

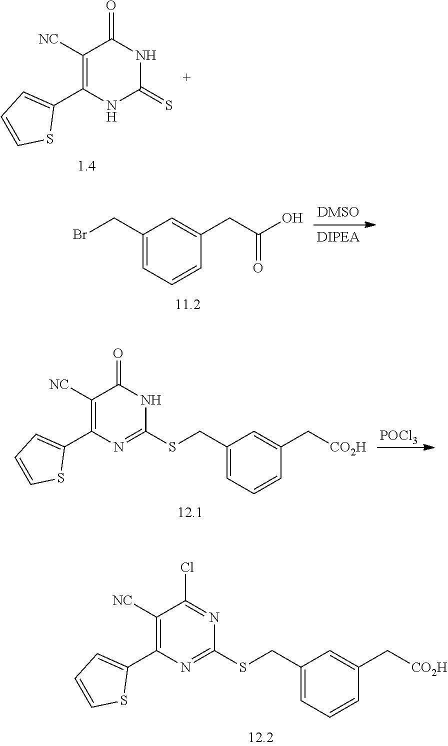

### Step 1: [3-(5-Cyano-6-oxo-4-thiophen-2-yl-1,6-dihydro-pyrimidin-2-ylsulfanylmethyl)-phenyl]-acetic acid (12.1)

To a stirred suspension of intermediate 1.4 (500 mg, 2.12 mmol) and DIPEA (0.4 mL, 2.12 mmol) in DMSO (5 mL) was added intermediate 11.2 (487 mg, 2.12 mmol). Stirring was continued overnight at room temperature. The crude reaction mixture was poured into water and the resulting aqueous mixture was washed with EtOAc, acidified to pH 3, and extracted with EtOAc (3×50 mL). The title intermediate 12.1 was obtained (200 mg, 0.52 mmol) as a pure yellowish solid after flash chromatography purification eluting with CH₂Cl₂/MeOH (10% for product) and shredding with a mixture of EtO₂/Acetone. Yield 25%. ¹H NMR (400 MHz, DMSO-d₆) δ 3.49 (s, 2H), 4.53 (s, 2H), 7.16 (d, J=6.8 Hz, 1H), 7.26 (t, J=7.2 Hz, 1H), 7.36 (m, 3H), 8.05 (d, J=4.4 Hz, 1H), 8.27 (s, 1H), 12.13 (s, 1H); ¹³C NMR (100 MHz, DMSO-d₆) δ 34.3, 40.9, 88.5, 116.8, 127.6, 128.9, 129.1, 129.8, 130.4, 131.9, 135.2, 135.8, 137.0, 139.9, 159.1, 161.6, 165.7, 172.9. HPLC 95.8%.

### Step 2: [3-(4-Chloro-5-cyano-6-thiophen-2-yl-pyrimidin-2-ylsulfanylmethyl)-phenyl]-acetic acid (12.2)

A mixture of intermediate 12.1 (300 mg, 0.78 mmol) and POCl₃ (6 ml) were heated at 80° C. 4 h. The crude reaction mixture was then poured in ice. The resulting yellow precipitate was collected and dried under reduced pressure affording the title intermediate 12.2 (250 mg, 0.62 mmol) as a yellowish solid. Yield 79%. ¹H NMR (400 MHz, DMSO-d₆) δ 3.53 (s, 2H), 4.50 (s, 2H), 7.16 (d, J=7.5 Hz, 1H), 7.27 (t, J=7.4 Hz, 1H), 7.36-7.39 (m, 3H), 8.13 (d, J=4.9 Hz, 1H), 8.3 (d, J=3.9 Hz, 1H), 12.25 (brs, 1H). ¹³C NMR (100 MHz, DMSO-d₆) δ 35.1, 40.9, 97.7, 115.5, 127.6, 128.8, 129, 130.2, 130.4, 133.3, 135.7, 136, 137, 138.6, 160.3, 163.2, 172.9, 174.

### Example 13: Intermediate 13.3. [3-(4-Chloro-5-cyano-6-thiophen-2-yl-pyrimidin-2-ylsulfanylmethyl)-benzoic acid

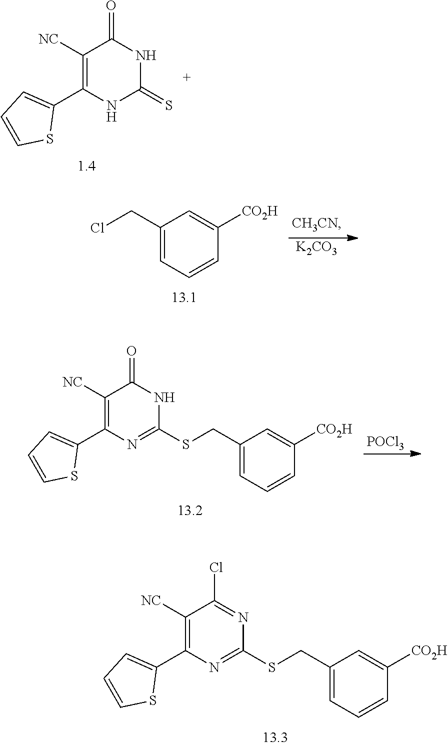

### Step 1: 3-(5-Cyano-6-oxo-4-thiophen-2-yl-1,6-dihydro-pyrimidin-2-ylsulfanylmethyl)-benzoic acid (13.2)

To a stirred suspension of intermediate 1.4 (250 mg, 1.06 mmol) and K₂CO₃ (440 mg, 3.18 mmol) in CH₃CN (15 mL) was added 3-(chloromethyl)benzoic acid 13.1 (180 mg, 1.06 mmol). Stirring was continued overnight at reflux. The volatiles were then removed under reduced pressure. The crude product was taken up in water, washed with EtOAc, acidified to pH 1, and extracted with EtOAc (3×50 mL). Shredding with hot acetone afforded the title intermediate 13.2 (45 mg, 0.12 mmol) as a yellowish solid. Yield 12%. ¹H NMR (400 MHz, DMSO-d₆) δ 4.62 (s, 2H), 7.33 (t, J=4.3 Hz, 1H), 7.44 (t, J=7.6 Hz, 1H), 7.72 (d, J 7.5 Hz, 1H), 7.82 (d, J 7.5 Hz, 1H), 8.05 (m, 2H), 8.26 (d, J=3.8 Hz, 1H), 12.99 (s, 1H); ¹³C NMR (100 MHz, DMSO) δ 33.9, 88.7, 116.5, 128.8, 129.3, 129.9, 130.2, 131.5, 132.1, 133.7, 135.4, 137.9, 139.7, 159.0, 161.2, 165.3, 167.4. HPLC: 97.2%

### Step 2: 3-(4-Chloro-5-cyano-6-thiophen-2-yl-pyrimidin-2-ylsulfanylmethyl)-benzoic acid (13.3)

A mixture of intermediate 13.2 (300 mg, 0.81 mmol) and POCl₃ (6 ml) were heated at 80° C. for 4 h. The reaction mixture was then poured into ice. The resulting yellow precipitate was collected and purified by flash chromatography eluting with DCM/MeOH (3% for product) to provide intermediate 13.3 (120 mg, 0.3 mmol) as a yellowish solid. Yield 79%. ¹³C NMR (100 MHz, DMSO-d₆) δ 33.8, 88.7, 116.5, 128.8, 129.3, 129.9, 130.2, 131.4, 132.1, 133.7, 135.5, 137.9, 139.6, 159, 161.1, 165.2, 167.3;

### Example 14: Intermediate 14.2. 3-Bromomethyl-benzonitrile

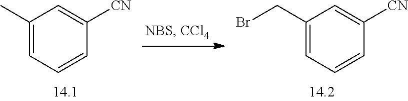

To a solution of compound 14.1 (2 mL, 17.07 mmol) in CCl₄ was added a mixture of NBS (2.9 g, 17.1 mmol) and BPO (16 mg, 0.06 mmol). Stirring was continued at reflux for 16 h and the reaction was then allowed to warm to rt. The resulting solid was collected, washed with CCl₄, and dried under reduced pressure. The title compound 14.2 was obtained as a white solid (2.84 g, 14.5 mmol). Yield 85%. GC−) 196.9 (M+).

### Example 15: Intermediate 15.1. 2-(3-Bromomethyl-phenyl)-ethanol

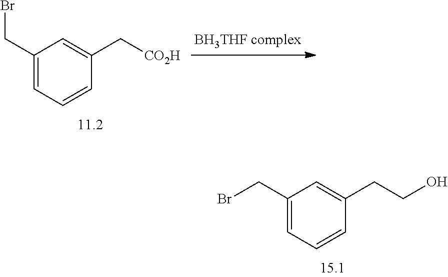 [SMILES: O=C(O)Cc1cccc(CBr)c1 ; OCCc1cccc(CBr)c1]

To a solution of intermediate 11.2 (500 mg, 2.17 mmol) in THF (10 mL) at 0° C. was added BH₃-THF (1M in THF, 2.8 mL) dropwise. The mixture was stirred at 0° C. for 1 h and then at r.t. for 12 h. The mixture was diluted with THF/H₂O (1:1 v:v, 15 mL) and washed with saturated aq. K₂CO₃. The phases were separated and the aqueous layer was extracted with EtOAc (3×20 mL). The combined organic phase was washed with brine and dried over Na₂SO₄. Flash chromatography purification of the crude product (eluting with DCM/MeOH) afforded the title intermediate 15.1 (400 mg, 1.85 mmol) as a white solid. Yield 85%. GC/MS (m/z) 214 (M+).

### Example 16: Intermediate 16.2. 2 (3-Bromomethyl-phenyl)-acetonitrile

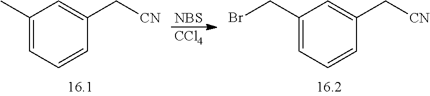

NBS (338 mg, 1.9 mmol) and BPO (70%) (28.7 mg, 0.11 mmol) were added to a solution of intermediate 16.1 (0.5 mL, 2.37 mmol) in CH₃CN (15 mL). Stirring was continued at reflux for 4 h. The volatiles were removed under reduced pressure. The crude product was partitioned between EtOAc (100 mL) and a saturated NaHCO₃ aqueous solution (100 mL). The organic phase was collected and dried over Na₂SO₄. The title compound 16.2 (250 mg, 1.18 mmol) was obtained as a yellowish solid after flash chromatography purification (eluting with PET/EtOAc). Yield 50%.

### Example 17: Intermediate 17.3. 5-(3-Bromomethyl-phenyl)-2-methyl-2H-tetrazole

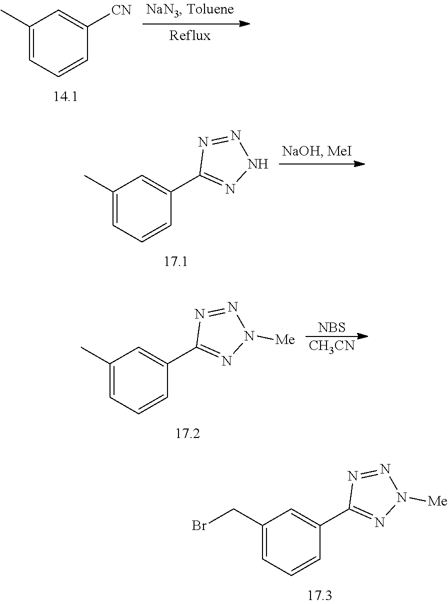

### Step 1: 5-m-Tolyl-2H-tetrazole (17.1)

A mixture of compound 14.1 (1.02 mL, 8.54 mmol), NaN₃ (832 mg, 12.8 mmol) and Et₃N·HCl (1.76 g, 12.8 mmol) in toluene (20 mL) was heated at reflux for 4 h. The solvent was then removed under reduced pressure. The crude product was poured into water and the resulting aqueous solution was acidified to pH 1 with 3N HCl and extracted with EtOAc (3×20 mL). The organic phase was washed with brine, dried over Na₂SO₄, and concentrated under reduced pressure. The title compound 17.1 (1.22 g, 7.6 mmol) was obtained as a white solid. Yield 89%. ¹H NMR (200 MHz, DMSO-d₆) δ 2.39 (s, 3H), 7.39 (m, 1H), 7.48 (t, J=7.58 Hz, 1H), 7.80 (s, 1H), 7.85 (m, 1H); GC/MS (m/z) 160.1 (M+).

### Step 2: 2-Methyl-5-m-tolyl-2H-tetrazole (17.2)

To a solution of intermediate 17.1 (1 g, 6.2 mmol) in water (5 mL) and NaOH (500 mg, 12.5 mmol) was added a solution of MeI (0.38 mL, 6.1 mmol) in acetone (10 mL). Stirring was continued at reflux for 6 h. The solvent was then removed under reduced pressure and the resulting residue was taken up in EtOAc and H₂O. The organic layer was separated, dried over Na₂SO₄ and evaporated to dryness in vacuo. Purification of the crude product afforded the title intermediate 17.2 (500 mg, 2.87 mmol) as a white solid. Yield 46%. ¹H NMR (400 MHz, CDCl₃) δ 4.37 (s, 3H), 7.26-7.39 (m, 1H), 7.35-7.39 (m, 1H), 7.91-7.96 (m, 2H).

### Step 3: 5-(3-Bromomethyl-phenyl)-2-methyl-2H-tetrazole (17.3)

To a suspension of compound 17.2 (200 mg, 1.15 mmol) in CH₃CN (15 mL) was added BPO (21 mg, 0.057 mmol) and NBS (163.5 mg, 0.92 mmol). Stirring was continued at 92° C. overnight. The solvent was removed under reduced pressure. The reaction mixture was taken up in water, extracted with EtOAc (3×20 mL), washed with brine, and dried over Na₂SO₄. The crude product was purified by flash chromatography eluting with CH₂Cl₂/MeOH (7% for product) to afford the title compound 17.3 (261 mg, 1.03 mmol) as a white solid. Yield 90%. ¹H NMR (400 MHz, CDCl₃) δ 4.41 (s, 3H), 4.56 (s, 2H), 7.46-7.52 (m, 1H), 8.07-8.09 (m, 1H), 8.19 (s, 1H).

### Example 18: Intermediate 18.1. 5-(3-Bromomethyl-phenyl)-1H-tetrazole

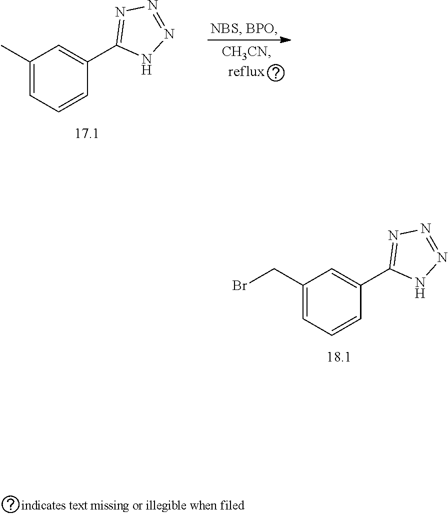

To a suspension of compound 17.1 (300 mg, 1.87 mmol) in CH₃CN (15 mL) was added AIBN (31 mg, 0.18 mmol) and NBS (333 mg, 1.87 mmol). Stirring was continued at reflux overnight. The solvent was removed under reduced pressure. The reaction was taken up with water, extracted with EtOAc (3×20 mL) washed with brine, and dried over Na₂SO₄. The crude was purified by flash chromatography, eluting with CH₂Cl₂/MeOH (7% for product) affording the title compound 18.1 (150 mg, 0.62 mmol) as a light yellow solid. Yield 34%.

### Example 19: Intermediate 19.5. 3-Bromomethyl-benzenesulfonamide

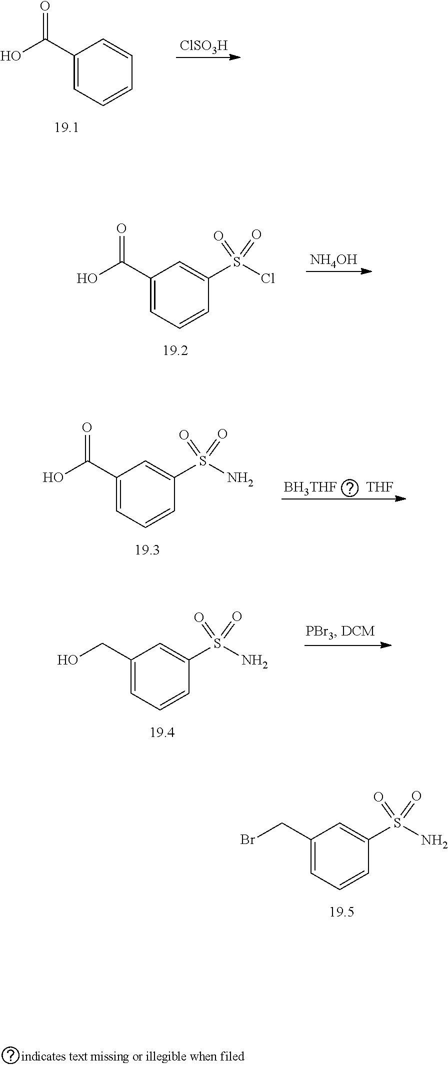

### Step 1: 3-Chlorosulfonyl-benzoic acid (19.2)

A mixture of compound 19.1 (1 g, 8.13 mmol) and chlorosulfonic acid (4 mL) was stirred at 125° C. for 2 h. The mixture was poured into ice water dropwise. The resulting solid was collected, solubilized in EtOAc, and washed with water (3×20 mL). The organic layer was dried over Na₂SO₄ and evaporated under reduced pressure. The title intermediate 19.2 (1.19 g, 5.39 mmol) was obtained as a white solid. Yield 65%. ¹H NMR (400 MHz, DMSO-d₆) δ 7.45 (t, J=7.69 Hz, 1H), 7.65 (d, J=7.8 Hz, 1H), 7.86 (d, J=7.6 Hz, 1H), 8.1 (s, 1H), 13.9 (brs, 1H).

### Step 2: 3-Sulfamoyl-benzoic acid (19.3)

To a cold solution of 25% NH₄OH (10 mL) was added portionwise intermediate 19.2 (1.10 g, 5.39 mmol). Stirring was continued at rt for 2 h and the resulting mixture was concentrated. The crude product was suspended in water (4 mL) and 37% HCl solution was then added dropwise to the mixture. The resulting precipitate was collected and dried under reduced pressure to afford the title intermediate 19.3 (943 mg, 4.6 mmol) as a white solid. Yield 87%. ¹H NMR (400 MHz, DMSO-d₆) δ 7.50 (brs, 2H), 7.71 (t, J=7.78 Hz, 1H), 8.04 (d, J 7.8 Hz, 1H), 8.13 (d, J 7.7 Hz, 1H), 8.38 (s, 1H), 13.4 (brs, 1H).

### Step 3: 3-Hydroxymethyl-benzenesulfonamide (19.4)

To a stirred solution of intermediate 19.3 (940 mg, 4.67 mmol) was added dropwise at 0° C. BH₃-THF complex (14 mL, 14.01 mmol) and stirring was continued for 4 h at rt. The reaction mixture was then cooled to 0° C., and quenched by the dropwise addition of MeOH. After 15 min, a 3N solution of HCl (37 mL) was added to the mixture and the volatiles were removed under reduced pressure. The aqueous phase was extracted with EtOAc (3×20 mL). The combined organic phase was washed with brine and dried over Na₂SO₄ to afford the title intermediate 19.4 (785 mg, 4.2 mmol) as a colorless oil. Yield 89%. ¹H NMR (400 MHz, DMSO-d₆) δ 4.51 (s, 2H), 7.34 (s, 2H), 7.5 (d, J=5 Hz, 2H), 7.68 (t, J=5.2 Hz, 1H), 7.8 (s, 1H).

### Step 4: 3-Bromomethyl-benzenesulfonamide (19.5)

To a stirred suspension of intermediate 19.4 (200 mg, 1.07 mmol) in DCM (3.5 mL) was added PBr₃ and stirring was continued at 20° C. for 16 h. Water was then added carefully to the mixture and the phases were separated. The aqueous phase was extracted with DCM (2×20 mL). The combined organic layers were washed with brine and dried over Na₂SO₄ to afford the title intermediate 19.5 (120 mg, 0.47 mmol) as a colorless oil. Yield 45%. ¹H NMR (400 MHz, CDCl₃) δ 4.53 (s, 2H), 7.54 (t, J=10.7 Hz, 1H), 7.69 (d, J=7.6 Hz, 1H), 7.88 (d, J=7.8 Hz, 1H), 7.97 (s, 1H).

### Example 20: Intermediate 20.2. 4-Benzyl-2-mercapto-6-oxo-1,6-dihydro-pyridine-3-carbonitrile

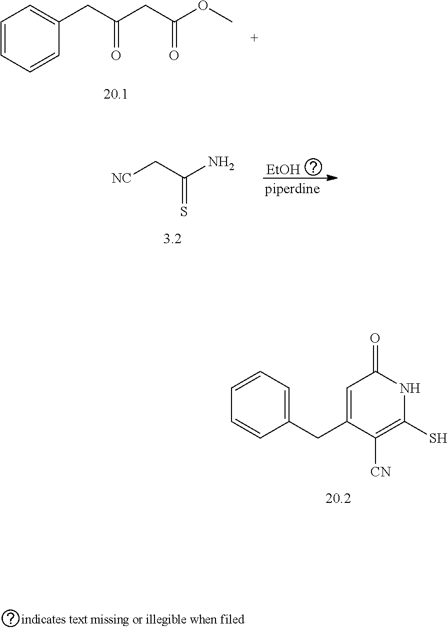 [SMILES: "EtOH" ; COC(=O)CC(=O)Cc1ccccc1 ; N#CCC(N)=S ; N#Cc1c(Cc2ccccc2)cc(=O)[nH]c1S]

To a solution of intermediate 20.1 (1.2 g, 6.24 mmol) and potassium tert butoxide (764 mg, 6.24 mmol) in DMF (15 mL) was added compound 3.2 (31 mg, 0.18 mmol). Stirring was continued at 85° C. overnight. The reaction was poured into water and the pH was acidified to 5 by the addition of AcOH followed by washing with EtOAc (3×20 mL). Then, pH was brought to 3 by the addition of 3N HCl solution. The aqueous phase was extracted with EtOAc (3×30 mL). The organic phase was washed with brine and dried over anhydrous Na₂SO₄. The title compound (600 mg, 2.47 mmol) was obtained as light yellow solid. Yield 40%. ¹H NMR (400 MHz, DMSO-d₆) δ 3.63 (s, 2H), 5.81 (s, 1H), 7.17-7.36 (m, 5H), 13.1 (brs, 1H).

### Example 21: [3-(5-Cyano-6-oxo-4-thiophen-2-yl-1,6-dihydro-pyrimidin-2-ylsulfanylmethyl)-phenyl]-acetic acid ethyl ester (Compound I-1)

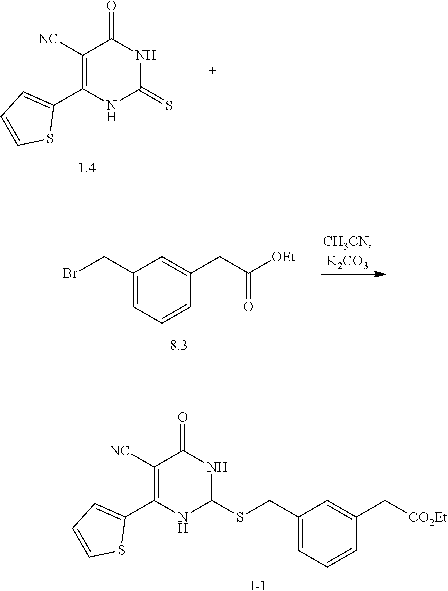 [SMILES: N#Cc1c(-c2cccs2)[nH]c(=S)[nH]c1=O ; CCOC(=O)Cc1cccc(CBr)c1 ; CCOC(=O)Cc1cccc(CSC2NC(=O)C(C#N)=C(c3cccs3)N2)c1 ; [I-]]

To a stirred suspension of intermediate 1.4 (2.13 g, 9.1 mmol) and K₂CO₃ (1.88 g, 13.6 mmol) in CH₃CN (80 mL) was added intermediate 8.3 (2.45 g, 9.52 mmol) and stirring was continued at a gentle reflux for 16 h. The solvent was then removed under reduced pressure. The crude product was taken up in water and the resulting aqueous solution was neutralized with 3N HCl solution. The resulting pale yellow solid was collected, washed with ice cold water, and dried under reduced pressure. The title compound I-1 (3.1 g, 7.46 mmol) was obtained as a grey solid after trituration with Et₂O. Yield 82%. ¹H NMR (400 MHz, DMSO-d₆) δ 1.15 (d, J=7.03 Hz, 3H), 3.62 (s, 2H), 4.03 (q, J=7.16 Hz, 2H), 4.55 (s, 2H), 7.17 (d, J=7.1 Hz, 1H), 7.28 (t, J=7.7 Hz, 1H), 7.38 (m, 3H), 8.1 (d, J=4.7 Hz, 1H), 8.29 (d, J=3.35 Hz, 1H). ¹³C NMR (100 MHz, DMSO-d₆) δ 14.4, 34.2, 60.7, 88.6, 116.6, 127.8, 129.1, 129.1, 129.9, 130.3, 132.1, 135.2, 135.5, 137.1, 139.7, 159.1, 161.1, 165.3, 171.4. HPLC>97.9%.

### Example 22: 3-(5-Cyano-6-oxo-4-thiophen-2-yl-1,6-dihydro-pyrimidin-2-ylsulfanylmethyl)-benzamide (Compound I-2)

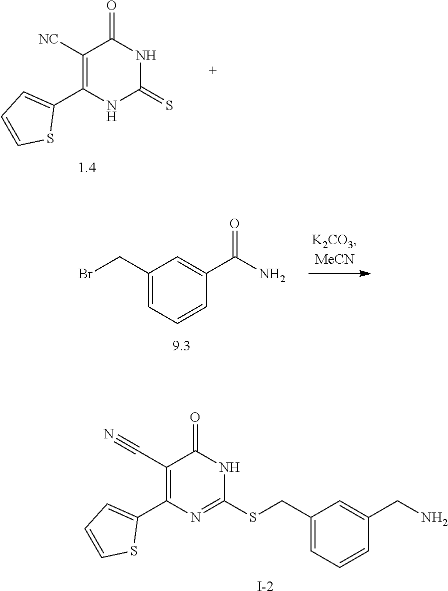

To a stirred suspension of intermediate 1.4 (182 mg, 0.78 mmol) and intermediate 9.3 (200 mg, 0.65 mmol) in CH₃CN (20 mL) was added K₂CO₃ (119 mg, 0.86 mmol) and stirring was continued at a gentle reflux for 16 h. The volatiles were removed under reduced pressure. The crude product was taken up in water, and the resulting aqueous mixture was acidified to pH 3 with a 3N HCl solution and extracted with EtOAc (3×20 mL). The combined organic phases were washed with brine and dried over Na₂SO₄ to afford the title compound I-2 (120 mg, 0.24 mmol) as a yellowish solid after trituration with hot Et₂O. Yield 42%. ¹H NMR (400 MHz, DMSO-d₆) δ 4.61 (s, 2H), 7.3 (m, 1H), 7.41 (m, 2H), 7.63 (d, J 7.12 Hz, 1H), 7.76 (d, J 7.3 Hz, 1H), 7.97 (s, 2H), 8.01 (d, J=4.47 Hz, 1H), 8.3 (d, J=2.8 Hz, 1H). ¹³C NMR (100 MHz, DMSO-d₆) δ 34.5, 88.1, 117.1, 127.3, 129.1, 129.3, 130.4, 132.5, 132.5, 135.5, 135.9, 137.7, 140.2, 159.4, 161.6, 165.7, 168.4. HPLC>94.2%.

### Example 23: 2-({[3-(3,5-difluoro-4-hydroxyphenyl)phenyl]methyl}sulfanyl)-6-oxo-4-(thiophen-2-yl)-1,6-dihydropyrimidine-5-carbonitrile (Compound I-3)

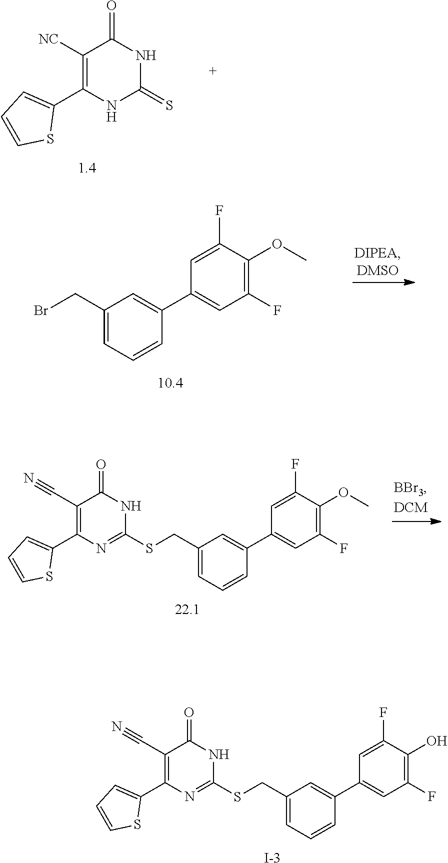

### Step 1: 2-({[3-(3,5-difluoro-4-hydroxyphenyl)phenyl]methyl}sulfanyl)-6-oxo-4-(thiophen-2-yl)-1,6-dihydropyrimidine-5-carbonitrile (22.1)

To a stirred solution of intermediate 1.4 (152 mg, 0.65 mmol) and intermediate 10.4 (250 mg, 0.77 mmol) in DMSO (6 mL) was added DIPEA (0.13 mL, 0.72 mmol) and stirring was continued at rt for 4 h. The crude mixture was poured into water and the resulting aqueous mixture was washed with EtOAc, acidified to pH 3, and extracted with EtOAc (3×50 mL). The combined organic phases were washed with brine and dried over Na₂SO₄ to afford intermediate 22.1 (180 mg, 0.0.38 mmol) as a pale yellow powder after flash chromatography purification eluting with CH₂Cl₂/MeOH (4% for product). Yield 55%. ¹H NMR (400 MHz, DMSO-d₆) δ 3.93 (s, 3H), 4.60 (s, 2H), 7.34-7.43 (m, 4H), 7.5 (d, J=4.3 Hz, 1H), 7.60 (d, J=7.5 Hz, 1H), 7.84 (s, 1H), 8.0 (d, J=4.9 Hz, 1H), 8.29 (d, J=4.9 Hz, 1H), 13.9 (brs, 1H).

### Step 2: 2-({[3-(3,5-difluoro-4-hydroxyphenyl)phenyl]methyl}sulfanyl)-6-oxo-4-(thiophen-2-yl)-1,6-dihydropyrimidine-5-carbonitrile (Compound I-3)

To a stirred suspension of intermediate 22.1 (170 mg, 0.36 mmol) in DCM (25 mL) was added a 1M solution of BBr₃ in DCM (0.72 mL, 0.72 mmol) and stirring was continued at reflux for 16 h. The reaction mixture was quenched by the addition of MeOH and the volatiles were removed under reduced pressure. The crude product was purified by flash chromatography eluting with DCM/MeOH (5% for product). The title compound I-3 (90 mg, 0.2 mmol) was obtained as a white solid after trituration with hot Et₂O. Yield 42%. ¹H NMR (400 MHz, DMSO) δ 4.59 (s, 3H), 7.31 (d, J=9.1 Hz, 2H), 7.36 (m, 1H), 7.39 (d, J=7.6 Hz, 1H), 7.45 (d, J=7.7 Hz, 1H), 7.56 (d, J=7.5 Hz, 1H), 7.81 (s, 1H), 8.07 (d, J=5 Hz, 1H), 8.3 (d, J=3.8 Hz, 1H), 10.34 (s, 1H). ¹³C NMR (100 MHz, DMSO-d₆) δ 88.7, 110.3 (²J_{CF}=15.1 Hz), 110.3 (²J_{CF}=15.5 Hz), 125.8, 127.4, 128.5, 129.7, 130, 130.5, 132.1, 133.5 (³J_{CF}=16 Hz), 133.7 (³J_{CF}=16 Hz), 135.3, 138.1, 138.4, 139.7, 152.9 (¹J_{CF}=239.9 Hz), 153.01 (¹J_{CF}=240.1 MHz), 159.0, 161.3, 165.5. HPLC: 98.4%.

### Example 24: [3-(5-Cyano-4-methoxy-6-thiophen-2-yl-pyrimidin-2-yl-pyrimidin-2-ylsulfanylmethyl)-phenyl]-acetic acid (Compound I-4)

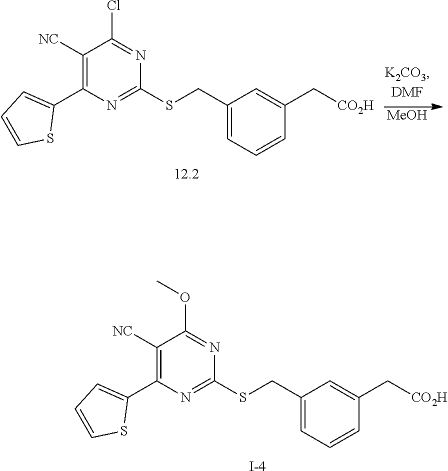

To a stirred solution of intermediate 12.2 (80 mg, 0.19 mmol) and MeOH (0.04 mL, 0.95 mmol) in DMF (3 mL) was added K₂CO₃ (60 mg, 0.43 mmol) and stirring was continued at rt for 16 h. The reaction mixture was poured into water and the resulting aqueous mixture was extracted with EtOAc (3×20 mL). The combined organic phase was washed with brine and dried over Na₂SO₄. Flash chromatography purification of the crude product (eluting with DCM/MeOH, 1.5% for product) afforded the title compound I-4 (45 mg, 0.11 mmol) as a white solid. Yield 58%; ¹H NMR (400 MHz, DMSO-d₆) δ 3.56 (s, 3H), 3.66 (s, 2H), 4.54 (s, 2H), 7.17 (d, J=7.2 Hz, 1H), 7.27 (t, J=7.6 Hz, 1H), 7.33-7.38 (m, 3H), 8.07 (d, J=4.8 Hz, 1H), 8.28 (d, J=3.6 Hz, 1H). ¹³C NMR (100 MHz, DMSO-d₆) δ 34.2, 40.3, 52.1, 88.5, 116.6, 127.8, 129, 129.1, 129.8, 130.3, 132, 135.1, 135.3, 137.1, 139.8, 159.1, 161.4, 165.5, 171.8. HPLC>97.1%.

### Example 25: [3-(4-Bromo-5-cyano-6-thiophen-2-yl-pyrimidin-2-ylsulfanylmethyl)-phenyl]-acetic acid (Compound I-5)

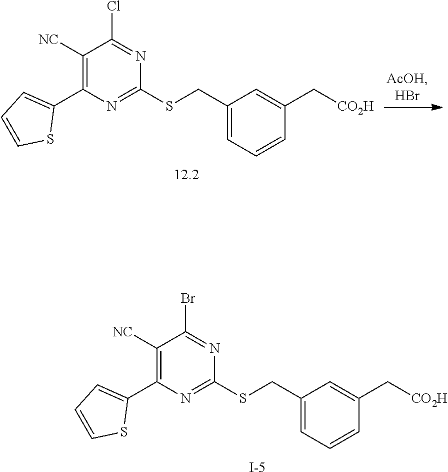

To a stirred solution of intermediate 12.2 (30 mg, 0.051 mmol) in AcOH (3.0 mL) was added HBr (36% solution in AcOH, 0.17 mL, 1.029 mmol) and the resulting mixture was stirred at 60° C. for 72 h. The reaction mixture was then diluted with DCM (10 mL), washed with H₂O (3×10 mL), brine (10 mL), dried over Na₂SO₄ and concentrated under reduced pressure. The crude product was purified by flash chromatography (DCM/MeOH/AcOH, from 99:1:0.1 92:8:0.1) to afford the title compound I-5 (17 mg, 0.038 mmol) as a yellow solid. Yield 75%. MS/MS ESI (+): 447.8, 401.9, 338.3. ¹H-NMR (CDCl₃, 400 MHz) δ: 3.63 (s, 2H), 4.45 (s, 2H), 7.21 (m, 1H), 7.31 (m, 2H), 7.39 (m, 2H), 7.70 (brs, 1H), 8.45 (brs, 1H) ¹³C-NMR (CDCl₃, 100 MHz) δ: 29.3, 40.7, 100.1, 116.0, 127.9, 128.7, 128.9, 129.4, 130.1, 133.1, 133.6, 134.6, 136.6, 138.7, 155.8, 159.7, 174.3, 177.1. HPLC>95%.

### Example 26: [3-(5-Cyano-4-cyclopropylamino-6-thiophen-2-yl-pyrimidin-2-ylsulfanylmethyl)-phenyl]-acetic acid (Compound I-6)

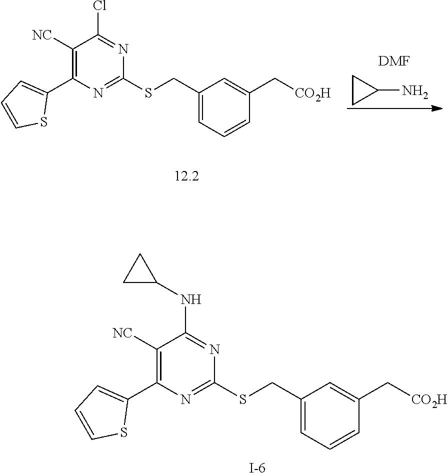

To a stirred solution of intermediate 12.2 (200 mg, 0.49 mmol) in DMF (3 mL) was added cyclopropylamine (0.037 mL, 0.55 mmol) and stirring was continued at r.t. for 16 h. The reaction mixture was quenched with brine, poured into water, and the resulting aqueous mixture was extracted with EtOAc (3×20 mL). The combined organic phases were washed with brine and dried over Na₂SO₄. The title compound I-6 (80 mg, 0.2 mmol) was obtained as a white solid after shredding with Et₂O. Yield 39%. ¹H NMR (400 MHz, DMSO-d₆) δ 0.73 (m, 4H), 2.95 (m, 1H), 3.53 (s, 2H), 4.45 (s, 2H), 7.13 (d, J=7.4 Hz, 1H), 7.23-7.29 (m, 2H), 7.33-7.35 (m, 2H), 7.93 (d, J=4.9 Hz, 1H), 8.18 (d, J=3.2 Hz, 1H), 8.21 (s, 1H), 12.3 (s, 1H). ¹³C NMR (100 MHz, DMSO-d₆) δ 6.6, 6.6, 25, 34.3, 40.8, 79.7, 116.7, 127.3, 128.5, 128.6, 129.2, 130.1, 130.7, 133.2, 135.4, 138.3, 140.1, 158.7, 162.8, 172.8, 172.8. HPLC>99.3%.

### Example 27: [3-(4-Amino-5-cyano-6-thiophen-2-yl-pyrimidin-2-ylsulfanylmethyl)-phenyl]-acetic acid (Compound I-7)

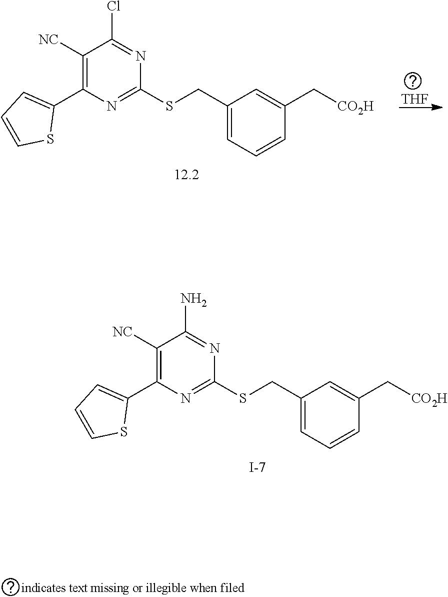

[3-(4-Cloro-5-cyano-6-thiophen-2-yl-pyrimidin-2-ylsulfanylmethyl)-phenyl]-acetic acid (12.2) (100 mg, 0.248 mmol) was dissolved in NH₃ 0.4 M in THF (18 mL, 7.466 mmol) and the resulting opalescent solution was stirred at room temperature for 72 hours. Then the mixture was poured in AcOEt (15 ml), washed with HCl 3 M (5 mL), aq. NaHCO_{3 ss}(10 mL), brine (10 mL) dried over Na₂SO₄ and concentrated under reduced pressure. The crude was purified by flash chromatography (CH₂Cl₂/MeOH/AcOH, from 99:1:0.1 90:10:0.1) to afford the title compound I-7 (86 mg, 0.22 mmol) as a white solid. Yield 94%; MS/MS ESI (+): 382.9. ¹H NMR (400 MHz, DMSO-d₆) δ 3.53 (s, 2H), 4.39 (s, 2H), 7.13 (d, J=7.3 Hz, 1H), 7.24 (t, J=7.1 Hz, 1H), 7.29 (m, 1H), 7.35 (m, 2H), 7.8 (brs, 1H), 7.94 (d, J=4.3 Hz, 1H), 8.20 (m, 1H), 12.32 (brs, 1H). ¹³C NMR (100 MHz, DMSO-d₆) δ 34.3, 40.9, 78.9, 116.9, 127.6, 128.6, 128.7, 129.3, 130.4, 130.8, 133.4, 135.5, 138.3, 140.4, 159.1, 163.7, 173. HPLC>97.9%.

### Example 28: [3-(5-Cyano-6-thiophen-2-yl-pyrimidin-2-ylsulfanylmethyl)-phenyl]-acetic acid (Compound I-8)

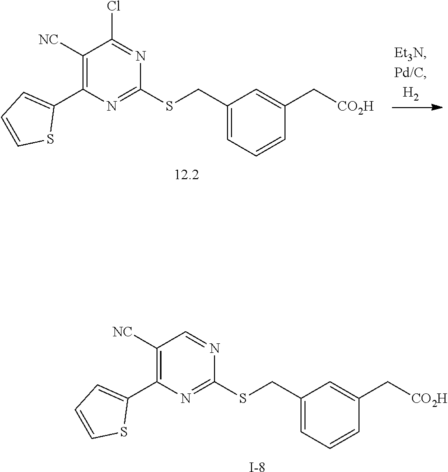

Et₃N (0.15 mL, 1.119 mmol) was added to a stirred solution of intermediate 12.2 (150 mg, 0.373 mmol) in THF (3.7 mL). The resulting solution was continuously hydrogenated for 12 h using the Thales Nano H-Cube Hydrogenator (Cartridge: Pd/C 10%, H₂ Pressure: 8 bar, temperature: 40° C., transporting solvent: THF, flowrate: 1.0 mL/min). The resulting reaction mixture (about 5 mL) was diluted with EtOAc (15 mL), washed with 3M HCl (5 mL) and brine (10 mL), dried over Na₂SO₄, filtered, and concentrated under reduced pressure. The crude product was purified by reverse-phase flash chromatography (column: RP-18, eluting with H₂O/MeOH 80/20 to 10/90) to give the title compound I-8 as a white powder. Yield 34%. MS/MS ESI (+): 368.1. ¹H-NMR (DMSO-d₆, 400 MHz) δ: 3.60 (s, 2H), 4.45 (s, 2H), 7.15 (m, 1H), 7.25 (m, 1H), 7.36 (m, 4H), 8.06 (br-s, 1H), 8.30 (ps-s, 1H), 9.03 (s, 1H).

### Example 29: [3-(5-Cyano-4-methylamino-6-thiophen-2-yl-pyrimidin-2-ylsulfanylmethyl)-phenyl]-acetic acid (Compound I-9

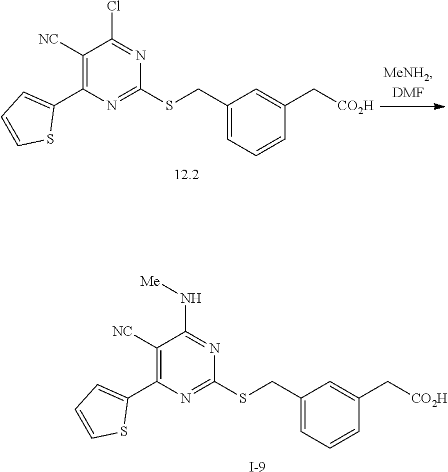

To a stirred solution of intermediate 12.2 (100 mg, 0.25 mmol) in DMF (3 mL) was added a 33% solution of MeNH₂ (0.03 mL, 0.27 mmol) in ethanol and stirring was continued at rt for 16 h. The reaction mixture was quenched with brine, poured into water, acidified to pH 6 by the addition of a 3M HCl solution, and then extracted with EtOAc (3×20 mL). The combined organic phase was washed with brine and dried over Na₂SO₄ to afford the title compound I-9 (80 mg, 0.2 mmol) as a white solid. Yield 80%. ¹H NMR (400 MHz, DMSO-d₆) δ 2.93 (d, J=4.5 Hz, 3H), 3.53 (s, 2H), 2.92 (s, 2H), 7.13 (d, J=7.6 Hz, 1H), 7.24 (d, J 7.5 Hz, 1H), 7.27-7.29 (m, 1H), 7.33 (m, 2H), 7.94 (d, J=5.1 Hz, 1H), 8.1 (q, J=4.5 Hz, 1H), 8.18 (d, J=3.8 Hz, 1H), 12.33 (s, 1H). ¹³C NMR (100 MHz, DMSO-d₆) δ 28.6, 34.5, 40.9, 79.6, 116.9, 127.4, 128.6, 128.7, 129.3, 130.2, 130.7, 133.3, 135.5, 138.3, 140.3, 158.5, 161.9, 172.9, 173.1. HPLC>98.1%.

### Example 30: 3-(5-Cyano-4-methylamino-6-thiophen-2-yl-pyrimidin-2-ylsulfanylmethyl)-benzoic acid (Compound I-10)

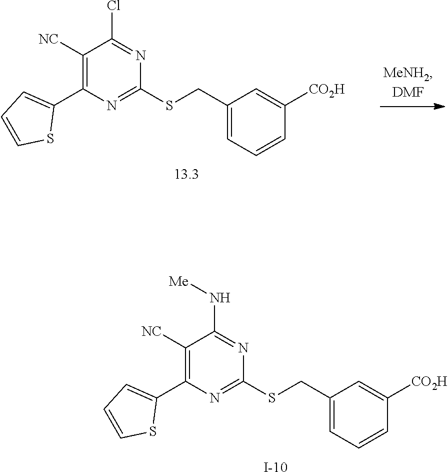

To a stirred solution of intermediate 13.3 (90 mg, 0.23 mmol) in DMF (3 mL) was added a 33% solution of MeNH₂ (0.03 mL, 0.25 mmol) in ethanol and stirring was continued at rt for 16 h. The reaction mixture was quenched with brine, poured into water, acidified to pH 6 by the addition of a 3M HCl solution, and then extracted with EtOAc (3×20 mL). The combined organic phase was washed with brine and dried over Na₂SO₄. The crude product was purified by flash chromatography eluting with DCM/MeOH (4% for product) to afford the title compound I-10 (45 mg, 0.12 mmol) as a white solid. Yield 51%. ¹H NMR (400 MHz, DMSO-d₆) δ 2.94 (d, J=4.4 Hz, 3H), 4.49 (s, 2H), 7.28 (t, J=4.4 Hz, 1H), 7.44 (t, J 7.7 Hz, 1H), 7.70 (d, J=7.5 Hz, 1H), 7.81 (d, J=7.7 Hz, 1H), 7.92 (d, J=5. Hz, 1H), 8.04 (q, J=4.5 Hz, 1H), 8.06 (s, 1H)), 8.18 (d, J=3.8 Hz, 1H), 12.92 (s, 1H). ¹³C NMR (100 MHz, DMSO-d₆) δ 28.6, 34.2, 79.8, 116.8, 128.3, 129.1, 129.2, 130, 130.7, 131.3, 133.3, 133.5, 139.3, 140.2, 158.5, 161.9, 167.4, 172.9. HPLC>95.1%.

### Example 31: 2-(3-Cyano-benzylsulfanyl)-6-oxo-4-thiophen-2-yl-1,6-dihydro-pyrimidine-5-carbonitrile (Compound I-11)

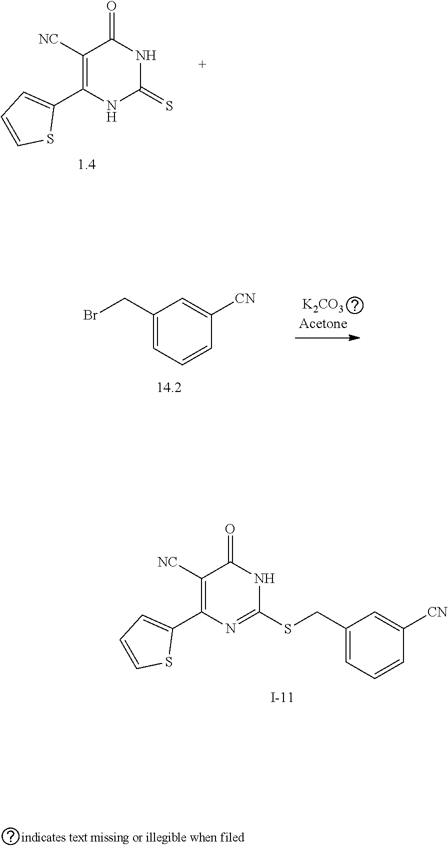

To a stirred solution of intermediate 1.4 (200 mg, 0.85 mmol) and K₂CO₃ (133 mg, 0.93 mmoL) in acetone (20 mL) was added intermediate 14.2 (133 mg, 0.93 mmol) and stirring was continued at rt for 16 h. The solvent was then removed under reduced pressure. The resulting mixture was poured into water, acidified to pH 6 by the addition of a 3M HCl solution, and then extracted with EtOAc (3×20 mL). The combined organic phase was washed with brine and dried over Na₂SO₄. The crude product was purified by flash chromatography eluting with DCM/MeOH to provide the title compound I-11 (50 mg, 0.17 mmol) as a white solid. Yield 17%. ¹H NMR (400 MHz, DMSO-d₆) δ 4.60 (s, 2H), 7.35 (d, J 4.8 Hz, 1H), 7.54 (t, J 7.8 Hz, 1H), 7.74 (d, J 7.7 Hz, 1H), 7.83 (d, J 7.9 Hz, 1H), 7.96 (s, 1H), 8.1 (d, J 5.02 Hz, 1H), 8.27 (d, J=3.9 Hz, 1H), 13.80 (brs, 1H). ¹³C NMR (100 MHz, DMSO-d₆) δ 33.2, 88.8, 111.8, 116.5, 118.9, 130, 130.2, 131.6, 132.1, 132.8, 134.1, 135.4, 139.3, 139.6, 159, 161.2, 165.1. HPLC>99.1%.

### Example 32: 2-[3-(2-Hydroxy-ethyl)-benzylsulfanyl]-6-oxo-4-thiophen-2-yl-1,6-dihydropyrimidine-5-carbonitrile (Compound I-12)

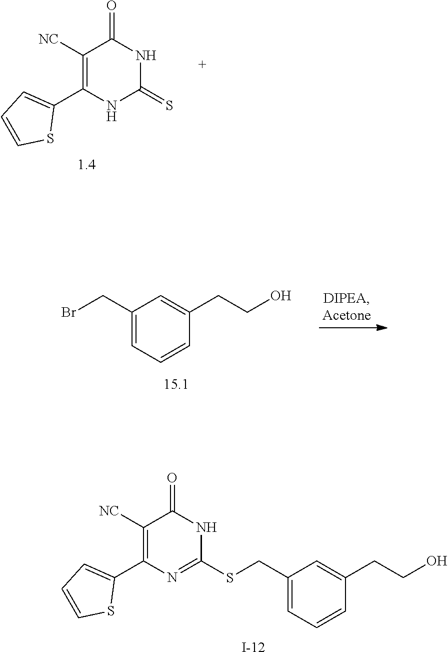

To a stirred solution of intermediate 1.4 (200 mg, 0.85 mmol) and DIPEA (0.16 mL, 0.93 mmoL) in acetone (15 mL) was added intermediate 15.1 (201 mg, 0.93 mmol) and stirring was continued at rt for 16 h. The solvent was then removed under reduced pressure. The resulting mixture was poured into water, acidified to pH 6 by the addition of a 3M HCl solution, and then extracted with EtOAc (3×20 mL). The combined organic phase was washed with brine and dried over Na₂SO₄. The crude product was purified by flash chromatography eluting with DCM/MeOH to provide the title compound I-12 (120 mg, 0.32 mmol) as a white solid. Yield 38%. ¹H NMR (400 MHz, DMSO-d₆) δ 2.47, (t, J=8.35 Hz, 2H), 2.67 (t, J=7.01 Hz, 2H), 4.48 (s, 2H), 4.51 (brs, 1H), 7.11 (d, J=7.5 Hz, 1H), 7.22 (t, J=7.5 Hz, 1H), 7.29 (d, J=7.7 Hz, 1H), 7.32-7.36 (m, 2H), 8.06 (d, J=4.9 Hz, 1H), 8.27 (d, J=3.9 Hz, 1H), 13.80 (brs, 1H). ¹³C NMR (100 MHz, DMSO-d₆) δ 34.3, 62.3, 62.3, 88.3, 116.9, 126.8, 128.5, 128.8, 129.8, 129.9, 131.8, 135.1, 136.9, 139.9, 140.3, 159, 162, 165.9. HPLC>96.1%.

### Example 33: 2-(3-Cyanomethyl-benzylsulfanyl)-6-oxo-4-thiophen-2-yl-1,6-dihydro-pyrimidine-5-carbonitrile (Compound I-13)

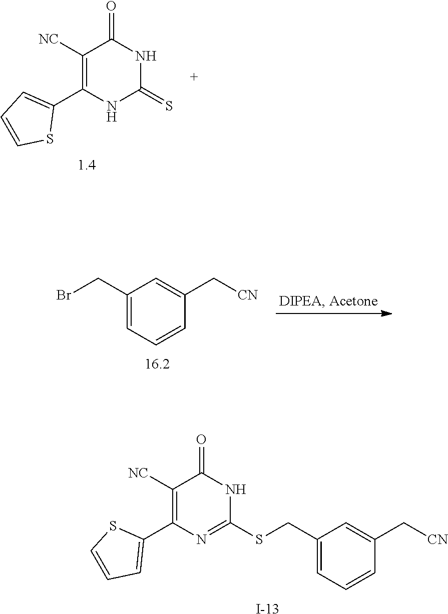

To a stirred solution of intermediate 1.4 (200 mg, 0.85 mmol) and DIPEA (0.2 mL, 0.94 mmoL) in acetone (20 mL) was added intermediate 16.2 (196 mg, 0.94 mmol) and stirring was continued at rt for 16 h. The solvent was then removed under reduced pressure. The resulting mixture was poured into water, acidified to pH 6 by the addition of a 3M HCl solution, and then extracted with EtOAc (3×20 mL). The combined organic phase was washed with brine and dried over Na₂SO₄. The crude product was purified by flash chromatography eluting with DCM/MeOH (2.5% for product) to provide the title compound I-13 (300 mg, 0.82 mmol) as a yellow solid. Yield 96%. ¹H NMR (400 MHz, DMSO-d₆) δ 4.01 (s, 2H), 4.52 (s, 2H), 7.24 (d, J=7.49 Hz, 1H), 7.31-7.36 (m, 2H), 7.43-7.45 (m, 2H), 8.0 (d, J=5 Hz, 1H), 8.23 (d, J=3.8 Hz, 1H), 13.80 (brs, 1H). ¹³C NMR (100 MHz, DMSO-d₆) δ 22.6, 33.9, 87.9, 117.5, 119.5, 127.5, 128.5, 128.9, 129.6, 129.6, 131.3, 131.9, 134.5, 138.6, 140.3, 159.1, 164.1, 166.7. HPLC>97.7%.

### Example 34: 2-[3-(2-Methyl-2H-tetrazol-5-yl)-benzylsulfanyl]-6-oxo-4-thiophen-2-yl-1,6-dihydro-pyrimidine-5-carbonitrile (Compound I-14)

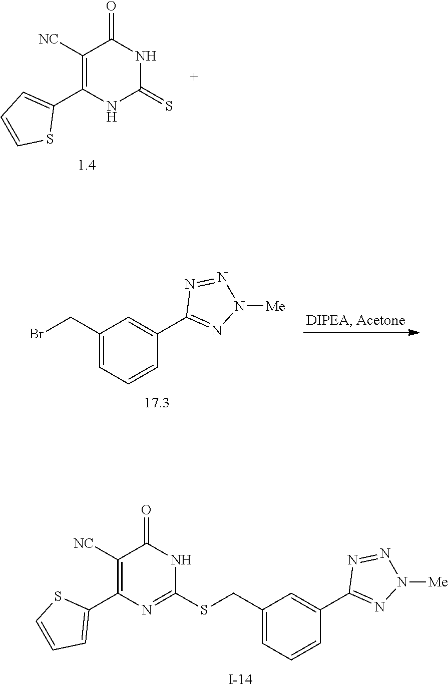

To a stirred solution of intermediate 1.4 (200 mg, 0.85 mmol) and DIPEA (0.17 mL, 0.93 mmoL) in acetone (15 mL) was added intermediate 17.3 (236 mg, 0.93 mmol) and stirring was continued at rt for 16 h. The solvent was then removed under reduced pressure. The resulting solid was collected and dried under reduced pressure to give the title compound I-14 (200 mg, 0.49 mmol) as a yellowish solid. Yield 58%. ¹H NMR (400 MHz, DMSO-d₆) δ 4.40 (s, 3H), 4.66 (s, 2H), 7.35 (m, 1H), 7.51 (t, J=7.5 Hz, 1H), 7.66 (d, J=6.9 Hz, 1H), 7.94 (d, J=7.2 Hz, 1H), 8.08 (d, J=4.1 Hz, 1H), 8.21 (s, 1H), 8.28 (s, 1H), 13.80 (s, 1H). ¹³C NMR (100 MHz, DMSO-d₆) δ 33.9, 40.5, 88.7, 116.5, 125.7, 127.2, 127.5, 129.9, 130, 131.3, 132.1, 135.4, 138.6, 139.7, 159.1, 161.1, 164.2, 165.2. HPLC>99.3%.

### Example 35: [3-(5-Cyano-4-morpholin-4-yl-6-thiophen-2-yl-pyrimidin-2-ylsulfanylmethyl)-phenyl]-acetic acid (Compound I-15)

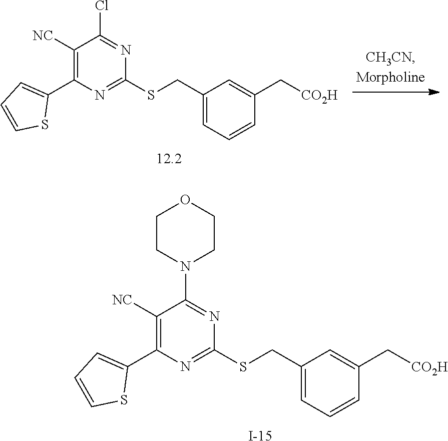

To a stirred suspension of intermediate 12.2 (100 mg, 0.25 mmol) in CH₃CN (10 mL) was added morpholine (0.023 mL, 0.27 mmol) and stirring was continued at rt for 16 h. The solvent was then removed under reduced pressure. The crude product was taken up in water, and the resulting aqueous mixture was extracted with EtOAc (3×20 mL). The combined organic phase was washed with brine and dried over Na₂SO₄ to provide the title compound I-15 (80 mg, 0.18 mmol) as a white solid. Yield 71%. ¹H NMR (400 MHz, CDCl₃) δ 3.60 (s, 2H), 3.79 (m, 4H), 3.93 (m, 4H), 4.41 (s, 2H), 7.17-7.20 (m, 2H), 7.27-7.36 (m, 2H), 7.37 (d, J=8.23 Hz, 2H), 7.61 (d, J=5.1 Hz, 1H), 8.32 (d, J 3.5 Hz, 1H). ¹³C NMR (100 MHz, CDCl₃) δ 35.1, 40.6, 47.7, 47.7, 66.5, 66.5, 80.6, 118.4, 127.7, 128.3, 128.6, 128.8, 129.7, 131.7, 132.4, 133.5, 137.6, 139.9, 161.6, 162.9, 172.6, 176.3. HPLC>98.1%.

### Example 36: [3-(5-Cyano-4-piperazin-1-yl-6-thiophen-2-yl-pyrimidin-2-ylsulfanylmethyl)-phenyl]-acetic acid (Compound I-16)

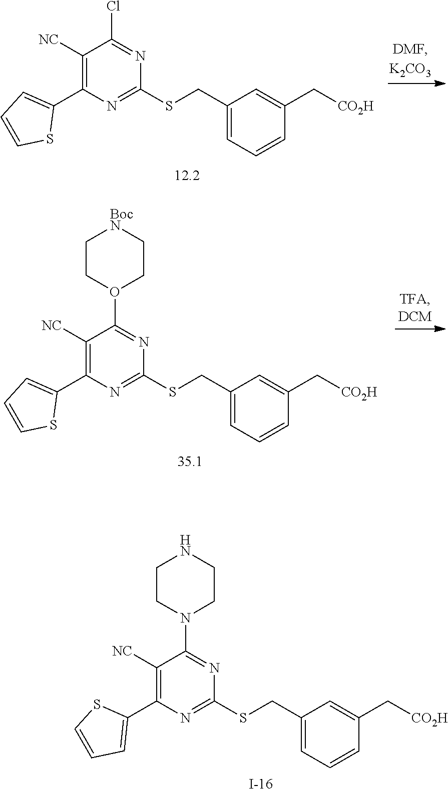

### Step 1. 4-[2-(3-Carboxymethyl-benzylsulfanyl)-5-cyano-6-thiophen-2-yl-pyrimidin-4-yl]-piperazine-1-carboxylic acid tert-butyl ester (36.1)

To a stirred suspension of intermediate 12.2 (250 mg, 0.62 mmol) and K₂CO₃ (128 mg, 0.93 mmol) in DMF (4 mL) was added 1-boc-piperazine (127 mg, 0.68 mmol) and stirring was continued at rt for 16 h. The solvent was then removed under reduced pressure. The resulting mixture was taken up in water, and the aquous mixture was extracted with EtOAc (3×20 mL). The combined organic phase was washed with brine and dried over Na₂SO₄. The crude product was purified by flash chromatography eluting with DCM/MeOH (4% for product) to provide the title intermediate 35.1 (60 mg, 0.11 mmol) as a yellowish solid. Yield 18%.

### Step 2. [3-(5-Cyano-4-piperazin-1-yl-6-thiophen-2-yl-pyrimidin-2-ylsulfanylmethyl)-phenyl]-acetic acid (I-16)

To a stirred solution of intermediate 35.1 (65 mg, 0.12 mmol) in DCM (15 mL) was added TFA (0.28 mL, 3.6 mmol) and stirring was continued at rt for 16 h. The solvent was then removed under reduced pressure. The crude mixture was taken up in water, and the resulting aqueous mixture was extracted with EtOAc (3×20 mL). The combined organic phase was washed with brine and dried over Na₂SO₄. The title compound I-16 (20 mg, 0.044 mmol) was obtained as a white solid after shredding with hot Et₂O. Yield 37%. ¹H NMR (400 MHz, DMSO-d₆) δ 2.83 (m, 4H), 3.51 (s, 2H), 3.81 (m, 4H), 4.39 (s, 3H), 7.13 (d, J=7.03 Hz, 1H), 7.24-7.33 (m, 4H), 7.95 (d, J=4.4 Hz, 1H), 8.20 (d, J=2.8 Hz, 1H). ¹³C NMR (100 MHz, DMSO-d₆) δ 34.6, 41.2, 45.5, 45.5, 48.4, 48.4, 118.7, 127.2, 128.6, 128.7, 129.2, 130.1, 131.8, 133.7, 135.8, 138.1, 140.1, 161.5, 162.4, 172, 173.1. HPLC>90.9%.

### Example 37. [3-(5-Cyano-1-methyl-4-oxo-6-thiophen-2-yl-1,4-dihydro-pyrimidin-2-ylsulfanylmethyl)-phenyl]-acetic acid ethyl ester (Compound I-17)

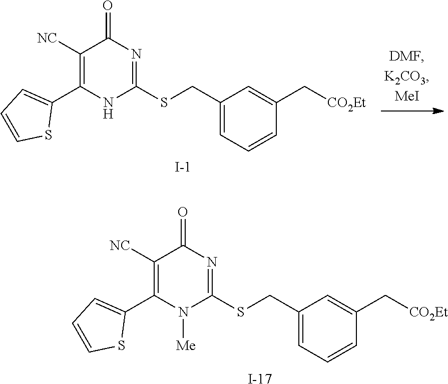

To a stirred suspension of compound I-1 (300 mg, 0.73 mmol) and K₂CO₃ (151 mg, 1.09 mmol) in DMF (15 mL) was added MeI (0.047 mL, 0.77 mmol) dropwise and stirring was continued at rt for 16 h. The resulting mixture was poured into water, and then extracted with EtOAc (3×20 mL). The combined organic phase was washed with brine and dried over Na₂SO₄ to provide the title compound I-17 (298 mg, 0.7 mmol) as a yellowish solid. Yield 96%. ¹H NMR (400 MHz, DMSO-d₆) δ 0.14 (t, J=7.12 Hz, 3H), 3.41 (s, 3H), 3.62 (s, 2H), 4.03 (q, J=7.1 Hz, 2H), 4.64 (s, 2H), 7.19 (d, J=7.6 Hz, 1H), 7.27-7.31 (m, 1H), 7.35 (t, J=4.1 Hz, 1H), 7.40 (m, 2H), 8.08 (d, J 4.9 Hz, 1H), 8.28 (d, J=3.8 Hz, 1H). ¹³C NMR (100 MHz, DMSO-d₆) δ 14.4, 31.1, 36.1, 40.5, 60.6, 87.5, 116.5, 127.9, 129.1, 129.2, 130, 130.4, 132.1, 135.3, 135.4, 136.2, 139.5, 157.1, 160.1, 166.4, 171.3. HPLC>95.1%.

### Example 38. 3-(5-Cyano-6-oxo-4-thiophen-2-yl-1,6-dihydro-pyrimidin-2-ylsulfanylmethyl)-benzenesulfonamide (Compound I-18)

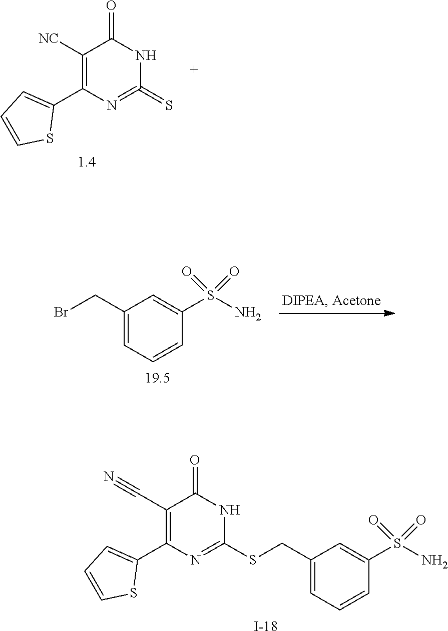

To a stirred solution of intermediate 1.4 (107 mg, 0.45 mmol) and DIPEA (0.08 mL, 0.49 mmoL) in acetone (15 mL) was added intermediate 19.5 (125 mg, 0.49 mmol) and stirring was continued at rt for 16 h. The solvent was then removed under reduced pressure. The crude mixture was taken up in water, and then extracted with EtOAc (3×20 mL). The combined organic phase was washed with brine and dried over Na₂SO₄. The title compound I-18 (50 mg, 0.12 mmol) was obtained as a yellowish solid after trituration with hot Et₂O. Yield 28%. ¹H NMR (400 MHz, DMSO-d₆) δ 4.65 (s, 2H), 7.34 (t, J=4.6 Hz, 1H), 7.40 (s, 2H), 7.52 (t, J=7.6 Hz, 1H), 7.72 (t, J=6.1 Hz, 2H), 7.94 (s, 1H), 8.06 (d, J=4.8 Hz, 1H), 8.27 (d, J=3.6 Hz, 1H), 13.8 (s, 1H). ¹³C NMR (100 MHz, DMSO-d₆) δ 33.7, 88.6, 116.6, 125.2, 126.1, 129.7, 129.9, 132, 132.5, 135.4, 138.4, 139.7, 144.8, 159.1, 161.4, 165.3. HPLC>95.1%.

### Example 39: [3-(3-Cyano-6-oxo-4-phenyl-1,6-dihydro-pyridin-2-ylsulfanylmethyl)-phenyl]-acetic acid (Compound I-19)

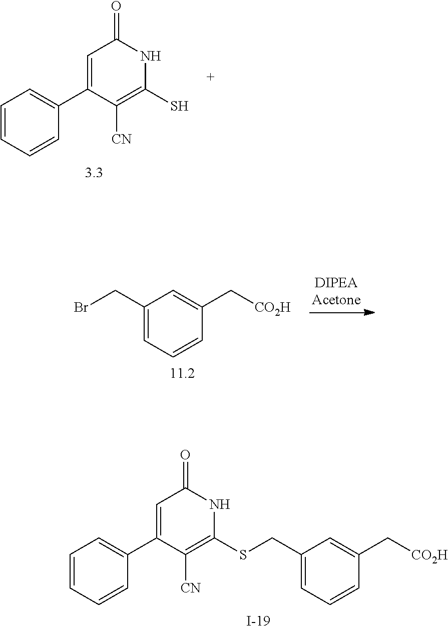

To a stirred suspension of intermediate 3.3 (100 mg, 0.44 mmol) and DIPEA (0.09 mL, 0.53 mmol) in acetone (15 mL) was added intermediate 11.2 (94 mg, 0.44 mmol). Stirring was continued overnight at rt. The mixture was then diluted with crushed ice and water and the pH was adjusted to 5 by the addition of AcOH. The precipitate was collected, washed with cold water and dried under vacuo. Compound I-19 (60 mg, 0.16 mmol) was obtained as a brownish powder. Yield 37%. ¹H NMR (400 MHz, DMSO-d₆) δ 3.55 (s, 2H), 4.5 (s, 2H), 6.51 (s, 1H), 7.16 (d, J=7.4 Hz, 1H), 7.27 (t, J=7.7 Hz, 1H), 7.37 (m, 2H), 7.51-7.53 (m, 3H), 7.55-7.56 (m, 2H), 12.17 (brs, 2H); ¹³C NMR (100 MHz, DMSO-d₆) δ 33.5, 40.6, 95.9, 108, 116.2, 127.6, 128.3, 128.3, 128.5, 128.5, 128.9, 128.9, 130, 130.3, 135.4, 135.9, 137.5, 155.9, 162.1, 164.9, 172.7; HPLC: 96.88%.

### Example 40: [3-(3-Cyano-6-oxo-4-thiophen-2-yl-1,6-dihydro-pyridin-2-ylsulfanylmethyl)-phenyl]-acetic acid (Compound I-20)

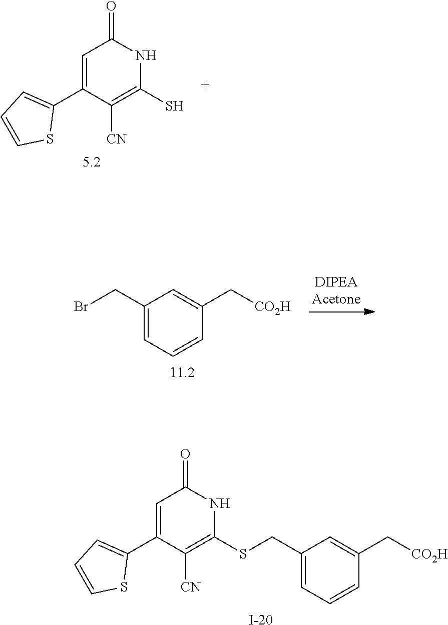

To a stirred suspension of intermediate 5.2 (153 mg, 0.56 mmol) and DIPEA (0.12 mL, 0.67 mmol) in DMSO/acetone (15/4 mL) was added intermediate 11.2 (121 mg, 0.56 mmol). Stirring was continued overnight at room temperature. The mixture was diluted with crushed ice and water. The pH was adjusted to 5 by the addition of AcOH. The precipitate was collected washed with cold water and dried under vacuo. Compound I-20 (90 mg, 0.22 mmol) was obtained as a brownish powder. Yield 42%. ¹H NMR (400 MHz, DMSO-d₆) δ 3.55 (s, 2H), 4.51 (s, 2H), 6.62 (s, 1H), 7.15 (d, J=7.4 Hz, 1H), 7.24-7.28 (m, 2H), 7.35 (d, J=6.4 Hz, 2H), 7.75 (d, J=3.5 Hz, 1H), 7.85 (d, J=4.9 Hz, 1H), 12.1 (brs, 1H); ¹³C NMR (100 MHz, DMSO-d₆) δ 33.6, 40.6, 93.8, 104.5, 116.5, 127.6, 128.6, 128.6, 128.7, 128.7, 129.5, 130.3, 130.3, 135.4, 136.6, 137.4, 147.4, 165.1, 172.7; HPLC: 96.5%.

### Example 41: [3-(3,5-Dicyano-6-oxo-4-thiophen-2-yl-1,6-dihydro-pyridin-2-ylsulfanylmethyl)-phenyl]-acetic acid (Compound I-21)

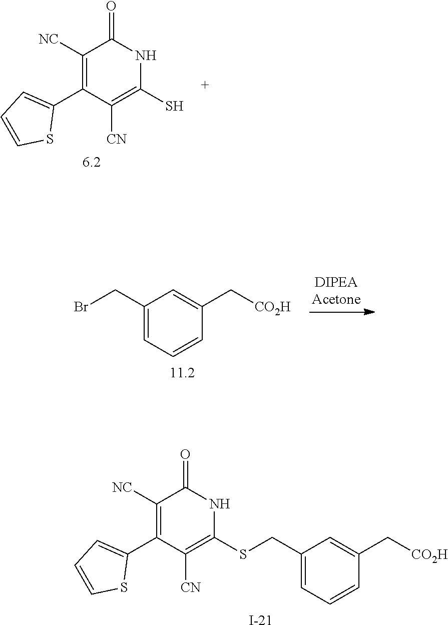

To a stirred solution of intermediate 6.2 (200 mg, 0.77 mmol) and DIPEA (0.16 mL, 0.92 mmol) in acetone (15 mL) was added intermediate 11.2 (165 mg, 0.77 mmol). Stirring was continued overnight at room temperature. The mixture was diluted with crushed ice and water. The pH was adjusted to 5 by the addition of AcOH. The precipitate was collected washed with cold water and dried under vacuo. Compound 1-21 (110 mg, 0.27 mmol) was obtained as a brownish powder. Yield 35%. ¹H NMR (400 MHz, DMSO-d₆) δ 3.56 (s, 2H), 4.48 (s, 2H), 7.63 (d, J=7.6 Hz, 1H), 7.24-7.28 (m, 2H), 7.38-7.40 (m, 2H), 7.54 (dd, J=1.1 Hz, J=3.6 Hz, 1H), 7.93 (dd, J=1.1 Hz, J=5 Hz, 1H), 8.12 (brs, 1H), 12.29 (brs, 1H); ¹³C NMR (100 MHz, DMSO-d₆) δ 33.3, 40.6, 85.8, 93.1, 115.5, 127.8, 128, 128, 128.6, 130.5, 130.9, 131.4, 131.4, 132.9, 135.3, 137.4, 150.8, 159.8, 166.9, 172.8; HPLC: 97.5%.

### Example 42: 2-Oxo-6-[3-(1H-tetrazol-5-yl)-benzylsulfanyl]-4-thiophen-2-yl-1,2-dihydro-pyridine-3,5-dicarbonitrile (Compound I-22)

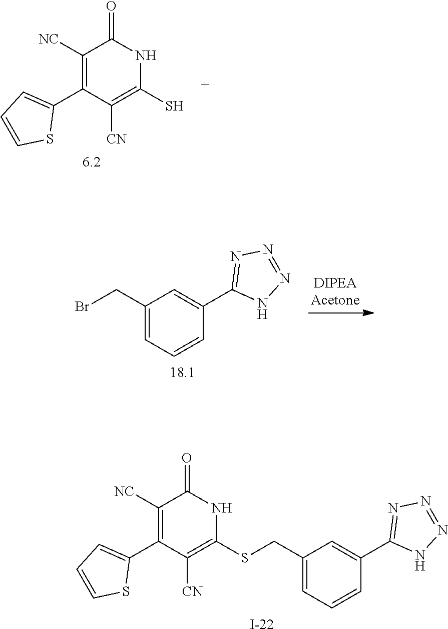

To a stirred solution of intermediate 6.2 (150 mg, 0.57 mmol) and DIPEA (0.18 mL, 0.68 mmol) in acetone (15 mL) was added intermediate 18.1 (138 mg, 0. 57 mmol). Stirring was continued overnight at room temperature. The mixture was diluted with crushed ice and water. pH was adjusted to 5 by the addition of AcOH. The precipitate was collected washed with cold water and dried under vacuo. Compound I-22 (90 mg, 0.27 mmol) was obtained as a yellowish powder. Yield 38%. ¹H NMR (400 MHz, DMSO-d₆) δ 4.6 (s, 2H), 7.26 (dd, J=5.0 Hz, J=3.6 Hz, 1H), 7.54-7.56 (m, 2H), 7.76 (d, J=7.7 Hz, 1H), 7.90-7.94 (m, 2H), 8.1 (s, 1H); ¹³C NMR (100 MHz, DMSO-d₆) δ 33.2, 86.2, 93.4, 115.7, 124.9, 126.2, 128.1, 128.2, 129.8, 131.2, 131.6, 131.6, 132.5, 133, 139.5, 151.1, 155.8, 160.1, 166.8; HPLC: 96.7%

### Example 43: [3-(4-Benzyl-3-cyano-6-oxo-1,6-dihydro-pyridin-2-ylsulfanylmethyl)-phenyl]-acetic acid (Compound I-23)

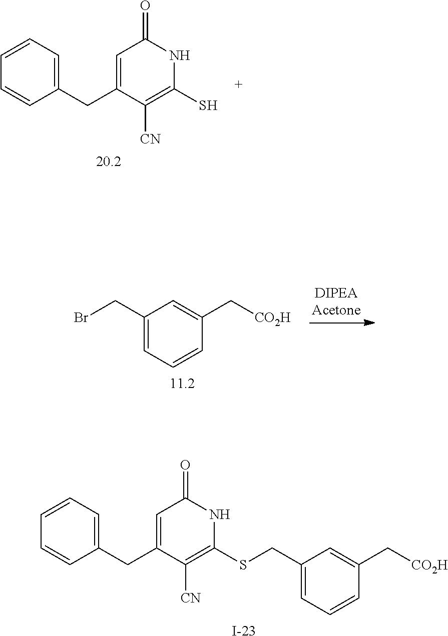

To a stirred solution of intermediate 20.2 (154 mg, 0.63 mmol) and DIPEA (0.12 mL, 0.7 mmol) in acetone (15 mL) was added intermediate 11.2 (150 mg, 0.7 mmol). Stirring was continued overnight at room temperature. The mixture was diluted with crushed ice and water. pH was adjusted to 5 by the addition of AcOH. The precipitate was collected washed with cold water and dried under vacuo. Compound I-23 (100 mg, 0.25 mmol) was obtained as a yellowish powder. Yield 40%). ¹H NMR (400 MHz, DMSO-d₆) δ 3.37 (s, 2H), 3.98 (s, 2H), 4.48 (s, 2H), 6.34 (s, 1H), 7.13-7.32 (m, 9H), 12.1 (brs, 1H); HPLC: 98.5%.

### Example 44: (5-Cyano-6-oxo-4-thiophen-2-yl-1,6-dihydro-pyrimidin-2-ylsulfanyl)-acetic acid (Compound I-24)

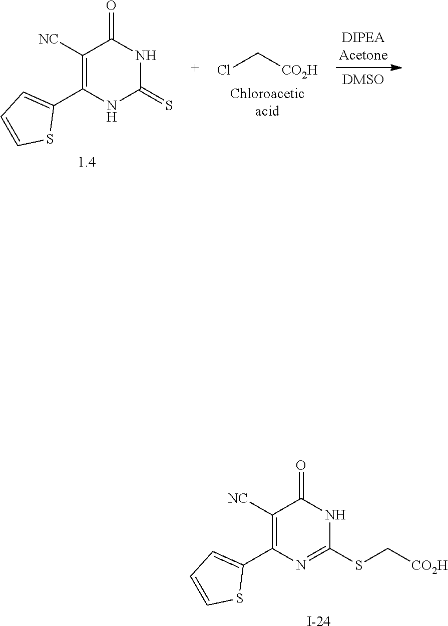

To a stirred solution of intermediate 1.4 (200 mg, 0.85 mmol) and DIPEA (0.18 mL, 1.02 mmol) in acetone/DMSO (20:2 mL) was added chloroacetic acid (80 mg, 0.85 mmol). Stirring was continued overnight at room temperature. Additional 0.3 equivalents of DIPEA and of chloroacetic acid were then added to complete the reaction. The mixture was diluted with crushed ice and water. pH was adjusted to 3 by the addition of 3N HCl. The precipitate was collected and purified by reverse flash chromatography, eluting with H₂O/MeOH from 10 to 80%. Compound I-24 (210 mg, 0.71 mmol) was obtained as a yellowish powder. Yield 83%. ¹H NMR (400 MHz, DMSO-d₆) δ 4.0 (s, 2H), 7.33 (t, J=4.7 Hz, 1H), 8.1 (d, J=4.9 Hz, 1H), 8.25 (d, J=3.8 Hz, 1H), 12.8 (brs, 1H); ¹³C NMR (100 MHz, DMSO-d₆) δ 33.4, 88.5, 116.5, 129.8, 132.3, 135.6, 139.5, 159, 161.1, 165.2, 169.3; HPLC: 99.6%.

### Example 45: 6-Oxo-2-(1H-tetrazol-5-ylmethylsulfanyl)-4-thiophen-2-yl-1,6-dihydro-pyrimidine-5-carbonitrile (Compound I-25)

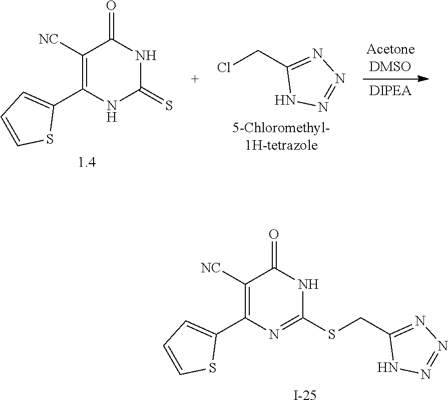

To a stirred solution of intermediate 1.4 (200 mg, 0.85 mmol) and DIPEA (0.18 mL, 1.02 mmol) in acetone/DMSO (20:2 mL) was added intermediate 5-chloromethyl-1H-tetrazole (101 mg, 0.85 mmol). Stirring was continued overnight at room temperature. Additional 0.3 equivalents of DIPEA and of 5-chloromethyl-1H-tetrazole were then added to complete the reaction. The mixture was diluted with crushed ice and water. pH was adjusted to 3 by the addition of 3N HCl. The precipitate was collected and purified by reverse flash chromatography, eluting with H₂O/MeOH from 10 to 80%. Compound I-25 (120 mg, 0.37 mmol) was obtained as a yellowish powder. Yield 44%. ¹H NMR (400 MHz, DMSO-d₆) δ 4.82 (s, 2H), 7.31 (t, J=4.2 Hz, 1H), 8.0 (d, J=4.9 Hz, 1H), 8.22 (d, J=3.8 Hz, 1H); ¹³C NMR (100 MHz, DMSO-d₆) δ 23.6, 88.7, 116.4, 129.9, 132.2, 135.6, 139.4, 157.1, 159.0, 161.4, 164.4; HPLC: 94.6%.

### Example 46: 2-(1H-Tetrazol-5-ylmethylsulfanyl)-6-trifluoromethyl-3H-pyrimidin-4-one (Compound I-26)

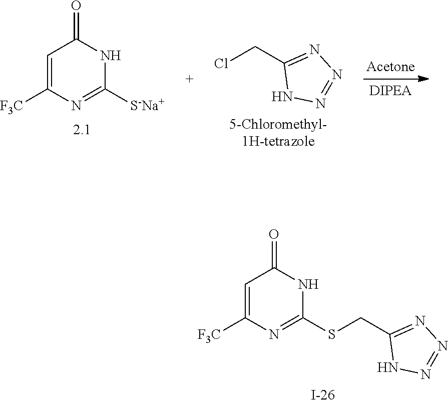

To a stirred solution of intermediate 2.1 (100 mg, 0.56 mmol) and DIPEA (0.13 mL, 0.73 mmol) in acetone (5 mL) was added 5-chloromethyl-1H-tetrazole (87 mg, 0.73 mmol). Stirring was continued overnight at room temperature. The mixture was diluted with crushed ice and water. pH was adjusted to 3 by the addition of 3N HCl. The aqueous phase was extracted with EtOAc (3×20 mL). The combined organic phase was washed with brine and dried over Na₂SO₄. Compound I-26 (60 mg, 0.19 mmol) was obtained as a white powder. Yield 35%. ¹H NMR (400 MHz, DMSO-d₆) δ 4.69 (s, 2H), 6.67 (s, 1H), 15.1 (brs, 1H); ¹³C NMR (100 MHz, DMSO-d₆) δ 23.1, 107.7, 120.6 (q, J_{CF}=2.7 Hz), 152, 154.3, 163.9, 164.8; HPLC: 97.9%

### Example 47: (6-Oxo-4-trifluoromethyl-1,6-dihydro-pyrimidin-2-ylsulfanyl)-acetic acid (Compound I-27)

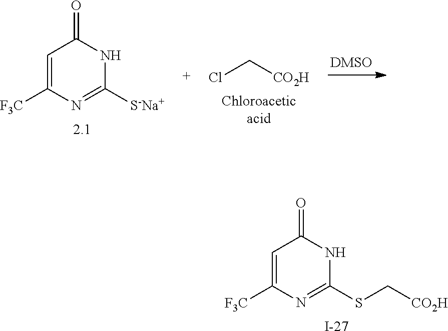

To a stirred solution of intermediate 2.1 (200 mg, 0.85 mmol) and DIPEA (0.16 mL, 0.94 mmol) in DMSO (5 mL) was added chloroacetic acid (89 mg, 0.94 mmol). Stirring was continued overnight at rt. The mixture was diluted with crushed ice and water. pH was adjusted to 3 by the addition of 3N HCl. The aqueous phase was extracted with EtOAc (3×20 mL). The combined organic phase was washed with brine and dried over Na₂SO₄. The crude of reaction was purified by reverse flash chromatography eluting with H₂O/MeOH from 5 to 65% for product. Compound I-27 (125 mg, 0.49 mmol) was obtained as a white powder. Yield 58%. ¹H NMR (400 MHz, DMSO-d₆) δ 3.96 (s, 2H), 6.63 (s, 1H); ¹³C NMR (100 MHz, DMSO-d₆) δ 33.1, 108, 120.6 (q, JCF=2.7 Hz), 163.1, 165.4, 169.5; HPLC: 95.9%.

### Example 48: [3-(6-Oxo-4-trifluoromethyl-1,6-dihydro-pyrimidin-2-ylsulfanylmethyl)-phenyl]-acetic acid (Compound I-28)

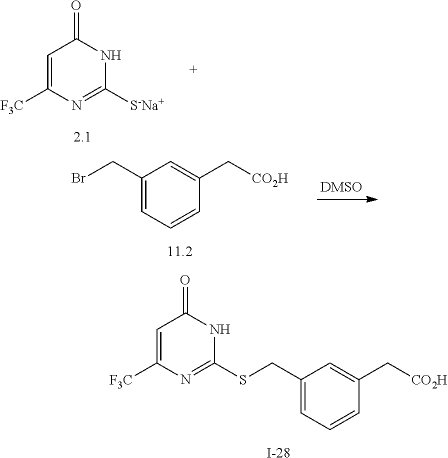

To a stirred solution of intermediate 2.1 (150 mg, 0.64 mmol) and DIPEA (0.12 mL, 0.71 mmol) in DMSO (5 mL) was added intermediate 11.2 (152 mg, 0.71 mmol). Stirring was continued overnight at rt. The mixture was diluted with crushed ice and water. pH was adjusted to 3 by the addition of 3N HCl. The aqueous phase was extracted with EtOAc (3×20 mL). The combined organic phase was washed with brine and dried over Na₂SO₄. The crude of reaction was purified by reverse flash chromatography eluting with H₂O/MeOH from 5 to 80% for product. Compound I-28 (100 mg, 0.29 mmol) was obtained as a white powder. Yield 45%. ¹H NMR (400 MHz, DMSO-d₆) δ 3.54 (s, 2H), 4.52 (s, 2H), 6.87 (s, 1H), 7.16 (d, J=7.3 Hz, 1H), 7.26 (t, J=7.4 Hz, 1H), 7.33-7.35 (m, 2H), 12.18 (brs, 1H); HPLC: 98.1%.

### Example 49: 2-[3-(1H-Tetrazol-5-yl)-benzylsulfanyl]-6-trifluoromethyl-3H-pyrimidin-4-one (Compound I-29)

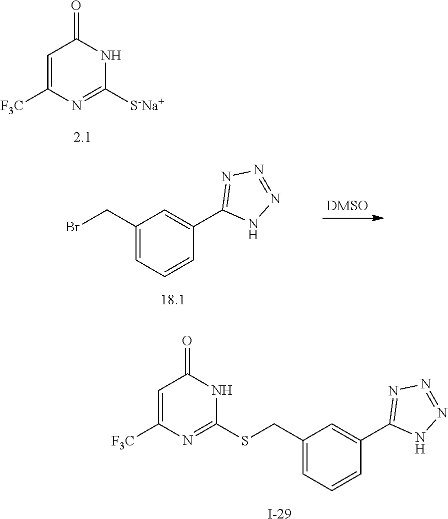

To a stirred solution of intermediate 2.1 (150 mg, 0.64 mmol) and DIPEA (0.12 mL, 0.71 mmol) in DMSO (5 mL) was added intermediate 18.1 (152 mg, 0.64 mmol). Stirring was continued overnight at rt. The mixture was diluted with crushed ice and water. pH was adjusted to 3 by the addition of 3N HCl. The white solid was collected and characterized as the title compound. Compound I-29 (160 mg, 0.45 mmol) was obtained as a white powder. Yield 70%. ¹H NMR (400 MHz, DMSO-d₆) δ 4.50 (s, 2H), 6.63 (s, 1H), 7.54 (t, J=7.7 Hz, 1H), 7.64 (d, J=7.5 Hz, 1H), 7.9 (d, J=7.6 Hz, 1H), 8.13 (s, 1H), 13.3 (brs, 1H); HPLC: 95.2%.

### Example 50: (4-Benzyl-5-cyano-6-oxo-1,6-dihydro-pyrimidin-2-ylsulfanyl)-acetic acid (Compound I-30)

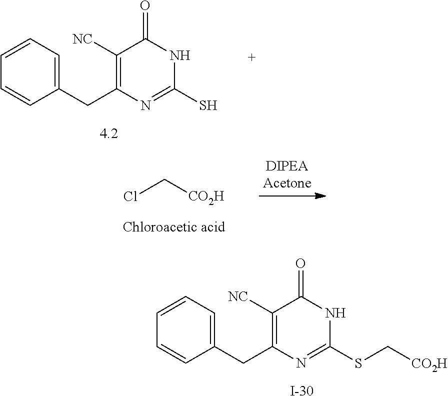

To a stirred solution of intermediate 4.2 (110 mg, 0.45 mmol) and DIPEA (0.086 mL, 0.5 mmol) in acetone (10 mL) was added chloroacetic acid (43 mg, 0.45 mmol). Stirring was continued overnight reflux. The mixture cooled to rt and it was diluted with crushed ice and water. pH was adjusted to 3 by the addition of 3N HCl. The aqueous phase was extracted with EtOAc (3×20 mL). The combined organic phase was washed with brine and dried over Na₂SO₄. The crude of reaction was purified by reverse phase flash chromatography eluting with H₂O/MeOH from 5 to 80% for product. Compound I-30 (95 mg, 0.32 mmol) was obtained as a white powder. Yield 69%. ¹H NMR (400 MHz, DMSO-d₆) δ 3.92 (s, 2H), 4.0 (s, 2H), 7.24-7.28 (m, 1H), 7.31-7.32 (4H); ¹³C NMR (100 MHz, DMSO-d₆) δ 33.4, 42.6, 95.0, 115.4, 127.4, 129, 129, 129.3, 129.3, 136.4, 160.7, 166.3, 169.4, 169.4; HPLC: 98.9%.

### Example 51: 3-(6-Oxo-4-trifluoromethyl-1,6-dihydro-pyrimidin-2-ylsulfanylmethyl)-benzoic acid (Compound I-31)

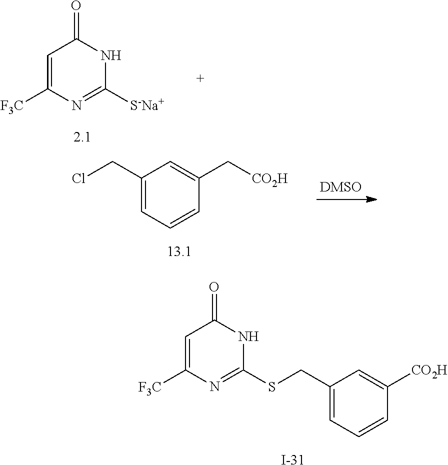

To a stirred solution of intermediate 2.1 (200 mg, 0.92 mmol) and DIPEA (0.19 mL, 1.1 mmol) in DMSO (5 mL) was added 3(2-chloromethyl) benzoic acid 13.1 (170 mg, 1 mmol). Stirring was continued overnight at rt. The mixture was diluted with crushed ice and water. pH was adjusted to 3 by the addition of 3N HCl. The white precipitate was collected and dried under vacuo. The crude was purified by reverse phase chromatography, eluting with H₂O/MeOH. from 4 to 80%. Compound 1-31 (120 mg, 0.36 mmol) was obtained as a white powder. Yield 40%. ¹H NMR (400 MHz, DMSO-d₆) δ 2.5 (s, 2H), 4.45 (s, 2H), 6.61 (s, 1H), 7.42 (t, J=7.6 Hz, 1H), 7.48 (d, J=7.4 Hz, 1H), 7.81 (d, J=7.58 Hz, 1H), 8.02 (s, 1H), 13.12 (brs, 1H); ¹³C NMR (100 MHz, DMSO-d₆) δ 33.8, 107.7, 120.6 (q, J_{CF}=2.7 Hz), 128.6, 128.9, 130.4, 131.3, 134, 138.3, 150, 163, 165, 167.4; HPLC: 98.8%.

### Example 52: 3-(6-Oxo-4-trifluoromethyl-1,6-dihydro-pyrimidin-2-ylsulfanylmethyl)-benzoic acid (Compound I-32)

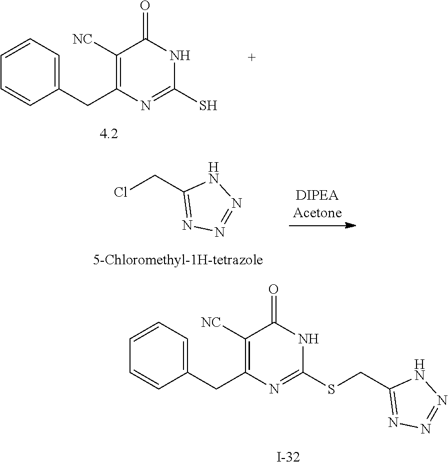

To a stirred solution of intermediate 4.2 (200 mg, 0.92 mmol) and DIPEA (0.16 mL, 0.9 mmol) in acetone (10 mL) was added 5 chloromethyl-1H-tetrazole (97 mg, 0.82 mmol). Stirring was continued overnight at reflux. The mixture was diluted with crushed ice and water. pH was adjusted to 3 by the addition of 3N HCl. The mixture was extracted with EtOAc (3×30 mL). The combined organic phase was washed with brine and dried over Na₂SO₄. Compound I-32 (58 mg, 0.18 mmol) was obtained as a white powder after purification by reverse phase flash chromatography, eluting with H₂O/MeOH from 5 to 80%. Yield 20%. ¹H NMR (400 MHz, DMSO-d₆) δ 3.90 (s, 2H), 4.74 (s, 2H), 7.13-7.21 (m, 5H); HPLC: 98.9%.

### Example 53: [3-(4-Benzyl-5-cyano-6-oxo-1,6-dihydro-pyrimidin-2-ylsulfanylmethyl)-phenyl]-acetic acid (Compound I-33)

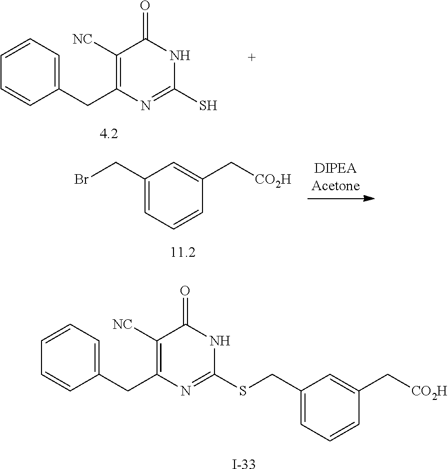

To a stirred solution of intermediate 4.2 (150 mg, 0.62 mmol) and DIPEA (0.12 mL, 0.68 mmol) in acetone (10 mL) was added intermediate 11.2 (146 mg, 0.68 mmol). Stirring was continued overnight reflux. The mixture cooled to rt and it was diluted with crushed ice and water. pH was adjusted to 5 by the addition of AcOH. The precipitate was collected and dried under vacuo. The title compound I-33 (80 mg, 0.2 mmol) was obtained as a white solid. Yield: 33%. ¹H NMR (400 MHz, DMSO-d₆) δ 3.5 (s, 2H), 4.0 (s, 2H), 4.37 (s, 2H), 7.12-7.32 (m, 9H); ¹³C NMR (100 MHz, DMSO-d₆) δ 34.2, 42.6, 54.1, 95.2, 116, 127.5, 127.8, 128.9, 129, 129.1, 129.1, 129.6, 129.6, 130.4, 135.7, 136.8, 137.5, 161, 166.7, 172.6, 173.1; HPLC: 90.2%

### Example 54: 4-Benzyl-6-oxo-2-[3-(1H-tetrazol-5-yl)-benzylsulfanyl]-1,6-dihydro-pyrimidine-5 carbonitrile (Compound I-34)

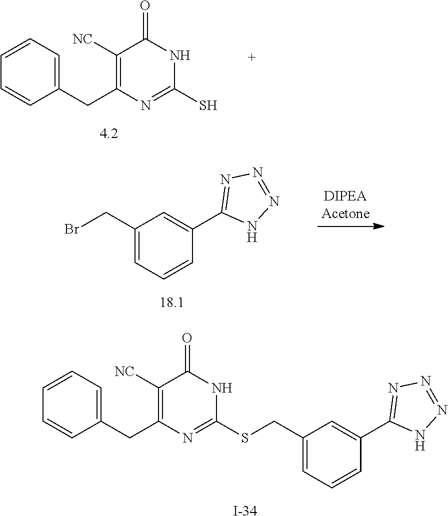

To a stirred solution of intermediate 4.2 (150 mg, 0.62 mmol) and DIPEA (0.12 mL, 0.68 mmol) in acetone (10 mL) was added intermediate 18.1 (162 mg, 0.68 mmol). Stirring was continued overnight reflux. The mixture cooled to rt and it was diluted with crushed ice and water. pH was adjusted to 5 by the addition of AcOH. The precipitate was collected and dried under vacuo. The crude was suspended in water, acidified to pH 3 by the addition of 3N HCl solution and extracted with EtOAc, (3×20 mL). The combined organic phase was washed with brine and dried over Na₂SO₄. The title compound I-34 (125 mg, 0.31 mmol) was obtained as a light yellow solid. Yield: 50%. ¹H NMR (400 MHz, DMSO-d₆) δ 4.0 (s, 2H), 4.51 (s, 2H), 7.17-7.28 (m, 5H), 7.41-7.47 (2H), 7.90 (d, J=6.82 Hz, 1H), 8.06 (s, 2H); ¹³C NMR (100 MHz, DMSO-d₆) δ 33.7, 42.5, 95.5, 115.5, 124.7, 126.2, 127.3, 128.8, 128.9, 128.9, 129.4, 129.4, 129.9, 132.1, 136.5, 139, 158, 160.6, 166.2, 172.7; HPLC: 93%.

### Example 55: 3-(4-Benzyl-5-cyano-6-oxo-1,6-dihydro-pyrimidin-2-ylsulfanylmethyl)-benzoic acid (Compound I-35)

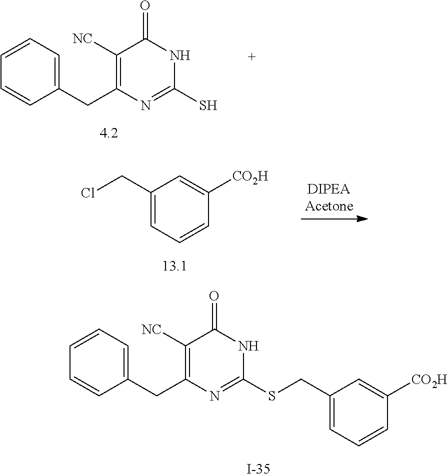

To a stirred solution of intermediate 4.2 (150 mg, 0.62 mmol) and DIPEA (0.14 mL, 0.82 mmol) in acetone (10 mL) was added 3(2-chloromethyl) benzoic acid 13.1 (116 mg, 0.68 mmol). Stirring was continued overnight at r.t. The mixture cooled tort and it was diluted with crushed ice and water. pH was adjusted to 3 by the addition of 3N HCl. The precipitate was collected and dried under vacuo. The title compound I-35 (160 mg, 0.42 mmol) was obtained as a light yellow solid. Yield: 68%. ¹H NMR (400 MHz, DMSO-d₆) δ 4.0 (s, 2H), 4.46 (s, 2H), 7.25-7.36 (m, 5H), 7.44 (d, J=7.3 Hz, 1H), 7.81 (d, J=7.6 Hz, 1H), 8.0 (s, 1H), 13.1 (brs, 1H); ¹³C NMR (100 MHz, DMSO-d₆) δ 33.7, 42.5, 95.5, 115.5, 127.4, 128.6, 129, 129, 129.1, 129.4, 129.4, 130.3, 131.2, 133.8, 136.5, 138.1, 160.5, 166.2, 167.4, 172.7; HPLC: 98%. HPLC: 98%.

### Example 56: [3-(3-cyano-6-oxo-4-trifluoromethyl-1,6-dihydro-pyridin-2-ylsulfanylmethyl)-phenyl]-acetic acid (Compound I-36)

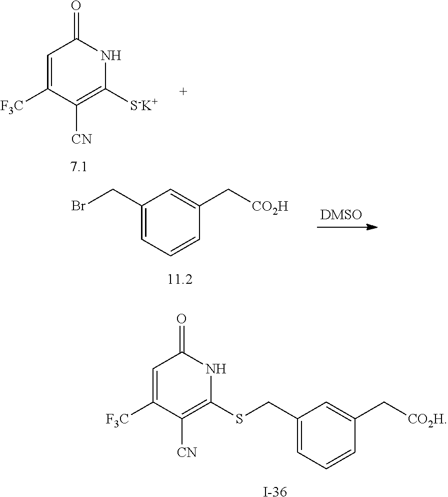

To a stirred solution of intermediate 7.1 (164 mg, 0.63 mmol) and DIPEA (0.12 mL, 0.71 mmol) in DMSO (5 mL) was added intermediate 11.2 (150 mg, 0.71 mmol). Stirring was continued overnight at r.t. The mixture was diluted with crushed ice and water. pH was adjusted to 3 by the addition of 3N HCl followed by extraction with EtOAc, (3×20 mL). The combined organic phase was washed with brine, dried over Na₂SO₄. The crude was purified by reverse phase chromatography eluting with H₂O/MeOH from 8% to 40% for product. The title compound I-36 (85 mg, 0.23 mmol) was obtained as a white powder. Yield: 37%. ¹H NMR (400 MHz, DMSO-d₆) δ 3.55 (s, 2H), 4.53 (s, 2H), 6.87 (s, 1H), 7.16 (m, 1H), 7.26 (m, 1H), 7.35 (m, 2H), 12.2 (brs, 1H). ¹³C NMR (100 MHz, DMSO-d₆) δ 34.1, 40.8, 91.5, 105.3, 113.8, 121.5 (q, JCF=2.7 Hz), 127.8, 128.8, 129, 130.6, 135.7, 137.1, 142 (q, JCF=0.3 Hz), 164.6, 165.6, 173; HPLC: 97.8%.

### Example 57: [3-(5-Cyano-6-oxo-4-thiophen-2-yl-1,6-dihydro-pyrimidin-2-ylsulfanyl)-propionic acid (Compound I-37)

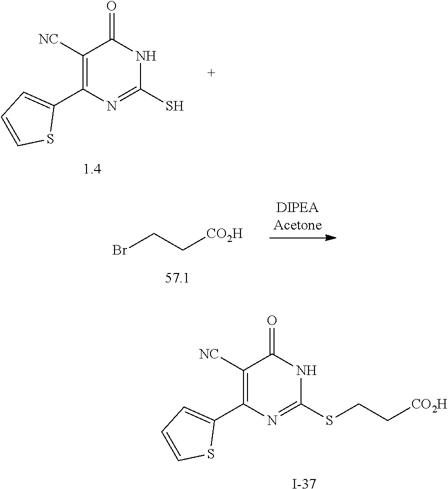

To a stirred solution of intermediate 1.4 (200 mg, 0.84 mmol) and DIPEA (0.16 mL, 0.93 mmol) in acetone (10 mL) was added 3-bromo-propionic acid (57.1) (142 mg, 0.93 mmol). Stirring was continued overnight at rt. The mixture was then diluted with crushed ice and water and the pH was adjusted to 3 by the addition of 3N HCl. The resulting precipitate was collected and dried under vacuo. The title compound I-37 (180 mg, 0.58 mmol) was obtained as a light yellow solid. Yield: 69%. ¹H NMR (400 MHz, DMSO-d₆) δ 2.75-2.80 (m, 2H), 3.34-3.39 (m, 2H), 7.32-7.36 (m, 1H), 8.0-8.24 (m, 1H), 8.24-8.27 (m, 1H), 12.18 (brs, 1H), 13.72 (brs, 1H); ¹³C NMR (100 MHz, DMSO-d₆) δ 26.2, 33.8, 40.7, 88.4, 1116.6, 130.1, 131.9, 135.4, 139.9, 158.9, 161.1, 165.6, 173.1; HPLC: 96.6%.

### Example 58: 3-(6-Oxo-4-trifluoromethyl-1,6-dihydro-pyrimidin-2-ylsulfanyl)-propionic acid (Compound I-38)

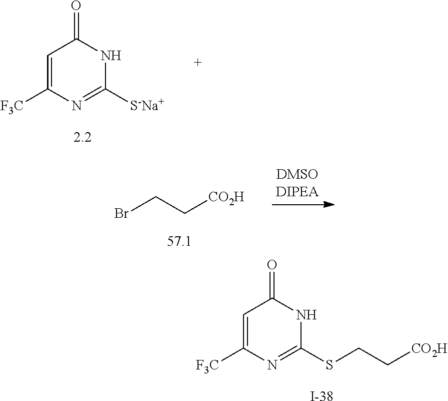

To a stirred solution of intermediate 2.2 (150 mg, 0.69 mmol) and DIPEA (0.13 mL, 0.75 mmol) in DMSO (5 mL) was added 3-bromo-propionic acid (57.1) (115 mg, 0.75 mmol). Stirring was continued overnight at rt. The mixture was diluted with crushed ice and water and the pH was adjusted to 3 by the addition of 3N HCl. The aqueous phase was extracted with EtOAc (3×20 mL). The combined organic phase was washed with brine, and dried over Na₂SO₄. The title compound I-38 (65 mg, 0.24 mmol) was obtained as a white powder. Yield: 35%. ¹H NMR (400 MHz, DMSO-d₆) δ 2.69 (t, J=6.68 Hz, 2H), 3.27 (t, J=6.7 Hz, 2H), 6.61 (s, 1H), 12.9 (brs, 2H); HPLC: 98.8%.

### Example 59: 2-(3,5-Difluoro-4-hydroxy-benzylsulfanyl)-6-oxo-4-thiophen-2-yl-1,6-dihydro-pyrimidine-5-carbonitrile (Compound I-39)

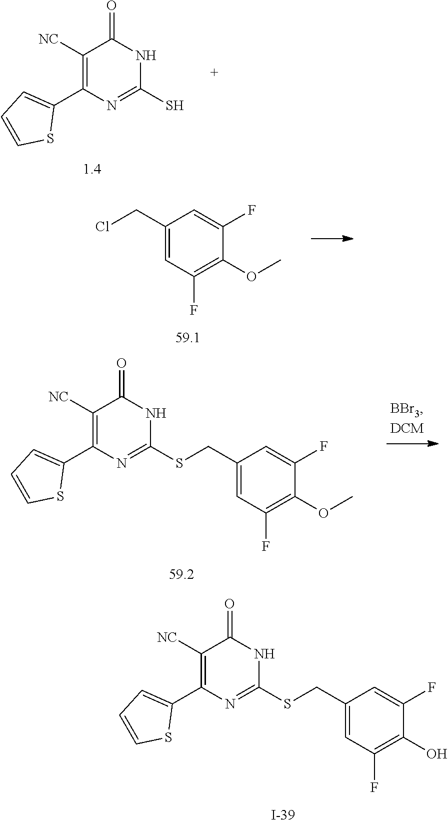

### Step 1: 2-(3,5-Difluoro-4-methoxy-benzylsulfanyl)-6-oxo-4-thiophen-2-yl-1,6-dihydro-pyrimidine-5-carbonitrile (59.2)

To a solution of intermediate 1.4 (150 mg, 0.64 mmol) in DMSO (5 mL) was added DIPEA (0.12 mL, 0.7 mmol) and intermediate 59.1 (135 mg, 0.7 mmol). Stirring was continued at rt 16 h. The crude was poured in water, acidified to pH 3 by a 3N solution of HCl. The aqueous phase was extracted with EtOAc (3×20 mL). The combined organic phase was washed with brine and dried over Na₂SO₄. The intermediate 59.2 (168 mg, 0.42 mmol) was obtained as a orange powder. Yield: 67%. ¹H NMR (400 MHz, DMSO-d₆) δ 3.87 (s, 2H), 4.5 (s, 3H), 7.28 (d, J=9.1 Hz, 2H), 7.36 (t, J=4.6 Hz, 1H), 8.1 (d, J=4.9 Hz, 1H), 8.28 (d, J 3.8 Hz, 1H).

### Step 2: 2-(3,5-Difluoro-4-hydroxy-benzylsulfanyl)-6-oxo-4-thiophen-2-yl-1,6-dihydro-pyrimidine-5-carbonitrile (I-39)

To a stirred suspension of intermediate 59.2 (160 mg, 0.41 mmol) in DCM (10 mL) was added at 0° C. a 1 M solution of BBr₃ in DCM (0.5 mL, 0.45 mmol). Stirring was continued at rt 16 h at rt. The reaction was quenched with MeOH and the solvents were removed under vacuo. The crude was purified by flash chromatography eluting with DCM/MeOH from 0 to 6% for product. The title compound I-39 (68 mg, 0.18 mmol) was obtained as a white powder after trituration with Et₂O. Yield: 44%. ¹H NMR (400 MHz, DMSO-d₆) δ 4.47 (s, 2H), 7.17 (d, J=8.3 Hz, 2H), 7.36 (t, J=4.6 Hz, 1H), 8.28 (d, J=3.9 Hz, 1H), 10.25 (s, 1H), 13.9 (brs, 1H); ¹³C NMR (100 MHz, DMSO-d₆) δ 33.3, 88.7, 112.7 (d, J_{CF}=28.6 Hz), 112.9 (d, J_{CF}=28.6 Hz), 116.5, 127.8 (t, J_{CF}=32.8 Hz), 130, 132.1, 133.4 (t, J_{CF}=63.7 Hz), 135.4, 139.7, 151.0 (d, J_{CF}=28.9 Hz), 153.4 (d, J_{CF}=28.6 Hz), 158.9, 161.2, 165.2; HPLC: 95.7%.

### Example 60: 2-(3,5-Difluoro-4-hydroxy-benzylsulfanyl)-6-trifluoromethyl-3H-pyrimidin-4-one (Compound I-40)

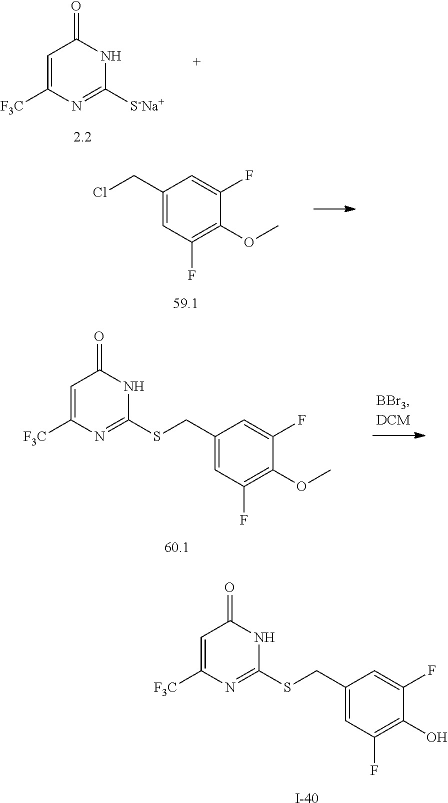

### Step 1: 2-(3,5-Difluoro-4-methoxy-benzylsulfanyl)-6-trifluoromethyl-3H-pyrimidin-4-one (60.1)

Following the procedure of Example 59 (Step 1) and starting from intermediate 2.2 (200 mg, 0.91 mmol), 59.1 (211.8 mg, 1.1 mmol) and DIPEA (0.19 mL, 1.1 mmol) in DMSO (5 mL) the title intermediate 60.1 (200 mg, 0.56 mmol) was obtained as white powder. Yield 62%. ¹H NMR (400 MHz, DMSO-d₆) δ 3.88 (s, 2H), 4.33 (s, 3H), 6.63 (s, 1H), 7.21 (d, J=9.2 Hz, 2H).

### Step 2: 2-(3,5-Difluoro-4-hydroxy-benzylsulfanyl)-6-trifluoromethyl-3H-pyrimidin-4-one (I-40)

To a stirred suspension of intermediate 60.1 (190 mg, 0.54 mmol) in DCM (10 mL) was added at 0° C. a 1 M solution of BBr₃ in DCM (0.6 mL, 0.59 mmol). Stirring was continued at rt 16 h at rt. The reaction was quenched with MeOH. The solvents were removed under vacuo. The crude was purified by flash chromatography eluting with DCM/MeOH from 0 to 6% for product. The title compound I-40 (61 mg, 0.18 mmol) was obtained as a white powder after trituration with Et₂O. Yield: 33%. ¹H NMR (400 MHz, DMSO-d₆) 4.29 (s, 2H), 6.63 (s, 1H), 7.11 (d, J=7.82 Hz, 2H), 10.19 (s, 1H), 13.15 (brs, 1H); ¹³C NMR (100 MHz, DMSO-d₆) δ 33.3, 113.1 (d, J_{CF}=28.2 Hz), 113.2 (d, J_{CF}=28.4 Hz), 119.6, 122.3, 128.2, 133.3 (t, J_{CF}=64.1 Hz), 150.9 (d, J_{CF}=28.3 Hz), 153.3 (d, J_{CF} 28.4 Hz); HPLC: 98.3%.

### Example 61: 4-Benzyl-2-(3,5-difluoro-4-hydroxy-benzylsulfanyl)-6-oxo-1,6-dihydro-pyrimidine-5-carbonitrile (Compound I-41)

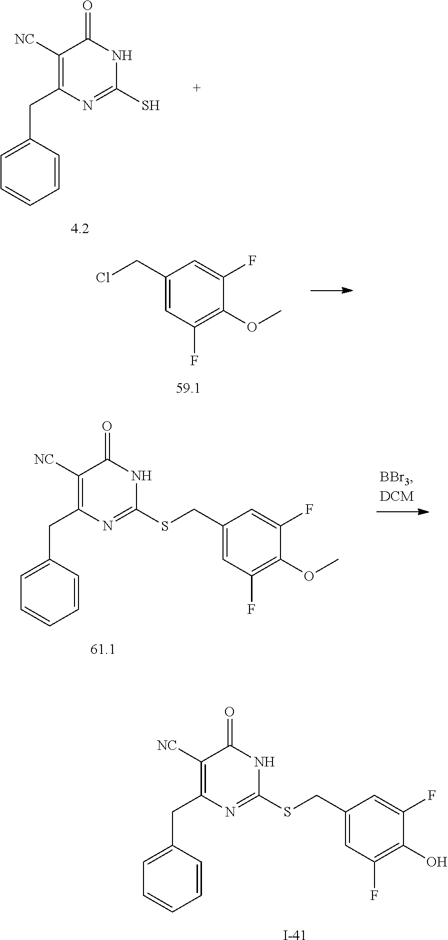

### Step 1: 4-Benzyl-2-(3,5-difluoro-4-methoxy-benzylsulfanyl)-6-oxo-1,6-dihydro-pyrimidine-5-carbonitrile (61.1)

Following the procedure of Example 59 (Step 1) and starting from intermediate 4.2 (200 mg, 0.82 mmol), 59.1 (189.9 mg, 0.98 mmol) and DIPEA (0.17 mL, 0.98 mmol) in DMSO (5 mL) the title intermediate 60.1 (200 mg, 0.5 mmol) was obtained as white powder. Yield 61%. ¹H NMR (400 MHz, DMSO-d₆) δ 3.87 (s, 2H), 4.02 (s, 2H), 4.35 (s, 2H), 7.0 (d, J=8.9 Hz, 2H), 7.3 (m, 5H).

### Step 2: 4-Benzyl-2-(3,5-difluoro-4-hydroxy-benzylsulfanyl)-6-oxo-1,6-dihydro-pyrimidine-5-carbonitrile (Compound I-41)

To a stirred suspension of intermediate 61.1 (190 mg, 0.54 mmol) in DCM (10 mL) was added at 0° C. a 1 M solution of BBr₃ in DCM (0.5 mL, 0.52 mmol). Stirring was continued at rt 16 h at rt. The reaction was quenched with MeOH. The solvents were removed under vacuo. The crude was purified by flash chromatography eluting with DCM/MeOH from 0 to 6% for product. The title compound I-41 (40 mg, 0.1 mmol) was obtained as a white powder after trituration with Et₂O. Yield: 22%. ¹H NMR (400 MHz, DMSO-d₆) 4.02 (s, 2H), 4.3 (s, 2H), 6.93 (d, J=7.6 Hz, 2H), 7.27 (m, 4H), 7.9 (brs, 1H), 10.2 (s, 1H), 13.92 (bras, 1H); HPLC 96.7%.

### Example 62: 6-Oxo-2-{[4-(1H-tetrazol-5-yl)-cyclohexylmethyl]-amino}-4-thiophen-2-yl-1,6-dihydro-pyrimidine-5-carbonitrile (Compound I-42)

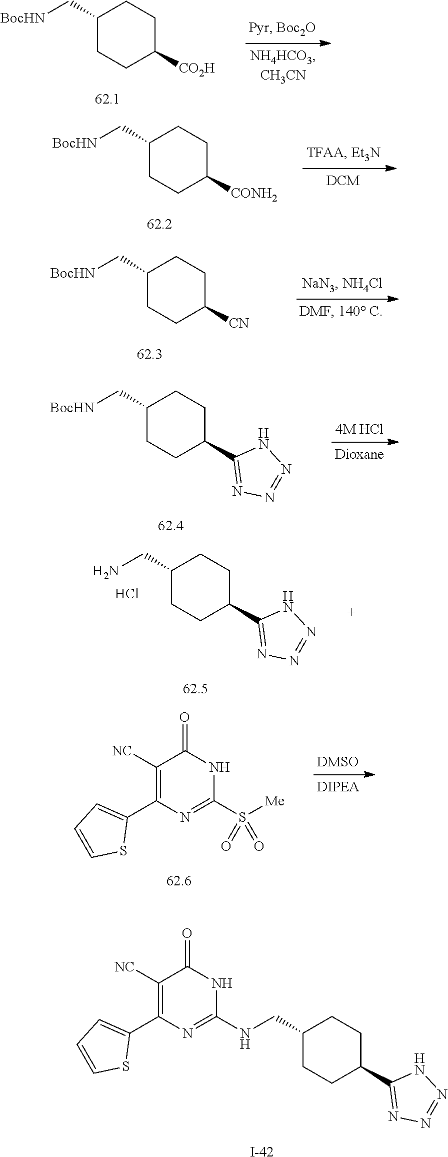

### Step 1: (4-Carbamoyl-cyclohexylmethyl)-carbamic acid tert-butyl ester (62.2)

To a solution of the starting intermediate 62.1 (1.3 g, 5.1 mmol) in CH₃CN (20 mL) was added pyridine (0.45 mL, 5.55 mmol), Boc₂O (1.67 g, 7.65 mmol) and ammonium bicarbonate (605 mg, 7.65 mmol). Stirring was continued at rt 16 h. The crude was poured in water. The aqueous phase was extracted with EtOAc (3×20 mL). The combined organic phase was washed with brine, and dried over Na₂SO₄. The title compound 62.2 (1 g, 3.9 mmol) was obtained as a white powder. Yield: 76%. ¹H NMR (400 MHz, CDCl₃) δ 0.8 (q, J=2.6 Hz, 2H), 1.45 (s, 9H), 1.63 (s, 2H), 1.86 (d, J=13.3 Hz, 2H), 1.97 (d, J=13.4 Hz, 2H), 2.11 (t, J=3.4 Hz, 1H), 2.99 (m, 2H), 4.5 (s, 1H), 5.38 (m, 2H).

### Step 2: (4-Cyano-cyclohexylmethyl)-carbamic acid tert-butyl ester (62.3)

A solution of the starting intermediate 62.2 (200 mg, 0.78 mmol) in DCM (10 ml) was added Et₃N (0.3 mL, 1.95 mmol). The mixture was cooled at 0° C. and TFAA (0.14 mL, 0.98 mmol) was added dropwise. Stirring was continued at rt 16 h. The reaction was poured in water, extracted with DCM (3×20 mL). The combined organic phase was washed with brine and were dried over Na₂SO₄ to give the title compound 62.3 (150 mg, 0.62 mmol) as yellow oil. Yield 84%. ¹H NMR (400 MHz, CDCl₃) δ 0.98-1 (m, 2H), 1.46 (s, 9H), 1.52 (m, 2H), 1.57-1.61 (m, 1H), 1.75-1.77 (m, 1H), 1.83-1.87 (m, 2H), 2.12-2.16 (m, 2H), 2.35-2.42 (m, 1H), 2.98 (t, J=6.4 Hz, 2H), 3.58 (d, J=6.8 Hz, 1H), 4.61 (brs, 1H).

### Step 3: [4-(1H-Tetrazol-5-yl)-cyclohexylmethyl]-carbamic acid tert-butyl ester (62.4)

To a solution of the starting intermediate 62.3 (200 mg, 0.84 mmol) in DMF (3 mL) was sodium azide (164 mg, 2.52 mmol) and NH4Cl (135 mg, 2.52 mmol). Stirring was continued at 140° C. for 25 h. The crude was poured in water, acidified to pH3 by the addition of 3M HCl solution. The aqueous phase was extracted with EtOAc (3×20 mL). The combined organic phase was washed with brine and dried over Na₂SO₄. The title compound 62.4 (160 mg, 0.56 mmol) was obtained as a white powder. Yield: 67%. ¹H NMR (200 MHz, DMSO-d₆) δ 1.01 (t, J 6.8 Hz, 2H), 1.36-1.52 (m, 11H), 1.76 (d, J 11.7 Hz, 2H), 2.01 (d, J=11.0 Hz, 2H), 2.72-2.96 (m, 3H), 6.59-6.87 (m, 1H), 14.1 (brs, 1H).

### Step 4: C-[4-(1H-Tetrazol-5-yl)-cyclohexyl]-methylamine (62.5)

To a solution of intermediate 62.4 (450 mg, 1.60 mmol) in dioxane (5 mL) was added a 4M solution of HCl in dioxane (13.2 mL). Stirring was continued at r.t 16 h. The solvent was removed under vacuo to give the title intermediate 62.5 (378 mg, 1.73 mmol) as a white powder chlorohydrate salt. Yield 95%. ¹H NMR (200 MHz, DMSO-d₆) δ 1.0-1.14 (m, 2H), 1.46-1.52 (m, 3H), 1.84 (d, J=13.1 Hz, 2H), 2.01 (d, J=13.4 Hz, 2H), 2.64-2.70 (m, 2H), 2.93 (m, 1H), 8.05 (brs, 3H).

### Step 5: 6-Oxo-2-{[4-(1H-tetrazol-5-yl)-cyclohexylmethyl]-amino-}4-thiophen-2-yl-1,6-dihydro-pyrimidine-5-carbonitrile (Compound I-42)

To a stirred solution of intermediate 62.6 (250 mg, 0.88 mmol) in DMSO (5 mL) was added DIPEA (0.3 mL, 1.76 mmol) and intermediate 62.5 (211 mg, 0.97 mmol). Stirring was continued at 80° C. 4 h. The crude was poured in water, acidified to pH 3 and extracted with EtOAc (3×20 mL). The crude product from the reaction was purified by flash chromatography eluting with DCM/MeOH (6% for product). The title compound 1-42 (24 mg, 0.06 mmol) was obtained as yellowish solid. Yield 7%. MS-ESI (−) m/z: 381.4 (M−H). HPLC: 88%

### Example 63: Preparation of Intermediate 1b.3

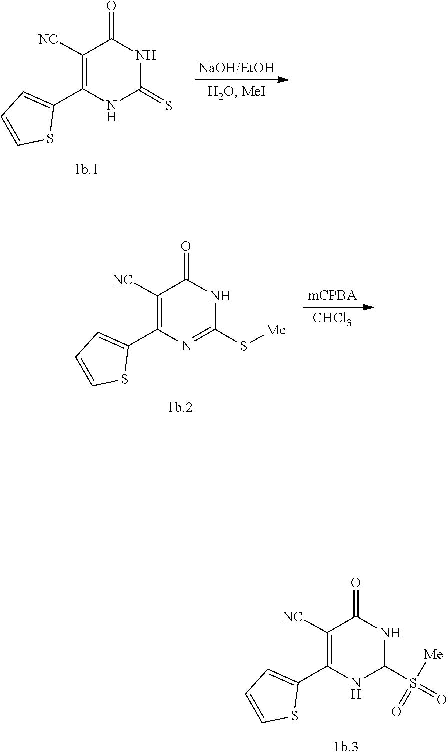

### Step 1: Preparation of Intermediate 1b.2

To a stirred solution of compound 1b.2 (500 mg, 2.13 mmol), in a mixture of H₂O/EtOH (2 mL+4 mL) was added NaOH (85 mg, 2.13 mmol) and MeI (0.12 mL, 2.13 mmol). Stirring was continued at 60° C. for 30 minutes. The title compound 1b.2 was collected as yellow powder upon filtration from the reaction medium (491 mg, 4.9 mmol). Yield 92%.

### Step 2: Preparation of Intermediate 1b.3

To a stirred solution of compound 1b.2 (200 mg, 0.8 mmol), in CHCl₃ (6 mL) was added mCPBA (207 mg, 1.2 mmol). Stirring was continued at rt for 16 h. The yellow solid was collected and washed with DCM and Et₂O. The title compound 1b.3 was obtained (190 mg, 0.67 mmol) as a pale yellow solid. Yield 84%.

### Example 64: Preparation of Intermediate 2b.2

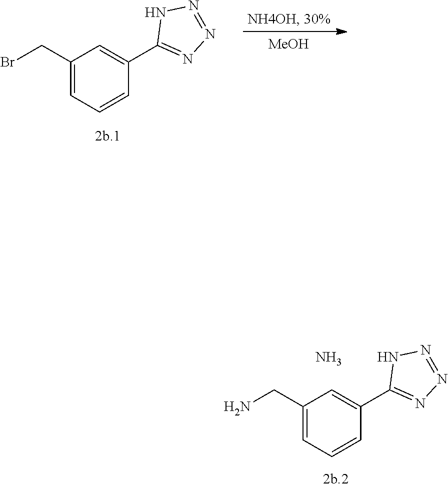

### Step 1: Preparation of Intermediate 2b.2

To a solution of the starting intermediate 2b.1 (200 mg, 0.83 mmol) in MeOH (3 mL) was added a 30% ammonia solution in water (2 mL). Stirring was continued at rt for 24 h. The solvent was then removed under vacuo. The title intermediate 2b.2 (143 mg, 0.74 mmol) was obtained as yellowish powder without further purifications. Yield 90%.

### Example 65: Preparation of Intermediate 3b.6

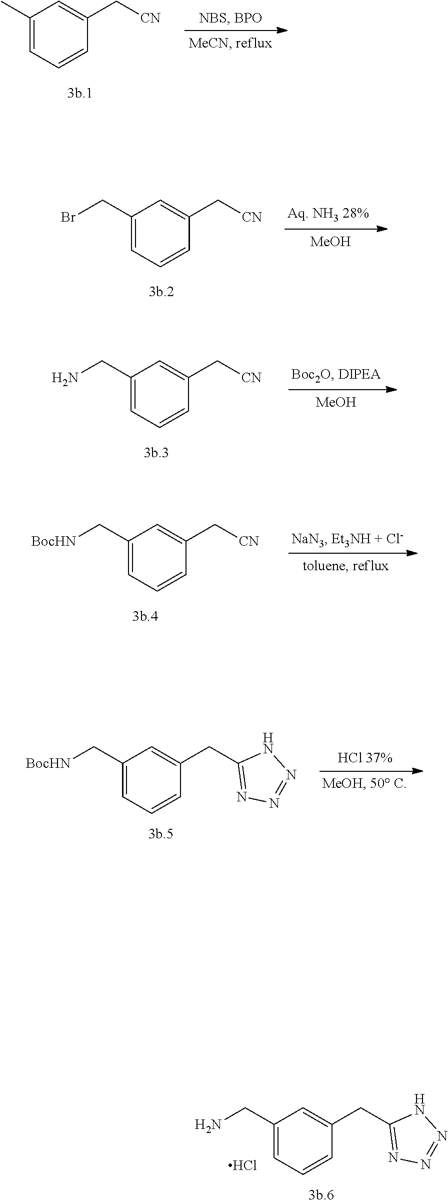

### Step 1: Preparation of Intermediate 3b.2

To a stirred and boiling solution of 3b.1 (1.00 g, 7.62 mmol) in MeCN (30 mL), a solution of NBS (1.42 g) and BPO 70% (13 mg, 0.04 mmol) in MeCN (10 mL) was added dropwise. After 30 min the mixture was slowly cooled to r.t. and poured in aq. NaHCO₃ ss (15 mL). The mixture was extracted with AcOEt (3×30 mL), washed with brine (50 mL), dried over Na₂SO₄, and concentrated under reduced pressure. The title intermediate 3b.2 was used directly (1.60 g) for the next step.

### Step 2: Preparation of Intermediate 3b.3

A solution of intermediate 3b.2 (1.60 g, crude of previous step) in MeOH (15 mL) was treated with aq. NH₃ 28% (15 mL), and the resulting mixture was stirred for 16 h. Volatiles were removed under reduced pressure and the crude was poured in H₂O (30 mL) and washed with AcOEt (3×30 mL). The aqueous phase was concentrated under reduced pressure. The title intermediate 3b.3 (1.17 g) was used as crude for the next step.

### Step 3: Preparation of Intermediate 3b.4

A stirred suspension of intermediate 3b.3 (1.17 g, crude of previous step) in CH₂Cl₂ (30 mL), Boc₂O (1.99 g, 9.14 mmol) and DIPEA (3.32 mL, 19.06 mmol) were added, and the mixture was reacted at r.t. for 2 h obtaining an opalescent solution. H₂O (20 mL) was added, the two phases were separated and the organic one was washed with aq. citric acid 0.5 M (2×20 mL), H₂O (20 mL), brine (20 mL), dried over Na₂SO₄ and concentrated under reduced pressure. The crude was purified by chromatographic purification (Petroleum ether/AcOEt from 9:1 to 7:3) to give the title intermediate 3b.4 as dense oil in 28% yield starting from 3.1. MS-ESI (+) m/z: 247.2 (M+H).

### Step 4: Preparation of Intermediate 3b.5

A mixture of intermediate 3b.4 (525 mg, 2.13 mmol), sodium azide (415 mg, 6.39 mmol) and triethylammonium chloride (880 mg, 6.39 mmol) in toluene (40 mL) was stirred and refluxed for 18 h. Once cooled at r.t., aq. NaHCO₃ ss (15 mL) was added, and the mixture was vigorously stirred for 10 min. The two phases were separated and the organic one was extracted with H₂O (3×30). All the aqueous phases were collected together and acidified up to pH=3 by adding citric acid 0.5 M. The resulting acid aqueous phase was extracted with CH₂Cl₂ (3×50 mL), washed with), brine (50 mL), dried over Na₂SO₄ and concentrated under reduced pressure. The title intermediate 3b.5 (214 mg, 0.74 mmol) was obtained as white solid. Yield: 35%. MS-ESI (−) m/z: 288.2 (M−H).

### Step 5: Preparation of Intermediate 3b.6

Intermediate 3b.5 (205 mg, 0.71 mmol) was dissolved in MeOH (10 mL) and treated with HCl 37% (0.29 mL, 3.35 mmol) at 50° C. for 2 h. The volatiles were removed under reduced pressure, to afford intermediate 3b.6 in nearly quantitative yield as hydrochloride salt. MS-ESI (+) m/z: 190.3 (M+H); MS-ESI (−) m/z: 188.2 (M−H).

### Example 66: Preparation of Intermediate 4b.4

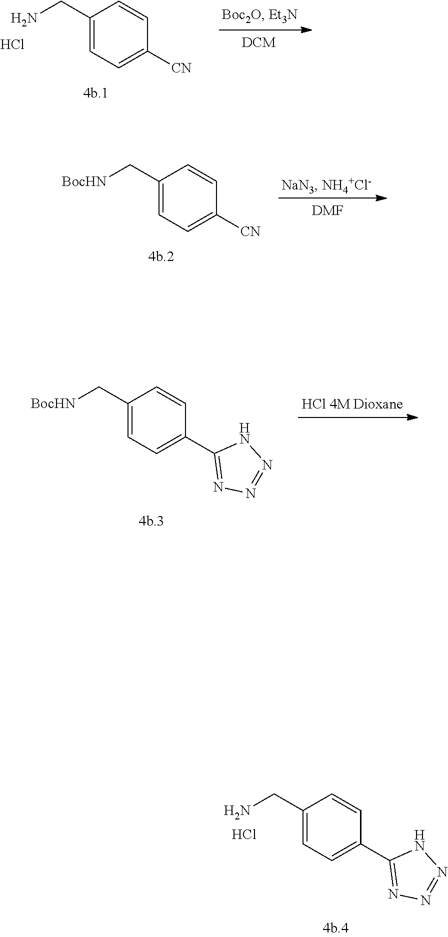

### Step 1: Preparation of Intermediate 4b.2

To a solution of intermediate 4b.1 (2 g, 11.86 mmol) in DCM (20 mL) was added TEA (1.99 mL, 14.2 mmol) and BOC₂O (2.717 g, 12.45 mmol). Stirring was continued at rt 5 h. The crude was poured in water and was extracted with DCM. The organic phase were washed with brine and dried over Na₂SO₄. The title intermediate 4b.2 (2 g, 8.61 mmol) was obtained as white solid. Yield 72%.

### Step 2: Preparation of Intermediate 4b.3

To a solution of the starting intermediate 4b.2 (2 g, 7.26 mmol) in DMF (6 mL) was added NaN₃ (839 mg, 12.91 mmol) and ammonium chloride (689 mg, 12.91 mmol). Stirring was continued at 140° C. for 6 h. The crude was poured in water and brine followed by extraction with EtOAc at pH=3. The organic phase were dried over Na₂SO₄ and evaporated under vacuo. The title intermediate 4b.3 (2.2 g, 8.0 mmol) was obtained as white solid. Yield 93%.

### Step 3: Preparation of Intermediate 4b.4

The starting intermediate 4b.3 (1.5 g, 5.48 mmol) was stirred overnight in a 4 M dioxane solution of HCl (10 mL). The solvent was removed under vacuo. The title intermediate 4b.4 (1.14 g, 5.38 mmol) was obtained as a white solid. Yield 98%.

### Example 67: Preparation of Intermediate 5b.2

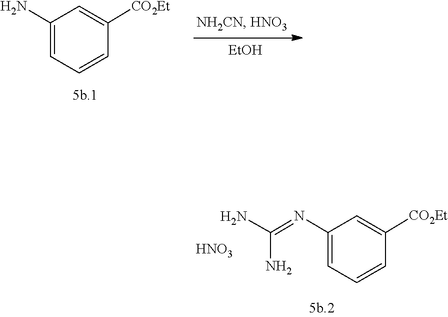 [SMILES: "EtOH" ; CCOC(=O)c1cccc(N)c1 ; "HNO3" ; CCOC(=O)c1cccc(N=C(N)N)c1]

To a solution of intermediate 5b.1 (1 g, 6.1 mmol) in EtOH (10 mL) was added NH₂CN (0.7 mL, 9.1 mmol) and HNO₃ (0.25 mL, 6.1 mmol). Stirring was continued at reflux for 16 h. The mixture was cooled to 0° C. and was added of Et₂O. The white precipitate was collected. The title intermediate 5b.2 (1 g, 3.7 mmol) was thus obtained as white solid as HNO₃ salt. Yield 60%.

### Example 68: Preparation of Intermediate 6b.2

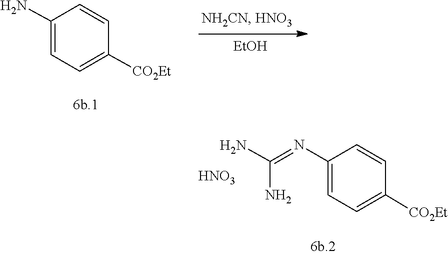 [SMILES: "EtOH" ; CCOC(=O)c1ccc(N)cc1 ; "HNO3" ; CCOC(=O)c1ccc(N=C(N)N)cc1]

To a solution of intermediate 6b.1 (1 g, 6.1 mmol) in EtOH (10 mL) was added NH₂CN (0.7 mL, 9.1 mmol) and HNO₃ (0.25 mL, 6.1 mmol). Stirring was continued at reflux for 16 h. The mixture was cooled to 0° C. and was added of Et₂O. The white precipitate was collected. The title intermediate 6b.2 (1.2 g, 4.4 mmol) was obtained as yellowish solid asHNO₃ salt. Yield 72%.

### Example 69: Preparation of Compound I-43

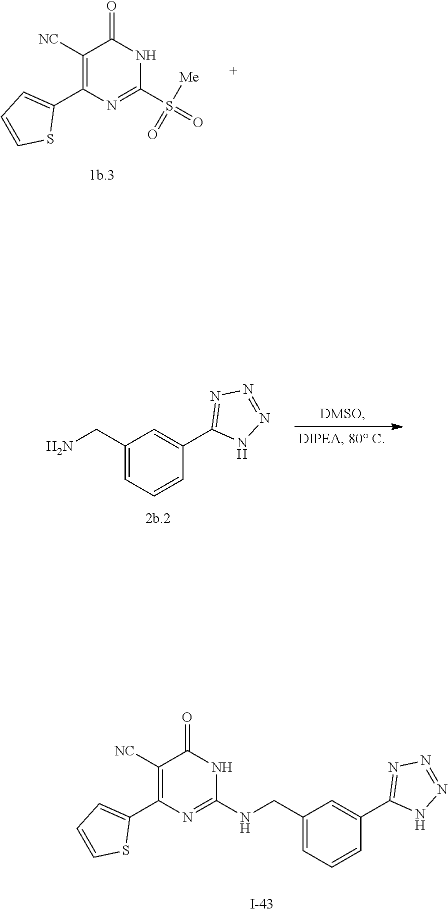

To a stirred suspension of intermediate 1b.3 (200 mg, 0.85 mmol) in DMSO (5 mL) was added DIPEA (0.29 mL, 1.7 mmol) and intermediate 2b.2 (164 mg, 0.94 mmol). Stirring was continued at 80° C. for 4 h. The crude was poured in water, acidified to pH 3 and extracted with EtOAc (3×20 mL). The crude of reaction was purified by flash chromatography eluting with DCM/MeOH (3% for product). The title compound I-43 (50 mg, 0.13 mmol) was obtained as yellowish solid. Yield 15%. ¹H NMR (400 MHz, DMSOd₆) δ 4.69 (2H), 7.25 (m, 1H), 7.58 (m, 2H), 7.91 (m, 2H), 8.0 (s, 1H), 8.16 (m, 1H), 11.94 (brs, 1H); ¹³C NMR (100 MHz, DMSOd₆) δ 44.2, 81.8, 117.8, 124.6, 126.1, 126.3, 129.3, 129.9, 130.7, 130.9, 130.9, 133.9, 140.5, 141.1, 154.4, 161.6, 162.1. HPLC 96.3%.

### Example 70: Preparation of Compound I-44

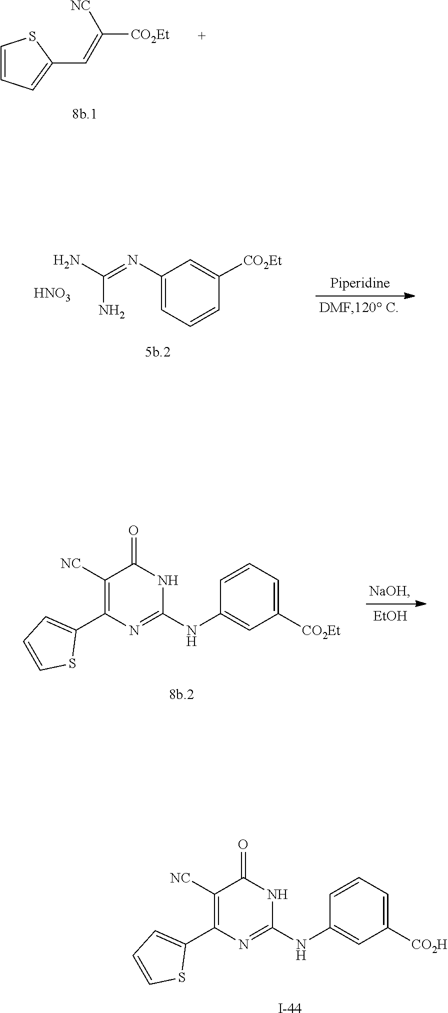

### Step 1: Synthesis of Intermediate 8b.2

A solution of intermediate 8b.1 (195 mg, 0.72 mmol) and intermediate 5b.2 (150 mg, 0.72 mmol) in DMF (2 mL), was added of piperidine (0.14 mL, 1.45 mmol). The mixture was sealed in a Q-tube apparatus and heated at 120° C. 16 h. The mixture was cooled to rt, poured in water, extracted with EtOAc, and purified by flash chromatography eluting with DCM/MeOH (5% for product). The title intermediate 8b.2 (200 mg, 0.39 mmol) has been obtained as brownish powder. Yield 54%

### Step 2: Synthesis of Compound I-54

To a solution of intermediate 8b.2 (150 mg, 0.41 mmol) in EtOH (10 mL) was added a 1 M solution of NaOH (1.2 mL). Stirring was continued at reflux gently for 16 h. The precipitate was collected by filtration and it was dissolved in water. The pH was adjusted to 3 by the addition of 3N HCl solution. The precipitate was collected and dried under vacuo to give the title compound I-44 (100 mg, 0.295 mmol) as a brown solid. Yield 72%. ¹H NMR (400 MHz, DMSOd₆) δ 7.31 (t, J 4.22 Hz, 1H), 7.50 (t, J 7.8 Hz, 1H), 7.72 (d, J=7.48 Hz, 1H), 7.88 (d, J=7.4 Hz, 1H), 7.99 (d, J=4.7 Hz, 1H), 8.25 (m, 2H), 10.1 (s, 1H), 11.9 (brs, 1H); ¹³C NMR (100 MHz, DMSOd₆) δ 83.5, 117.4, 122.4, 125.3, 125.7, 129.5, 129.6, 131.2, 131.8, 134.3, 137.9, 140.8, 152.5, 161.4, 161.9, 167.3. HPLC: 98.4%

### Example 71: Preparation of Compound I-45

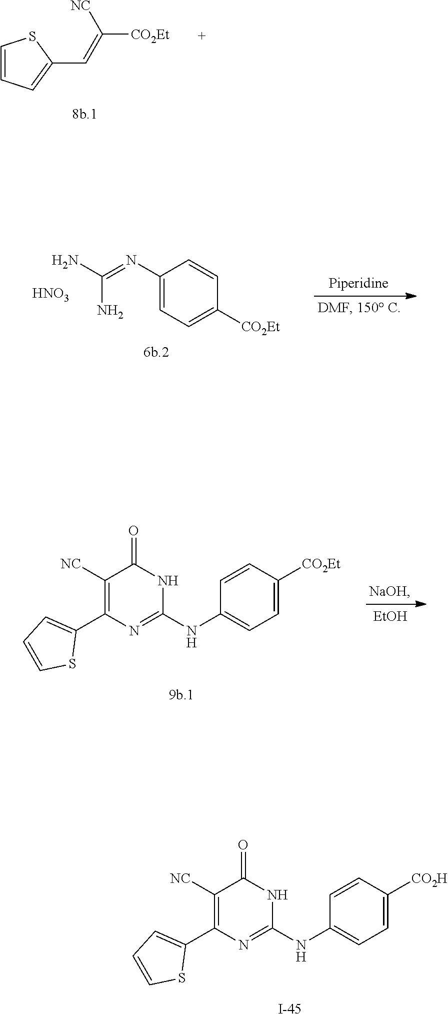

### Step 1: Synthesis of Intermediate 9b.1

A solution of intermediate 8b.1 (990 mg, 0.72 mmol) and intermediate 6b.2 (1.18 g, 4.34 mmol) in DMF (10 mL), was added of piperidine (0.86 mL, 8.68 mmol). The mixture was heated at 150° C. for 16 h. The mixture was cooled to rt, poured in water, the pH was adjusted to 3 by the addition of HCl (3N). The solid was collected and washed with acetone. The title intermediate 9b.1 (250 mg, 0.68 mmol) has been obtained as grey powder. Yield 14%

### Step 2: Synthesis of Compound I-45

To a solution of intermediate 9b.1 (150 mg, 0.41 mmol) in EtOH (10 mL) was added a 1 M solution of NaOH (1.2 mL). Stirring was continued at reflux gently 16 h. The solvent was removed under vacuo. The solid was suspended in EtOH (5 mL) sonicated and filtered. The sodium salt was dissolved in water and ice, pH was adjusted to 3 by the addition of 3N HCl solution. The gummy precipitate was collected and dried under vacuo to give the title compound I-45 (90 mg, 0.21 mmol) brown solid. Yield 65%. ¹H NMR (400 MHz, DMSOd₆) δ 7.32 (d, J=4.1 Hz, 1H), 7.79 (d, J=8.6 Hz, 2H), 7.96 (d, J=8.6 Hz, 2H), 8.0 (d, J=4.9 Hz, 1H), 8.24 (d, J=3.8 Hz, 1H), 10.23 (s, 1H), 11.9 (brs, 1H); ¹³C NMR (100 MHz, DMSOd₆) δ 83.9, 117.2, 120.5, 120.5, 126.2, 129.7, 130.7, 130.7, 131.3, 134.5, 140.7, 141.9, 152.5, 161.3, 167.2; HPLC: 95.56%.

### Example 72: Preparation of Compound I-46

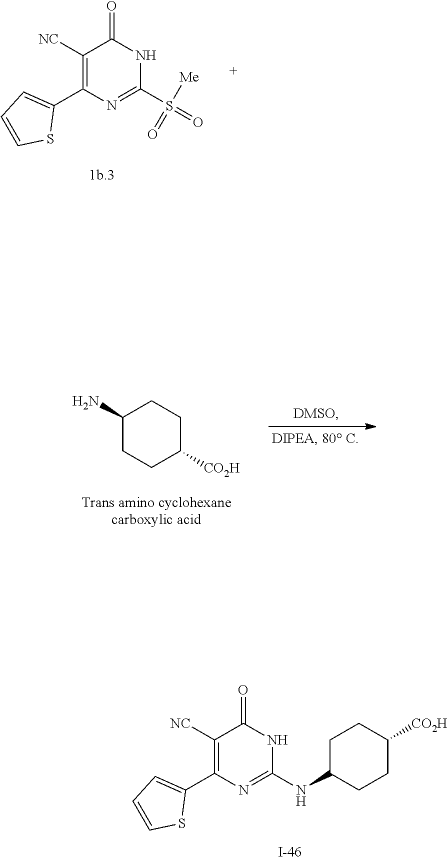

To a solution of intermediate 1b.3 (100 mg, 0.36 mmol) in DMSO (3 mL) was added 1,4-trans amino cyclohexane (51 mg, 0.36 mmol). Stirring was continued at 80° C. for 16 h. The mixture was poured in water, the pH was adjusted to 3 by the addition of HCl (3N solution). The gummy precipitate was collected and dried under vacuo. The crude was purified by flash chromatography eluting with DCM/MeOH 15% and acetone due to the low compound solubility. The title compound I-46 (40 mg, 0.12 mmol) was obtained as yellowish powder. Yield 32%. ¹H NMR (200 MHz, DMSOd₆) δ 1.34 (m, 4H), 1.92 (m, 4H), 2.20 (brs, 1H), 3.73 (brs, 1H), 7.27 (t, J=4.2 Hz, 1H), 7.61 (s, 1H), 7.91 (d, J 4.9 Hz, 1H), 8.15 (m, 1H), 12.0 (brs, 1H). ¹³C NMR (100 MHz, DMSOd₆) δ 27.8, 27.8, 31.1, 31.1, 41.7, 41.7, 50.2, 81.05, 118.0, 129.4, 130.7, 133.9, 141.3, 153.9, 161.6, 176.8; HPLC: 96.3%.

### Example 73: Preparation of Compound I-47

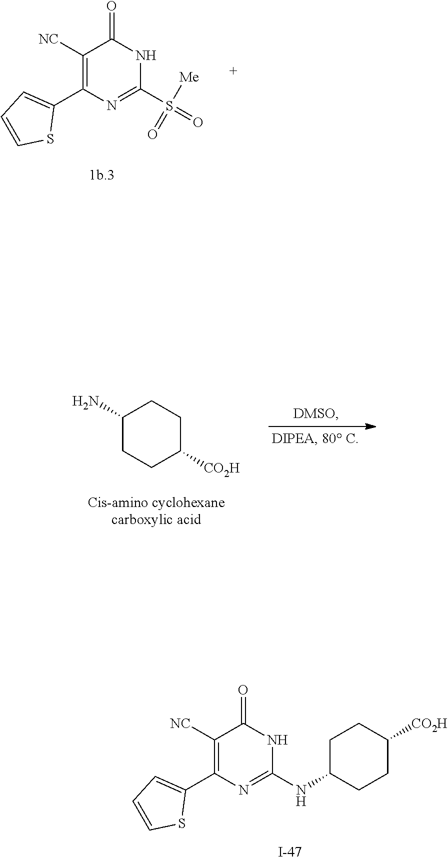

To a solution of intermediate 1b.3 (150 mg, 0.53 mmol) in DMSO (5 mL) was added 1,4-cis amino cyclohexane (76 mg, 0.53 mmol). Stirring was continued at 80° C. for 16 h. Ice was added to the mixture under stirring. The white solid was collected and then purified by flash chromatography, eluting with DCM/MeOH. The title compound I-47 (70 mg, 0.2 mmol) was obtained as white solid. Yield 38%. ¹H NMR (400 MHz, DMSOd₆) δ 1.71 (m, 8H), 2.4 (brs, 1H), 4.05 (s, 1H), 7.28 (t, J=4.2 Hz, 1H), 7.3 (m, 1H), 7.93 (d, J=4.6 Hz, 1H), 8.18 (d, J=3.23 Hz, 1H), 10.9 (brs, 1H), 12.1 (brs, 1H); ¹³C NMR (100 MHz, DMSOd₆) δ 24.6, 28.8, 47.6, 48.9, 55.3, 55.3, 81.2, 117.8, 129.4, 130.8, 133.9, 141.2, 153.6, 161.6, 161.7, 176.5; HPLC: 98.51%.

### Example 74: Preparation of Compound I-48

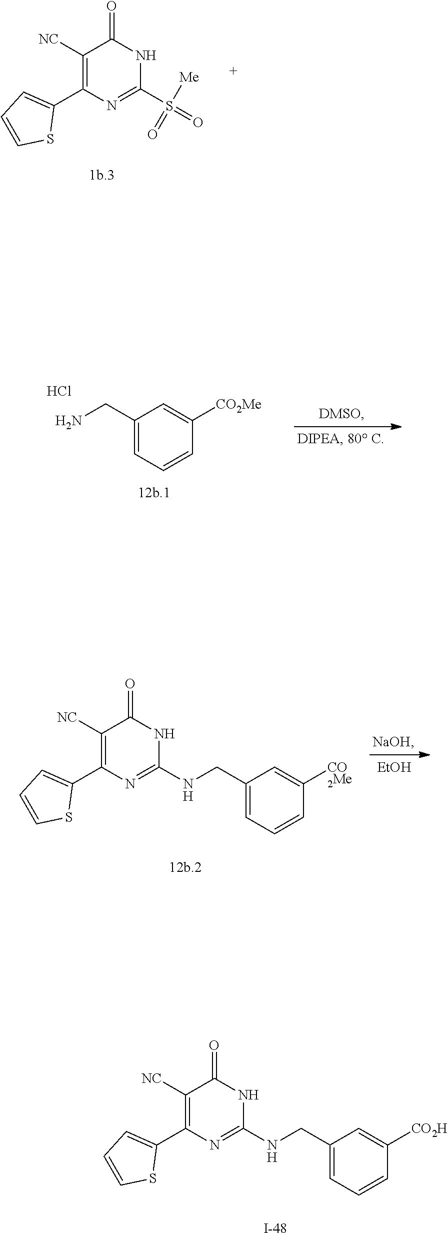

### Step 1: Synthesis of Intermediate 12b.2

To a solution of the starting intermediate 1b.3 (400 mg, 1.42 mmol) in DMSO (5 mL) was added DIPEA (0.36 mL, 2.13 mmol) and intermediate 12b.1 (344 mg, 1.7 mmol). Stirring was continued at 80° C. for 16 h. The crude was poured in water. The solution was adjusted to pH3 by the addition of HCl (3N solution). The aqueous phase was extracted with EtOAc. The mixture was purified by flash chromatography, eluting with DCM/MeOH. The title intermediate 12b.2 (200 mg, 0.54 mmol) was obtained as yellowish powder. Yield 38%.

### Step 2: Synthesis of Compound I-48

To a solution of intermediate 12b.2 (200 mg, 0.52 mmol) in MeOH (15 mL) was added a 1 M solution of NaOH (3 mL). Stirring was continued at reflux gently for 16 h. The solvent was removed under vacuo. The sodium salt was dissolved in water and ice, pH was adjusted to 3 by the addition of 3N HCl solution. The gelly precipitate was collected and dried under vacuo to give the title compound I-48 (150 mg, 0.42 mmol) as yellowish solid after trituration with Et₂O. Yield 82%. MS-ESI (+) m/z: 353.3 (M+H).

### Example 75: Preparation of Compound I-49

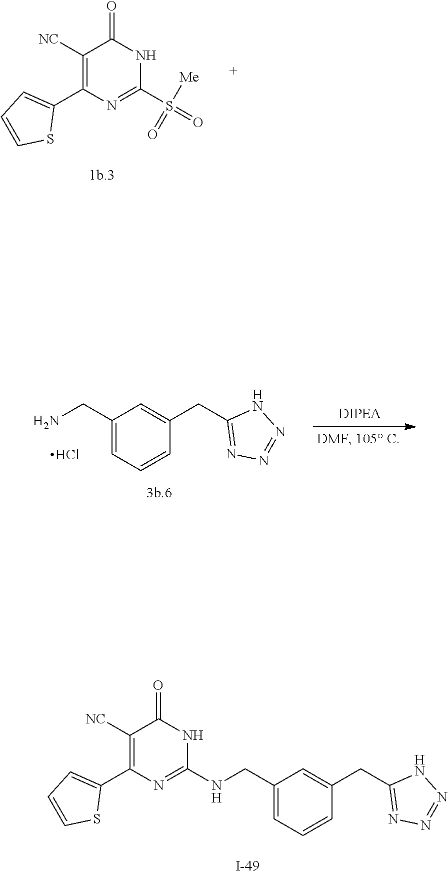

To a stirred solution of Intermediate 1b.3 (150 mg, 0.53 mmol) in DMF (10 mL), Intermediate 3b.6 (120 mg, 0.53 mmol) and DIPEA (0.46 mL, 2.65 mmol) were added, and the mixture was reacted at 105° C. for 6 h. Once cooled at r.t. it was poured in H₂O (25 mL) and washed with Et₂O (2×20 mL). HCl 3.0 M was added to the aqueous solution up to pH=1 and the mixture was extracted with CH₂Cl₂/MeOH 9:1 (vol/vol, 3×30 mL). The collected organic phases were concentrated under reduced pressure, and the crude was purified by RP-flash chromatography (H₂O/MeCN from 8:2 to 1:9). The collected impure compound (15 mg) was tritured with cold acetone, to afford 10 mg of pure Compound I-49 (Yield: 5%). MS-ESI (−) m/z: 389.4 (M−H). ¹H NMR (400 MHz, DMSOd₆) δ 4.25 (s, 2H), 4.55 (d, J=4.74 Hz, 2H), 7.17 (s, 1H), 7.26 (m, 4H), 7.91 (d, J=4.8 Hz, 1H), 7.94 (brs, 1H), 8.16 (d, J=3.3 Hz, 1H); ¹³C NMR (100 MHz, DMSOd₆) δ 29.4, 44.3, 81.5, 117.9, 126.5, 127.9, 128.2, 129.1, 129.3, 130.8, 133.9, 136.8, 139.4, 141.1, 154.6, 155.8, 161.6, 162.4; HPLC: 99.5%.

### Example 76: Preparation of Compound I-50

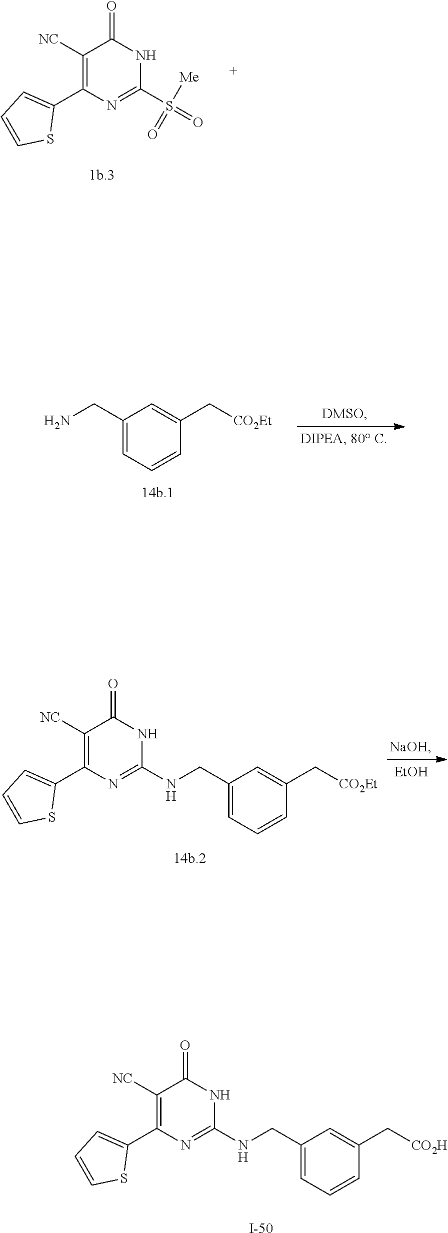

### Step 1: Synthesis of Intermediate 14b.2

To a solution of intermediate 1b.3 (200 mg, 0.71 mmol) in DMSO (5 mL) was added DIPEA (0.18 mL, 1.07 mmol) and intermediate 14b.1 (164 mg, 0.85 mmol). Stirring was continued at 80° C. for 16 h. The mixture was poured in water and extracted with EtOAc (3×20 mL). The mixture was purified by flash chromatography eluting with DCM/MeOH 1.5% for product. The title intermediate 14b.2 (173 mg, 0.43 mmol) was obtained as yellow solid. Yield 62%. ¹H NMR (400 MHz, DMSOd₆) δ 1.13 (t, J=7.1 Hz, 3H), 3.63 (s, 2H), 4.02 (q, J=7.0 Hz, 2H), 4.56 (d, J=5.8 Hz, 2H), 7.16 (m, 1H), 7.27 (m, 4H), 7.93 (m, 1H), 7.94 (d, J=4.0 Hz, 1H), 8.17 (d, J=3.8 Hz, 1H), 11.91 (brs, 1H).

### Step 2: Synthesis of Compound I-50

To a solution of intermediate 14b.2 (165 mg, 0.42 mmol) in EtOH (6 mL) was added a 1 M solution of NaOH (1.3 mL). Stirring was continued at reflux gently 16 h. The mixture was cooled to room temperature and the precipitate was collected. The sodium salt was dissolved in water and ice, pH was adjusted to 1 by the addition of 3N HCl solution. The precipitate was collected and dried under vacuo to give the title compound I-50 (75 mg, 0.21 mmol) as white solid. Yield 49%. ¹H NMR (400 MHz, DMSOd₆) δ 3.55 (s, 2H), 4.57 (d, J=5.8 Hz, 2H), 7.16 (m, 1H), 7.27 (m, 4H), 7.84 (m, 1H), 7.92 (d, J=4.5 Hz, 1H), 8.17 (d, J=3.7 Hz, 1H), 11.73 (brs, 1H), 12.49 (brs, 1H); ¹³C NMR (100 MHz, DMSOd₆) δ 41.0, 44.3, 81.6, 117.8, 126.3, 128.7, 129.1, 129.1, 129.3, 130.9, 134.0, 135.5, 138.8, 141.1, 154.3, 161.7, 161.9, 173.0; HPLC: 95.2%.

### Example 77: Preparation of Compound I-51

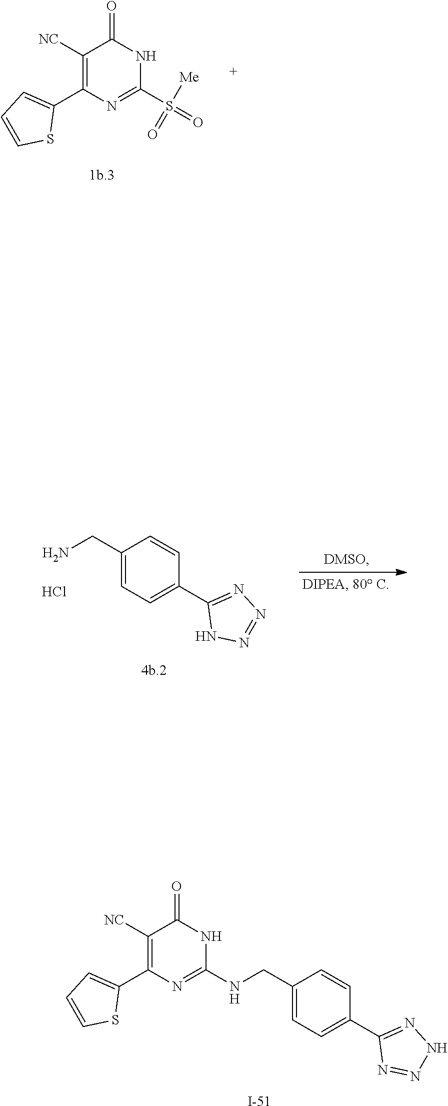

To a solution of intermediate 4b.2 (211.6 mg, 1 mmol) in DMSO (5 mL) was added DIPEA and stirring was continued at rt 10 minutes. Then a solution of compound 1b.3 in DMSO was added. Stirring was continued at 90° C. for 16 h. The crude was collected, dried and purified by flash chromatography eluting with DCM/MeOH-6% for product. The title compound I-51 was obtained (25 mg, 0.06 mmol) as yellow solid after trituration with EtOAc. Yield 7%. ¹H NMR (400 MHz, DMSOd₆) δ 4.68 (d, J=5.7 Hz, 2H), 7.27 (t, J 4.1 Hz, 1H), 7.60 (d, J=7.7 Hz, 2H), 7.9 (d, J=4.9 Hz, 1H), 8.0 (d, J=7.7 Hz, 2H), 8.18 (d, J=3.6 Hz, 1H), 12.1 (brs, 1H); ¹³C NMR (100 MHz, DMSOd₆) δ 44.2, 53.9, 81.7, 117.8, 123.6, 127.4, 127.4, 128.7, 129.3, 130.9, 134.0, 141.0, 142.2, 154.5, 155.7, 161.6, 162.1; HPLC: 96.6%.

### ACMSD and Acute Inflammation

### Cellular Assays

Assay Protocol for Mouse Kupffer Cell Transfection with ACMSD Plasmid and Cytokine Analysis after LPS Induction

Kupffer cells (immortalized mouse Kupffer cell line, (cat #SCC119 (ImKC) Merck Millipore) were plated out in a tissue culture 24 well plate with 150,000 cells/well in RPMI medium+10% FBS. At 24 h after plating, the cells were transfected with Fugene HD (Promega), pCDNA3.1 mouse ACMSD, and pCDNA3.1 empty vectors each at the concentration of 0.75 μg/well for 18 hrs.

Cell stimulation with the ACMSD inhibitor (e.g. compounds of present disclosure, compound of Formula I and II) was performed using the following concentrations of test compound, 0.5, 5, and 50 μM, with cells where only DMSO at the final concentration of 0.5% was added used as control. All the wells were normalized with DMSO at 0.5% final concentration. Cells were treated with final concentration of 50 ng/ml of LPS in cell medium for 18 hrs, the supernatants were then collected for analysis of mouse cytokine secretion (IL-1α, IL1β, IL-2, IL-3, IL-4, IL-5, IL6, IL-9, IL-10, IL-12 (p40), IL-12 (p70), IL-13, IL-17, IFN-γ, Rantes, Eotaxin, MCP-1, MIP-1α, MIP-1β, G-CSF, GM-CSF, TNFα, and KC (Keratinocyte chemoattractant)) using the Bio-Plex Pro mouse cytokine 23-plex assay (cat #M60009RDPD). The cells were centrifuged and the resulting pellets were collected for either RT PCR analysis or ATP measure (Promega Cell-Titer-Glo cat #G7571).

### Assay Protocol for ACMSD Silencing

Kupffer cells plated out in a 24 well plate were transfected with 5 pmol of either ACMSD siRNA or scrambled siRNA as a negative control (siRNA ACMSD cat #4390771 and scrambled siRNA cat #4390843, both Ambion). After 18 hrs of incubation the cells were treated with the ACMSD inhibitor (e.g. compounds of present disclosure, compound of Formula I and II) (5 μM) or DMSO as vehicle, and then after an additional 1 hr with final concentration of 50 ng/ml of LPS in cell medium, followed by overnight incubation before cytokine secretion was then measured using the Bio-Plex kit. All the wells were normalized with DMSO at 0.5% final concentration.

### Cytokine Secretion Measurement

200,000 Kupffer cells were plated out in a 24 well plate and the ACMSD inhibitor (e.g. compounds of present disclosure, compound of Formula I and II) (5 μM) was added 1 hr before the cells were stimulated with LPS overnight song/ml (1 mg/mL stock solution in H₂O, dispensed at 50 ng/mL in the well) final concentration. Cells treated with DMSO were used as control at the final concentration of 0.5% in medium. All the wells were normalized with DMSO at 0.5% final concentration. The supernatant was collected to measure cytokine secretion using a Bio-Plex Pro mouse cytokine 23-plex assay system (cat #M60009RDPD) using a Bio-Plex Instrument. The cells were washed once with PBS, and then lysed to extract total RNA for performing RT-PCR to evaluate ACMSD modulation of IL-10, SIRT1, and STAT3 gene expression.

Briefly, total RNA was extracted from the cells using the manufacturer's protocol for Qiagen RNeasy Plus Mini Kit (cat. 74134). The total RNA concentration was quantified using an Implen Instrument, and the purity was evaluated by measuring the ratio of A260/A280. Isolated RNA had an A260/A280 ratio of (1.8-2.0 range).

Total RNA (1 μg) was reverse transcribed using the manufacturer's protocol for SuperScript IV VILO Master Mix (ThermoFisher cat. #. 11756500). Real-time PCR was then performed in CFX 96 Real Time System (Bio-Rad) as follows: denaturation at 95° C. for 2 minutes, followed by 40 cycles at 95° C. for 10 s, and hybridization at 60° C. for 20 s. QPCR was performed using the 2× QuantiNova SYBR Green PCR Master Mix (Qiagen). Mouse β2 microglobulin (m β2M) was used as a reference gene.

All cytokine secretion values were normalized to the luminescent signal relative to the measuring of the cellular ATP (CellTiter-Glo cat #G7571)).

### Primer Sequences

mSTAT3_FW CACATGCCACGTTGGTGTTT
mSTAT3_RW ACGATCCGGGCAATTTCCAT
mIL10_FW CAGTACAGCCGGGAAGACAAT
mIL10_RW TTGGCAACCCAAGTAACCCT
mSIRT1_FW TATCTATGCTCGCCTTGCGG
mSIRT1_RW GACACAGAGACGGCTGGAAC
mACMSD_FW GCAGATGGATGGACGAATGG
mACMSD_RW CGAAGCACACTTTGAGTTTGG
mB2M_FW CTCGGTGACCCTGGTCTTTC
mB2M_RW GGATTTCAATGTGAGGCGGG

### Human Liver Microtissues (Organoids) Protocol

3D InSight™ Human Liver Microtissues (organoids) composed of multi-donor primary human hepatocytes in co-culture with Non-Parenchymal Cells (NPC), containing primary human Kupffer cells and primary Liver Endothelial Cells (LEC) were provided by Insphero, Switzerland. These human liver organoids to evaluate the anti-inflammatory effect of ACMSD inhibition.

The organoids were treated with free fatty acids (palmitic acid and oleic acid according to manufacturer's instructions) and lipopolisaccaride (LPS) in a diabetic medium (Insphero) at the concentration of 10 μg/mL to induce lipid accumulation in hepatocytes and pro-inflammatory cytokine secretion including; TNF-α, IL-6, IL-8, MCP-1, and MIP1α. Vehicle control solution was prepared as negative control. DMSO was maintained constant concentration of 0.1% total solution.

Two concentrations of compound I-34 (10 μM and 50 μM) were used and added together with the NASH stimuli mix. Cytokine secretion was measured in cell medium at day 4 following treatment using the Bio-Plex Pro Human Cytokine Screening Panel (Bio-Rad) according to manufacturer's instructions.

ACMSD Inhibition Protects Cells from Inflammatory Damage in Kupffer Cells

Following inflammation induced by LPS (Kupffer cells treated with LPS 50 ng/mL 16-18° C. incubation time) ACMSD inhibition, with compound I-34, reverses the changes in the expression of inflammatory modulators. Expression of pro-inflammatory genes is reduced (TNF-α, IL-6, and iNOS) and that of anti-inflammatory genes is increased (Arg-1, IL-10, and MRC2) by ACMSD inhibition.

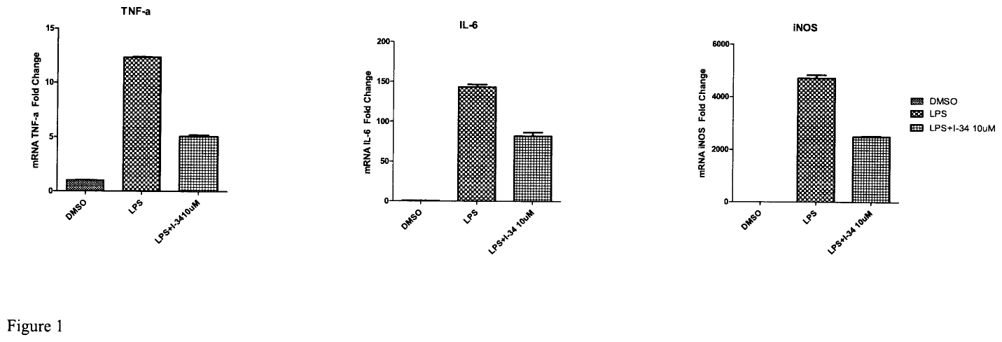

FIG. 1 shows the fold reduction in pro-inflammatory gene expression in Kupffer cells treated with LPS (50 ng/mL) followed by inhibition of ACMSD with compound I-34 (10 μM).

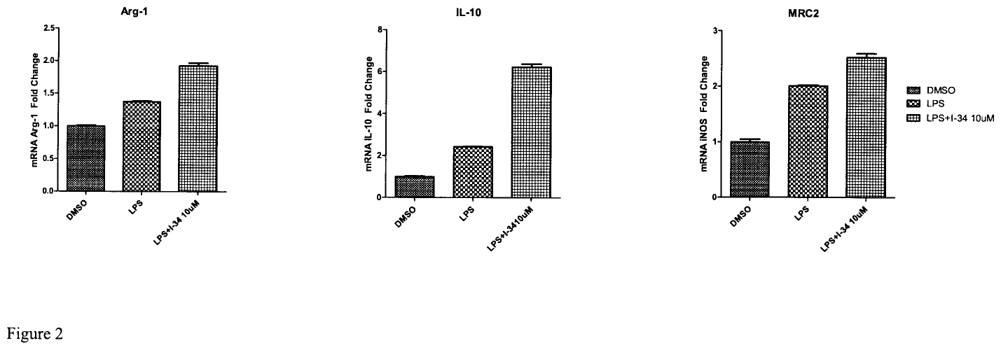

FIG. 2 shows increase of anti-inflammatory gene expression. FIG. 2 shows the fold increase in anti-inflammatory gene expression in Kupffer cells treated with LPS (50 ng/mL) followed by inhibition of ACMSD with compound I-34 (10 μM).

Overall these data support a shift from an M1 to and an M2 macrophage phenotype.

Modulation of QPRT Gene Expression, a Downstream Gene from ACMSD in the NAD⁺ Biosynthesis Pathway, in HK-2 and Kupffer Cells

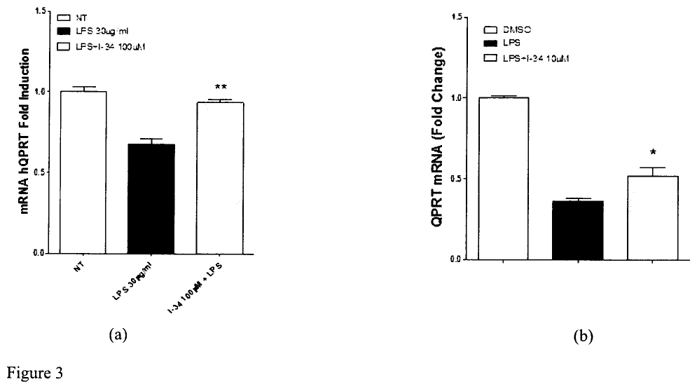

FIG. 3 shows that ACMSD inhibition, with compound I-34, reversed LPS-induced reduction of QPRT expression in both HK-2 cells at 100 μM and Kupffer cells at 10 μM. FIG. 3 shows (a) fold change in expression of QPRT measured in HK-2 cells treated with LPS (30 mg/ml) for 18 h, in the presence of LPS and LPS with I-34; and (b) fold change in expression of QPRT in Kupffer cells incubated in the absence (DMSO) and in the presence of LPS and LPS with I-34. ((N=3) One way Anova: Dunnett's test: **p<0.01 vs pathological control.)

ACMSD inhibition, with compound I-34, reversed LPS-induced reduction of QPRT expression in cells following an 18 h stimulus with 30 μg/ml of LPS but does not significantly affect ACMSD expression under the same conditions.

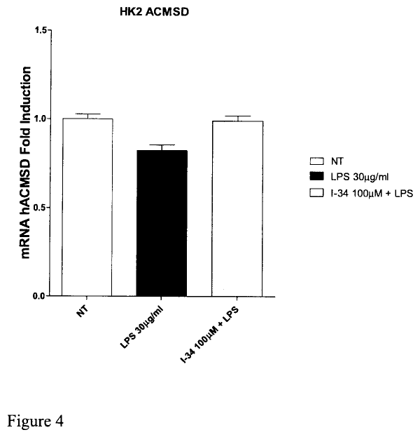

FIG. 4 shows fold change in expression of ACMSD in HK-2 cells treated with LPS (30 μg/ml) followed by treatment with an ACMSD inhibitor (compound I-34 100 μM).

### ACMSD Inhibition Reduces Inflammatory Gene Expression in HK-2 Cells

ACMSD inhibition, with compound I-34, decreased inflammatory gene expression (IL-6 and TNF-α) in HK-2 cells following an 18 h stimulus with 30 μg/ml of LPS.

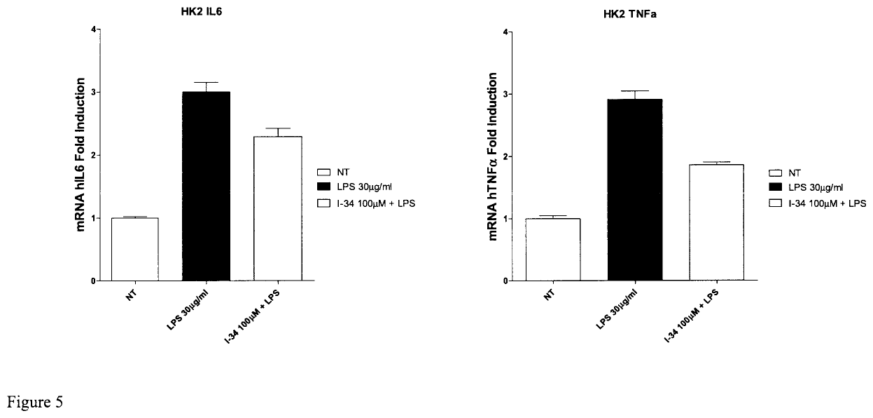

FIG. 5 shows fold change in expression of IL6 and TNF-α measured in HK-2 cells treated with LPS (30 μg/ml) for 18 h, followed by treatment with an ACMSD inhibitor (compound I-34, 100 μM).

ACMSD inhibition, with compound I-34, decreased inflammatory gene expression (IL-6 and TNF-α) in a dose response manner in HK-2 cells following an 18 h stimulus with 30 μg/ml of LPS.

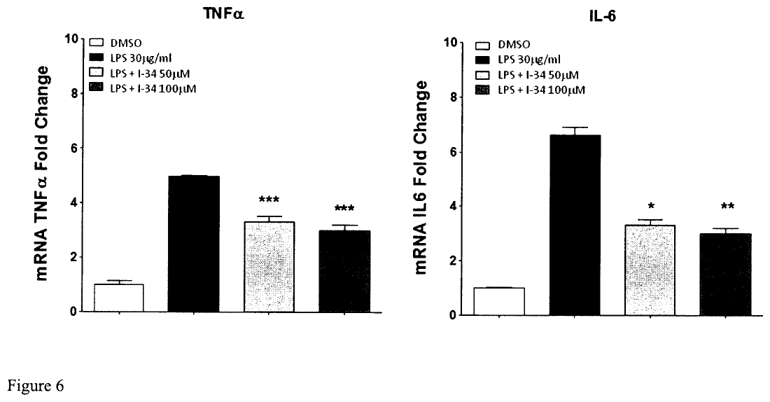

In FIG. 6, the expression of genes involved in inflammation was performed in HK-2 cells incubated in the absence (DMSO) and in the presence of LPS or LPS with compound I-34. ((N=3). One way Anova: Dunnett's test: *p<0.05; **p<0.01***p<0.001 vs pathological control).

### ACMSD Inhibition Decreased the Secretion of Pro-Inflammatory Cytokines in Kupffer Cells

ACMSD inhibition, with compound I-34, decreased the secretion of pro inflammatory cytokines in liver macrophage (Kupffer) cells exposed to LPS (song/ml), suggesting a protective effect against liver inflammation.

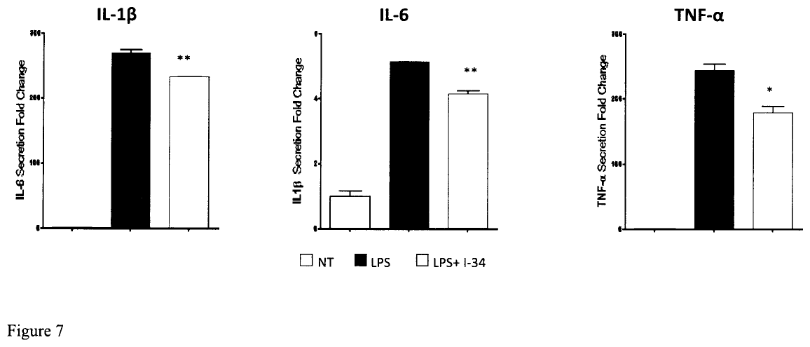

FIG. 7 shows fold change in secretion of inflammatory cytokines, IL-13, IL-6, and TNF-α measured in Kupffer cells treated with LPS (50 nmg/ml) for 24 h, followed by treatment with an ACMSD inhibitor (1 μM). (One way Anova: Dunnett's test: **p<0.01, ***p<0.001; vs pathological control.)

ACMSD Inhibition Protects Renal (HK-2) Cells from TGFβ-Induced Inflammatory Damage.

Pre-treatment with an ACMSD inhibitor (compound I-34) decreased fibrotic gene expression (Fibronectin and TIMP2) in a dose response manner following in vitro TGFβ insult at 10 ng/mL.

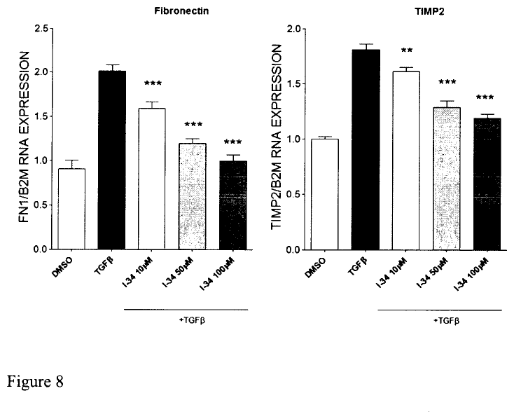

FIG. 8 shows the expression of genes involved in fibrosis performed in HK-2 cells incubated in the absence (DMSO) and in the presence of TGF-β or TGF-β together with compound I-34. ((N=3) One way Anova: Dunnett's test: **p<0.01, ***p<0.001; vs pathological control.)

TGF-β signaling is an important link between inflammation and fibrogenesis, ACMSD inhibition provides an approach for modulation of this pathway.

ACMSD Inhibition, with Compound I-34, Reduces Both Pro-Fibrotic and Pro-Inflammatory Genes in Hepatic Stellate Cells that were Co-Cultured with Mouse Primary Hepatocytes

Mouse primary hepatocytes and murine stellate cells were co-cultured with FFA to induce fibrosis as observed in NASH patients. Compound I-34 decreased the expression of pro-fibrotic and pro-inflammatory genes in liver cells exposed to free fatty acids, suggesting a protective effect from liver fibrosis and steatosis.

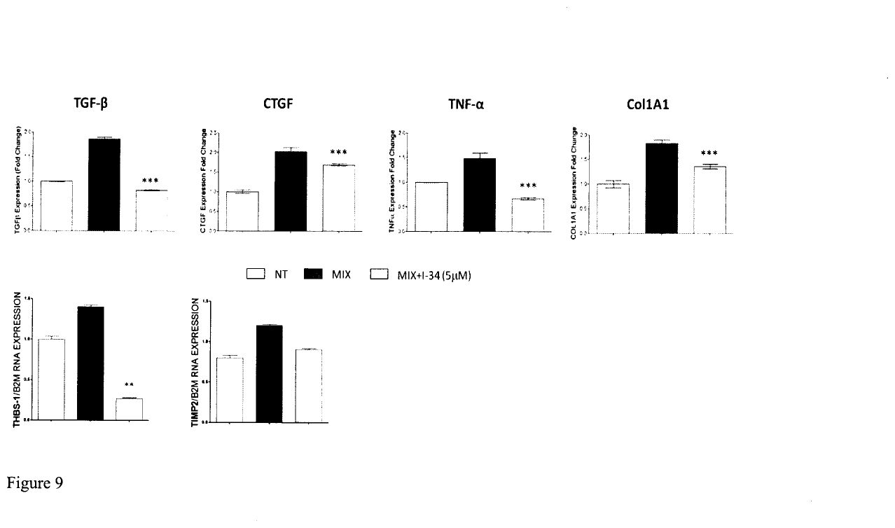

FIG. 9 shows fold change in expression of pro-fibrotic and pro-inflammatory genes measured in cocultured mouse primary hepatocytes and murine stellate cells treated with MIX or MIX together with an ACMSD inhibitor, compound I-34. (One way Anova: Dunnett's test: **p<0.01, ***p<0.001; vs pathological control.)

### ACMSD Inhibition Reduces Inflammatory Cytokine Expression and Secretion in 3D Human Liver Microtissues (Organoids)

3D human liver microtissues were treated with vehicle (DMSO), a mix of free fatty acids and LPS (10 μg/mL) and mix together with two concentrations of compound I-34 (10 and 50 μM). Mix-stimulated microtissues showed hepatic inflammation and increased cytokine secretion as a consequence of lipid accumulation. Compound I-34 reduced hepatic inflammation in a dose-response manner, reducing both cytokine expression and cytokine secretion.

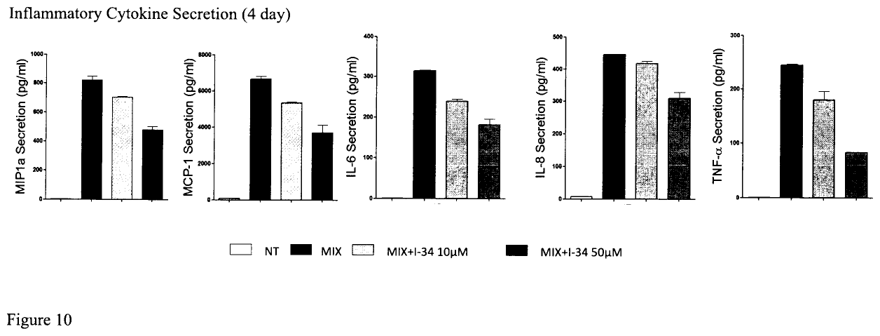

FIG. 10 shows fold change in secretion of pro-inflammatory genes measured in 3D human liver microtissues treated with MIX and LPS or MIX and LPS together with an ACMSD inhibitor.

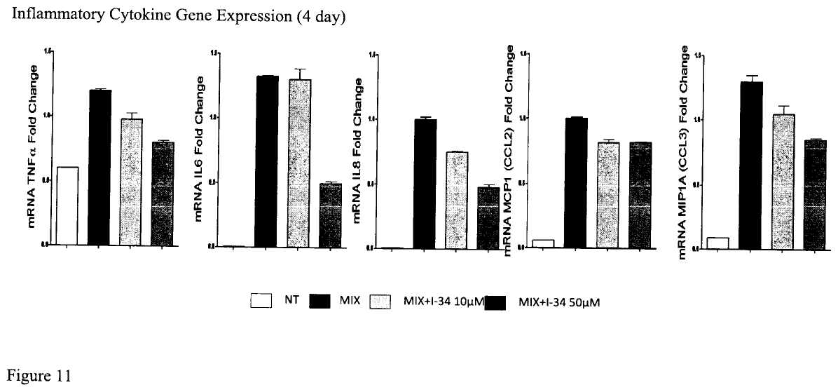

FIG. 11 shows fold change in expression of pro-inflammatory genes measured in 3D human liver microtissues treated with MIX and LPS or MIX and LPS together with an ACMSD inhibitor.

ACMSD Inhibition Protects Proximal Tubule Cells from Cisplatin-Induced Apoptosis

ACMSD inhibition, with compound I-34, provides protection from Cisplatin-induced apoptosis in HK-2 proximal tubule cells when the inhibitor is added 1 hr before Cisplatin injury (therapeutic treatment).

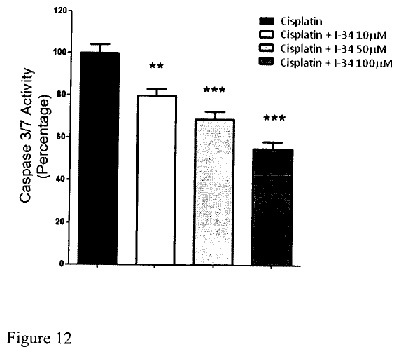

FIG. 12 shows Caspases 3/7 activity in HK-2 cells induced by Cisplatin (50 μM): Three different doses 10, 50 and 100 μM of compound I-34 were used 1 h before Cisplatin injury. ((N=3) One way Anova: Dunnett's test: **p<0.01, ***p<0.001; vs pathological control.)

### EQUIVALENTS

Those skilled in the art will recognize, or be able to ascertain using no more than routine experimentation, many equivalents to the specific embodiments and methods described herein. Such equivalents are intended to be encompassed by the scope of the present disclosure.

1. A method of treating an acute inflammatory condition in a subject comprising administering to the subject a therapeutically effective amount of a compound represented by Formula (II): 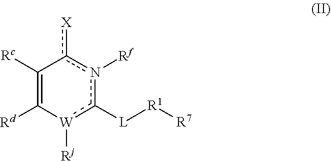
or a pharmaceutically acceptable salt or tautomer thereof, wherein:
X is H, S, SR², NR², NR²R^{2′}, OH, OR^{h}, F, Br, or Cl;
W is N or C;
(i) when W is N, then: L is —(C(R⁵)₂)ₘCH═CH(C(R⁵)₂)ₚ—, 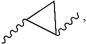
—(C(R⁵)₂)ₘY¹(C(R⁵)₂)ₚ—, —(C(R⁵)₂)ₘY¹(C(R⁵)₂)ₚ-cyclopropyl-, —(C(R⁵)₂)ₘY¹CH═CH—, —(C(R⁵)₂)ₘNR³C═(O)(C(R⁵)₂)ₚ—, —(C(R⁵)₂)ₘphenyl(C(R⁵)₂)ₚ—, —(C(R⁵)₂)ₘpyridinyl(C(R⁵)₂)ₚ—, or —(C(R⁵)₂)ₘthiophenyl(C(R⁵)₂)ₚ—;
(ii) when W is C, then: L is —(C(R⁵)₂)ₘCH═CH(C(R⁵)₂)ₚ—, —(C(R⁵)₂)ₒ—, —(C(R⁵)₂)ₘY¹(C(R⁵)₂)ₚ—, 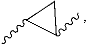
(C(R⁵)₂)ₘ Y¹CH═CH—, —(C(R⁵)₂)ₘC═(O)(CH₂)ₚ—, —(C(R⁵)₂)ₘC═(O)O(C(R⁵)₂)ₚ—, —(C(R⁵)₂)ₘC═(O)NR³(C(R⁵)₂)ₚ—, —(C(R⁵)₂)ₘNR³C═(O)(C(R⁵)₂)ₚ—, —(C(R⁵)₂)ₘphenyl(C(R⁵)₂)ₚ—, —(C(R⁵)₂)ₘpyridinyl(C(R⁵)₂)ₚ—, or —(C(R⁵)₂)ₘthiophenyl(C(R⁵)₂)ₚ—;
Y¹ is O, NR⁴, or S(O)_{q};
each Y² is independently O, NH or S;
R¹ is absent, C₆-C₁₀ arylene, heteroarylene, or C₃-C₈cycloalkylene, wherein the heteroarylene comprises one or two 5- to 7-membered rings and 1-4 heteroatoms selected from N, O and S, and wherein the C₆-C₁₀ arylene, heteroarylene, and C₃-C₈cycloalkylene are optionally substituted with one to two R^{e};
R² is H or C₁-C₄ alkyl;
R^{2′} is H, C₁-C₄ alkyl, or C₃-C₇ cycloalkyl; or
R² and R^{2′} together with the nitrogen atom to which they are attached form a 3- to 7-membered heterocycloalkyl ring comprising 1-3 additional heteroatoms selected from N, O and S;
R³ is H or C₁-C₄ alkyl;
R⁴ is H or C₁-C₄ alkyl;
each R⁵ is independently at each occurrence H or C₁-C₄ alkyl;
each R⁶ is independently at each occurrence H or C₁-C₄ alkyl;
R⁷ is H, A, B, or C;
A is —(C(R⁶)₂)ᵣCO₂R^{x}, —Y²(C(R⁶)₂)ᵣCO₂R^{x}, —(C(R⁶)₂)ᵣtetrazole, —(C(R⁶)₂)ᵣoxadiazolone, —(C(R⁶)₂)ᵣtetrazolone, —(C(R⁶)₂)ᵣthiadiazolol, —(C(R⁶)₂)ᵣ isoxazol-3-ol, —(C(R⁶)₂)ᵣP(O)(OH)OR^{x}, —(C(R⁶)₂)ᵣS(O)₂OH, —(C(R⁶)₂)ᵣC(O)NHCN, or —(C(R⁶)₂)ᵣC(O)NHS(O)₂alkyl, wherein —(C(R⁶)₂)ᵣtetrazole, —(C(R⁶)₂)ᵣoxadiazolone, —(C(R⁶)₂)ᵣtetrazolone, —(C(R⁶)₂)ᵣthiadiazolol, —(C(R⁶)₂)ᵣ isoxazol-3-ol are optionally substituted with C₁-C₆ alkyl,
B is —(C(R⁶)₂)ᵣS(O)₂OC₁-C₄ alkyl, —O(C(R⁶)₂)ᵣS(O)₂OC₁-C₄ alkyl, —Y²(C(R⁶)₂)ᵣC(O)NR^{g}R^{g′}, —Y²(C(R⁶)₂)ᵣS(O)₂NR^{g}R^{g′}, —(C(R⁶)₂)ᵣC(O)NR^{g}R^{g′}, —(C(R⁶)₂)ᵣS(O)₂NR^{g}R^{g′}, —(C(R⁶)₂)ᵣC(O)NHS(O)₂NR^{g}R^{g′}, —(C(R⁶)₂)ᵣCO₂Rⁱ,
(C(R⁶)₂)ᵣNH₂CO₂R^{x}, —(C(R⁶)₂)ᵣP(O)(OR^{x})₂, —O(C(R⁶)₂)ᵣP(O)(OR^{x})₂, —(C(R⁶)₂)ᵣS(O)₂OH, —O(C(R⁶)₂)ᵣS(O)₂OH, —(C(R⁶)₂)ᵣP(O)₂OR^{x}, or —O(C(R⁶)₂)ᵣP(O)₂OR^{x}, C is —(CH₂)ᵣCN, —(CH₂)ₛOH, halogen, —(C(R⁶)₂)ᵣC₆-C₁₀ aryl, —(C(R⁶)₂)ᵣS—C₆-C₁₀ aryl, —(C(R⁶)₂)ᵣheteroaryl, —O(C(R⁶)₂)ᵣheteroaryl, —O(C(R⁶)₂)ᵣheterocycloalkyl, —O(C(R⁶)₂)ᵣOH, —OR^{y}, —(C(R⁶)₂)ᵣC(O)NHCN, —CH═CHCO₂R^{x}, or —(C(R⁶)₂)ᵣC(O)NHS(O)₂C₁-C₄ alkyl, wherein the aryl and heteroaryl are substituted with one to three substituents each independently selected from C₁-C₆ alkyl, C₁-C₆ haloalkyl, halogen, and OH, and wherein the heterocycloalkyl is substituted with one to two ═O or ═S;
R^{c} is H, C₁-C₆ alkyl, C₁-C₆ haloalkyl, halogen, —CN, —OR^{x}, or —CO₂R^{x};
R^{d} is methyl, CF₃, CR^{f}F₂, —(C(R⁶)₂)ₜC₆-C₁₀ aryl, —(C(R⁶)₂)ₜ-5- or 6-membered heteroaryl, —(C(R⁶)₂)ₜ-5- or 6-membered cycloalkyl, optionally substituted C₆-C₁₀ aryl, optionally substituted 5- or 6-membered heteroaryl, or optionally substituted 5- or 6-membered cycloalkyl;
each R^{e} is independently at each occurrence C₁-C₆ alkyl, C₂-C₆ alkenyl, C₂-C₆ alkynyl, halogen, C₁-C₆ haloalkyl, —NHR^{z}, —OH, or —CN;
R^{f} is absent, H, or methyl;
R^{g} is H, C₁-C₆ alkyl, OH, —S(O)₂(C₁-C₆ alkyl), or S(O)₂N(C₁-C₆ alkyl)₂;
R^{g′} is H, C₁-C₆ alkyl, C₃-C₇ cycloalkyl, 4- to 7-membered heterocycloalkyl ring comprising 1-3 heteroatoms selected from N, O and S, C₆-C₁₀ aryl, or 5- to 7-membered heteroaryl comprising 1-3 heteroatoms selected from N, O and S, wherein the alkyl is optionally substituted with one or more substituents independently selected from halogen and —OH, and wherein the cycloalkyl, heterocycloalkyl, aryl and heteroaryl are optionally substituted with one or more substituents independently selected from C₁-C₆ alkyl, halogen, and —OH;
R^{h} is H, C₁-C₄ alkyl, or 3- to 7-membered heterocycloalkyl ring comprising 1-3 heteroatoms selected from N, O and S, wherein the alkyl is optionally substituted with one or more substituents each independently selected from NH₂, C₁-C₄ alkylamino, C₁-C₄ dialkylamino, and C(O)NH₂; and wherein the heterocycloalkyl is optionally substituted with one or more substituents each independently selected from C₁-C₆ alkyl and C₁-C₆ haloalkyl;
Rⁱ is (i) —(CH₂)ₛOC(O)C₁-C₆ alkyl, wherein the alkyl is substituted with one or more NH₂; (ii) (CH₂CH₂O)ₙCH₂CH₂OH; or (iii) C₁-C₆ alkyl substituted with one or more substituents each independently selected from OH and 4- to 7-membered heterocycloalkyl comprising 1 to 3 heteroatoms selected from O, N, or S;
R^{j} is absent, H, C₁-C₆ alkyl, or —CN;
each R^{x} is independently at each occurrence H, C₁-C₆ alkyl, or C₆-C₁₀ aryl;
each R^{y} and R^{z} is independently H, C₁-C₆ alkyl, or C₁-C₆ haloalkyl;
each m, p, q, r, and t is independently 0, 1 or 2;
n is 0, 1, 2, or 3;
s is 1 or 2;
o is 0, 1, 2, 3, or 4; and [symbol]
represents a single bond or a double bond; and
provided that
when X is O; R^{f} is H; W is C; R^{j} is —CN; L is —SCH₂—; R¹ is phenylene or pyridine; then R⁷ is not —COOH;
when X is O; R^{f} is H; W is C; R^{j} is —CN; L is —SCH₂—; R¹ is phenylene or pyridine; and R⁷ is tetrazole; then R^{c} is not H;
when X is O; R^{f} is H; W is C; R^{j} is —CN; L is —S—C(R⁵)₂ or —SCH₂CH₂—; R¹ is absent; then R⁷ is not COOH or tetrazole;
when X is O, R^{f} is H; W is N; R^{j} is absent; R^{d} is methyl, optionally substituted 5- to 10-membered aryl, optionally substituted 5- or 6-membered heteroaryl, or optionally substituted 5- or 6-membered cycloalkyl; L is —SCH₂— or —OCH₂—; and R¹ is phenylene; then R⁷ is not —COOH, —CH₂COOH, 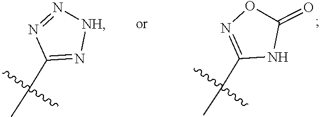 [SMILES: *c1nn[nH]n1 ; *c1noc(=O)[nH]1]
and
when X is O, R^{f} is H, W is N, R^{j} is absent, L is —NHCH₂—, —CH₂NH—, or —NH—C(O)—, and R¹ is phenylene, then R^{d} is not phenyl.

2. (canceled)

3. The method of claim 1, wherein X is O, OH, OR^{h}, F, Br, or Cl.

4. The method of claim 1, wherein X is H, S, SR², NR², or NR²R².

5. The method of claim 1, wherein R^{f} is absent.

6. The method of claim 1, wherein R^{f} is H or methyl.

7. The method of claim 1, wherein W is N.

8. The method of claim 7, wherein R^{j} is absent.

9. The method of claim 1, wherein W is C.

10. The method of claim 9, wherein R^{j} is H, C₁-C₆ alkyl, or —CN.

11. The method of claim 9, wherein R^{j} is —CN.

12. The method of claim 1, wherein R^{c} is C₁-C₆ alkyl, —CN, or halogen.

13. The method of claim 1, wherein R^{c} is —CN or halogen.

14. The method of claim 1, wherein R^{c} is —CN.

15. The method of claim 1, wherein R^{d} is methyl.

16. The method of claim 1, wherein R^{d} is optionally substituted 5- to 10-membered aryl.

17. The method of claim 1, wherein R^{d} is optionally substituted 5- or 6-membered heteroaryl.

18. The method of claim 1, wherein R^{d} is optionally substituted 5- or 6-membered cycloalkyl.

19. The method of claim 1, wherein R^{d} is methyl, cyclohexyl, pyridinyl, thiazolyl, phenyl, or thienyl.

20. The method of claim 1, wherein R^{d} is methyl, cyclohexyl, pyridinyl, thiazolyl, thienyl, or optionally substituted phenyl.

21. The method of claim 1, wherein R^{d} is methyl.

22. The method of claim 1, wherein R^{d} is —CF₃.

23. The method of claim 1, wherein R^{d} is CR^{f}F₂.

24. The method of claim 1, wherein R^{d} is —(C(R⁶)₂)ₜC₆-C₁₀ aryl, —(C(R⁶)₂)ₜ-5- or 6-membered heteroaryl, —(C(R⁶)₂)ₜ-5- or 6-membered cycloalkyl.

25. The method of claim 1, wherein R^{d} is —(C(R⁶)₂)ₜC₆-C₁₀ aryl.

26. The method of claim 1, wherein L is —(C(R⁵)₂)ₘY¹(C(R⁵)₂)ₚ—.

27. The method of claim 26, wherein Y¹ is S.

28. The method of claim 1, wherein L is —(C(R⁵)₂)ₘNR³C═(O)(C(R⁵)₂)ₚ— or —(C(R⁵)₂)ₘY¹(C(R⁵)₂)ₚ-cyclopropyl-.

29. The method of claim 1, wherein R¹ is C₆-C₁₀ arylene.

30. The method of claim 1, wherein R¹ is heteroarylene.

31. The method of claim 1, wherein R¹ is absent.

32. The method of claim 1, wherein R⁷ is A.

33. The method of claim 32, wherein A is —(C(R⁶)₂)ᵣCO₂R^{x} or —(CH₂)ᵣtetrazole, wherein the —(CH₂)ᵣtetrazole is optionally substituted with C₁-C₆ alkyl.

34. The method of claim 1, wherein R⁷ is B.

35. The method of claim 34, wherein B is —(CH₂)ᵣC(O)NR^{g}R^{g′}, or —(CH₂)ᵣS(O)₂NR^{g}R^{g′}.

36. The method of claim 1, wherein R⁷ is C.

37. The method of claim 36, wherein C is —(CH₂)ᵣCN, —(CH₂)ₛOH, or —(C(R⁶)₂)ᵣC₆-C₁₀ aryl, wherein the aryl is substituted with one to three substituents each independently selected from C₁-C₆ alkyl, C₁-C₆ haloalkyl, halogen, and OH.

38. The method of claim 1, wherein the compound is

| | |
|---|---|
| I-34 | 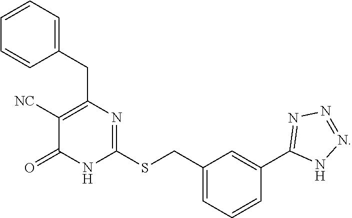 [SMILES: N#Cc1c(Cc2ccccc2)nc(SCc2cccc(-c3nnn[nH]3)c2)[nH]c1=O] |

39. The method of claim 1, wherein the acute inflammatory condition is a systemic inflammatory condition.

40. The method of claim 1, wherein the acute inflammatory condition is an organ-specific condition.

41. The method of claim 1, wherein the acute inflammatory condition is cytokine storm or hypercytokinemia, systemic inflammatory response syndrome (SIRS), graft versus host disease (GVHD), acute respiratory distress syndrome (ARDS), severe acute respiratory distress syndrome (SARS), catastrophic anti-phospholipid syndrome, viral infections, bacterial infections, fungal infections, influenza, pneumonia, shock, or sepsis.

42. The method of claim 1, wherein the acute inflammatory condition is acute pancreatitis, hepatitis, respiratory condition, or enterocolitis.

43. The method of claim 1, wherein the method reduces a pro-inflammatory cytokine or increases an anti-inflammatory cytokine.

44. The method of claim 43, wherein the pro-inflammatory cytokine is IL-1β, IL-6, IL-18, TNF-α, or TGF-β.

45. The method of claim 43, wherein pro-inflammatory cytokine is MCP-1, TNF-α, or IL-1β and the pro-inflammatory cytokine is increased.

46. The method of claim 43, wherein pro-inflammatory cytokine is IL-6 and the pro-inflammatory cytokine is increased.

47. The method of claim 43, wherein the anti-inflammatory cytokine is IL-10.

48. The method of claim 1, wherein expression of sirtuin-1 modulated genes sod2, tfam, dda1 genes are increased in the liver.

49. The method of claim 1, wherein the administration to the subject occurs at least 12 hours after an injury.

50. The method of claim 1, wherein the administration to the subject occurs for at least 6 days after an injury.
